SEQUENCE LISTING (361 sequences)

SEQ ID NO: 1 - DNA, 404 bp, Artificial Sequence
  Operational Taxonomic Unit (OTU) consensus sequence 1
tggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggaagacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacagg

SEQ ID NO: 2 - DNA, 404 bp, Artificial Sequence
  Operational Taxonomic Unit (OTU) consensus sequence 5
tggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 3 - DNA, 429 bp, Artificial Sequence
  Operational Taxonomic Unit (OTU) consensus sequence 6
tggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacagg

SEQ ID NO: 4 - DNA, 406 bp, Artificial Sequence
  Operational Taxonomic Unit (OTU) consensus sequence 7
tggggaatattgcacaatgggggaaaccctgatgcagcgacgccgcgtgagcgaagaagtatttcggtatgtaaagctctatcagcagggaagaagaaatgacggtacctgactaagaagcaccggctaaatacgtgccagcagccgcggtaatacgtatggtgcaagcgttatccggatttactgggtgtaaagggagcgcaggcggaaggctaagtctgatgtgaaagcccggggctcaaccccggtactgcattggaaactggtcatctagagtgtcggaggggtaagtggaattcctagtgtagcggtgaaatgcgtagatattaggaggaacaccagtggcgaaggcggcttactggacgataactgacgctgaggctcgaaagcgtggggagcaaacagg

SEQ ID NO: 5 - DNA, 404 bp, Artificial Sequence
  Operational Taxonomic Unit (OTU) consensus sequence 8
tggggaatattgcacaatggaggaaactctgatgcagcgacgccgcgtgagtgaagaagtagttcgctatgtaaagctctatcagcagggaagatagtgacggtacctgactaagaagctccggctaaatacgtgccagcagccgcggtaatacgtatggagcaagcgttatccggatttactgggtgtaaagggagtgtaggtggccaggcaagtcagaagtgaaagcccggggctcaaccccgggactgcttttgaaactgcagggctagagtgcaggaggggcaagtggaattcctagtgtagcggtgaaatgcgtagatattaggaggaacaccagtggcgaaggcggcttgctggactgtaactgacactgaggctcgaaagcgtggggagcaaacagg

SEQ ID NO: 6 - DNA, 404 bp, Artificial Sequence
  Operational Taxonomic Unit (OTU) consensus sequence 9
tggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacagg

SEQ ID NO: 7 - DNA, 424 bp, Artificial Sequence
  Operational Taxonomic Unit (OTU) consensus sequence 2
tgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 8 - DNA, 424 bp, Artificial Sequence
  Operational Taxonomic Unit (OTU) consensus sequence 3
tgaggaatattggtcaatgggcgagagcctgaaccagccaagtagcgtgcaggatgacggccctatgggttgtaaactgcttttataagggaataaagtgagtctcgtgagactttttgcatgtaccttatgaataaggaccggctaattccgtgccagcagccgcggtaatacggaaggtccgggcgttatccggatttattgggtttaaagggagcgtaggccggagattaagcgtgttgtgaaatgtagacgctcaacgtctgcactgcagcgcgaactggtttccttgagtacgcacaaagtgggcggaattcgtggtgtagcggtgaaatgcttagatatcacgaagaactccgattgcgaaggcagctcactggagcgcaactgacgctgaagctcgaaagtgcgggtatcgaacagg

SEQ ID NO: 9 - DNA, 424 bp, Artificial Sequence
  Operational Taxonomic Unit (OTU) consensus sequence 4
tgaggaatattggtcaatggacgagagtctgaaccagccaagtagcgtgaaggatgaaggtcctacggattgtaaacttcttttataagggaataaaccctcccacgtgtgggagcttgtatgtaccttatgaataagcatcggctaactccgtgccagcagccgcggtaatacggaggatgcgagcgttatccggatttattgggtttaaagggagcgcagacgggtcgttaagtcagctgtgaaagtttggggctcaaccttaaaattgcagttgatactggcgtccttgagtgcggttgaggtgtgcggaattcgtggtgtagcggtgaaatgcttagatatcacgaagaactccgattgcgaaggcagcacactaatccgtaactgacgttcatgctcgaaagtgtgggtatcaaacagg

SEQ ID NO: 10 - DNA, 407 bp, Artificial Sequence
  Operational Taxonomic Unit (OTU) consensus sequence 10
tggggaatattggacaatggaccaaaagtctgatccagcaattctgtgtgcacgatgaagtttttcggaatgtaaagtgctttcagttgggacgaagtaagtgacggtaccaacagaagaagcgacggctaaatacgtgccagcagccgcggtaatacgtatgtcgcaagcgttatccggatttattgggcgtaaagcgcgtctaggcggtttggtaagtctgatgtgaaaatgcggggctcaactccgtattgcgttggaaactgccaaactagagtactggagaggtgggcggaactacaagtgtagaggtgaaattcgtagatatttgtaggaatgccgatggggaagccagcccactggacagatactgacgctaaagcgcgaaagcgtgggtagcaaacagg

SEQ ID NO: 11 - DNA, 424 bp, Artificial Sequence
  Operational Taxonomic Unit (OTU) consensus sequence 14
tgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacagg

SEQ ID NO: 12 - DNA, 404 bp, Artificial Sequence
  Operational Taxonomic Unit (OTU) consensus sequence 15
tggggaatattgcacaatgggggaaaccctgatgcagcaacgccgcgtgagtgaagaagtatttcggtatgtaaagctctatcagcaggaaagaaaatgacggtacctgactaagaagccccggctaactacgtgccagcagccgcggtaatacgtagggggcaagcgttatccggatttactgggtgtaaagggagcgtagacggttttgcaagtctgaagtgaaagcccggggcttaaccccgggactgctttggaaactgtaggactagagtgcaggagaggtaagtggaattcctagtgtagcggtgaaatgcgtagatattaggaggaacaccagtggcgaaggcggcttactggactgtaactgacgttgaggctcgaaagcgtggggagcaaacagg

SEQ ID NO: 13 - DNA, 404 bp, Artificial Sequence
  Operational Taxonomic Unit (OTU) consensus sequence 16
tggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 14 - DNA, 404 bp, Artificial Sequence
  Operational Taxonomic Unit (OTU) consensus sequence 17
tggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtgaggctagagtgcaggaggggtgagtggaattcctagtgtagcggtgaaatgcgtagatattaggaggaacaccagtggcgaaggcggctcactggactgtaactgacactgaggctcgaaagcgtggggagcaaacagg

SEQ ID NO: 15 - DNA, 406 bp, Artificial Sequence
  Operational Taxonomic Unit (OTU) consensus sequence 18
tggggaatattgggcaatgggcgcaagcctgacccagcaacgccgcgtgaaggaagaaggctttcgggttgtaaacttcttttaagagggaagagcagaagacggtacctctagaataagccacggctaactacgtgccagcagccgcggtaatacgtaggtggcaagcgttgtccggatttactgggtgtaaagggcgtgcagccgggtctgcaagtcagatgtgaaatccatgggctcaacccatgaactgcatttgaaactgtagatcttgagtgtcggaggggcaatcggaattcctagtgtagcggtgaaatgcgtagatattaggaggaacaccagtggcgaaggcggattgctggacgataactgacggtgaggcgcgaaagtgtggggagcaaacagg

SEQ ID NO: 16 - DNA, 404 bp, Artificial Sequence
  Operational Taxonomic Unit (OTU) consensus sequence 19
tggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcggagcaagtctgaagtgaaagcccggggctcaaccccgggactgctttggaaactgttctgctagagtgctggagaggtaagtggaattcctagtgtagcggtgaaatgcgtagatattaggaggaacaccagtggcgaaggcggcttactggacagtaactgacgttgaggctcgaaagcgtggggagcaaacagg

SEQ ID NO: 17 - DNA, 424 bp, Artificial Sequence
  Operational Taxonomic Unit (OTU) consensus sequence 11
tgaggaatattggtcaatgggcgagagcctgaaccagccaagtagcgtgaaggatgaaggtcctacggattgtaaacttcttttatacgggaataaagtttcctacgtgtaggattttgtatgtaccgtatgaataagcatcggctaactccgtgccagcagccgcggtaatacggaggatgcgagcgttatccggatttattgggtttaaagggagcgcagacgggagattaagtcagttgtgaaagtttgcggctcaaccgtaaaattgcagttgatactggtttccttgagtgcagttgaggcaggcggaattcgtggtgtagcggtgaaatgcttagatatcacgaagaaccccgattgcgaaggcagcttgctaaactgtaactgacgttcatgctcgaaagtgtgggtatcaaacagg

SEQ ID NO: 18 - DNA, 424 bp, Artificial Sequence
  Operational Taxonomic Unit (OTU) consensus sequence 12
tgaggaatattggtcaatggacgagagtctgaaccagccaagtagcgtgaaggatgactgccctatgggttgtaaacttcttttatacgggaataaagtgagccacgtgtggctttttgtatgtaccgtatgaataaggatcggctaactccgtgccagcagccgcggtaatacggaggatccgagcgttatccggatttattgggtttaaagggagcgtaggcgggttgttaagtcagttgtgaaagtttgcggctcaaccgtaaaattgcagttgatactggcgaccttgagtgcaacagaggtaggcggaattcgtggtgtagcggtgaaatgcttagatatcacgaagaactccgattgcgaaggcagcttactggattgtaactgacgctgatgctcgaaagtgtgggtatcaaacagg

SEQ ID NO: 19 - DNA, 404 bp, Artificial Sequence
  Operational Taxonomic Unit (OTU) consensus sequence 13
tggggaatattgcacaatgggcgaaagcctgatgcagcaacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgcagacggcactgcaagtctgaagtgaaagcccggggctcaaccccgggactgctttggaaactgtagagctagagtgctggagaggcaagcggaattcctagtgtagcggtgaaatgcgtagatattaggaagaacaccagtggcgaaggcggcttgctggacagtaactgacgttcaggctcgaaagcgtggggagcaaacagg

SEQ ID NO: 20 - DNA, 404 bp, Artificial Sequence
  Operational Taxonomic Unit (OTU) consensus sequence 21
tggggaatattgcacaatgggggaaaccctgatgcagcgacgccgcgtgagtgatgaagtatttcggtatgtaaaactctatcagcagggaagataatgacggtacctgactaagaagcaccggctaaatacgtgccagcagccgcggtaatacgtatggtgcaagcgttatccggatttactgggtgtaaagggtgcgtaggtggtatggcaagtcagaagtgaaaggctggggctcaaccccgggactgcttttgaaactgtcaaactagagtacaggagaggaaagcggaattcctagtgtagcggtgaaatgcgtagatattaggaggaacaccagtggcgaaggcggctttctggactgaaactgacactgaggcacgaaagcgtggggagcaaacagg

SEQ ID NO: 21 - DNA, 429 bp, Artificial Sequence
  Operational Taxonomic Unit (OTU) consensus sequence 24
tggggaatcttccgcaatggacgaaagtctgacggagcaacgccgcgtgagtgatgaaggatttcggtctgtaaagctctgttgtttatgacgaacgtgcagtgtgtgaacaatgcattgcaatgacggtagtaaacgaggaagccacggctaactacgtgccagcagccgcggtaatacgtaggtggcgagcgttgtccggaattattgggcgtaaagagcatgtaggcggcttaataagtcgagcgtgaaaatgcggggctcaaccccgtatggcgctggaaactgttaggcttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacggg

SEQ ID NO: 22 - DNA, 429 bp, Artificial Sequence
  Operational Taxonomic Unit (OTU) consensus sequence 25
tggggaatcttccgcaatgggcgaaagcctgacggagcaacgccgcgtgaacgatgaaggtcttaggatcgtaaagttctgttgttagggacgaagggtaagaatcataataaggtttttatttgacggtacctaacgaggaagccacggctaactacgtgccagcagccgcggtaatacgtaggcggcaagcgttgtccggaattattgggcgtaaagggagcgcaggcgggaaactaagcggatcttaaaagtgcggggctcaaccccgtgatggggtccgaactggttttcttgagtgcaggagaggaaagcggaattcccagtgtagcggtgaaatgcgtagatattgggaagaacaccagtggcgaaggcggctttctggactgtaactgacgctgaggctcgaaagctagggtagcgaacggg

SEQ ID NO: 23 - DNA, 404 bp, Artificial Sequence
  Operational Taxonomic Unit (OTU) consensus sequence 26
tgggggatattgcacaatgggggaaaccctgatgcagcgacgccgcgtggaggaagaaggttttcggattgtaaactcctgtcgttagggacgataatgacggtacctaacaagaaagcaccggctaactacgtgccagcagccgcggtaaaacgtagggtgcaagcgttgtccggaattactgggtgtaaagggagcgcaggcgggaagacaagttggaagtgaaaaccatgggctcaacccatgaattgctttcaaaactgtttttcttgagtagtgcagaggtagatggaattcccggtgtagcggtggaatgcgtagatatcgggaggaacaccagtggcgaaggcggtctactgggcaccaactgacgctgaggctcgaaagcatgggtagcaaacagg

SEQ ID NO: 24 - DNA, 407 bp, Artificial Sequence
  Operational Taxonomic Unit (OTU) consensus sequence 27
tggggaatattgcacaatgggggaaaccctgatgcagcaacgccgcgtgaaggatgacggttttcggattgtaaacttcttttcttagtgacgaagacagtgacggtagctaaggaataagcatcggctaactacgtgccagcagccgcggtaatacgtaggatgcaagcgttatccggatttactgggtgtaaagggagcgcaggcgggactgcaagttggatgtgaaataccgtggcttaaccacggaactgcatccaaaactgtagttcttgagtgaagtagaggcaagcggaattccgagtgtagcggtgaaatgcgtagatattcggaggaacaccagtggcgaaggcggcttgctgggctttaactgacgctgaggctcgaaagtgtggggagcaaacagg

SEQ ID NO: 25 - DNA, 405 bp, Artificial Sequence
  Operational Taxonomic Unit (OTU) consensus sequence 28
tggggaatattgcacaatggaggaaactctgatgcagcgatgccgcgtgagggaagaaggttttaggattgtaaacctctgtcttcagggacgaaaaaagacggtacctgaggaggaagctccggctaactacgtgccagcagccgcggtaatacgtagggagcgagcgttgtccggaattactgggtgtaaagggagcgtaggcgggatcgcaagtcagatgtgaaaactatgggcttaacccataaactgcatttgaaactgtggttcttgagtgaagtagaggtaagcggaattcctagtgtagcggtgaaatgcgtagatattaggaggaacatcagtggcgaaggcggcttactgggctttaactgacgctgaggctcgaaagcgtggggagcaaacagg

SEQ ID NO: 26 - DNA, 404 bp, Artificial Sequence
  Operational Taxonomic Unit (OTU) consensus sequence 20
tggggaatattgcacaatgggggaaaccctgatgcagcgacgccgcgtgagcgaagaagtatttcggtatgtaaagctctatcagcagggaagataatgacggtacctgactaagaagccccggctaactacgtgccagcagccgcggtaatacgtagggggcaagcgttatccggatttactgggtgtaaagggagcgtagacggcaaggcaagtctgatgtgaaaacccagggcttaaccctgggactgcattggaaactgtctggctcgagtgccggagaggtaagcggaattcctagtgtagcggtgaaatgcgtagatattaggaagaacaccagtggcgaaggcggcttactggacggtaactgacgttgaggctcgaaagcgtggggagcaaacagg

SEQ ID NO: 27 - DNA, 404 bp, Artificial Sequence
  Operational Taxonomic Unit (OTU) consensus sequence 30
tgggggatattgcacaatgggggaaaccctgatgcagcgacgccgcgtggaggaagaaggttttcggattgtaaactcctgtcgttagggacgataatgacggtacctaacaagaaagcaccggctaactacgtgccagcagccgcggtaaaacgtagggtgcaagcgttgtccggaattactgggtgtaaagggagcgcaggcggaccggcaagttggaagtgaaaactatgggctcaacccataaattgctttcaaaactgctggccttgagtagtgcagaggtaggtggaattcccggtgtagcggtggaatgcgtagatatcgggaggaacaccagtggcgaaggcgacctactgggcaccaactgacgctgaggctcgaaagcatgggtagcaaacagg

SEQ ID NO: 28 - DNA, 424 bp, Artificial Sequence
  Operational Taxonomic Unit (OTU) consensus sequence 31
tgaggaatattggtcaatggacgcaagtctgaaccagccatgccgcgtgcaggatgacggctctatgagttgtaaactgcttttgtacgagggtaaacgcagatacgtgtatctgtctgaaagtatcgtacgaataaggatcggctaactccgtgccagcagccgcggtaatacggaggattcaagcgttatccggatttattgggtttaaagggtgcgtaggcggtttgataagttagaggtgaaatttcggggctcaaccctgaacgtgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacagg

SEQ ID NO: 29 - DNA, 424 bp, Artificial Sequence
  Operational Taxonomic Unit (OTU) consensus sequence 22
tgaggaatattggtcaatggacgagagtctgaaccagccaagtagcgtgaaggatgactgccctatgggttgtaaacttcttttatatgggaataaagtattccacgtgtgggattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacagg

SEQ ID NO: 30 - DNA, 424 bp, Artificial Sequence
  Operational Taxonomic Unit (OTU) consensus sequence 33
tgaggaatattggtcaatggacgagagtctgaaccagccaagtagcgtgaaggatgaaggttctatggattgtaaacttcttttatacgggaataaacggatccacgtgtggatttttgcatgtaccgtatgaataaggatcggctaactccgtgccagcagccgcggtaatacggaggatccgagcgttatccggatttattgggtttaaagggagcgtagatgggttgttaagtcagttgtgaaagtttgcggctcaaccgtaaaattgcaattgatactggcagtcttgagtacagttgaggtaggcggaattcgtggtgtagcggtgaaatgcttagatatcacgaagaactccgattgcgaaggcagcttactaacctgtaactgacattgatgctcgaaagtgtgggtatcaaacagg

SEQ ID NO: 31 - DNA, 404 bp, Artificial Sequence
  Operational Taxonomic Unit (OTU) consensus sequence 34
tgggggatattgcacaatggaggaaactctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgacgcaagtctgaagtgaaatacccgggctcaacctgggaactgctttggaaactgtgttgctagagtgctggagaggtaagcggaattcctagtgtagcggtgaaatgcgtagatattaggaagaacaccagtggcgaaggcggcttactggacagtaactgacgttgaggctcgaaagcgtggggagcaaacagg

SEQ ID NO: 32 - DNA, 407 bp, Artificial Sequence
  Operational Taxonomic Unit (OTU) consensus sequence 35
tggggaatattgggcaatgggggaaaccctgacccagcaacgccgcgtgaaggaagaaggctttcgggttgtaaacttcttttaccagggacgaaggacgtgacggtacctggagaaaaagccacggctaactacgtgccagcagccgcggtaatacgtaggtggcaagcgttgtccggatttactgggtgtaaagggcgtgtaggcggagaagcaagtcagaagtgaaatccatgggcttaacccatgaactgcttttgaaactgtttcccttgagtatcggagaggcaggcggaattcctagtgtagcggtgaaatgcgtagatattaggaggaacaccagtggcgaaggcggcctgctggacgacaactgacgctgaggcgcgaaagcgtggggagcaaacagg

SEQ ID NO: 33 - DNA, 406 bp, Artificial Sequence
  Operational Taxonomic Unit (OTU) consensus sequence 36
tggggaatattgggcaatgggcgcaagcctgacccagcaacgccgcgtgaaggaagaaggctttcgggttgtaaacttcttttgtcagggacgagtagaagacggtacctgacgaataagccacggctaactacgtgccagcagccgcggtaatacgtaggtggcaagcgttgtccggatttactgggtgtaaagggcgtgtagccgggagggcaagtcagatgtgaaatccacgggctcaactcgtgaactgcatttgaaactactcttcttgagtatcggagaggcaatcggaattcctagtgtagcggtgaaatgcgtagatattaggaggaacaccagtggcgaaggcggattgctggacgacaactgacggtgaggcgcgaaagcgtggggagcaaacagg

SEQ ID NO: 34 - DNA, 404 bp, Artificial Sequence
  Operational Taxonomic Unit (OTU) consensus sequence 37
tggggaatattgcacaatgggggaaaccctgatgcagcaacgccgcgtgagtgatgacggccttcgggttgtaaagctctgtcttcagggacgataatgacggtacctgaggaggaagccacggctaactacgtgccagcagccgcggtaatacgtaggtggcgagcgttgtccggatttactgggcgtaaagggagcgtaggcggacttttaagtgagatgtgaaatacccgggctcaacttgggtgctgcatttcaaactggaagtctagagtgcaggagaggagaatggaattcctagtgtagcggtgaaatgcgtagagattaggaagaacaccagtggcgaaggcgattctctggactgtaactgacgctgaggctcgaaagcgtggggagcaaacagg

SEQ ID NO: 35 - DNA, 403 bp, Artificial Sequence
  Operational Taxonomic Unit (OTU) consensus sequence 38
tggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggtggtttcttaagtcagaggtgaaaggctacggctcaaccgtagtaagcctttgaaactgggaaacttgagtgcaggagaggagagtggaattcctagtgtagcggtgaaatgcgtagatattaggaggaacaccagttgcgaaggcggctctctggactgtaactgacactgaggcacgaaagcgtggggagcaaacagg

SEQ ID NO: 36 - DNA, 404 bp, Artificial Sequence
  Operational Taxonomic Unit (OTU) consensus sequence 32
tggggaatattgcacaatggaggaaactctgatgcagcgacgccgcgtgagtgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtaggtggcaaggcaagccagaagtgaaaacccggggctcaaccgcgggattgcttttggaactgtcatgctagagtgcaggaggggtgagcggaattcctagtgtagcggtgaaatgcgtagatattaggaggaacaccggaggcgaaggcggctcactggactgtaactgacactgaggctcgaaagcgtggggagcaaacagg

SEQ ID NO: 37 - DNA, 404 bp, Artificial Sequence
  Operational Taxonomic Unit (OTU) consensus sequence 39
tggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggttgtgtaagtctgatgtgaaagcccggggctcaaccccgggactgcattggaaactatgtaactagagtgtcggagaggtaagcggaattcctagtgtagcggtgaaatgcgtagatattaggaggaacaccagtggcgaaggcggcttactggacgatcactgacgttgaggctcgaaagcgtggggagcaaacagg

SEQ ID NO: 38 - DNA, 424 bp, Artificial Sequence
  Operational Taxonomic Unit (OTU) consensus sequence 23
tgaggaatattggtcaatggccgagaggctgaaccagccaagtcgcgtgaaggaagaaggatctatggtttgtaaacttcttttataggggaataaagtggaggacgtgtccttttttgtatgtaccctatgaataagcatcggctaactccgtgccagcagccgcggtaatacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtggtgtagcggtgaaatgcatagatatcacgcagaactccgattgcgaaggcagcttactaaaccataactgacactgaagcacgaaagcgtggggatcaaacagg

SEQ ID NO: 39 - DNA, 429 bp, Artificial Sequence
  Operational Taxonomic Unit (OTU) consensus sequence 29
tggggaatattgcacaatgggcgcaagcctgatgcagccatgccgcgtgtgtgaagaaggccttcgggttgtaaagcactttcagcggggaggaaggcggtgaggttaataacctcatcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 40 - DNA, 424 bp, Artificial Sequence
  Operational Taxonomic Unit (OTU) consensus sequence 43
tgaggaatattggtcaatgggcgctagcctgaaccagccaagtagcgtgaaggatgaaggctctatgggtcgtaaacttcttttatataagaataaagtgcagtatgtatactgttttgtatgtattatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacagg

SEQ ID NO: 41 - DNA, 425 bp, Artificial Sequence
  Operational Taxonomic Unit (OTU) consensus sequence 44
tgaggaatattggtcaatgggcgagagcctgaaccagccaagtagcgtgaaggatgacggccctacgggttgtaaacttcttttgtgcgggaataaaggaacctacgtgtaggtttttgcatgtaccgtaacgaataagcatcggctaactccgtgccagcagccgcggtaatacggaggatgcgagcgttatccggatttattgggtttaaagggagcgtagacgggtttttaagtcagctgtgaaagtttggggctcaaccttaaaattgcagttgaaactggagaccttgagtacggttgaggcaggcggaattcgtggtgtagcggtgaaatgcttagatatcacgaagaaccccgattgcgaaggcagcctgctaagccgccactgacgttgaggctcgaaagtgcgggtatcaaacagg

SEQ ID NO: 42 - DNA, 406 bp, Artificial Sequence
  Operational Taxonomic Unit (OTU) consensus sequence 40
tggggaatattgggcaatgggcgaaagcctgacccagcaacgccgcgtgaaggaagaaggccttcgggttgtaaacttcttttaagagggacgaagaagtgacggtacctcttgaataagccacggctaactacgtgccagcagccgcggtaatacgtaggtggcgagcgttatccggatttactgggtgtaaagggcgcgtaggcgggaatgcaagtcagatgtgaaatccaagggctcaacccttgaactgcatttgaaactgtatttcttgagtgtcggagaggttgacggaattcctagtgtagcggtgaaatgcgtagatattaggaggaacaccagtggcgaaggcggtcaactggacgataactgacgctgaggcgcgaaagcgtggggagcaaacagg

SEQ ID NO: 43 - DNA, 424 bp, Artificial Sequence
  Operational Taxonomic Unit (OTU) consensus sequence 45
tgaggaatattggtcaatggacgagagtctgaaccagccaagtagcgtgcaggatgacggccctatgggttgtaaactgcttttatagggggataaagtgtgccacgtgtggcatattgcaggtaccctatgaataaggaccggctaattccgtgccagcagccgcggtaatacggaaggtccgggcgttatccggatttattgggtttaaagggagcgtaggccgtttggtaagcgtgttgtgaaatgtcggggctcaacctgggcattgcagcgcgaactgccagacttgagtgcgcaggaagtaggcggaattcgtcgtgtagcggtgaaatgcttagatatgacgaagaactccgattgcgaaggcagcctgctgtagcgcaactgacgctgaagctcgaaagcgtgggtatcgaacagg

SEQ ID NO: 44 - DNA, 407 bp, Artificial Sequence
  Operational Taxonomic Unit (OTU) consensus sequence 41
tgggggatattgcgcaatgggggaaaccctgacgcagcaacgccgcgtgaaggatgaaggtcttcggattgtaaacttcttttattaaggacgaagaaagtgacggtacttaatgaataagctccggctaactacgtgccagcagccgcggtaatacgtagggagcaagcgttgtccggatttactgggtgtaaagggtgcgtaggcggctttgcaagtcagatgtgaaatctatgggctcaacccatagcctgcatttgaaactgcagagcttgagtgaagtagaggcaggcggaattccccgtgtagcggtgaaatgcgtagagatggggaggaacaccagtggcgaaggcggcctgctgggctttaactgacgctgaggcacgaaagcgtgggtagcaaacagg

SEQ ID NO: 45 - DNA, 424 bp, Artificial Sequence
  Operational Taxonomic Unit (OTU) consensus sequence 47
tgaggaatattggtcaatggccgagaggctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgattgcgaaggcagcctgccaagccattactgacgctgatgcacgaaagcgtggggatcaaacagg

SEQ ID NO: 46 - DNA, 404 bp, Artificial Sequence
  Operational Taxonomic Unit (OTU) consensus sequence 46
tggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacagg

SEQ ID NO: 47 - DNA, 404 bp, Artificial Sequence
  Operational Taxonomic Unit (OTU) consensus sequence 42
tggggaatattgcacaatgggggaaaccctgatgcagcgacgccgcgtgagcgatgaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggagtggcaagtctgatgtgaaaacccggggctcaaccccgggactgcattggaaactgtcaatctagagtaccggagaggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 48 - DNA, 424 bp, Artificial Sequence
  Operational Taxonomic Unit (OTU) consensus sequence 49
tgaggaatattggtcaatgggcggaagcctgaaccagccaagtagcgtgcaggaagacggccctccgggttgtaaactgcttttagttgggaataaaacggggctcgtgagcccccttgcatgtaccatcagaaaaaggaccggctaattccgtgccagcagccgcggtaatacggaaggtccgggcgttatccggatttattgggtttaaagggagcgcaggcggacctttaagtcagctgtgaaatacggcggctcaaccgtcgaactgcagttgatactggaggtcttgagtgcacacagggatgctggaattcatggtgtagcggtgaaatgctcagatatcatgaagaactccgatcgcgaaggcaggcatccggggtgcaactgacgctgaggctcgaaagtgcgggtatcaaacagg

SEQ ID NO: 49 - DNA, 423 bp, Artificial Sequence
  Operational Taxonomic Unit (OTU) consensus sequence 52
tgaggaatattggtcaatgggcgggagcctgaaccagccaagtagcgtgaaggatgacggccctacgggttgtaaacttcttttataagggaataaagttcgccacgtgtggtgttttgtatgtaccttatgaataagcatcggctaattccgtgccagcagccgcggtaatacggaagatgcgagcgttatccggatttattgggtttaaagggagcgtaggcgggcttttaagtcagcggtcaaatgccacggctcaaccgtggccagccgttgaaactgcaagccttgagtctgcacagggcacatggaattcgtggtgtagcggtgaaatgcttagatatcacgaagaactccgatcgcgaaggcattgtgccggggcagcactgacgctgaggctcgaaagtgcgggtatcaaacagg

SEQ ID NO: 50 - DNA, 404 bp, Artificial Sequence
  Operational Taxonomic Unit (OTU) consensus sequence 53
tggggaatattgcacaatgggggaaaccctgatgcagcgacgccgcgtgagtgaagaagtatttcggtatgtaaagctctatcagcagggaagaaagtgacggtacctgaataagaagccccggctaactacgtgccagcagccgcggtaatacgtagggggcaagcgttatccggatttactgggtgtaaagggagcgtagacggcaaggcaagtctgaagtgaaagcccggtgcttaacgccgggactgctttggaaactgtttggctggagtgccggagaggtaagcggaattcctagtgtagcggtgaaatgcgtagatattaggaagaacaccagtggcgaaggcggcttactggacggtaactgacgttgaggctcgaaagcgtggggagcaaacagg

SEQ ID NO: 51 - DNA, 407 bp, Artificial Sequence
  Operational Taxonomic Unit (OTU) consensus sequence 54
tggggaatattggacaatggaccaaaagtctgatccagcaattctgtgtgcacgatgacggtcttaggattgtaaagtgctttcaatcgggaaaaagaaagtgatggtaccgatagaagaagcgacggctaaatacgtgccagcagccgcggtaatacgtatgtcgcaagcgttatccggatttattgggcgtaaagcgcgtctaggcggtctggtaagtctgatgtggaaatgcggggctcaactccgtattgcgttggaaactgccagactagagtactggagaggtgggcggaactacaagtgtagaggtgaaattcgtagatatttgtaggaatgccgatagagaagtcagctcactggacagatactgacgctgaagcgcgaaagcatggggagcaaacagg

SEQ ID NO: 52 - DNA, 404 bp, Artificial Sequence
  Operational Taxonomic Unit (OTU) consensus sequence 55
tggggaatattgcacaatggaggaaactctgatgcagcgacgccgcgtgagtgaagaagtatttcggtatgtaaagctctatcagcagggaagacagtgacggtacctgactaagaagctccggctaaatacgtgccagcagccgcggtaatacgtatggagcaagcgttatccggatttactgggtgtaaagggagtgtaggtggtatcacaagtcagaagtgaaagcccggggctcaaccccgggactgcttttgaaactgtggaactggagtgcaggagaggtaagtggaattcctagtgtagcggtgaaatgcgtagatattaggaggaacaccagtggcgaaggcggcttactggactgtaactgacactgaggctcgaaagcgtggggagcaaacagg

SEQ ID NO: 53 - DNA, 407 bp, Artificial Sequence
  Operational Taxonomic Unit (OTU) consensus sequence 56
tggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacagg

SEQ ID NO: 54 - DNA, 429 bp, Artificial Sequence
  Operational Taxonomic Unit (OTU) consensus sequence 57
tggggaatattgcgcaatgggggcaaccctgacgcagccatgccgcgtgaatgaagaaggccttcgggttgtaaagttctttcggtagcgaggaaggcatttagtttaatagactaggtgattgacgttaactacagaagaagcaccggctaactccgtgccagcagccgcggtaatacggagggtgcgagcgttaatcggaataactgggcgtaaagggcacgcaggcggtgacttaagtgaggtgtgaaagccccgggcttaacctgggaattgcatttcatactgggtcgctagagtactttagggaggggtagaattccacgtgtagcggtgaaatgcgtagagatgtggaggaataccgaaggcgaaggcagccccttgggaatgtactgacgctcatgtgcgaaagcgtggggagcaaacagg

SEQ ID NO: 55 - DNA, 406 bp, Artificial Sequence
  Operational Taxonomic Unit (OTU) consensus sequence 58
tggggaatattgggcaatggacgcaagtctgacccagcaacgccgcgtgaaggaagaaggctttcgggttgtaaacttcttttgtcagggaagagtagaagacggtacctgacgaataagccacggctaactacgtgccagcagccgcggtaatacgtaggtggcaagcgttgtccggatttactgggtgtaaagggcgtgcagccgggccggcaagtcagatgtgaaatctggaggcttaacctccaaactgcatttgaaactgtaggtcttgagtaccggagaggttatcggaattccttgtgtagcggtgaaatgcgtagatataaggaagaacaccagtggcgaaggcggataactggacggcaactgacggtgaggcgcgaaagcgtggggagcaaacagg

SEQ ID NO: 56 - DNA, 424 bp, Artificial Sequence
  Operational Taxonomic Unit (OTU) consensus sequence 59
tgaggaatattggtcaatgggcgagagcctgaaccagccaagtagcgtgcaggatgacggccctatgggttgtaaactgcttttataggggaataaagtgagccacgtgtggttttttgcatgtaccctatgaataaggaccggctaattccgtgccagcagccgcggtaatacggaaggtccgggcgttatccggatttattgggtttaaagggagcgtaggccggagattaagcgtgttgtgaaatgtagatgctcaacatctgaactgcagcgcgaactggtttccttgagtacgcataaagtgggcggaattcgtggtgtagcggtgaaatgcttagatatcacgaagaactccgattgcgaaggcagctcactggggcgcaactgacgctgaagctcgaaagcgcgggtatcgaacagg

SEQ ID NO: 57 - DNA, 405 bp, Artificial Sequence
  Operational Taxonomic Unit (OTU) consensus sequence 60
tggggaatattgcacaatgggcgcaagcctgatgcagcaacgccgcgtgaaggaagacggttttcggattgtaaacttctgttcttagtgaagaataatgacggtagctaaggagcaagccacggctaactacgtgccagcagccgcggtaatacgtaggtggcaagcgttgtccggaattactgggtgtaaagggagcgcaggcgggtgatcaagtcagctgtgaaaactacgggcttaacccgtagactgcagttgaaactgttcatcttgagtgaagtagaggttggcggaattccgagtgtagcggtgaaatgcgtagatattcggaggaacaccggtggcgaaggcggccaactgggctttaactgacgctgaggctcgaaagtgtggggagcaaacagg

SEQ ID NO: 58 - DNA, 407 bp, Artificial Sequence
  Operational Taxonomic Unit (OTU) consensus sequence 61
tggggaatattgcgcaatgggggcaaccctgacgcagcaacgccgcgtgcaggaagaaggtcttcggattgtaaactgttgtcgcaagggaagaagacagtgacggtaccttgtgagaaagtcacggctaactacgtgccagcagccgcggtaatacgtaggtgacaagcgttgtccggatttactgggtgtaaagggcgcgtaggcggactgtcaagtcagtcgtgaaataccggggcttaaccccggggctgcgattgaaactgacagccttgagtatcggagaggaaagcggaattcctagtgtagcggtgaaatgcgtagatattaggaggaacaccagtggcgaaggcggctttctggacgacaactgacgctgaggcgcgaaagtgtggggagcaaacagg

SEQ ID NO: 59 - DNA, 404 bp, Artificial Sequence
  Operational Taxonomic Unit (OTU) consensus sequence 62
tgggggatattggacaatgggggcaaccctgatccagcgacgccgcgtgagggaagaaggttttcggattgtaaacctctgttgacggagaaaaaaatgatggtatccgtttagaaagccacggctaactacgtgccagcagccgcggtaatacgtaggtggcaagcgttgtccggaattactgggtgtaaagggagtgtaggcgggatatcaagtcagaagtgaaaattacgggctcaactcgtaacctgcttttgaaactgacattcttgagtgaagtagaggcaagcggaattcctagtgtagcggtgaaatgcgtagatattaggaggaacaccagtggcgaaggcggcttgctgggcttttactgacgctgaggctcgaaagcgtggggagcaaacagg

SEQ ID NO: 60 - DNA, 406 bp, Artificial Sequence
  Operational Taxonomic Unit (OTU) consensus sequence 63
tggggaatattgcacaatgggcgaaagcctgatgcagcaacgccgcgtgagggaagacggttttcggattgtaaacctctgttttcggtgacgaacaaatgacggtaaccgagtaggaagccacggctaactacgtgccagcagccgcggtaatacgtaggtggcaagcgttatccggaattactgggtgtaaagggagcgcaggcgggatagcaagtcagctgtgaaaactatgggctcaacccataaactgcagttgaaactgttattcttgagtggagtagaggcaagcggaattccgagtgtagcggtgaaatgcgtagatattcggaggaacaccagtggcgaaggcggcttgctgggctctaactgacgctgaggctcgaaagtgtggggagcaaacagg

SEQ ID NO: 61 - DNA, 428 bp, Artificial Sequence
  Operational Taxonomic Unit (OTU) consensus sequence 64
tagggaattttcggcaatgggggaaaccctgaccgagcaacgccgcgtgaaggaagaagtaattcgttatgtaaacttctgtcatagaggaagaacggtggatatagggaatgatatccaagtgacggtactctataagaaagccacggctaactacgtgccagcagccgcggtaatacgtaggtggcgagcgttatccggaattattgggcgtaaagagggagcaggcggcactaagggtctgtggtgaaagatcgaagcttaacttcggtaagccatggaaaccgtagagctagagtgtgtgagaggatcgtggaattccatgtgtagcggtgaaatgcgtagatatatggaggaacaccagtggcgaaggcgacgatctggcgcataactgacgctcagtcccgaaagcgtggggagcaaatagg

SEQ ID NO: 62 - DNA, 405 bp, Artificial Sequence
  Operational Taxonomic Unit (OTU) consensus sequence 65
tggggaatattgcgcaatgggggaaaccctgacgcagcaacgccgcgtgattgaagaaggccttcgggttgtaaagatctttaattcgggacgaattttgacggtaccgaaagaataagctccggctaactacgtgccagcagccgcggtaatacgtagggagcaagcgttatccggatttactgggtgtaaagggcgcgcaggcgggccggcaagttggaagtgaaatccgggggcttaacccccgaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacagg

SEQ ID NO: 63 - DNA, 405 bp, Artificial Sequence
  Operational Taxonomic Unit (OTU) consensus sequence 66
tggggaatcttgcgcaatggggggaaccctgacgcagcgacgccgcgtgcgggacggaggccttcgggtcgtaaaccgctttcagcagggaagagtcaagactgtacctgcagaagaagccccggctaactacgtgccagcagccgcggtaatacgtagggggcgagcgttatccggattcattgggcgtaaagcgcgcgtaggcggcccggcaggccgggggtcgaagcggggggctcaaccccccgaagcccccggaacctccgcggcttgggtccggtaggggagggtggaacacccggtgtagcggtggaatgcgcagatatcgggtggaacaccggtggcgaaggcggccctctgggccgagaccgacgctgaggcgcgaaagctgggggagcgaacagg

SEQ ID NO: 64 - DNA, 428 bp, Artificial Sequence
  Operational Taxonomic Unit (OTU) consensus sequence 67
tagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgccagcagccgcggtaatacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctggtctgtaactgacactgaggcacgaaagcgtggggagcaaatagg

SEQ ID NO: 65 - DNA, 429 bp, Artificial Sequence
  Operational Taxonomic Unit (OTU) consensus sequence 68
tggggaatattgcgcaatgggcgaaagcctgacgcagcgacgccgcgtgagggatgaaggtcttcggatcgtaaacctctgtcagaagggaagaaactagggtgctctaatcatcatcctactgacggtaccttcaaaggaagcaccggctaactccgtgccagcagccgcggtaatacggagggtgcaagcgttaatcggaatcactgggcgtaaagcgcacgtaggctgttatgtaagtcaggggtgaaatcccacggctcaaccgtggaactgcccttgatactgcacgacttgaatccgggagagggtggcggaattccaggtgtaggagtgaaatccgtagatatctggaggaacatcagtggcgaaggcggccacctggaccggtattgacgctgaggtgcgaaagcgtggggagcaaacagg

SEQ ID NO: 66 - DNA, 429 bp, Artificial Sequence
  Operational Taxonomic Unit (OTU) consensus sequence 69
tggggaatcttccgcaatggacgaaagtctgacggagcaacgccgcgtgagtgatgacggccttcgggttgtaaagctctgtgatcggggacgaatggctggtatgctaataccatatcagagtgacggtacccgaatagcaagccacggctaactacgtgccagcagccgcggtaatacgtaggtggcaagcgttgtccggaattattgggcgtaaagcgcgcgcaggcggcttcttaagtccatcttaaaagtgcggggcttaaccccgtgatgggatggaaactgggaggctggagtatcggagaggaaagtggaattcctagtgtagcggtgaaatgcgtagagattaggaagaacaccggtggcgaaggcgactttctggacgacaactgacgctgaggcgcgaaagcgtggggagcaaacagg

SEQ ID NO: 67 - DNA, 405 bp, Artificial Sequence
  Operational Taxonomic Unit (OTU) consensus sequence 70
tggggaatattgcacaatgggggaaaccctgatgcagcaacgccgcgtgaaggaagacggttttcggattgtaaacttctgttcttagtgaagaataatgacggtagctaaggagcaagccacggctaactacgtgccagcagccgcggtaatacgtaggtggcaagcgttgtccggaattactgggtgtaaagggagcgtaggcgggatgccaagtcagctgtgaaaactatgggcttaacctgtagactgcagttgaaactggtattcttgagtgaagtagaggttggcggaattccgagtgtagcggtgaaatgcgtagatattcggaggaacaccggtggcgaaggcggccaactgggctttaactgacgctgaggctcgaaagtgtggggagcaaacagg

SEQ ID NO: 68 - DNA, 406 bp, Artificial Sequence
  Operational Taxonomic Unit (OTU) consensus sequence 50
tggggaatattgggcaatgggcgcaagcctgacccagcaacgccgcgtgaaggaagaaggctttcgggttgtaaacttcttttaagtgggaagagtagaagacggtaccacttgaataagccacggctaactacgtgccagcagccgcggtaatacgtaggtggcaagcgttgtccggatttactgggtgtaaagggcgtgcagccgggcatgcaagtcagatgtgaaatctcagggcttaaccctgaaactgcatttgaaactgtatgtcttgagtgccggagaggtaatcggaattccttgtgtagcggtgaaatgcgtagatataaggaagaacaccagtggcgaaggcggattactggacggtaactgacggtgaggcgcgaaagcgtggggagcgaacagg

SEQ ID NO: 69 - DNA, 424 bp, Artificial Sequence
  Operational Taxonomic Unit (OTU) consensus sequence 72
tgaggaatattggtcaatggacgcaagtctgaaccagccatgccgcgtgcaggaagacggctctatgagttgtaaactgcttttgtacgagggtaaactcacctacgtgtaggtgactgaaagtatcgtacgaataaggatcggctaactccgtgccagcagccgcggtaatacggaggattcaagcgttatccggatttattgggtttaaagggtgcgtaggcggtttgataagttagaggtgaaatcccggggcttaactccggaactgcctctaatactgttagactagagagtagttgcggtaggcggaatgtatggtgtagcggtgaaatgcttagagatcatacagaacaccgattgcgaaggcagcttaccaaactatatctgacgttgaggcacgaaagcgtggggagcaaacagg

SEQ ID NO: 70 - DNA, 407 bp, Artificial Sequence
  Operational Taxonomic Unit (OTU) consensus sequence 71
tggggaatattgggcaatgggcgcaagcctgacccagcaacgccgcgtgaaggaagaaggctttcgggttgtaaacttcttttctcagggacgaagcaagtgacggtacctgaggaataagccacggctaactacgtgccagcagccgcggtaatacgtaggtggcaagcgttatccggatttactgggtgtaaagggcgtgtaggcgggatcgcaagtcagatgtgaaaactggaggctcaacctccagcctgcatttgaaactgtggttcttgagtactggagaggcagacggaattcctagtgtagcggtgaaatgcgtagatattaggaggaacaccagtggcgaaggcggtctgctggacagcaactgacgctgaggcgcgaaagcgtggggagcaaacagg

SEQ ID NO: 71 - DNA, 407 bp, Artificial Sequence
  Operational Taxonomic Unit (OTU) consensus sequence 73
tggggaatattgggcaatgggggaaaccctgacccagcaacgccgcgtgaaggaagaaggccttcgggttgtaaacttcttttaccagggacgaaaaaagtgacggtacctggagaaaaagcaacggctaactacgtgccagcagccgcggtaatacgtaggttgcaagcgttgtccggatttactgggtgtaaagggcgtgtaggcggagatgcaagttgggagtgaaatccatgggctcaacccatgaactgctctcaaaactgtatcccttgagtatcggagaggcaagcggaattcctagtgtagcggtgaaatgcgtagatattaggaggaacaccagtggcgaaggcggcttgctggacgacaactgacgctgaggcgcgaaagcgtggggagcaaacagg

SEQ ID NO: 72 - DNA, 404 bp, Artificial Sequence
  Operational Taxonomic Unit (OTU) consensus sequence 74
tggggaatattggacaatgggcgaaagcctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggttaagcaagtctgaagtgaaagcccggggctcaaccccggtactgctttggaaactgtttgacttgagtgcaggagaggtaagtggaattcctagtgtagcggtgaaatgcgtagatattaggaggaacaccagtggcgaaggcggcttactggactgtaactgacgttgaggctcgaaagcgtggggagcaaacagg

SEQ ID NO: 73 - DNA, 429 bp, Artificial Sequence
  Operational Taxonomic Unit (OTU) consensus sequence 48
tggggaattttggacaatgggggcaaccctgatccagccatgccgcgtgcgggaagaaggccttcgggttgtaaaccgcttttgtcagggacgaaaaggtgcgggttaagagctagcactgatgacggtacctgaagaataagcaccggctaactacgtgccagcagccgcggtaatacgtagggtgcgagcgttaatcggaattactgggcgtaaagcgtgcgcaggcggttgggtaagacagatgtgaaatccccgggcttaacctgggaactgcatttgtgactgtccgactggagtatgtcagaggggggtggaattccaagtgtagcagtgaaatgcgtagatatttggaagaacaccgatggcgaaggcagccccctggggcaaaactgacgctcatgcacgaaagcgtggggagcaaacagg

SEQ ID NO: 74 - DNA, 405 bp, Artificial Sequence
  Operational Taxonomic Unit (OTU) consensus sequence 76
tgggggatattgcacaatgggggaaaccctgatgcagcgacgccgcgtgagggaagacggtcctctggattgtaaacctctgtcttcggggacgaaacgagacggtacccgaggaggaagccacggctaactacgtgccagcagccgcggtaatacgtaggtggcaagcgttgtccggaattactgggtgtaaagggagcgtaggcgggcaggcaagtcaggcgtgaaatatatcggctcaaccggtaacggcgcttgaaactgcaggtcttgagtgaagtagaggttggcggaattcctagtgtagcggtgaaatgcgtagatattaggaggaacaccagtggcgaaggcggccaactgggcttttactgacgctgaggctcgaaagtgtggggagcaaacagg

SEQ ID NO: 75 - DNA, 429 bp, Artificial Sequence
  Operational Taxonomic Unit (OTU) consensus sequence 78
tggggaattttggacaatgggcgcaagcctgatccagctattccgcgtgtgggatgaaggccctcgggttgtaaaccacttttgtagagaacgaaaagacatcttcgaataaaggatgttgctgacggtactctaagaataagcaccggctaactacgtgccagcagccgcggtaatacgtagggtgcgagcgttaatcggaattactgggcgtaaagggtgcgcaggcggttgagtaagacagatgtgaaatccccgagcttaactcgggaatggcatatgtgactgctcgactagagtgtgtcagagggaggtggaattccacgtgtagcagtgaaatgcgtagatatgtggaagaacaccgatggcgaaggcagcctcctgggacataactgacgctcaggcacgaaagcgtggggagcaaacagg

SEQ ID NO: 76 - DNA, 407 bp, Artificial Sequence
  Operational Taxonomic Unit (OTU) consensus sequence 77
tggggaatattgcacaatgggcgaaagcctgatgcagcaacgccgcgtgaaggatgaagggtttcggctcgtaaacttctatcaatagggaagaaacaaatgacggtacctaaataagaagccccggctaactacgtgccagcagccgcggtaatacgtagggggcaagcgttatccggaattactgggtgtaaagggagcgtaggcggcatggtaagccagatgtgaaagccttgggcttaacccgaggattgcatttggaactatcaagctagagtacaggagaggaaagcggaattcctagtgtagcggtgaaatgcgtagatattaggaagaacaccagtggcgaaggcggctttctggactgaaactgacgctgaggctcgaaagcgtggggagcaaacagg

SEQ ID NO: 77 - DNA, 429 bp, Artificial Sequence
  Operational Taxonomic Unit (OTU) consensus sequence 75
tggggaatcttccgcaatgggcgaaagcctgacggagcaatgccgcgtgagtgatgaaggaattcgttccgtaaagctcttttgtttatgacgaatgtgcagattgtaaataatgatctgtaatgacggtagtaaacgaataagccacggctaactacgtgccagcagccgcggtaatacgtaggtggcgagcgttgtccggaattattgggcgtaaagagcatgtaggcggttttttaagtctggagtgaaaatgcggggctcaaccccgtatggctctggatactggaagacttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacggg

SEQ ID NO: 78 - DNA, 404 bp, Artificial Sequence
  Operational Taxonomic Unit (OTU) consensus sequence 79
tggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtcaagcaagtcagaagtgaaaggctggggctcaaccccgggactgcttttgaaactgtttgactggagtgctggagaggtaagcggaattcctagtgtagcggtgaaatgcgtagatattaggaggaacaccagtggcgaaggcggcttactggacagtaactgacgttgaggctcgaaagcgtggggagcaaacagg

SEQ ID NO: 79 - DNA, 407 bp, Artificial Sequence
  Operational Taxonomic Unit (OTU) consensus sequence 82
tggggaatattgcacaatgggggaaaccctgatgcagcaacgccgcgtgaaggaagaagtatttcggtatgtaaacttctatcgacagggaagaaacaaatgacggtacctgaataagaagcaccggctaaatacgtgccagcagccgcggtaatacgtatggtgcaagcgttatccggatttactgggtgtaaagggtgagtaggcggtcatgcaagtcatatgtgaaatgtcagggcttaaccttggcgctgcataagaaactgtatgactagagtgcaggagaggtaagcggaattcctagtgtagcggtgaaatgcgtagatattaggaagaacaccggtggcgaaggcggcttactggactgttactgacgctgagtcacgaaagcgtggggagcaaacagg

SEQ ID NO: 80 - DNA, 407 bp, Artificial Sequence
  Operational Taxonomic Unit (OTU) consensus sequence 83
tggggaatattgcgcaatgggggaaaccctgacgcagcaacgccgcgtgattgaagaaggccttcgggttgtaaagatctttaatcagggacgaaacaaatgacggtacctgaagaataagctccggctaactacgtgccagcagccgcggtaatacgtagggagcaagcgttatccggatttactgggtgtaaagggcgcgcaggcgggccggcaagttggaagtgaaatctatgggcttaacccataaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacagg

SEQ ID NO: 81 - DNA, 429 bp, Artificial Sequence
  Operational Taxonomic Unit (OTU) consensus sequence 84
tggggaattttggacaatgggggcaaccctgatccagccatgccgcgtgcaggatgaaggccttcgggttgtaaactgcttttgtcagggacgaaaaggaccgtgttaataccatggtctgctgacggtacctgaagaataagcaccggctaactacgtgccagcagccgcggtaatacgtagggtgcaagcgttaatcggaattactgggcgtaaagcgtgcgcaggcggttctgtaagacagatgtgaaatccccgggctcaacctgggaattgcatttgtgactgcaggactagagttcatcagaggggggtggaattccaagtgtagcagtgaaatgcgtagatatttggaagaacaccaatggcgaaggcagccccctgggatgcgactgacgctcatgcacgaaagcgtggggagcaaacagg

SEQ ID NO: 82 - DNA, 404 bp, Artificial Sequence
  Operational Taxonomic Unit (OTU) consensus sequence 85
tggggaatattgcacaatgggcgaaagcctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagatggcatggcaagtctgaagtgaaagcccggggcttaaccccgggactgctttggaaactgttaagctagagtgcaggagaggtaagcggaattcctagtgtagcggtgaaatgcgtagatattaggaggaacaccggtggcgaaggcggcttactggactgtaactgacattgaggctcgaaagcgtggggagcaaacagg

SEQ ID NO: 83 - DNA, 404 bp, Artificial Sequence
  Operational Taxonomic Unit (OTU) consensus sequence 80
tggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacagg

SEQ ID NO: 84 - DNA, 404 bp, Artificial Sequence
  Operational Taxonomic Unit (OTU) consensus sequence 51
tggggaatattgcacaatgggggaaaccctgatgcagcgacgccgcgtgagtgatgaagtatttcggtatgtaaagctctatcagcagggaagataatgacggtacctgactaagaagcaccggctaaatacgtgccagcagccgcggtaatacgtatggtgcaagcgttatccggatttactgggtgtaaagggtgcgtaggtggtgagacaagtctgaagtgaaaatccggggcttaaccccggaactgctttggaaactgcctgactagagtacaggagaggtaagtggaattcctagtgtagcggtgaaatgcgtagatattaggaggaacaccagtggcgaaggcgacttactggactgctactgacactgaggcacgaaagcgtggggagcaaacagg

SEQ ID NO: 85 - DNA, 424 bp, Artificial Sequence
  Operational Taxonomic Unit (OTU) consensus sequence 86
tgaggaatattggtcaatggacgagagtctgaaccagccaagtagcgtgaaggatgactgccctatgggttgtaaacttcttttatacgggaataaagtggagtatgcatactcctttgtatgtaccgtatgaataaggatcggctaactccgtgccagcagccgcggtaatacggaggatccgagcgttatccggatttattgggtttaaagggagcgtaggcgggtgcttaagtcagttgtgaaagtttgcggctcaaccgtaaaattgcagttgatactgggtaccttgagtgcagcataggtaggcggaattcgtggtgtagcggtgaaatgcttagatatcacgaagaactccgattgcgaaggcagcttactggactgtaactgacgctgatgctcgaaagtgtgggtatcaaacagg

SEQ ID NO: 86 - DNA, 411 bp, Artificial Sequence
  Operational Taxonomic Unit (OTU) consensus sequence 81
tggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacagg

SEQ ID NO: 87 - DNA, 404 bp, Artificial Sequence
  Operational Taxonomic Unit (OTU) consensus sequence 88
tggggaatattgcacaatgggggaaaccctgatgcagcaacgccgcgtgagtgatgaaggttttcggatcgtaaagctctgtctttggggaagataatgacggtacccaaggaggaagccacggctaactacgtgccagcagccgcggtaatacgtaggtggcgagcgttatccggatttactgggcgtaaagggagcgtaggcggatgattaagtgggatgtgaaatacccgggctcaacttgggtgctgcattccaaactggttatctagagtgcaggagaggagagtggaattcctagtgtagcggtgaaatgcgtagagattaggaagaacaccagtggcgaaggcgactctctggactgtaactgacgctgaggctcgaaagcgtggggagcaaacagg

SEQ ID NO: 88 - DNA, 404 bp, Artificial Sequence
  Operational Taxonomic Unit (OTU) consensus sequence 91
tggggaatattgcacaatgggggaaaccctgatgcagcgatgccgcgtggaggaagaaggttttcggattgtaaactcctgttgaagaggacgataatgacggtactcttttagaaagctccggctaactacgtgccagcagccgcggtaatacgtagggagcgagcgttgtccggaattactgggtgtaaagggagcgtaggcgggatggcaagtcagatgtgaaaactatgggctcaacccatagactgcatttgaaactgttgttcttgagtgaggtagaggtaagcggaattcctggtgtagcggtgaaatgcgtagagatcaggaggaacatcggtggcgaaggcggcttactgggcctttactgacgctgaggctcgaaagcgtggggagcaaacagg

SEQ ID NO: 89 - DNA, 404 bp, Artificial Sequence
  Operational Taxonomic Unit (OTU) consensus sequence 92
tggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcagtgcaagtctgaagtgaaagcccggggctcaaccccgggactgctttggaaactgtgcagctagagtgtcggagaggcaagcggaattcctagtgtagcggtgaaatgcgtagatattaggaggaacaccagtggcgaaggcggcttgctggacgatgactgacgttgaggctcgaaagcgtggggagcaaacagg

SEQ ID NO: 90 - DNA, 424 bp, Artificial Sequence
  Operational Taxonomic Unit (OTU) consensus sequence 93
tgaggaatattggtcaatggacgagagtctgaaccagccaagtagcgtgaaggatgactgccctatgggttgtaaacttcttttatatgggaataaagtgagccacgtgtggctttttgtatgtaccatacgaataaggatcggctaactccgtgccagcagccgcggtaatacggaggatccgagcgttatccggatttattgggtttaaagggagcgtaggcggactattaagtcagctgtgaaagtttgcggctcaaccgtaaaattgcagttgatactggtcgtcttgagtgcagtagaggtaggcggaattcgtggtgtagcggtgaaatgcttagatatcacgaagaactccgattgcgaaggcagcttactggactgtaactgacgctgatgctcgaaagtgtgggtatcaaacagg

SEQ ID NO: 91 - DNA, 408 bp, Artificial Sequence
  Operational Taxonomic Unit (OTU) consensus sequence 94
tggggaatattgcacaatgggggaaaccctgatgcagcaacgccgcgtgaaggaagaaggttttcggatcgtaaacttctatcaacagggacgaagaaagtgacggtacctgaataagaagccccggctaactacgtgccagcagccgcggtaatacgtagggggcaagcgttatccggaattactgggtgtaaagggagcgtaggcggcacgccaagccagatgtgaaagcccgaggcttaacctcgcggattgcatttggaactggcgagctagagtacaggagaggaaagcggaattcctagtgtagcggtgaaatgcgtagatattaggaagaacaccagtggcgaaggcggctttctggactgaaactgacgctgaggctcgaaagcgtggggagcaaacagg

SEQ ID NO: 92 - DNA, 429 bp, Artificial Sequence
  Operational Taxonomic Unit (OTU) consensus sequence 89
tggggaattttggacaatgggggaaaccctgatccagccatgccgcgtgcaggatgaaggtcttcggattgtaaactgcttttgtcagggacgaaaaggtttcggttaatacccgaaactgctgacggtacctgaagaataagcaccggctaactacgtgccagcagccgcggtaatacgtagggtgcaagcgttaatcggaattactgggcgtaaagcgtgcgcaggcggttccgtaagatagatgtgaaatccccgggcttaacctgggaattgcatttatgactgcggaactggagtttatcagaggggggtggaattccaagtgtagcagtgaaatgcgtagatatttggaagaacaccaatggcgaaggcagccccctgggatacgactgacgctcatgcacgaaagcgtggggagcaaacagg

SEQ ID NO: 93 - DNA, 424 bp, Artificial Sequence
  Operational Taxonomic Unit (OTU) consensus sequence 96
tgaggaatattggtcaatgggcgagagcctgaaccagccaagtagcgtgcaggacgacggccctatgggttgtaaactgcttttatgcggggataaagtgagccacgtgtggcttattgcaggtaccgcatgaataaggaccggctaattccgtgccagcagccgcggtaatacggaaggtccgggcgttatccggatttattgggtttaaagggagcgtaggccgtctgataagcgtgttgtgaaatgtcggggctcaacctgggcattgcagcgcgaactgtgagacttgagtgcgcgggaagtaggcggaattcgtcgtgtagcggtgaaatgcttagatatgacgaagaactccgattgcgaaggcagcctgctgtagcgcaactgacgctgaagctcgaaagcgtgggtatcgaacagg

SEQ ID NO: 94 - DNA, 429 bp, Artificial Sequence
  Operational Taxonomic Unit (OTU) consensus sequence 95
tggggaatcttccgcaatggacgaaagtctgacggagcaacgccgcgtgaacgatgacggccttcgggttgtaaagttctgttatacgggacgaatggtacgacggtcaatacccgtcgtaagtgacggtaccgtaagagaaagccacggctaactacgtgccagcagccgcggtaatacgtaggtggcaagcgttgtccggaattattgggcgtaaagggcgcgcaggcggcgtcgtaagtcggtcttaaaagtgcggggcttaaccccgtgaggggaccgaaactgcgatgctagagtatcggagaggaaagcggaattcctagtgtagcggtgaaatgcgtagatattaggaggaacaccagtggcgaaagcggctttctggacgacaactgacgctgaggcgcgaaagccaggggagcaaacggg

SEQ ID NO: 95 - DNA, 430 bp, Artificial Sequence
  Operational Taxonomic Unit (OTU) consensus sequence 98
tggggaatattgcgcaatgggcgaaagcctgacgcagcgacgccgcgtgagggatgaaggttctcggatcgtaaacctctgtcaggggggaagaaaccccctcgtgtgaataatgcgagggcttgacggtacccccaaaggaagcaccggctaactccgtgccagcagccgcggtaatacggagggtgcaagcgttaatcggaatcactgggcgtaaagcgcacgtaggcggcttggtaagtcaggggtgaaatcccacagcccaactgtggaactgcctttgatactgccaggcttgagtaccggagagggtggcggaattccaggtgtaggagtgaaatccgtagatatctggaggaacaccggtggcgaaggcggccacctggacggtaactgacgctgaggtgcgaaagcgtgggtagcaaacagg

SEQ ID NO: 96 - DNA, 410 bp, Artificial Sequence
  Operational Taxonomic Unit (OTU) consensus sequence 99
tcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggcggtttcgtaagtcgtgtgtgaaaggcgggggctcaacccccggactgcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 97 - DNA, 404 bp, Artificial Sequence
  Operational Taxonomic Unit (OTU) consensus sequence 97
tagggaatattgcacaatgggggaaaccctgatgcagcgacgccgcgtgaaggaagaagtatttcggtatgtaaacttctatcagcaaggaagaaaatgacggtacttgactaagaagccccggctaaatacgtgccagcagccgcggtaatacgtatggggcaagcgttatccggatttactgggtgtaaagggagcgtaggcggcatggcaagtcagaagtgaaagcctggggctcaaccccggaattgcttttgaaactgtcaggctagagtgtcggaggggtaagcggaattcctagtgtagcggtgaaatgcgtagatattaggaggaacaccggtggcgaaggcggcttactggacgattactgacgctgaggctcgaaagcgtggggagcaaacagg

SEQ ID NO: 98 - DNA, 404 bp, Artificial Sequence
  Operational Taxonomic Unit (OTU) consensus sequence 101
tggggaatattgcacaatgggggaaaccctgatgcagcgacgccgcgtgagtgaagaagtatttcggtatgtaaagctctatcagcaaggaagataatgacggtacttgactaagaagccccggctaactacgtgccagcagccgcggtaatacgtagggggcaagcgttatccggatttactgggtgtaaagggagcgtagacggtatggtaagtcagatgtgaaagcccggggcttaaccccggaactgcatttgaaactatcaaactagagtgtcggagaggtaagtggaattcctagtgtagcggtgaaatgcgtagatattaggaggaacaccagtggcgaaggcggcttactggacgataactgacgttgaggctcgaaagcgtggggagcaaacagg

SEQ ID NO: 99 - DNA, 403 bp, Artificial Sequence
  Operational Taxonomic Unit (OTU) consensus sequence 102
tggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggatttactgggcgtaaagggtgcgtaggcggtcttttaagtcaggagtgaaaggctacggctcaaccgtagtaagctcttgaaactggaggacttgagtgcaggagaggagagtggaattcctagtgtagcggtgaaatgcgtagatattaggaggaacaccagtagcgaaggcggctctctggactgtaactgacgctgaggcacgaaagcgtggggagcaaacagg

SEQ ID NO: 100 - DNA, 429 bp, Artificial Sequence
  Operational Taxonomic Unit (OTU) consensus sequence 103
tagggaatcttcggcaatgggggcaaccctgaccgagcaacgccgcgtgagtgaagaaggttttcggatcgtaaagctctgttgtaagtcaagaacgagtgtgagagtggaaagttcacactgtgacggtagcttaccagaaagggacggctaactacgtgccagcagccgcggtaatacgtaggtcccgagcgttgtccggatttattgggcgtaaagcgagcgcaggcggtttgataagtctgaagttaaaggctgtggctcaaccatagttcgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacagg

SEQ ID NO: 101 - DNA, 424 bp, Artificial Sequence
  Operational Taxonomic Unit (OTU) consensus sequence 100
tgaggaatattggtcaatggacgagagtctgaaccagccaagtagcgtgaaggatgaaggtcctatggattgtaaacttcttttatacgggaataaagtgcagtatgcatactgttttgtatgtaccgtatgaataaggatcggctaactccgtgccagcagccgcggtaatacggaggatccgagcgttatccggatttattgggtttaaagggagcgtaggcggatgcttaagtcagttgtgaaagtttgcggctcaaccgtaaaattgcagttgatactgggcgtcttgagtacagtagaggcaggcggaattcgtggtgtagcggtgaaatgcttagatatcacgaagaactccgattgcgaaggcagcctgctggactgtcactgacgctgatgctcgaaagtgtgggtatcaaacagg

SEQ ID NO: 102 - DNA, 406 bp, Artificial Sequence
  Operational Taxonomic Unit (OTU) consensus sequence 87
tggggaatattgggcaatgggcgcaagcctgacccagcaacgccgcgtgaaggaagaaggctttcgggttgtaaacttcttttaagagggaagagcagaagactgtacctctagaataagccacggctaactacgtgccagcagccgcggtaatacgtaggtggcaagcgttgtccggatttactgggtgtaaagggcgtgcagccgggaatgcaagtcagatgtgaaatccatgggcttaacccatgaactgcatttgaaactgtatttcttgagtactggagaggcaatcggaattcctagtgtagcggtgaaatgcgtagatattaggaggaacaccagtggcgaaggcggattgctggacagcaactgacggtgaggcgcgaaagtgtggggagcaaacagg

SEQ ID NO: 103 - DNA, 405 bp, Artificial Sequence
  Operational Taxonomic Unit (OTU) consensus sequence 104
tcgggaatattgcgcaatggaggaaactctgacgcagtgacgccgcgtataggaagaaggttttcggattgtaaactattgtcgttagggaagagaaaggacagtacctaaggaggaagctccggctaactacgtgccagcagccgcggtaatacgtagggagcgagcgttatccggaattattgggtgtaaagggtgcgtagacgggaagataagttagttgtgaaatccctcggcttaactgaggaactgcaactaaaactgtttttcttgagtgcaggagaggtaagtggaattcctagtgtagcggtgaaatgcgtagatattaggaggaacaccagtggcgaaggcgacttactggactgtaactgacgttgaggcacgaaagtgtggggagcaaacagg

SEQ ID NO: 104 - DNA, 404 bp, Artificial Sequence
  Operational Taxonomic Unit (OTU) consensus sequence 90
tggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtaggcggcggagcaagtcagaagtgaaagcccggggctcaaccccgggacggcttttgaaactgccctgcttgatttcaggagaggtaagcggaattcctagtgtagcggtgaaatgcgtagatattaggaggaacaccagtggcgaaggcggcttactggactgacaatgacgctgaggctcgaaagcgtggggagcaaacagg

SEQ ID NO: 105 - DNA, 428 bp, Artificial Sequence
  Operational Taxonomic Unit (OTU) consensus sequence 108
tagggaattttcggcaatgggggaaaccctgaccgagcaacgccgcgtgaaggaagaaggttttcggattgtaaacttctgttataaaggaagaacggcggctacaggaaatggtagccgagtgacggtactttattagaaagccacggctaactacgtgccagcagccgcggtaatacgtaggtggcaagcgttatccggaattattgggcgtaaagagggagcaggcggcagcaagggtctgtggtgaaagcctgaagcttaacttcagtaagccatagaaaccaggcagctagagtgcaggagaggatcgtggaattccatgtgtagcggtgaaatgcgtagatatatggaggaacaccagtggcgaaggcgacgatctggcctgcaactgacgctcagtcccgaaagcgtggggagcaaatagg

SEQ ID NO: 106 - DNA, 406 bp, Artificial Sequence
  Operational Taxonomic Unit (OTU) consensus sequence 105
tggggaatattgggcaatggaggaaactctgacccagcaacgccgcgtggaggaagaaggttttcggatcgtaaactcctgtccttggagacgagtagaagacggtatccaaggaggaagccccggctaactacgtgccagcagccgcggtaatacgtagggggcaagcgttgtccggaataattgggcgtaaagggcgcgtaggcggctcggtaagtctggagtgaaagtcctgcttttaaggtgggaattgctttggatactgtcgggcttgagtgcaggagaggttagtggaattcccagtgtagcggtgaaatgcgtagagattgggaggaacaccagtggcgaaggcgactaactggactgtaactgacgctgaggcgcgaaagtgtggggagcaaacagg

SEQ ID NO: 107 - DNA, 424 bp, Artificial Sequence
  Operational Taxonomic Unit (OTU) consensus sequence 106
tgaggaatattggtcaatggacgaaagtctgaaccagccaagtagcgtgcaggatgacggccctctgggttgtaaactgcttttagttgggaataaaaagagggacgtgtcccttattgtatgtaccatcagaaaaaggaccggctaattccgtgccagcagccgcggtaatacggaaggtccaggcgttatccggatttattgggtttaaagggagcgcaggcggcggcgtaagtcagttgtgaaatcgtgcggcttaaccgtgcaattgcagttgatactgcgtcgcttgagtgcacacagggatgttggaattcatggtgtagcggtgaaatgcttagatatcatgaagaactccgatcgcgaaggcatatgtccggagtgcaactgacgctgaggctcgaaagtgtgggtatcaaacagg

SEQ ID NO: 108 - DNA, 406 bp, Artificial Sequence
  Operational Taxonomic Unit (OTU) consensus sequence 111
tggggaatattgcacaatgggggaaaccctgatgcagcgacgccgcgtgagtgaagaagtatttcggtatgtaaagctctatcagcagggaagaagaaatgacggtacctgactaagaagccccggctaactacgtgccagcagccgcggtaatacgtagggggcaagcgttatccggatttactgggtgtaaagggagcgtagacggtgaagcaagtctgaagtgaaaggttggggctcaaccccgaaactgctttggaaactgtttaactggagtacaggagaggtaagtggaattcctagtgtagcggtgaaatgcgtagatattaggaggaacaccagtggcgaaggcggcttactggactgtaactgacgttgaggctcgaaagcgtggggagcaaacagg

SEQ ID NO: 109 - DNA, 429 bp, Artificial Sequence
  Operational Taxonomic Unit (OTU) consensus sequence 112
tggggaatcttccgcaatgggcgaaagcctgacggagcaacgccgcgtgagtgatgacggccttcgggttgtaaaactctgtgatccgggacgaaaaggcagagtgcgaagaacaaactgcattgacggtaccggaaaagcaagccacggctaactacgtgccagcagccgcggtaatacgtaggtggcaagcgttgtccggaattattgggcgtaaagcgcgcgcaggcggcttcccaagtccctcttaaaagtgcggggcttaaccccgtgatgggaaggaaactgggaagctggagtatcggagaggaaagtggaattcctagtgtagcggtgaaatgcgtagagattaggaagaacaccggtggcgaaggcgactttctggacgaaaactgacgctgaggcgcgaaagcgtggggagcaaacagg

SEQ ID NO: 110 - DNA, 407 bp, Artificial Sequence
  Operational Taxonomic Unit (OTU) consensus sequence 107
tagggaatattgggcaatgggcgcaagcctgacccagcaacgccgcgtgagggaagaaggttttcggattgtaaacctctgtcctatgtgacgaaggaagtgacggtagcataggaggaagccccggctaactacgtgccagcagccgcggtaatacgtagggggcgagcgttgtccggaattactgggcgtaaagggtgcgtaggcggtttggtaagttggatgtgaaatacccgggcttaacttgggggctgcatccaatactgtcggacttgagtgcaggagaggaaagcggaattcctagtgtagcggtgaaatgcgtagatattaggaggaacaccggtggcgaaggcggctttctggactgtaactgacgctgaggcacgaaagcgtggggagcaaacagg

SEQ ID NO: 111 - DNA, 428 bp, Artificial Sequence
  Operational Taxonomic Unit (OTU) consensus sequence 109
tagggaattttcgtcaatggggggaaccctgaacgagcaatgccgcgtgagtgaggaaggtcttcggatcgtaaagctctgttgtaagagaaaaacgacattcatagggaatgatgagtgagtgatggtatcttaccagaaagtcacggctaactacgtgccagcagccgcggtaatacgtaggtggcgagcgttatccggaatgattgggcgtaaagggtgcgtaggtggcagaacaagtctggagtaaaaggtatgggctcaacccgtactggctctggaaactgttcagctagagaacagaagaggacggcggaactccatgtgtagcggtaaaatgcgtagatatatggaagaacaccggtggcgaaggcggccgtctggtctgttgctgacactgaagcacgaaagcgtggggagcaaatagg

SEQ ID NO: 112 - DNA, 429 bp, Artificial Sequence
  Operational Taxonomic Unit (OTU) consensus sequence 110
tggggaattttggacaatgggggcaaccctgatccagccatgccgcgtgcaggatgaaggccttcgggttgtaaactgcttttgtcagggacgaaaaggtttccgctaataccggagactgctgacggtacctgaagaataagcaccggctaactacgtgccagcagccgcggtaatacgtagggtgcaagcgttaatcggaattactgggcgtaaagcgtgcgcaggcggtttcgtaagacagatgtgaaatccccgggcttaacctgggaattgcatttgtgactgcgggactagagtttggcagagggaggtggaattccaagtgtagcagtgaaatgcgtagatatttggaagaacaccgatggcgaaggcagcctcctgggccaagactgacgctcatgcacgaaagcgtggggagcaaacagg

SEQ ID NO: 113 - DNA, 407 bp, Artificial Sequence
  Operational Taxonomic Unit (OTU) consensus sequence 114
tggggaatattgggcaatgggcgaaagcctgacccagcaacgccgcgtgagggaagaagggtttcggctcgtaaacctctgtcctatgggacgaaggaagtgacggtaccataggaggaagccccggctaactacgtgccagcagccgcggtaatacgtagggggcgagcgttgtccggaatgattgggcgtaaagggcgcgtaggcggcctgctaagtctggagtgaaagtcctgctttcaaggtgggaagtgctttggatactggtgggctggagtgcaggagaggaaagcggaattaccggtgtagcggtgaaatgcgtagagatcggtaggaacaccagtggcgaaggcggctttctggactgaaactgacgctgaggcgcgaaagcgtggggagcaaacagg

SEQ ID NO: 114 - DNA, 404 bp, Artificial Sequence
  Operational Taxonomic Unit (OTU) consensus sequence 113
tggggaatattgcacaatgggggaaaccctgatgcagcgacgccgcgtgagtgaagaagtatttcggtatgtaaagctctatcagcagggaagataatgacggtacctgactaagaagccccggctaactacgtgccagcagccgcggtaatacgtagggggcaagcgttatccggatttactgggtgtaaagggtgcgtaggtggcaaggcaagtcagatgtgaaagcccggggctcaaccccggtactgcatttgaaactgtctagctagagtgcaggagaggtaagcggaattcctagtgtagcggtgaaatgcgtagatattaggaggaacaccagtggcgaaggcggcttactggactgtaactgacactgaggcacgaaagcgtggggagcaaacagg

SEQ ID NO: 115 - DNA, 407 bp, Artificial Sequence
  Operational Taxonomic Unit (OTU) consensus sequence 117
tggggaatattgggcaatgggcgcaagcctgacccagcaacgccgcgtgagggaagaaggttttcggattgtaaacctctgtcgcagaagacgaaggaagtgacggtattctgtgaggaagccccggctaactacgtgccagcagccgcggtaatacgtagggggcgagcgttgtccggaattactgggcgtaaagggagcgtaggcggtctgataagttggatgtgaaatacccgggcttaacttggggggtgcatccaatactgttggactagagtacaggagaggaaagcggaattcctagtgtagcggtgaaatgcatagatattaggaggaacatcggtggcgaaggcggctttctggactgcaactgacgctgaggctcgaaagcgtggggagcaaacagg

SEQ ID NO: 116 - DNA, 404 bp, Artificial Sequence
  Operational Taxonomic Unit (OTU) consensus sequence 119
tggggaatattgcacaatgggggaaaccctgatgcagcgacgccgcgtggaggaagaaggtcttcggattgtaaactcctgtcccaggggacgataatgacggtaccctgggaggaagcaccggctaactacgtgccagcagccgcggtaaaacgtagggtgcaagcgttgtccggaattactgggtgtaaagggagcgcaggcggattggcaagttgggagtgaaatctatgggctcaacccataaattgctttcaaaactgtcagtcttgagtggtgtagaggtaggcggaattcccggtgtagcggtggaatgcgtagatatcgggaggaacaccagtggcgaaggcggcctactgggcactaactgacgctgaggctcgaaagcatgggtagcaaacagg

SEQ ID NO: 117 - DNA, 424 bp, Artificial Sequence
  Operational Taxonomic Unit (OTU) consensus sequence 120
tgaggaatattggtcaatggacgcaagtctgaaccagccatgccgcgtgcaggaagacggctctatgagttgtaaactgcttttgtacgagagtaaacgctcttacgtgtaagagcctgaaagtatcgtacgaataaggatcggctaactccgtgccagcagccgcggtaatacggaggatccaagcgttatccggatttattgggtttaaagggtgcgtaggcggtttgataagttagaggtgaaataccggtgcttaacaccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacagg

SEQ ID NO: 118 - DNA, 404 bp, Artificial Sequence
  Operational Taxonomic Unit (OTU) consensus sequence 116
tggggaatattgcacaatgggggaaaccctgatgcagcaacgccgcgtgagtgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggtgcgtaggtggcagtgcaagtcagatgtgaaaggccggggctcaaccccggagctgcatttgaaactgctcggctagagtacaggagaggcaggcggaattcctagtgtagcggtgaaatgcgtagatattaggaggaacaccagtggcgaaggcggcctgctggactgttactgacactgaggcacgaaagcgtggggagcaaacagg

SEQ ID NO: 119 - DNA, 404 bp, Artificial Sequence
  Operational Taxonomic Unit (OTU) consensus sequence 115
tggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgtttttctagagtgccggagaggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 120 - DNA, 422 bp, Artificial Sequence
  Operational Taxonomic Unit (OTU) consensus sequence 118
tgaggaatattggtcaatggacgagagtctgaaccagccaagtcgcgtgaaggaagacggatctatggtttgtaaacttctttagtgcgggaacaaagcggcgtcgtgacgccggatgagtgtaccgcaagaataagcatcggctaactccgtgccagcagccgcggtaatacggaggatgcgagcgttatccggatttattgggtttaaagggagcgcaggctgcgaggcaagtcagcggtcaaatgtcggggctcaaccccggcctgccgttgaaactgtcttgctagagttcgagtgaggtatgcggaatgcgttgtgtagcggtgaaatgcatagatatgacgcagaactccgattgcgaaggcagcataccaactcgcgactgacgctgaggctcgaaagcgtgggtatcgaacagg

SEQ ID NO: 121 - DNA, 404 bp, Artificial Sequence
  Operational Taxonomic Unit (OTU) consensus sequence 122
tggggaatattgcacaatggaggaaactctgatgcagcgatgccgcgtgagggaagaaggttttcggattgtaaacctctgtcttcagggacgataatgacggtacctgaggaggaagctccggctaactacgtgccagcagccgcggtaatacgtagggagcgagcgttgtccggaattactgggtgtaaagggagcgtaggcgggatcttaagtcaggtgtgaaaactatgggctcaacccatagactgcacttgaaactgaggttcttgagtgaagtagaggcaggcggaattcctagtgtagcggtgaaatgcgtagatattaggaggaacatcagtggcgaaggcggcctgctgggcttttactgacgctgaggctcgaaagcgtggggagcaaacagg

SEQ ID NO: 122 - DNA, 423 bp, Artificial Sequence
  Operational Taxonomic Unit (OTU) consensus sequence 125
tgaggaatattggtcaatgggcgcgagcctgaaccagccaagtagcgtggaggacgacggccctacgggttgtaaactccttttataaggggataaagttggccatgtatggccatttgcaggtaccttatgaataagcatcggctaattccgtgccagcagccgcggtaatacggaagatgcgagcgttatccggatttattgggtttaaagggagcgtaggcgggcagtcaagtcagcggtcaaatggcgcggctcaaccgcgttccgccgttgaaactggcagccttgagtatgcacagggtacatggaattcgtggtgtagcggtgaaatgcttagatatcacgaggaactccgatcgcgcaggcattgtaccggggcattactgacgctgaggctcgaaggtgcgggtatcaaacagg

SEQ ID NO: 123 - DNA, 428 bp, Artificial Sequence
  Operational Taxonomic Unit (OTU) consensus sequence 126
tagggaattttcggcaatgggggaaaccctgaccgagcaacgccgcgtgagcgaagaaggccttcgggtcgtaaagctctgttgtaaaggaagaacggcgcatacaggaaatggtatgcgagtgacggtactttaccagaaagccacggctaactacgtgccagcagccgcggtaatacgtaggtggcgagcgttatccggaatcattgggcgtaaagagggagcaggcggccgcaagggtctgtggtgaaagaccgaagctaaacttcggtaagccatggaaaccgggcggctagagtgcggaagaggatcgtggaattccatgtgtagcggtgaaatgcgtagatatatggaggaacaccagtggcgaaggcgacggtctgggccgcaactgacgctcattcccgaaagcgtggggagcaaatagg

SEQ ID NO: 124 - DNA, 407 bp, Artificial Sequence
  Operational Taxonomic Unit (OTU) consensus sequence 127
tgggggatattgcacaatggaggaaactctgatgcagcaacgccgcgtgagggaagaaggttttcggattgtaaacctctgtttttagtgaagaaacaaatgacggtagctaaagaggaagccacggctaactacgtgccagcagccgcggtaatacgtaggtggcaagcgttgtccggaattactgggtgtaaagggtgcgcaggcgggattgcaagttggatgtgaaataccggggcttaaccccggagctgcatccaaaactgtagttcttgagtggagtagaggtaagcggaattccgagtgtagcggtgaaatgcgtagatattcggaggaacaccagtggcgaaggcggcttactgggctctaactgacgctgaggcacgaaagcatgggtagcaaacagg

SEQ ID NO: 125 - DNA, 409 bp, Artificial Sequence
  Operational Taxonomic Unit (OTU) consensus sequence 128
tggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacagg

SEQ ID NO: 126 - DNA, 424 bp, Artificial Sequence
  Operational Taxonomic Unit (OTU) consensus sequence 123
tgaggaatattggtcaatggacgcaagtctgaaccagccaagtagcgtgcaggatgacggccctctgggttgtaaactgcttttagttgggaataaagtgcaccacgtgtggtgttttgtatgtaccatcagaaaaaggaccggctaattccgtgccagcagccgcggtaatacggaaggtccaggcgttatccggatttattgggtttaaagggagcgcaggcggacctttaagtcagctgtgaaatacggcggctcaaccgtcgaactgcagttgatactggaggtcttgagtgcacacagggatactggaattcatggtgtagcggtgaaatgctcagatatcatgaagaactccgatcgcgaaggcaggtatccggggtgcaactgacgctgaggctcgaaagtgcgggtatcaaacagg

SEQ ID NO: 127 - DNA, 404 bp, Artificial Sequence
  Operational Taxonomic Unit (OTU) consensus sequence 129
tggggaatattgcacaatgggcgcaagcctgatgcagcgatgccgcgtgagggaagaaggttctcggattgtaaacctctgtcttcagggacgataatgacggtacctgaggaggaagctccggctaactacgtgccagcagccgcggtaatacgtagggagcaagcgttgtccggaattactgggtgtaaagggagtgtaggcgggatggtaagtcagatgtgaaatttatgggctcaacccataacctgcatttgaaactgctgttcttgagtgaagtagaggttggcggaattcctagtgtagcggtgaaatgcgtagatattaggaggaacatcagtggcgaaggcggccaactgggcttttactgacgctgaggctcgaaagcgtggggagcaaacagg

SEQ ID NO: 128 - DNA, 404 bp, Artificial Sequence
  Operational Taxonomic Unit (OTU) consensus sequence 131
tggggaatattgcacaatgggggaaaccctgatgcagcgacgccgcgtgagtgaagaagttattcgtaacgtaaagctctatcagcagggaagaaaatgacggtacctgactaagaagccccggctaactacgtgccagcagccgcggtaatacgtagggggcaagcgttatccggatttactgggtgtaaagggagcgtagacggcacagcaagtctgatgtgaaagcccggggcccaaccccggaactgcattggaaactgctgggcttgagtgcaggagaggtaagcggaattcctagtgtagcggtgaaatgcgtagatattaggaggaacaccagtggcgaaggcggcttactggactgtaactgacgttgaggctcgaaagcgtggggagcaaacagg

SEQ ID NO: 129 - DNA, 405 bp, Artificial Sequence
  Operational Taxonomic Unit (OTU) consensus sequence 132
tgggggatattgcacaatgggggaaaccctgatgcagcgatgccgcgtggaggaagaaggttttcggattgtaaactcctgtcgtaagggaagaggaaggactgtaccttacaagaaagctccggctaactacgtgccagcagccgcggtaatacgtagggagcgagcgttgtccggaatgactgggtgtaaagggagcgtaggcgggatggcaagtcagatgtgaaacctgagggctcaaccttcagactgcatttgaaactgctgttcttgagtgaagtagaggtaagcggaattcctggtgtagcggtgaaatgcgtagagatcaggaggaacatcggtggcgaaggcggcttactgggcttttactgacgctgaggctcgaaagcgtggggagcaaacagg

SEQ ID NO: 130 - DNA, 408 bp, Artificial Sequence
  Operational Taxonomic Unit (OTU) consensus sequence 133
tggggaatattgggcaatgggcgaaagccttacccagcaatgccgcgtgagtgaagaaggtcttcggattgtaaagctctttgatcagggacgaacacaatgacggtacctgaagaacaagccccggctaactacgtgccagcagccgcggtaatacgtagggggcaagcgttgtccggaatgactgggcgtaaagggtgtgtaggcgggctcgcaagttggatgtgtaatacccagagcttaactcgggtgctgcatctgaaactacgagtcttgagtgtcggagaggtaagtggaattcctagtgtagcggtggaatgcgtagatattaggaggaacatcagtggcgaaggcgacttactggacgataactgacgctgaggcacgaaagcgtggggagcaaacagg

SEQ ID NO: 131 - DNA, 423 bp, Artificial Sequence
  Operational Taxonomic Unit (OTU) consensus sequence 130
tgaggaatattggtcaatggacgtaagtctgaaccagccaagtcgcgtgagggaagactgccctatgggttgtaaacctcttttataagggaagaataagttctacgtgtagaatgatgcctgtaccttatgaataagcatcggctaactccgtgccagcagccgcggtaatacggaggatgcgagcgttatccggatttattgggtttaaagggtgcgtaggcggtttattaagttagtggttaaatatttgagctaaactcaattgtgccattaatactggtaaactggagtacagacgaggtaggcggaataagttaagtagcggtgaaatgcatagatataacttagaactccgatagcgaaggcagcttaccagactgtaactgacgctgatgcacgagagcgtgggtagcgaacagg

SEQ ID NO: 132 - DNA, 407 bp, Artificial Sequence
  Operational Taxonomic Unit (OTU) consensus sequence 121
tggggaatattgggcaatgggcgaaagcctgacccagcaacgccgcgtgaaggaagaaggtcttcggattgtaaacttcttttatgagggacgaaggaagtgacggtacctcatgaataagccacggctaactacgtgccagcagccgcggtaatacgtaggtggcaagcgttgtccggatttactgggtgtaaagggcgcgtaggcgggatggcaagtcagatgtgaaatccatgggctcaacccatgaactgcatttgaaactgtcgttcttgagtatcggagaggcaagcggaattcctagtgtagcggtgaaatgcgtagatattaggaggaacaccagtggcgaaggcggcttgctggacgacaactgacgctgaggcgcgaaagcgtggggagcaaacagg

SEQ ID NO: 133 - DNA, 406 bp, Artificial Sequence
  Operational Taxonomic Unit (OTU) consensus sequence 124
tggggaatattgcacaatgggcgaaagcctgatgcagcgacgccgcgtgaacgaagaagtatttcggtatgtaaagttctatcagcagggaagaagaaatgacggtacctgactaagaagctccggctaaatacgtgccagcagccgcggtaatacgtatggagcaagcgttatccggatttactgggtgtaaagggagcgtaggcggtcttataagtctgatgtgaaagcccggggctcaaccccgggactgcattggaaactgtaggactagagtgtcggaggggtaagtggaattcctagtgtagcggtgaaatgcgtagatattaggaggaacaccagtggcgaaggcggcttactggacgattactgacgctgaggctcgaaagcgtggggagcaaacagg

SEQ ID NO: 134 - DNA, 403 bp, Artificial Sequence
  Operational Taxonomic Unit (OTU) consensus sequence 135
tgaggaatattgggcaatggaggcaactctgacccagccatgccgcgtgagtgagaaggttttcgaattgtaaagctctttcgggtgtgaagatgatgacggtaacaccagaagaagccccggcaaacttcgtgccagcagccgcggtaatacgaagggggcgagcgttgttcggaattactgggcgtaaagggtgtgtaggcggttaagtaagatagtggtgaaatgccggggctcaacctcggaattgccattatgactatttagctagaatgatgcagaggatagcggaatacccagtgtagaggtgaaattcgtagatattgggtagaacaccagaggcgaaggcggctatctgggcattgattgacgctgaggcacgaaagcatggggatcaaacagg

SEQ ID NO: 135 - DNA, 405 bp, Artificial Sequence
  Operational Taxonomic Unit (OTU) consensus sequence 138
tgggggatattgcacaatgggggaaaccctgatgcagcgacgccgcgtgagggaagacggccttcgggttgtaaacctctgtcttcggggacgaataaatgacggtacccgaggaggaagccacggctaactacgtgccagcagccgcggtaatacgtaggtggcaagcgttgtccggaattactgggtgtaaagggagcgtaggcggggaggcaagttgaatgtctaaactatcggctcaactgatagtcgcgttcaaaactgccactcttgagtgcagtagaggtaggcggaattcctagtgtagcggtgaaatgcgtagatattaggaggaacaccagtggcgaaggcggcctactgggctgtaactgacgctgaggctcgaaagcgtgggtagcaaacagg

SEQ ID NO: 136 - DNA, 429 bp, Artificial Sequence
  Operational Taxonomic Unit (OTU) consensus sequence 139
tagggaatcttccacaatgggcgcaagcctgatggagcaacaccgcgtgagtgaagaagggtttcggctcgtaaagctctgttgttagagaagaacgtgcgtgagagcaactgttcacgcagtgacggtatctaaccagaaagtcacggctaactacgtgccagcagccgcggtaatacgtaggtggcaagcgttatccggatttattgggcgtaaagcgagcgcaggcggtttgataagtctgatgtgaaagcctttggcttaaccaaagaagtgcatcggaaactgtcagacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacagg

SEQ ID NO: 137 - DNA, 424 bp, Artificial Sequence
  Operational Taxonomic Unit (OTU) consensus sequence 140
tgaggaatattggtcaatggacggaagtctgaaccagccatgccgcgtgcaggaagacggctctatgagttgtaaactgcttttgtacgagggtaaacgcagatacgtgtatctgcctgaaagtatcgtacgaataaggatcggctaactccgtgccagcagccgcggtaatacggaggatccaagcgttatccggatttattgggtttaaagggtgcgtaggcggtttagtaagtcagcggtgaaattttggtgcttaacaccaaacgtgccgttgatactgctgggctagagagtagttgcggtaggcggaatgtatggtgtagcggtgaaatgcttagagatcatacagaacaccgattgcgaaggcagcttaccaaactatatctgacgttgaggcacgaaagcgtggggagcaaacagg

SEQ ID NO: 138 - DNA, 406 bp, Artificial Sequence
  Operational Taxonomic Unit (OTU) consensus sequence 141
tggggaatattgcacaatgggggaaaccctgatgcagcgacgccgcgtgagtgaggaagtatttcggtatgtaaagctctatcagcagggaagaagaaatgacggtacctgactaagaagccccggctaactacgtgccagcagccgcggtaatacgtagggggcaagcgttatccggatttactgggtgtaaagggagcgcaggcggcatgataagtctgatgtgaaaacccaaggctcaaccatgggactgcattggaaactgtcgtgctggagtgtcggagaggtaagcggaattcctagtgtagcggtgaaatgcgtagatattaggaggaacaccagtggcgaaggcggcttactggacgatgactgacgctgaggctcgaaagcgtggggagcaaacagg

SEQ ID NO: 139 - DNA, 429 bp, Artificial Sequence
  Operational Taxonomic Unit (OTU) consensus sequence 142
tggggaatcttccgcaatggacgaaagtctgacggagcaacgccgcgtgagtgatgacggccttcgggttgtaaagctctgttaatcgggacgaaaggccttcttgcgaatagttagaaggattgacggtaccggaatagaaagccacggctaactacgtgccagcagccgcggtaatacgtaggtggcaagcgttgtccggaattattgggcgtaaagcgcgcgcaggcggatcagtcagtctgtcttaaaagttcggggcttaaccccgtgatgggatggaaactgctgatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacggg

SEQ ID NO: 140 - DNA, 407 bp, Artificial Sequence
  Operational Taxonomic Unit (OTU) consensus sequence 143
tgggggatattgcgcaatgggggaaaccctgacgcagcaacgccgcgtgaaggaagaaggtcttcggattgtaaacttcttttgtcagggacgaagaaagtgacggtacctgacgaataagctccggctaactacgtgccagcagccgcggtaatacgtagggagcgagcgttgtccggatttactgggtgtaaagggtgcgtaggcggccgagcaagtcagttgtgaaaactatgggcttaacccataacgtgcaattgaaactgtccggcttgagtgaagtagaggtaggcggaattcccggtgtagcggtgaaatgcgtagagatcgggaggaacaccagtggcgaaggcggcctactgggctttaactgacgctgaggcacgaaagcatgggtagcaaacagg

SEQ ID NO: 141 - DNA, 408 bp, Artificial Sequence
  Operational Taxonomic Unit (OTU) consensus sequence 144
tggggaatattgggcaatgggcgcaagcctgacccagcaacgccgcgtgagggaagaaggttttcggattgtaaacctctgtcgcaggggacgaaggaagtgacggtaccctgtaagaaagccccggctaactacgtgccagcagccgcggtaatacgtagggggcgagcgttgtccggaattactgggcgtaaagggagcgtaggcggtcgattaagttagatgtgaaacccccgggcttaacttggggactgcatctaatactggttgacttagagtacaggagagggaagcggaattcctagtgtagcggtgaaatgcgtagatattaggaggaacaccagtggcgaaggcggctttctggactgacactgacgctgaggctcgaaagcgtggggagcaaacagg

SEQ ID NO: 142 - DNA, 429 bp, Artificial Sequence
  Operational Taxonomic Unit (OTU) consensus sequence 134
tggggaatcttccgcaatgggcgaaagcctgacggagcaatgccgcgtgagtgaagaaggtcttcggaccgtaaagctctgttgttcatgacgaacgtgcagggggtgaataatttcctgtaatgacggtagtgaacgaggaagccacggctaactacgtgccagcagccgcggtaatacgtaggtggcgagcgttgtccggaattattgggcgtaaagagcatgtaggcggttttttaagtctggagtgaaaatgcggggctcaaccccgtatggctttggatactggaagacttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacggg

SEQ ID NO: 143 - DNA, 407 bp, Artificial Sequence
  Operational Taxonomic Unit (OTU) consensus sequence 146
tagggaatattgcacaatggaggaaactctgatgcagccatgccgcgtgtgtgaagaaggccttcgggttgtaaagcactttcggaggggaggaagaaaatgacgttaccctcagaagaagcaccggctaactccgtgccagcagccgcggtaatacggagggtgcaagcgttaatcggaataactgggcgtaaagggcatgcaggcggttcatcaagtaggatgtgaaatccccgggctcaacctgggaacagcatactaaactggtggactagagtattgcagggggagacggaattccaggtgtagcggtggaatgcgtagatatctggaagaacaccaaaggcgaaggcagtctcctgggcaaatactgacgctcatatgcgaaagcgtgggtagcaaacagg

SEQ ID NO: 144 - DNA, 429 bp, Artificial Sequence
  Operational Taxonomic Unit (OTU) consensus sequence 145
tggggaatcttccgcaatgggcgaaagcctgacggagcaacgccgcgtgagtgatgacggccttcgggttgtaaagctctgtgatcggggacgaatgagcagcgtgccaataccacgctgaaatgacggtacccgaaaagcaagccacggctaactacgtgccagcagccgcggtaatacgtaggtggcaagcgttgtccggaattattgggcgtaaagcgcgcgcaggcggtttgctaagtccatcttaaaagtgcggggcttaaccccgtgatgggatggaaactggcagactggagtatcggagaggaaagcggaattcctagtgtagcggtgaaatgcgtagagattaggaagaacaccggtggcgaaggcggctttctggacgacaactgacgctgaggcgcgaaagcgtggggagcaaacagg

SEQ ID NO: 145 - DNA, 408 bp, Artificial Sequence
  Operational Taxonomic Unit (OTU) consensus sequence 147
tggggaatattgcacaatgggggaaaccctgatgcagcaacgccgcgtgaaggaagacggttttcggattgtaaacttctatcaatagggacgaagaaagtgactgtacctaaataagaagccccggctaactacgtgccagcagccgcggtaatacgtagggggcaagcgttatccggaattactgggtgtaaagggtgagtaggcggcatggcaagtaagatgtgaaagcccgaggcttaacctcgggattgcattttaaactgctaagctagagtacaggagaggaaagcggaattcctagtgtagcggtgaaatgcgtagatattaggaagaacaccagtggcgaaggcggctttctggactggaaactgacgctgaggcacgaaagcgtggggagcgaacagg

SEQ ID NO: 146 - DNA, 429 bp, Artificial Sequence
  Operational Taxonomic Unit (OTU) consensus sequence 148
tggggaattttggacaatgggcgaaagcctgatccagccatgccgcgtgtgggatgaaggccttcgggttgtaaaccacttttgtcagggacgaaaaggttcaggctaataccttgaactgctgacggtacctgaagaataagcaccggctaactacgtgccagcagccgcggtaatacgtagggtgcaagcgttaatcggaattactgggcgtaaagcgtgcgcaggcggttctgtaagatagatgtgaaatccccgggcttaaccttggaattgcatttatgactgcaggactcgagtttgtcagaggggggtggaattccaagtgtagcagtgaaatgcgtagatatttggaagaacaccgatggcgaaggcagccccctgggacatgactgacgctcatgcacgaaagcgtggggagcaaacagg

SEQ ID NO: 147 - DNA, 429 bp, Artificial Sequence
  Operational Taxonomic Unit (OTU) consensus sequence 149
tggggaattttggacaatgggggcaaccctgatccagccatgccgcgtgcgggaagaaggccttcgggttgtaaaccgcttttgttaggaacgaaaaggtatctgtgaacaacaggtattgctgacggtacctaaagaataagcaccggctaactacgtgccagcagccgcggtaatacgtagggtgcgagcgttaatcggaattactgggcgtaaagcgtgcgcaggcggttgggtaagacaggtgtgaaatccccgagcttaacttgggaactgcacttgtgactgctcaactagagtatgtcagagggaggtggaattccaagtgtagcagtgaaatgcgtagatatttggaagaacaccgatggcgaaggcagcctcctgggataatactgacgctcatgcacgaaagcgtggggagcaaacagg

SEQ ID NO: 148 - DNA, 404 bp, Artificial Sequence
  Operational Taxonomic Unit (OTU) consensus sequence 151
tgaggaatattgggcaatggaggcaactctgacccagccatgccgcgtgagtgaagaaggttttcggattgtaaagctctttcggatgtgacgatgatgacggtagcatctaaagaagccccggctaacttcgtgccagcagccgcggtaatacgaagggggcgagcgttgttcggaattactgggcgtaaagggtgtgtaggcggttatgtaagatagcggtgaaatcccggggcttaacctcggaatagccgttataactatgtagctagagttatggagaggatagcggaatacccagtgtagaggtgaaattcgtagatattgggtagaacaccggtggcgaaggcggctatctggccatatactgacgctgaggcacgaaagcatggggatcaaacagg

SEQ ID NO: 149 - DNA, 429 bp, Artificial Sequence
  Operational Taxonomic Unit (OTU) consensus sequence 152
tggggaattttggacaatgggggcaaccctgatccagccatgccgcgtgcaggatgaaggtcttcggattgtaaactgcttttgtcagggacgaaaagggatgcgataacaccgcattccgctgacggtacctgaagaataagcaccggctaactacgtgccagcagccgcggtaatacgtagggtgcaagcgttaatcggaattactgggcgtaaagcgtgcgcaggcggttctgtaagatagatgtgaaatccccgggctcaacctgggaattgcatatatgactgcaggacttgagtttgtcagaggagggtggaattccacgtgtagcagtgaaatgcgtagatatgtggaagaacaccgatggcgaaggcagccctctgggacatgactgacgctcatgcacgaaagcgtggggagcaaacagg

SEQ ID NO: 150 - DNA, 404 bp, Artificial Sequence
  Operational Taxonomic Unit (OTU) consensus sequence 136
tggggaatattgcacaatgggggaaaccctgatgcagcaacgccgcgtgagtgaagaagtatttcggtatgtaaagctctatcagcagggaagaaagtgacggtacctgactaagaagccccggctaactacgtgccagcagccgcggtaatacgtagggggcaagcgttatccggatttactgggtgtaaagggagcgtagacggcacagcaagtctgaagtgaaatccccgggctcaacccgggaactgctttggaaactgttgggctagagtgctggagaggcaagcggaattcctagtgtagcggtgaaatgcgtagatattaggaggaacaccagtggcgaaggcggcttgctggacagtaactgacgttgaggctcgaaagcgtggggagcaaacagg

SEQ ID NO: 151 - DNA, 406 bp, Artificial Sequence
  Operational Taxonomic Unit (OTU) consensus sequence 150
tggggaatattgggcaatggacgcaagtctgacccagcaacgccgcgtgaaggaagaaggctttcgggttgtaaacttcttttgtcagggaacagtagaagagggtacctgacgaataagccacggctaactacgtgccagcagccgcggtaatacgtaggtggcaagcgttgtccggatttactgggtgtaaagggcgtgcagccgggctggcaagtcaggcgtgaaatcccagggctcaaccctggaactgcgtttgaaactgctggtcttgagtaccggagaggtcatcggaattccttgtgtagcggtgaaatgcgtagatataaggaagaacaccagtggcgaaggcggatgactggacggcaactgacggtgaggcgcgaaagcgtggggagcaaacagg

SEQ ID NO: 152 - DNA, 404 bp, Artificial Sequence
  Operational Taxonomic Unit (OTU) consensus sequence 155
tggggaatattggacaatgggcgaaagcctgatccagcgacgccgcgtgagcgatgaagtatttcggtatgtaaagctctatcagcagggaagaaaacgacggtacctgactaagaagccccggctaactacgtgccagcagccgcggtaatacgtagggggcaagcgttatccggatttactgggtgtaaagggagcgtagacggcatcacaagtcagaagtgaaaatccggggctcaaccccggaactgcttttgaaactgtggagctggagtgcaggagaggtaagcggaattcctagtgtagcggtgaaatgcgtagatattaggaggaacaccagtggcgaaggcggcttactggactgtaactgacgttgaggctcgaaagcgtggggagcaaacagg

SEQ ID NO: 153 - DNA, 424 bp, Artificial Sequence
  Operational Taxonomic Unit (OTU) consensus sequence 156
tgaggaatattggtcaatgggcgagagcctgaaccagccaagtagcgtgaaggatgaaggtcctacggattgtaaacttcttttataagggaataaaacgctccacgtgtggagccttgtatgtaccttatgaataagcatcggctaactccgtgccagcagccgcggtaatacggaggatgcgagcgttatccggatttattgggtttaaagggagcgcagacgggatgttaagtcagctgtgaaagtttgcggctcaaccgtaaaattgcagttgatactggcgttcttgagtgcagttgaggtgtgcggaattcgtggtgtagcggtgaaatgcttagatatcacgaagaactccgattgcgaaggcagctcactaaactgtaactgacgttcatgctcgaaagtgtgggtatcaaacagg

SEQ ID NO: 154 - DNA, 407 bp, Artificial Sequence
  Operational Taxonomic Unit (OTU) consensus sequence 157
tggggaatattgggcaatgggcgaaagcctgacccagcaacgccgcgtgaaggaagaaggtcttcggattgtaaacttcttttatgagggacgaaggacgtgacggtacctcatgaataagccacggctaactacgtgccagcagccgcggtaatacgtaggtggcaagcgttatccggatttactgggtgtaaagggcgcgtaggcggggatgcaagtcagatgtgaaatctatgggcttaacccataaactgcatttgaaactgtatctcttgagtgctggagaggtagatggaattccttgtgtagcggtgaaatgcgtagatataaggaagaacaccagtggcgaaggcgatctactggacagtaactgacgctgaggcgcgaaagcgtggggagcaaacagg

SEQ ID NO: 155 - DNA, 424 bp, Artificial Sequence
  Operational Taxonomic Unit (OTU) consensus sequence 154
tgaggaatattggtcaatgggcgagagcctgaaccagccaagtcgcgtgaaggatgaaggatctatggtttgtaaacttcttttatatgggaataaagtgaggaacgtgttcctttttgtatgtaccatatgaataagcatcggctaactccgtgccagcagccgcggtaatacggaggatgcgagcgttatccggatttattgggtttaaagggtgcgtaggtggttaattaagtcagcggtgaaagtttgtggctcaaccataaaattgccgttgaaactggttgacttgagtatatttgaggtaggcggaatgcgtggtgtagcggtgaaatgcatagatatcacgcagaactccgattgcgaaggcagcttactaaactataactgacactgaagcacgaaagcgtggggatcaaacagg

SEQ ID NO: 156 - DNA, 404 bp, Artificial Sequence
  Operational Taxonomic Unit (OTU) consensus sequence 158
tgggggatattgcacaatgggggaaaccctgatgcagcaacgccgcgtgagggaagaaggttttcggattgtaaacctctgttcttagtgacgataatgacggtagctaaggagaaagctccggctaactacgtgccagcagccgcggtaatacgtagggagcgagcgttgtccggatttactgggtgtaaagggtgcgtaggcggcgaggcaagtcaggcgtgaaatctatgggcttaacccataaactgcgcttgaaactgtcttgcttgagtgaagtagaggtaggcggaattcccggtgtagcggtgaaatgcgtagagatcgggaggaacaccagtggcgaaggcggcctactgggctttaactgacgctgaagcacgaaagcatgggtagcaaacagg

SEQ ID NO: 157 - DNA, 404 bp, Artificial Sequence
  Operational Taxonomic Unit (OTU) consensus sequence 159
tgggggatattggacaatgggggaaaccctgatccagcgatgccgcgtgagggaagaaggttttcggattgtaaacctctgtggacagcgacgataatgacggtagctgtttagaaagccacggctaactacgtgccagcagccgcggtaatacgtaggtggcgagcgttgtccggaattactgggtgtaaagggagtgcaggcgggactgcaagtcagaagtgaaaattatgggcttaacccataacctgcttttgaaactgtagttcttgagtgaggcagaggcaagcggaattcctagtgtagcggtgaaatgcgtagatattaggaggaacaccagtggcgaaggcggcttgctgggcctttactgacgctgaggctcgaaagcgtggggagcaaacagg

SEQ ID NO: 158 - DNA, 404 bp, Artificial Sequence
  Operational Taxonomic Unit (OTU) consensus sequence 160
tggggaatattgcacaatgggggaaaccctgatgcagcgatgccgcgtggaggaagaaggttttcggattgtaaactcctgtcttaaaggacgataatgacggtactttaggaggaagctccggctaactacgtgccagcagccgcggtaatacgtagggagcgagcgttgtccggaattactgggtgtaaagggagcgtaggcgggacggcaagtcagatgtgaaatacatgggctcaacccatgggctgcatttgaaactgctgttcttgagtgaagtagaggtaagcggaattcctggtgtagcggtgaaatgcgtagatatcaggaggaacaccggtggcgaaggcggcttactgggcttttactgacgctgaggctcgaaagcgtggggagcaaacagg

SEQ ID NO: 159 - DNA, 406 bp, Artificial Sequence
  Operational Taxonomic Unit (OTU) consensus sequence 161
tggggaatattgggcaatgggcgcaagcctgacccagcgacgccgcgtgagggaagacagccttcgggttgtaaacctctgttgcaggggaagaaggacgtgacggtaccctgcgaggaagctccggctaactacgtgccagcagccgcggtaatacgtagggagcgagcgttgtccggaattactgggcgtaaagggcgcgtaggcggcgcttcaagtcgtctgtcaaaagccgaggctcaacctcggtgcgcagacgaaactggagagcttgagaagcagagaggcaaacagaattcctggtgtagcggtgaaatgcgtagatatcaggaagaataccagtggcgaaggcggtttgctggctgcatactgacgctgaagcgcgaaagccaggggagcaaacggg

SEQ ID NO: 160 - DNA, 405 bp, Artificial Sequence
  Operational Taxonomic Unit (OTU) consensus sequence 153
tggggaatattgcacaatgggggaaaccctgatgcagcaacgccgcgtgaaggaagacggtcttcggattgtaaacttttgttcttggtgaagaaaaatgacggtagccaaggaggaagccacggctaactacgtgccagcagccgcggtaatacgtaggtggcaagcgttgtccggaattactgggtgtaaagggagcgcaggcgggaaatcaagttggatgtgaaatgtcggggcttaaccccggaactgcatccaaaactgatattcttgagtgaagtagaggtaggcggaattccgagtgtagcggtgaaatgcgtagatattcggaggaacaccagtggcgaaggcggcctactgggctttaactgacgctgaggctcgaaagtgtggggagcaaacagg

SEQ ID NO: 161 - DNA, 429 bp, Artificial Sequence
  Operational Taxonomic Unit (OTU) consensus sequence 137
tggggaatcttccgcaatgggcgaaagcctgacggagcaacgccgcgtgagtgatgacggccttcgggttgtaaagctctgtgaccggggacgaacggtctgtaagctaataacttatggaagtgacggtacccggatagcaagccacggctaactacgtgccagcagccgcggtaatacgtaggtggcaagcgttgtccggaattattgggcgtaaagcgcgcgcaggcggcttcctaagtccatcttaaaagtgcggggcttaaccccgtgatgggatggaaactgggaagctggagtatcggagaggaaagtggaattcctagtgtagcggtgaaatgcgtagagattaggaagaacaccggtggcgaaggcgactttctggacgacaactgacgctgaggcgcgaaagcgtggggagcaaacagg

SEQ ID NO: 162 - DNA, 405 bp, Artificial Sequence
  Operational Taxonomic Unit (OTU) consensus sequence 164
tcgggaatattgcgcaatggaggaaactctgacgcagtgacgccgcgtataggaagaaggttttcggattgtaaactattgtcgttagggaagatacaagacagtacctaaggaggaagctccggctaactacgtgccagcagccgcggtaatacgtagggagcaagcgttatccggatttattgggtgtaaagggtgcgtagacgggacaacaagttagttgtgaaatccctcggcttaactgaggaactgcaactaaaactattgttcttgagtgttggagaggaaagtggaattcctagtgtagcggtgaaatgcgtagatattaggaggaacaccggtggcgaaggcgactttctggacaataactgacgttgaggcacgaaagtgtggggagcaaacagg

SEQ ID NO: 163 - DNA, 424 bp, Artificial Sequence
  Operational Taxonomic Unit (OTU) consensus sequence 167
tgaggaatattggtcaatgggcggaagcctgaaccagccaagtagcgtgagggaagactgccctatgggttgtaaacctcttttgtgcggggataaagtgtgggacgtgtcccatattgcaggtaccgcacgaataaggaccggctaattccgtgccagcagccgcggtaatacggaaggtccgggcgttatccggatttattgggtttaaagggagcgcaggccgtcctttaagcgtgctgtgaaatgccgcggctcaaccgtggcactgcagcgcgaactggaggacttgagtacgcacgaggtaggcggaattcgtggtgtagcggtgaaatgcttagatatcacgaagaactccgattgcgaaggcagcttaccggagcgcaactgacgctgaggctcgaaagcgcgggtatcgaacagg

SEQ ID NO: 164 - DNA, 407 bp, Artificial Sequence
  Operational Taxonomic Unit (OTU) consensus sequence 168
tggggaatattgggcaatgggggaaaccctgacccagcaacgccgcgtgaaggaagaaggccttcgggttgtaaacttcttttaccagggacgaagaaagtgacggtacctggagaaaaagccacggctaactacgtgccagcagccgcggtaatacgtaggtggcaagcgttgtccggaattactgggtgtaaagggcgtgtaggcggagtagcaagtcaggagtgaaatctaagggctcaacccttaaactgcttttgaaactgctacccttgagtatcggagaggcaggcggaattcctagtgtagcggtgaaatgcgtagatattaggaggaacaccagtggcgaaggcggcctgctggacgacaactgacgctgaggcgcgaaagcgtggggagcaaacagg

SEQ ID NO: 165 - DNA, 429 bp, Artificial Sequence
  Operational Taxonomic Unit (OTU) consensus sequence 169
tggggaattttggacaatgggggaaaccctgatccagccatgccgcgtgcgggatgaaggccttcgggttgtaaaccgcttttgtcagagacgaaaagggacgtacgaataatacgttccgctgacggtatctgaagaataagcaccggctaactacgtgccagcagccgcggtaatacgtagggtgcaagcgttaatcggaattactgggcgtaaagggtgcgcaggcggctgtgcaagacagatgtgaaatccccgggcttaacctgggaactgcatttgtgactgcacggctagagtttgtcagaggagggtggaattccgcgtgtagcagtgaaatgcgtagatatgcggaagaacaccaatggcgaaggcagccctctgggacatgactgacgctcatgcacgaaagcgtggggagcaaacagg

SEQ ID NO: 166 - DNA, 423 bp, Artificial Sequence
  Operational Taxonomic Unit (OTU) consensus sequence 170
tgaggaatattggtcaatggacgagagtctgaaccagccaagtagcgtgaaggatgactgccctatgggttgtaaacttcttttatatgggaataaaaaaggtcacgtgtggcctattgtatgtaccttatgaataagcatcggctaattccgtgccagcagccgcggtaatacggaagatgcgagcgttatccggatttattgggtttaaagggagcgtaggcgggcgattaagtcagcggtaaaatagtgtggctcaaccatgctctgccgttgatactggttgccttgagtgcacacaaggaagatggaattcgtggtgtagcggtgaaatgcttagatatcacgaagaactccgattgcgaaggcagtcttctggggtgttactgacgctgaggctcgaaagtgcgggtatcaaacagg

SEQ ID NO: 167 - DNA, 424 bp, Artificial Sequence
  Operational Taxonomic Unit (OTU) consensus sequence 171
tgaggaatattggtcaatgggcgcgagcctgaaccagccaagtagcgtgaaggatgaaggtcctacggattgtaaacttcttttataagggaataaagtcacctacgtgtaggtgtttgtatgtaccttatgaataagcatcggctaactccgtgccagcagccgcggtaatacggaggatgcgagcgttatccggatttattgggtttaaagggagcgtagacgggtcgttaagtcagctgtgaaagtttggggctcaaccttgaaattgcagttgatactggcgtccttgagtacggttgaggcaggcggaattcgtggtgtagcggtgaaatgcttagatatcacgaagaaccccgattgcgaaggcagcctgctaagccgcgactgacgttgaggctcgaaagtgtgggtatcaaacagg

SEQ ID NO: 168 - DNA, 424 bp, Artificial Sequence
  Operational Taxonomic Unit (OTU) consensus sequence 172
tgaggaatattggtcaatggtcggcagactgaaccagccaagtcgcgtgagggaagacggccctacgggttgtaaacctcttttgtcggagagtaaagtacgctacgtgtagcgtattgcaagtatccgaagaaaaagcatcggctaactccgtgccagcagccgcggtaatacggaggatgcgagcgttatccggatttattgggtttaaagggtgcgtaggcggcacgccaagtcagcggtgaaatttccgggctcaacccggagtgtgccgttgaaactggcgagctagagtgcacaagaggcaggcggaatgcgtggtgtagcggtgaaatgcatagatatcacgcagaaccccgattgcgaaggcagcctgctagggtgaaacagacgctgaggcacgaaagcgtgggtatcgaacagg

SEQ ID NO: 169 - DNA, 404 bp, Artificial Sequence
  Operational Taxonomic Unit (OTU) consensus sequence 173
tggggaatattgcacaatggagggaactctgatgcagcgatgccgcgtggaggaagaaggttttcggattgtaaactccttttatcagggacgataatgacggtacctgaagaaaaagctccggctaactacgtgccagcagccgcggtaatacgtagggagcgagcgttgtccggaattactgggtgtaaagggagcgtaggcgggatagcaagtcagatgtgaaaactatgggctcaacctgtagattgcatttgaaactgttgttcttgagtgaagtagaggtaagcggaattcctagtgtagcggtgaaatgcgtagatattaggaggaacatcggtggcgaaggcggcttactgggcttttactgacgctgaggctcgaaagcgtggggagcaaacagg

SEQ ID NO: 170 - DNA, 404 bp, Artificial Sequence
  Operational Taxonomic Unit (OTU) consensus sequence 174
tggggaatcttgcgcaatggggggaaccctgacgcagcgacgccgcgtgcgggacgaaggccctcgggtcgtaaaccgctttcagcagggatgagacaagacggtacctgcagaagaagccccggctaactacgtgccagcagccgcggtaatacgtagggggcgagcgttatccggattcattgggcgtaaagcgcgcgtaggcggcccggcaggcagggggtcaaatggcggggctcaaccccgtcccgccccctgaaccgccgggctcgggtccggtaggggagggtggaacacccggtgtagcggtggaatgcgcagatatcgggtggaacaccggtggcgaaggcggccctctgggccgagaccgacgctgaggcgcgaaagctgggggagcgaacagg

SEQ ID NO: 171 - DNA, 424 bp, Artificial Sequence
  Operational Taxonomic Unit (OTU) consensus sequence 175
tgaggaatattggtcaatggacgggagtctgaaccagccaagtagcgtgaaggatgactgccctatgggttgtaaacttcttttatatgggaataaagtgcagtatgtatactgttttgtatgtaccatatgaataaggatcggctaactccgtgccagcagccgcggtaatacggaggatccgagcgttatccggatttattgggtttaaagggagcgtaggcggaagcttaagtcagttgtgaaagtttgcggctcaaccgtaaaattgcagttgatactgggtttcttgagtgcagtagaggtaggcggaattcgtggtgtagcggtgaaatgcttagatatcacgaagaactccgattgcgaaggcagcttactggactgtaactgacgctgatgctcgaaagtgtgggtatcaaacagg

SEQ ID NO: 172 - DNA, 404 bp, Artificial Sequence
  Operational Taxonomic Unit (OTU) consensus sequence 176
tggggaatattgcacaatgggggaaaccctgatgcagcaacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcagcgcaagtctgaagtgaaagcccggggctcaaccccggaatggctttggaaactgtgcggctagagtaccggaggggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 173 - DNA, 423 bp, Artificial Sequence
  Operational Taxonomic Unit (OTU) consensus sequence 177
tgaggaatattggtcaatgggcgagagcctgaaccagccaagtcgcgtgagggaagaatggtctatggcctgtaaacctcttttgtcagggaagaataaggatgacgagtcattcgatgccagtacttgacgaataagcatcggctaactccgtgccagcagccgcggtaatacgggggatgcgagcgttatccggatttattgggtttaaagggcgcgtaggcgggacgtcaagtcagcggtaaaagactgcagctaaactgtagcacgccgttgaaactggcgccctggagacgagacgagggaggcggaacaagtgaagtagcggtgaaatgcatagatatcacttggaaccccgatagcgaaggcagcttcccaggctcgttctgacgctgatgcgcgagagcgtgggtagcgaacagg

SEQ ID NO: 174 - DNA, 406 bp, Artificial Sequence
  Operational Taxonomic Unit (OTU) consensus sequence 178
tggggaatattgggcaatgggcgcaagcctgacccagcaacgccgcgtgaaggaagaaggctttcgggttgtaaacttcttttctcagggacgaacaaatgacggtacctgaggaataagccacggctaactacgtgccagcagccgcggtaatacgtaggtggcaagcgttatccggatttactgggtgtaaagggcgtgtaggcgggaaggcaagtcagatgtgaaaactatgggctcaacccatagcctgcatttgaaactgtttttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacagg

SEQ ID NO: 175 - DNA, 425 bp, Artificial Sequence
  Operational Taxonomic Unit (OTU) consensus sequence 179
tgaggaatattggtcaatgggcgcgagcctgaaccagccaagtagcgtgaaggatgactgccctatgggttgtaaacttcttttgtccgggaataaaaccgcctacgtgtaggcgcttgtatgtaccggtacgaataagcatcggctaactccgtgccagcagccgcggtaatacggaggatgcgagcgttatccggatttattgggtttaaagggagcgcagacgggtttttaagtcagctgtgaaagtttggggctcaaccttaaaattgcagttgatactggagaccttgagtgcagttgaggcaggcggaattcgtggtgtagcggtgaaatgcttagatatcacgaagaactccgattgcgaaggcagcttgctaaagtgtaactgacgttcatgctcgaaagtgtgggtatcaaacagg

SEQ ID NO: 176 - DNA, 424 bp, Artificial Sequence
  Operational Taxonomic Unit (OTU) consensus sequence 181
tgaggaatattggtcaatgggcggaagcctgaaccagccaagtagcgtgcaggatgacggccctacgggttgtaaactgcttttatgcggggataaagttgcccacgcgtgggtttttgcaggtaccgcatgaataaggaccggctaattccgtgccagcagccgcggtaatacggaaggtccgggcgttatccggatttattgggtttaaagggagcgcaggccgccgtgcaagcgtgccgtgaaaagcagcggcccaaccgctgccctgcggcgcgaactgcttggcttgagtgcgccggaagcgggcggaattcgtggtgtagcggtgaaatgcttagatatcacgaagaaccccgattgcgaaggcagcccgctgtggcgccactgacgctgaggctcgaaggtgcgggtatcgaacagg

SEQ ID NO: 177 - DNA, 404 bp, Artificial Sequence
  Operational Taxonomic Unit (OTU) consensus sequence 183
tgggggatattgcacaatgggggaaaccctgatgcagcaatgccgcgtgagggaagaaggtcttcggattgtaaacctaagtagccagggacgataatgacggtacctggagagtaagctccggctaactacgtgccagcagccgcggtaatacgtagggagcgagcgttgtccggatttactgggtgtaaagggtgcgtaggcgggatggcaagtcagatgtgaaataccggggcttaaccccggggctgcatttgaaactgtcgttcttgagtgaagtagaggcaggcggaattcctagtgtagcggtgaaatgcgtagatattaggaggaacaccagtggcgaaggcggcctgctgggctttaactgacgctgaggcacgaaagcatggggagcaaacagg

SEQ ID NO: 178 - DNA, 405 bp, Artificial Sequence
  Operational Taxonomic Unit (OTU) consensus sequence 184
tgggggatattgcgcaatgggggaaaccctgacgcagcaacgccgcgtgaaggatgaaggttttcggattgtaaacttcttttatttaggacgaagaatgacggtactaaatgaataagctccggctaactacgtgccagcagccgcggtaatacgtagggagcaagcgttatccggatttactgggtgtaaagggtgcgtaggcggcttggtaagtcagatgtgaaatgtatgggctcaacccatgcactgcatttgaaactattgagcttgagtgaagtagaggtaggcggaattccctgtgtagcggtgaaatgcgtagagatagggaggaacaccagtggcgaaggcggcctactgggctttaactgacgctgaggcacgaaagcgtgggtagcaaacagg

SEQ ID NO: 179 - DNA, 407 bp, Artificial Sequence
  Operational Taxonomic Unit (OTU) consensus sequence 185
tggggaatattgggcaatgggcgaaagcctgacccagcgacgccgcgtgagggaagaaggtcttcggattgtaaaccttagttagcagggaagaagaaagtgacggtacctgcagagaaagccacggctaactacgtgccagcagccgcggtaatacgtaggtggcgagcgttatccggaattactgggtgtaaagggtgtgtaggcgggcagacaagtcagatgtgaaaactatgggcttaacccatagcctgcatttgaaactgtatgtcttgaggatgggagaggtaaatggaattcccggtgtagcggtgaaatgcgtagatatcgggaggaacaccagtggcgaaggcggtttactggaccattactgacgctgagacacgaaagcgtggggagcaaacagg

SEQ ID NO: 180 - DNA, 407 bp, Artificial Sequence
  Operational Taxonomic Unit (OTU) consensus sequence 186
tggggaatattgggcaatgggcgcaagcctgacccagcaacgccgcgtgaaggaagaaggctttcgggttgtaaacttcttttatgagggacgaaggaagtgacggtacctcatgaataagctccggctaactacgtgccagcagccgcggtaatacgtagggagcgagcgttatccggatttactgggtgtaaagggcgtgtaggcggggaagcaagtcagatgtgaaaaccagtggctcaaccactggcctgcatttgaaactgtttttcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacagg

SEQ ID NO: 181 - DNA, 406 bp, Artificial Sequence
  Operational Taxonomic Unit (OTU) consensus sequence 188
tgggggatattgcacaatgggggaaaccctgatgcagcgacgccgcgtgagggaagacggttttcggattgtaaacctctgtctttagggacgaaaaaaatgacggtacctaaggaggaagccacggctaactacgtgccagcagccgcggtaatacgtaggtggcaagcgttgtccggaattactgggtgtaaagggagcgtaggcggggagacaagttgaatgtctaaactatcggcttaactgatagtcgcgttcaaaactatcactcttgagtgcagtagaggtaggcggaattcctagtgtagcggtgaaatgcgtagatattaggaggaacaccagtggcgaaggcggcctactgggctgtaactgacgctgaggctcgaaagcgtgggtagcaaacagg

SEQ ID NO: 182 - DNA, 424 bp, Artificial Sequence
  Operational Taxonomic Unit (OTU) consensus sequence 190
tgaggaatattggtcaatgggcgagagcctgaaccagccaagtagcgtgaaggatgaccgccctatgggttgtaaacttcttttatatgggaataaagggtgccacgtgtggcattttgtatgtaccatatgaataaggatcggctaactccgtgccagcagccgcggtaatacggaggatccgagcgttatccggatttattgggtttaaagggagcgtaggtggacatgtaagtcagttgtgaaagtttgcggctcaaccgtaaaattgcagttgaaactgcgtgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacagg

SEQ ID NO: 183 - DNA, 423 bp, Artificial Sequence
  Operational Taxonomic Unit (OTU) consensus sequence 165
tgaggaatattggtcaatgggcgagagcctgaaccagccaagtagcgtgcgggacgacggccctatgggttgtaaaccgcttttgattgggaacaaagagcgccacgtgtggtgcgttgcgtgtacctttcgaataagcatcggctaattccgtgccagcagccgcggtaatacggaagatgcgagcgttatccggatttattgggtttaaagggagcgtaggcgggctgttaagtcagcggtcaaatgtcagggcccaaccttggcatgccgttgatactggcggccttgagttcacacaaggaaggtggaattcgtcgtgtagcggtgaaatgcttagatatgacgaagaactccgattgcgaaggcagccttctggggtgttactgacgctgaggctcgaaagtgcgggaatcaaacagg

SEQ ID NO: 184 - DNA, 406 bp, Artificial Sequence
  Operational Taxonomic Unit (OTU) consensus sequence 192
tggggaatattgcacaatgggcgcaagcctgatgcagcaacgccgcgtgagggaagacggttttcggattgtaaacctctgtctttggtgacgaagaagtgacggtagccaaggaggaagccacggctaactacgtgccagcagccgcggtaatacgtaggtggcaagcgttatccggaattactgggtgtaaagggagcgcaggcgggatagcaagtcagcggtgaaatgcatgggcttaactcatgagctgccgttgaaactgttattcttgagtggagtagaggcaggcggaattccgagtgtagcggtgaaatgcgtagatattcggaggaacaccagtggcgaaggcggcctgctgggctctaactgacgctgaggctcgaaagtgtggggagcaaacagg

SEQ ID NO: 185 - DNA, 404 bp, Artificial Sequence
  Operational Taxonomic Unit (OTU) consensus sequence 193
tggggaatattgcacaatgggggaaaccctgatgcagcgacgccgcgtgaaggatgaagtatttcggtatgtaaacttctatcagcagggaagaagatgacggtacctgactaagaagccccggctaactacgtgccagcagccgcggtaatacgtagggggcaagcgttatccggatttactgggtgtaaagggagcgtagacggcgatgcaagccagatgtgaaagcccggggctcaaccccgggactgcatttggaactgcgtggctggagtgtcggagaggcaggcggaattcctagtgtagcggtgaaatgcgtagatattaggaggaacaccagtggcgaaggcggcctgctggacgatgactgacgttgaggctcgaaagcgtggggagcaaacagg

SEQ ID NO: 186 - DNA, 403 bp, Artificial Sequence
  Operational Taxonomic Unit (OTU) consensus sequence 194
tggggaatattgcacaatgggcgaaagcctgatgcagcaacgccgcgtgaaggaagaaggtcttcggatcgtaaacttctgtccttggggaagataatgacggtacccttggaggaagccccggctaactacgtgccagcagccgcggtaatacgtagggggcaagcgttatccggaattattgggcgtaaagagtgcgtaggtggttacctaagcagggggtgaaaggcactggcttaaccaatgtcagccccctgaactgggtaccttgagtgcaggagaggaaagcggaattcctagtgtagcggtgaaatgcgtagatattaggaggaacaccagtggcgaaggcggctttctggactgttactgacactgaggcacgaaagtgtggggagcaaacagg

SEQ ID NO: 187 - DNA, 407 bp, Artificial Sequence
  Operational Taxonomic Unit (OTU) consensus sequence 195
tggggaatattgggcaatgggggaaaccctgacccagcaacgccgcgtgagggaagaaggtcttcggattgtaaacctcttttaccagggaagaagaaagtgacggtacctggagaaaaagccacggctaactacgtgccagcagccgcggtaatacgtaggtggcaagcgttgtccggatttactgggtgtaaagggcgtgtaggcgggaagacaggtcagatgtgaaatgccggggctcaactccggagctgcatttgaaaccgtttttcttgagtatcggagaggcaggcggaattcctagtgtagcggtgaaatgcgtagatattaggaggaacaccagtggcgaaggcggcctgctggacgacaactgacgctgaggcgcgaaagcgtggggagcaaacagg

SEQ ID NO: 188 - DNA, 424 bp, Artificial Sequence
  Operational Taxonomic Unit (OTU) consensus sequence 196
tgaggaatattggtcaatgggcgcaagcctgaaccagccaagtagcgtgagggaagactgccctacgggttgtaaacctcttttgtttgggaataaagtgcgggacgtgtcccgcattgcatgtaccatttgaataaggaccggctaattccgtgccagcagccgcggtaatacggaaggtccgggcgttatccggatttattgggtttaaagggagcgcaggccgtgggttaagcgtgtcgtgaaattccgtcgctcaacggcggacgtgcggcgcgaactggtccacttgagtacgcgggacgttggcggaattcgtggtgtagcggtgaaatgcttagatatcacgaagaactccgattgcgaaggcagctgacggtagcgcaactgacgctgaggctcgaaagtgcgggtatcgaacagg

SEQ ID NO: 189 - DNA, 407 bp, Artificial Sequence
  Operational Taxonomic Unit (OTU) consensus sequence 197
tggggaatattgggcaatgggcgaaagcctgacccagcgacgccgcgtgaaggaagaaggccttcgggttgtaaactttagtaagcagggaagaagaaagtgacggtacctgcagagtaagccacggctaactacgtgccagcagccgcggtaatacgtaggtggcgagcgttatccggaattactgggtgtaaagggtgtgtaggcgggacttcaagtcagatgtgaaaattgcgggctcaacccgcaacctgcatttgaaactgaggttcttgagagtcggagaggtaaatggaattcccggtgtagcggtgaaatgcgtagatatcgggaggaacaccagtggcgaaggcgatttactggacgacaactgacgctgagacacgaaagcgtggggagcaaacagg

SEQ ID NO: 190 - DNA, 430 bp, Artificial Sequence
  Operational Taxonomic Unit (OTU) consensus sequence 166
tagggaatcttcggcaatggacgcaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacagg

SEQ ID NO: 191 - DNA, 407 bp, Artificial Sequence
  Operational Taxonomic Unit (OTU) consensus sequence 163
tggggaatattggacaatggaccaaaagtctgatccagcaattctgtgtgcacgatgacgtttttcggaatgtaaagtgctttcagttgggaagaaaaaaatgacggtaccaacagaagaagtgacggctaaatacgtgccagcagccgcggtaatacgtatgtcacaagcgttatccggatttattgggcgtaaagcgcgtctaggtggttatgtaagtctgatgtgaaaatgcagggctcaactctgtattgcgttggaaactgcatgactagagtactggagaggtaagcggaactacaagtgtagaggtgaaattcgtagatatttgtaggaatgccgatggggaagccagcttactggacagatactgacgctaaagcgcgaaagcgtgggtagcaaacagg

SEQ ID NO: 192 - DNA, 425 bp, Artificial Sequence
  Operational Taxonomic Unit (OTU) consensus sequence 191
tgaggaatattggtcaatgggcggaagcctgaaccagccaagtagcgtgagggatgactgccctatgggttgtaaacctcttttataagggaataaaatacgggacgtgtcctgttttgcatgtaccttatgaataaggaccggctaattccgtgccagcagccgcggtaatacggaaggtccgggcgttatccggatttattgggtttaaagggagcgcaggcggtcttataagcgtgacgtgaaatgcagcggctcaaccgtatgatgtgcgtcgcgaactgtgagacttgagtgtattcgatgtcagcggaatttgtggtgtagcggtgaaatgcttagatatcacgaagaactccgattgcgaaggcagctgacaaggctacaactgacgctaaagctcgaaagtgcgggtatcgaacagg

SEQ ID NO: 193 - DNA, 425 bp, Artificial Sequence
  Operational Taxonomic Unit (OTU) consensus sequence 198
tgaggaatattggtcaatggacgagagtctgaaccagccaagtagcgtgcaggatgacggccctatgggttgtaaactgcttttgcgcggggataacaccctccacgtgctggaggtctgcaggtaccgcgcgaataaggaccggctaattccgtgccagcagccgcggtaatacggaaggtccgggcgttatccggatttattgggtttaaagggagcgtaggccgtgaggtaagcgtgttgtgaaatgtaggcgcccaacgtctgcactgcagcgcgaactgccccacttgagtgcgcgcaacgccggcggaactcgtcgtgtagcggtgaaatgcttagatatgacgaagaaccccgattgcgaaggcagctggcgggagcgtaactgacgctgaagctcgaaagcgcgggtatcgaacagg

SEQ ID NO: 194 - DNA, 404 bp, Artificial Sequence
  Operational Taxonomic Unit (OTU) consensus sequence 201
tggggaatattgcacaatgggggaaaccctgatgcagcgacgccgcgtgaaggaagaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtaggcggtcagacaagtcagaagtgaaagcccggggctcaactccgggactgcttttgaaactgcctgactagattgcaggagaggtaagtggaattcctagtgtagcggtgaaatgcgtagatattaggaggaacaccagtggcgaaggcggcttactggactgtaaatgacgctgaggctcgaaagcgtggggagcaaacagg

SEQ ID NO: 195 - DNA, 429 bp, Artificial Sequence
  Operational Taxonomic Unit (OTU) consensus sequence 204
tggggaatattgcgcaatgggcgaaagcctgacgcagcgacgccgcgtgagggatgaaggtcttcggatcgtaaacctctgtcagaagggaaaaatgtacagtgctccaatcaacactgtattgatggtaccttcagaggaagcaccggctaactccgtgccagcagccgcggtaatacggagggtgcaagcgttaatcggaattactgggcgtaaagcgcgcgtaggttgttttgtaagtcagaggtgtaatcccacggcttaaccgtggaactgcctttgatactgcataacttggatccgggagaggacagcggaattccaggtgtaggagtgaaatccgtagatatctggaagaacatcagtggcgaaggcggctgtctggaccggtattgacgctgaggcgcgaaagcgtgggtagcaaacagg

SEQ ID NO: 196 - DNA, 407 bp, Artificial Sequence
  Operational Taxonomic Unit (OTU) consensus sequence 205
tggggaatattgggcaatgggcgaaagcctgacccagcgacgccgcgtgagggaagaaggtcttcggattgtaaacctctttcagcagggaagaagaaagtgacggtacctgcagaagaagtcacggctaactacgtgccagcagccgcggtaatacgtaggtggcgagcgttatccggaattactgggtgtaaagggtgtgtaggcggggtgtcaagtcagatgtgaaaactgtgggctcaacccacaaactgcatttgaaactgatactcttgagagtgggagaggtaaacggaattcctggtgtagtagtgaaatgcgtagatatcaggaggaacaccggtggcgaaggcggtttactggaccacaactgacgctgagacacgaaagcgtggggagcaaacagg

SEQ ID NO: 197 - DNA, 407 bp, Artificial Sequence
  Operational Taxonomic Unit (OTU) consensus sequence 202
tggggaatattgggcaatggagggaactctgacccagcaacgccgcgtgaatgatgaaggtcttcggattgtaaagttctgtgacgggggacgaagaaagtgacggtaccccgaaagcaagctacggctaactacgtgccagcagccgcggtaatacgtaggtagcaagcgttgtccggaatgactgggcgtaaagggtgcgtaggtggctgggcaagttggtagtgaaattccggggcttaactccggcgctactaccaagactgttcagcttgagtacaggagaggtaagtggaattcctagtgtagcggtggaatgcgtagatattaggaggaacaccggtggcgaaagcgacttactggcctgcaactgacactgaggcacgaaagcgtggggagcaaacagg

SEQ ID NO: 198 - DNA, 429 bp, Artificial Sequence
  Operational Taxonomic Unit (OTU) consensus sequence 207
tagggaatcttccgcaatggacgcaagtctgacggagcaaccccgcgtgagtgaagaaggttttcggatcgtaaaactctgttgttagagaagaatagggataagagtaactgcttatcttgtgacggtatctaacgaggaagccacggctaactacgtgccagcagccgcggtaatacgtaggtggcgagcgttgtccggaattattgggcgtaaagcgagcgcaggtggtcttttaagtctgatgtgaaatcccccggctcaactggggaaggtcattggaaactgggagacttgagtgcagaagaggaaagtggaattccatgtgtagcggtaaaatgcgtagatatatggaggaacaccagtggcgaaggcgactttctggtctgaaactgacactgaggctcgaaagcgtggggagcaaacagg

SEQ ID NO: 199 - DNA, 406 bp, Artificial Sequence
  Operational Taxonomic Unit (OTU) consensus sequence 209
tgggggatattgcacaatgggggaaaccctgatgcagcgatgccgcgtggaggaagaaggttttcggattgtaaactcctgtcgtaagggacgaagaagtgacggtaccttacaagaaagctccggctaactacgtgccagcagccgcggtaatacgtagggagcgagcgttgtccggaattactgggtgtaaagggagcgtaggcgggatggtaagtcagatgtgaaaactatgggctcaacccatagactgcatttgaaactgctgttcttgagtgaagtagaggtaagcggaattcctagtgtagcggtgaaatgcgtagatattaggaggaacatcggtggcgaaggcggcttactgggcttttactgacgctgaggctcgaaagcgtggggagcaaacagg

SEQ ID NO: 200 - DNA, 405 bp, Artificial Sequence
  Operational Taxonomic Unit (OTU) consensus sequence 210
tcgggaatattgcgcaatggaggaaactctgacgcagtgacgccgcgtgcaggaagaaggttttcggattgtaaactgctttagacagggaagagaaaggacagtacctgtagaataagctccggctaactacgtgccagcagccgcggtaatacgtagggagcgagcgttatccggatttattgggtgtaaagggtgcgtagacgggaatacaagttagttgtgaaatacctcggcttaactgaggaactgcaactaaaactatatttcttgagtacaggagaggtaagtggaattcctagtgtagcggtgaaatgcgtagatattaggaggaacaccagtggcgaaggcgacttactggactgaaactgacgttgaggcacgaaagtgtggggagcaaacagg

SEQ ID NO: 201 - DNA, 424 bp, Artificial Sequence
  Operational Taxonomic Unit (OTU) consensus sequence 211
tgaggaatattggtcaatggacggaagtctgaaccagccaagtagcgtgcaggatgacggccctctgggttgtaaactgcttttagttgggaataaagtgcggtacgtgtaccgttttgtatgtaccatcagaaaaaggaccggctaattccgtgccagcagccgcggtaatacggaaggtccaggcgttatccggatttattgggtttaaagggagcgcaggcggactcttaagtcagttgtgaaatacggcggctcaaccgtcggactgcagttgatactgggagtcttgagtacacgcagagatactggaattcatggtgtagcggtgaaatgctcagatatcatgaggaactccgatcgcgaaggcaggtatctggagtgtaactgacgctgaggctcgaaagtgcgggtatcaaacagg

SEQ ID NO: 202 - DNA, 429 bp, Artificial Sequence
  Operational Taxonomic Unit (OTU) consensus sequence 208
tagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtacgctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaacagg

SEQ ID NO: 203 - DNA, 404 bp, Artificial Sequence
  Operational Taxonomic Unit (OTU) consensus sequence 203
tggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggctttgcaagtctgacgtgaaactccggggctcaactccggaactgcgttggaaactgtaaggcttgagtgccggagaggtaagcggaattcctagtgtagcggtgaaatgcgtagatattaggaggaacaccagtggcgaaggcggcttactggacggcaactgacgttgaggctcgaaagcgtggggagcaaacagg

SEQ ID NO: 204 - DNA, 406 bp, Artificial Sequence
  Operational Taxonomic Unit (OTU) consensus sequence 216
tgggggatattgcacaatgggcgaaagcctgatgcagcgacgccgcgtgagggaagacggccttcgggttgtaaacctctgtcattcgggacgaatatatgacggtaccgaagaaggaagctccggctaactacgtgccagcagccgcggtaatacgtagggagcgagcgttgtccggaattactgggtgtaaagggagcgtaggcgggaaagcaagttggaagtgaaatgcatgggcttaacccatgagctgctttcaaaactgtttttcttgagtgaagtagaggcaggcggaattcctagtgtagcggtgaaatgcgtagatattaggaggaacaccagtggcgaaggcggcctgctgggctttaactgacgctgaggctcgaaagcgtgggtagcaaacagg

SEQ ID NO: 205 - DNA, 429 bp, Artificial Sequence
  Operational Taxonomic Unit (OTU) consensus sequence 217
tggggaatcttccgcaatggacgaaagtctgacggagcaacgccgcgtgagtgatgaaggtcttcggattgtaaaactctgttgttagggacgaaagcaccgtgttcgaacaggtcatggtgttgacggtacctaacgaggaagccacggctaactacgtgccagcagccgcggtaatacgtaggtggcaagcgttgtccggaattattgggcgtaaagagcatgtaggcgggcttttaagtctgacgtgaaaatgcggggcttaaccccgtatggcgttggatactggaagtcttgagtgcaggagaggaaaggggaattcccagtgtagcggtgaaatgcgtagatattgggaggaacaccagtggcgaaggcgcctttctggactgtgtctgacgctgagatgcgaaagccagggtagcaaacggg

SEQ ID NO: 206 - DNA, 405 bp, Artificial Sequence
  Operational Taxonomic Unit (OTU) consensus sequence 218
tcgggaatattgcgcaatggaggaaactctgacgcagtgacgccgcgtgcaggaagaaggttttcggattgtaaactgctttagacagggaagaacaaagacagtacctgtagaataagctccggctaactacgtgccagcagccgcggtaatacgtagggagcgagcgttatccggatttattgggtgtaaagggtgcgtagacgggaagtcaagttagttgtgaaatccctcggcttaactgaggaactgcaactaaaactgattttcttgagtactggagaggaaagtggaattcctagtgtagcggtgaaatgcgtagatattaggaggaacaccggtggcgaaggcgactttctggacagaaactgacgttgaggcacgaaagtgtggggagcaaacagg

SEQ ID NO: 207 - DNA, 429 bp, Artificial Sequence
  Operational Taxonomic Unit (OTU) consensus sequence 214
tggggaatcttccgcaatggacgaaagtctgacggagcaacgccgcgtgaacgatgaaggtcttcggattgtaaagttctgtgatccgggacgaaggcatcagttgagaacattgattgatgttgacggtaccggaaaagcaagccacggctaactacgtgccagcagccgcggtaatacgtaggtggcaagcgttgtccggaattattgggcgtaaagcgcgcgcaggcggccgtgcaagtccatcttaaaagcgtggggcttaaccccatgaggggatggaaactgcagggctggagtgtcggaggggaaagtggaattcctagtgtagcggtgaaatgcgtagagattaggaagaacaccggtggcgaaggcgactttctagacgacaactgacgctgaggcgcgaaagcgtggggagcaaacagg

SEQ ID NO: 208 - DNA, 429 bp, Artificial Sequence
  Operational Taxonomic Unit (OTU) consensus sequence 213
tggggaatcttccgcaatggacgaaagtctgacggagcaacgccgcgtgagtgatgacggccttcgggttgtaaagctctgttaatcgggacgaatggttcttgtgcgaatagtgcgaggatttgacggtaccggaatagaaagccacggctaactacgtgccagcagccgcggtaatacgtaggtggcaagcgttgtccggaattattgggcgtaaagcgcgcgcaggcggattggtcagtctgtcttaaaagttcggggcttaaccccgtgatgggatggaaactgccaatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacggg

SEQ ID NO: 209 - DNA, 407 bp, Artificial Sequence
  Operational Taxonomic Unit (OTU) consensus sequence 219
tggggaatattgggcaatgggcgaaagcctgacccagcaacgccgcgtgaaggaagaaggctttcgggttgtaaacttcttttatcagggacgaaggatgtgacggtacctgatgaataagccacggctaactacgtgccagcagccgcggtaatacgtaggtggcaagcgttgtccggatttactgggtgtaaagggcgcgtaggcggagagacaagtcagatgtgaaatctatgggcttaacccataaactgcatttgaaactatctcccttgagtgatggagaggcaagcggaattcctagtgtagcggtgaaatgcgtagatattaggaggaacaccagtggcgaaggcggcttgctggacattaactgacgctgaggcgcgaaagcgtggggagcaaacagg

SEQ ID NO: 210 - DNA, 429 bp, Artificial Sequence
  Operational Taxonomic Unit (OTU) consensus sequence 215
tggggaatcttccgcaatgggcgaaagcctgacggagcaacgccgcgtgagtgaagaaggtcttcggattgtaaagctctgttgtacatgacgaatgtgccggttgtgaataatggctggtaatgacggtagtgtacgaggaagccacggctaactacgtgccagcagccgcggtaatacgtaggtggcaagcgttgtccggaattattgggcgtaaagagcatgtaggcggcctattaagtcgggcgtgaaaatgcggggctcaaccccgtatggcgcccgatactggtgggcttgagtgcaggagaggaaaggggaattcccagtgtagcggtgaaatgcgtagatattgggaggaacaccagtggcgaaggcgcctttctggactgtgtctgacgctgagatgcgaaagccaggggagcgaacggg

SEQ ID NO: 211 - DNA, 424 bp, Artificial Sequence
  Operational Taxonomic Unit (OTU) consensus sequence 220
tgaggaatattggtcaatgggcgagagcctgaaccagccaagtcgcgtgaaggatgaaggatctatggttcgtaaacttcttttataagggaataaagtgcgggacgtgtcctgttttgtatgtaccttatgaataaggatcggctaactccgtgccagcagccgcggtaatacggaggatccgagcgttatccggatttattgggtttaaagggtgcgtaggtggtttattaagtcagcggtgaaagtttgtggctcaaccataaaattgccgttgaaactggttaacttgagtatatttgaggtaggcggaatgcgtggtgtagcggtgaaatgcatagatatcacgcagaactccaattgcgaaggcagcttactaaactataactgacactgaagcacgaaagcgtggggatcaaacagg

SEQ ID NO: 212 - DNA, 407 bp, Artificial Sequence
  Operational Taxonomic Unit (OTU) consensus sequence 222
tggggaatattgcacaatgggggaaaccctgatgcagcgacgccgcgtgagtgaagaagtatttcggtatgtaaagctctatcagcagggaagaaaacaatgacggtacctgactaagaagccccggctaactacgtgccagcagccgcggtaatacgtagggggcaagcgttatccggatttactgggtgtaaagggagcgtagacggtagaccaagtctgaagtgaaagcccggggctcaaccccggaactgctttggaaactggtaaactagagtgcaggagaggtaagtggaattcctagtgtagcggtgaaatgcgtagatattaggaggaacaccagtggcgaaggcggcttactggactgtaactgacgttgaggctcgaaagcgtggggagcaaacagg

SEQ ID NO: 213 - DNA, 429 bp, Artificial Sequence
  Operational Taxonomic Unit (OTU) consensus sequence 223
tagggaatcttcggcaatgggcgaaagcctgaccgagcaacgccgcgtgaatgatgaaggccttcgggttgtaaaattctgttataagggaagaacgactttagtaggaaatggctagagtgtgacggtaccttatgagaaagccacggctaactacgtgccagcagccgcggtaatacgtaggtggcgagcgttatccggaattattgggcgtaaagagcgcgcaggtggttgattaagtctgatgtgaaagcccacggcttaaccgtggagggtcattggaaactggtcgacttgagtgcagaagagggaagtggaattccatgtgtagcggtgaaatgcgtagagatatggaggaacaccagtggcgaaggcggcttcctggtctgtaactgacactgaggcgcgaaagcgtggggagcaaacagg

SEQ ID NO: 214 - DNA, 424 bp, Artificial Sequence
  Operational Taxonomic Unit (OTU) consensus sequence 227
tgaggaatattggtcaatgggcgcgagcctgaaccagccaagtagcgtgcaggaagacggccctatgggttgtaaactgcttttgcaggaggataatatgtcccacgtgtgggatattgcaggtatcctgcgaataaggaccggctaattccgtgccagcagccgcggtaatacggaaggtccgggcgttatccggatttattgggtttaaagggagcgtaggcgggagatcaagtcagttgtgaaaagcagccgctcaacggttgtcgtgcagttgatactggttttcttgagtgcgcgcgaggatggtggaatttgtggtgtagcggtgaaatgcttagatatcacaaagaactccgattgcgaaggcagctgtccggagcgcaactgacgctgaggctcgaaggtgcgggtatcaaacagg

SEQ ID NO: 215 - DNA, 428 bp, Artificial Sequence
  Operational Taxonomic Unit (OTU) consensus sequence 228
tagggaattttcggcaatgggggaaaccctgaccgagcaacgccgcgtgagggaagaagtatttcggtatgtaaacctctgttataaaggaagaacggtatgaataggaaatgattcataagtgacggtactttatgagaaagccacggctaactacgtgccagcagccgcggtaatacgtaggtggcgagcgttatccggaatcattgggcgtaaagagggagcaggcggcaatagaggtctgcggtgaaagcctgaagctaaacttcagtaagccgtggaaaccaaatagctagagtgcagtagaggatcgtggaattccatgtgtagcggtgaaatgcgtagatatatggaggaacaccagtggcgaaggcgacgatctgggctgcaactgacgctcagtcccgaaagcgtggggagcaaatagg

SEQ ID NO: 216 - DNA, 424 bp, Artificial Sequence
  Operational Taxonomic Unit (OTU) consensus sequence 225
tgaggaatattggtcaatggacggaagtctgaaccagccaagtagcgtgcaggatgacggccctatgggttgtaaactgcttttatgtgggaataaagtgagggacgtgtccctttttgtaggtaccacatgaataaggaccggctaattccgtgccagcagccgcggtaatacggaaggtccgggcgttatccggatttattgggtttaaagggagcgtaggccgtcttttaagcgtgttgtgaaatactgtcgctcaacgacagaggtgcagcgcgaactgggagacttgagtgcgcggaatgcaggcggaattcgtcgtgtagcggtgaaatgcttagatatgacgaagaactccgattgcgaaggcagcttgcagtagcgtaactgacgctgaagctcgaaagtgcgggtatcgaacagg

SEQ ID NO: 217 - DNA, 423 bp, Artificial Sequence
  Operational Taxonomic Unit (OTU) consensus sequence 224
tgaggaatattggtcaatggacgagagtctgaaccagccaagtagcgtgaaggacgactgccctatgggttgtaaacttcttttatatgggaataaaaaagtccacgtgtggattcttgtatgtaccatatgaataagcatcggctaattccgtgccagcagccgcggtaatacggaagatgcgagcgttatccggatttattgggtttaaagggagcgtaggccggcggttaagtcagcggtcaaattgggtggctcaaccatcccccgccgttgatactggccgccttgagtgtattcaaggcagatggaattcgtggtgtagcggtgaaatgcttagatatcacgaagaactccgattgcgaaggcagtctgctgggttacaactgacgctgaggctcgaaagtgcgggtatcaaacagg

SEQ ID NO: 218 - DNA, 404 bp, Artificial Sequence
  Operational Taxonomic Unit (OTU) consensus sequence 229
tggggaatattgcacaatgggggaaaccctgatgcagcgacgccgcgtgaaggaagaagtatttcggtatgtaaacttctatcagcagggaagaagatgacggtacctgagtaagaagcaccggctaaatacgtgccagcagccgcggtaatacgtatggtgcaagcgttatccggatttactgggtgtaaagggagcgtagacggataggcaagtctggagtgaaaacccagggctcaaccctgggactgctttggaaactgcagatctggagtgccggagaggtaagcggaattcctagtgtagcggtgaaatgcgtagatattaggaggaacaccagtggcgaaggcggcttactggacggtgactgacgttgaggctcgaaagcgtggggagcaaacagg

SEQ ID NO: 219 - DNA, 424 bp, Artificial Sequence
  Operational Taxonomic Unit (OTU) consensus sequence 230
tgaggaatattggtcaatgggcggaagcctgaaccagccatgccgcgtgaaggactaaggccctatgggtcgtaaacttctttagacgcagagcaataagggcctcgcgaggtccgatgagagtatgcgtagaataagcatcggctaactccgtgccagcagccgcggtaatacgggggatgcgagcgttatccggatttattgggtttaaagggtgcgtaggcggcgaattaagtcagcggtgaaagaccggggctcaaccctggaagtgccgttgatactgattggctagaatacccttgccgtgggaggaatgagtggtgtagcggtgaaatgcatagatatcactcagaacaccgattgcgaaggcatctcacgaaggggcgattgacgctgaggcacgaaagcgtggggatcgaacagg

SEQ ID NO: 220 - DNA, 407 bp, Artificial Sequence
  Operational Taxonomic Unit (OTU) consensus sequence 231
tggggaatattgggcaatggagggaactctgacccagcaatgccgcgtgagtgaagaaggttttcggattgtaaaactctttaagcagggacgaagaaagtgacggtacctgcagaataagcatcggctaactacgtgccagcagccgcggtaatacgtaggatgcaagcgttatccggaatgactgggcgtaaagggtgcgtaggcggtaaatcaagttggcagcgtaattccggggcttaactccggaactactgccaaaactggtgaactagagtgtgtcaggggtaagtggaattcctagtgtagcggtggaatgcgtagatattaggaggaacaccggaggcgaaagcgacttactggggcacaactgacgctgaggcacgaaagcgtggggagcaaacagg

SEQ ID NO: 221 - DNA, 404 bp, Artificial Sequence
  Operational Taxonomic Unit (OTU) consensus sequence 232
tggggaatattgggcaatgggcgaaagcctgacccagcaacgccgcgtgaaggaagaaggttttcggatcgtaaacttctatccttggtgaagataatgacggtagccaagaaggaagccccggctaactacgtgccagcagccgcggtaatacgtagggggcaagcgttgtccggaatgattgggcgtaaagggcgcgtaggcggccaactaagtctggagtgaaagtcctgcttttaaggtgggaattgctttggaaactggatggcttgagtgcaggagaggtaagcggaattcccggtgtagcggtgaaatgcgtagagatcgggaggaacaccagtggcgaaggcggcttactggactgtaactgacgctgaggcgcgaaagtgtggggagcaaacagg

SEQ ID NO: 222 - DNA, 429 bp, Artificial Sequence
  Operational Taxonomic Unit (OTU) consensus sequence 236
tggggaattttggacaatgggggcaaccctgatccagccatgccgcgtgcaggatgaaggccttcgggttgtaaactgcttttgttaggaacgaaacggtggatgttaataccatctactaatgacggtacctaaagaataagcaccggctaactacgtgccagcagccgcggtaatacgtagggtgcaagcgttaatcggaattactgggcgtaaagcgtgcgcaggcggcttgataagacaggtgtgaaatccccgagctcaacttgggaatagcacttgtgactgtcaggctagagtatgtcagagggaggtggaattccaagtgtagcagtgaaatgcgtagatatttggaagaacaccgatggcgaaggcagcctcctgggataatactgacgctcatgcacgaaagcgtggggagcaaacagg

SEQ ID NO: 223 - DNA, 424 bp, Artificial Sequence
  Operational Taxonomic Unit (OTU) consensus sequence 233
tgaggaatattggtcaatggacgcaagtctgaaccagccatgccgcgtgcaggatgaatgtgctatgcattgtaaactgcttttgtacgagggtaaaaacaggtacgtgtacctggttgaaagtatcgtacgaataagggtcggctaactccgtgccagcagccgcggtaatacggaggacccgagcgttatccggatttattgggtttaaagggtgcgtaggcggattggtaagttagaggtgaaagctcagcgcttaacgttgaaactgcctctgatactgtcggtctagagtatagttgcggaaggcggaatgtgtggtgtagcggtgaaatgcttagatatcacacagaacaccgattgcgaaggcagctttccaagctatcactgacgctgaggcacgaaagcgtggggagcgaacagg

SEQ ID NO: 224 - DNA, 404 bp, Artificial Sequence
  Operational Taxonomic Unit (OTU) consensus sequence 237
tggggaatattgcacaatggggggaaccctgatgcagcgatgccgcgtggaggaagaaggttttcggattgtaaactcctgtcgtaagggacgataatgacggtaccttacaagaaagctccggctaactacgtgccagcagccgcggtaatacgtagggagcgagcgttgtccggaattactgggtgtaaagggagcgtaggcgggacggcaagtcagatgtgaaatatacgtgctcaacatgtagactgcatttgaaactgtcgttcttgagtgaggtagaggtaagcggaattcctggtgtagcggtgaaatgcgtagagatcaggaggaacatcggtggcgaaggcggcttactgggcctttactgacgctgaggctcgaaagcgtggggagcaaacagg

SEQ ID NO: 225 - DNA, 404 bp, Artificial Sequence
  Operational Taxonomic Unit (OTU) consensus sequence 234
tgaggaatattgggcaatggaggcaactctgacccagccatgccgcgtgagtgaagaaggttttcggattgtaaagctctttcgggtgtgacgatgatgacggtagcacctaaagaagccccggctaacttcgtgccagcagccgcggtaatacgaagggggcaagcgttgttcggaattactgggcgtaaagggagtgtaggcggttatgtaagatagtggtgaaatcccagagcttaactttggaattgccattatgactatgtggctagaattacagagaggatagtggaatacccagtgtagaggtgaaattcgtagatattgggtagaacaccagtggcgaaggcgactatctggctgtatattgacgctgaggctcgaaagcatggggatcaaacagg

SEQ ID NO: 226 - DNA, 404 bp, Artificial Sequence
  Operational Taxonomic Unit (OTU) consensus sequence 241
tggggaatattgcacaatggggggaaccctgatgcagcgatgccgcgtggaggaagaaggttttcggattgtaaactccttttaacagggacgataatgacggtacctgaagaaaaagctccggctaactacgtgccagcagccgcggtaatacgtagggagcgagcgttgtccggaattactgggtgtaaagggagcgtaggcgggacggtaagtcaggtgtgaaatatacgtgctcaacatgtagactgcacttgaaactgctgttcttgagtgaagtagaggtaagcggaattcctagtgtagcggtgaaatgcgtagatattaggaggaacatcggtggcgaaggcggcttactgggcttttactgacgctgaggctcgaaagcgtggggagcaaacagg

SEQ ID NO: 227 - DNA, 423 bp, Artificial Sequence
  Operational Taxonomic Unit (OTU) consensus sequence 239
tgaggaatattggtcaatgggcgcaggcctgaaccagccaagtcgcgtgagggaagacggtcctacggattgtaaacctcttttgtcggggagtaacgtgcgggacgcgtcccgtattgagagtacccgaagaaaaagcatcggctaactccgtgccagcagccgcggtaatacggaggatgcgagcgttatccggatttattgggtttaaagggtgcgcaggcggcgcgccaagtcagcggtcaaagttccgggctcaacccggtgtcgccgttgaaactggcgtgctcgagtgcgtgcgaggaaggcggaatgcgttgtgtagcggtgaaatgcatagatatgacgcagaactccgattgcgaaggcagctttccagcgcgctactgacgctgaggcacgaaagcgtggggatcgaacagg

SEQ ID NO: 228 - DNA, 407 bp, Artificial Sequence
  Operational Taxonomic Unit (OTU) consensus sequence 243
tggggaatattgcgcaatgggggcaaccctgacgcagcaacgccgcgtgattgatgaaggtcttcggattgtaaaaatctttaatcagggacgaagaaaatgacggtacctgaagaataagctccggctaactacgtgccagcagccgcggtaatacgtagggagcaagcgttatccggatttactgggtgtaaagggcgtgtaggcgggcttgcaagttggaagtgaaatccaggggcttaacccctgaactgctttcaaaactgcgagtcttgagtgatggagaggcaggcggaattcccagtgtagcggtgaaatgcgtagatattgggaggaacaccagtggcgaaggcggcctgctggacattaactgacgctgaggcgcgaaagcgtggggagcaaacagg

SEQ ID NO: 229 - DNA, 407 bp, Artificial Sequence
  Operational Taxonomic Unit (OTU) consensus sequence 246
tggggaatattgggcaatgggcgaaagcctgacccagcgacgccgcgtgagggaagaaggtcttcggattgtaaaccttagttatcggggaagaagcaagtgacggtacccgaagagaaagccacggctaactacgtgccagcagccgcggtaatacgtaggtggcgagcgttatccggaattactgggtgtaaagggtgtgtaggcgggatagcaagtcagatgtgaaaattatgggcttaacccataacctgcatttgaaactgttattcttgagtgtcggagaggtaaatggaattcccggtgtagcggtgaaatgcgtagatatcgggaggaacaccagtggcgaaggcggtttactggacgacaactgacgctgagacacgaaagcgtggggagcaaacagg

SEQ ID NO: 230 - DNA, 429 bp, Artificial Sequence
  Operational Taxonomic Unit (OTU) consensus sequence 247
tagggaatcttcggcaatgggggcaaccctgaccgagcaacgccgcgtgagtgaagaaggttttcggatcgtaaagctctgttgttagagaagaacgttggtgggagtggaaaatccatcaagtgacggtaactaaccagaaagggacggctaactacgtgccagcagccgcggtaatacgtaggtcccgagcgttgtccggatttattgggcgtaaagcgagcgcaggcggtttcgtaagtctgaagttaaaggcagtggcttaaccattgttcgctttggaaactgcgagacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacagg

SEQ ID NO: 231 - DNA, 428 bp, Artificial Sequence
  Operational Taxonomic Unit (OTU) consensus sequence 240
tagggaattttcgtcaatggggggaaccctgaacgagcaatgccgcgtgagtgaagaaggtcttcggatcgtaaagctctgttgtaagtgaagaacggtcagtagaggaaatgatactgaagtgacggtagcttaccagaaagccacggctaactacgtgccagcagccgcggtaatacgtaggtggcgagcgttatccggaatcattgggcgtaaagggtgcgcaggtggtacattaagtccgaagtaaaaggcagcagctcaactgctgttggctttggaaactggtgaactggagtgcaggagagggcgatggaattccatgtgtagcggtaaaatgcgtagatatatggaggaacaccagtggcgaaggcggtcgcctggcctgcaactgacactgaggcacgaaagcgtggggagcaaatagg

SEQ ID NO: 232 - DNA, 429 bp, Artificial Sequence
  Operational Taxonomic Unit (OTU) consensus sequence 244
tggggaatattgcacaatgggcgcaagcctgatgcagccatgccgcgtgtatgaagaaggccttcgggttgtaaagtactttcagcgaggaggaaggcattaaggttaataaccttagtgattgacgttactcgcagaagaagcaccggctaactccgtgccagcagccgcggtaatacggagggtgcaagcgttaatcggaattactgggcgtaaagcgcacgcaggcggtctgttaagtcagatgtgaaatccccgggctcaacctgggaactgcatttgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 233 - DNA, 404 bp, Artificial Sequence
  Operational Taxonomic Unit (OTU) consensus sequence 242
tggggaatattggacaatgggggaaaccctgatccagcgacgccgcgtgagtgaagaagtatttcggtatgtaaagctctatcagcagggaagataatgacagtacctgactaagaagccccggctaactacgtgccagcagccgcggtaatacgtagggggcaagcgttatccggatttactgggtgtaaagggagcgtaggtggcatggcaagtcagaagtgaaagcccagggctcaaccctgggactgcttttgaaactgtcaagctagagtgcaggagaggtaagtggaattcctagtgtagcggtgaaatgcgtagatattaggaggaacaccagtggcgaaggcggcttactggactgtaactgacactgaggctcgaaagcgtggggagcaaacagg

SEQ ID NO: 234 - DNA, 404 bp, Artificial Sequence
  Operational Taxonomic Unit (OTU) consensus sequence 248
tggggaatattggacaatgggggagaccctgatccagccatgccgcgtgagtgaagacggccttcgggttgtaaagctcttttacatgggaagatgatgacggtaccatgagaataagcaccggcaaacttcgtgccagcagccgcggtaatacgaagggtgcaagcgttgttcggaattactgggtgtaaagggcgtgtaggctggcgatcaagttagtggtgaaacccctgggcttaacctgggacctgccattgatactgatagcctggagtatcggagaggataacggaatatccagtgtagaggtgaaattcgtagatattggatagaacaccggtggcgaaggcggttatctggccggttactgacgctgaggcgcgagagcgtggggagcaaacagg

SEQ ID NO: 235 - DNA, 424 bp, Artificial Sequence
  Operational Taxonomic Unit (OTU) consensus sequence 250
tgaggaatattggtcaatgggcgcaggcctgaaccagccaagtagcgtgaaggatgactgccctatgggttgtaaacttcttttatatgggaataaagttttccacgtgtggaattttgtatgtaccatatgaataaggatcggctaactccgtgccagcagccgcggtaatacggaggatccgagcgttatccggagttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacagg

SEQ ID NO: 236 - DNA, 425 bp, Artificial Sequence
  Operational Taxonomic Unit (OTU) consensus sequence 254
tgaggaatattggtcaatggacggaggtctgaaccagccaagtagcgtgcaggattgacggccctatgggttgtaaactgcttttgttggggagtaaagttgggcacgcgtgcctttttgcatttacccttcgaataaggaccggctaattccgtgccagcagccgcggtaatacggaaggtccaggcgttatccggatttattgggtttaaagggagtgtaggcggtctgttaagcgtgttgtgaaatttaggtgctcaacatctaccttgcagcgcgaactggcggacttgagtgcacgcaacgtatgcggaattcatggtgtagcggtgaaatgcttagatatcatgacgaactccgattgcgaaggcagcgtacgggagtgttactgacgcttaagctcgaaggtgcgggtatcgaacagg

SEQ ID NO: 237 - DNA, 406 bp, Artificial Sequence
  Operational Taxonomic Unit (OTU) consensus sequence 252
tggggaatattgcacaatgggcgcaagcctgatgcagcgacgccgcgtgaaggatgaaggtcttcggatcgtaaacttctatcagcagggaagaaaccatgacggtacctgactaagaagccccggctaactacgtgccagcagccgcggtaatacgtagggggcaagcgttatccggaattactgggtgtaaagggtgcgtaggcggcgatttaagtcagatgtgaaaactcagggctcaaccttgagactgcatctgaaactgagttgctagagtgcaggagaggaaagcggaattccgagtgtagcggtgaaatgcgtagagattcggaggaacaccagtagcgaaggcggctttctggactgtaactgacgctgaggcacgaaagcgtggggagcgaacagg

SEQ ID NO: 238 - DNA, 407 bp, Artificial Sequence
  Operational Taxonomic Unit (OTU) consensus sequence 253
tggggaatattgggcaatggacgaaagtctgacccagcgacgccgcgtgagggaagaaggtcttcggattgtaaaccttagtcaacagggaagaagaaagtgacggtacctgtggaggaagccacggctaactacgtgccagcagccgcggtaatacgtaggtggcgagcgttatccggatttactgggtgtaaagggtgtgtaggcgggaaggcaagtcagatgtgaaaactatgggctcaacccatagcctgcatttgaaactgtttttcttgagagtcggagaggtaagtggaattcccggtgtagcggtgaaatgcgtagatatcgggaggaacatctgtggcgaaggcgacttactggacgattactgacgctgagacacgaaagcgtggggagcaaacagg

SEQ ID NO: 239 - DNA, 424 bp, Artificial Sequence
  Operational Taxonomic Unit (OTU) consensus sequence 255
tgaggaatattggtcaatgggcggaagcctgaaccagccaagtagcgtgaaggatgactgccctatgggttgtaaacttcttttataggggaataaaatgagccacgtgtggctttttgtatgtaccctatgaataaggatcggctaactccgtgccagcagccgcggtaatacggaggatccgagcgttatccggatttattgggtttaaagggagcgtaggtggacatgtaagtcagttgtgaaagtttgcggctcaaccgtaaaattgcagttgatactgtgtgtcttgagtacagtagaggtgggcggaattcgtggtgtagcggtgaaatgcttagatatcacgaagaactccgattgcgaaggcagctcactggactgttactgacactgaggctcgaaagtgtgggtatcaaacagg

SEQ ID NO: 240 - DNA, 424 bp, Artificial Sequence
  Operational Taxonomic Unit (OTU) consensus sequence 258
tgaggaatattggtcaatgggcgagagcctgaaccagccaagtagcgtgtgggacgaatgccctatgggttgtaaaccacttttgcaggagggtaaaatgcttcacgtgtggagtattgcaagtatcctgcgaataaggaccggctaattccgtgccagcagccgcggtaatacggaaggtccgggcgttatccggatttattgggtttaaagggagcgtaggcggggagtcaagtcagttgtgaaaagccgcggcccaaccgtggtcgtgcagttgaaactggttctcttgagtgcgcacgaggacggtggaattcgtggtgtagcggtgaaatgcttagatatcacgaagaactccgattgcgaaggcagccgtccggagcgttactgacgctgaggctcgaaggtgcgggtatcaaacagg

SEQ ID NO: 241 - DNA, 428 bp, Artificial Sequence
  Operational Taxonomic Unit (OTU) consensus sequence 260
tagggaattttcggcaatgggcgaaagcctgaccgagcaacgccgcgtgagtgaagaaggccttcgggttgtaaagctctgttgtgaaggaagaacggctcatacagggaatggtatgggagtgacggtactttaccagaaagccacggctaactacgtgccagcagccgcggtaatacgtaggtggcgagcgttatccggaattattgggcgtaaagggtgcgcaggcggtttgttaagtttaaggtgaaagcgtggggcttaaccccatatagccttagaaactgacagactagagtacaggagagggcaatggaattccatgtgtagcggtaaaatgcgtagatatatggaggaacaccagtggcgaaggcggttgcctggcctgtaactgacgctcatgcacgaaagcgtggggagcaaatagg

SEQ ID NO: 242 - DNA, 424 bp, Artificial Sequence
  Operational Taxonomic Unit (OTU) consensus sequence 263
tgaggaatattggtcaatgggcggtagcctgaaccagccaagtagcgtgaaggatgaaggttctatggattgtaaacttcttttataaaggaataaagtgaggcacgtgtgcctttttgtatgtactttatgaataaggatcggctaactccgtgccagcagccgcggtaatacggaggatccgagcgttatccggatttattgggtttaaagggagcgtagatgggttgttaagtcagttgtgaaagtttgcggctcaaccgtaaaattgcaattgatactggcgtccttgagtacagttgaggtgggcggaattcgtggtgtagcggtgaaatgcttagatatcacgaagaactcctattgcgaaggcagctcactaaactgcaactgacattgaggctcgaaagtgtgggtatcaaacagg

SEQ ID NO: 243 - DNA, 429 bp, Artificial Sequence
  Operational Taxonomic Unit (OTU) consensus sequence 268
tagggaatcttccacaatggacgcaagtctgatggagcaacgccgcgtgagtgaagaaggtcttcggatcgtaaaactctgttgttagagaagaacacgagtgagagtaactgttcattcgatgacggtatctaaccagcaagtcacggctaactacgtgccagcagccgcggtaatacgtaggtggcaagcgttgtccggatttattgggcgtaaagggaacgcaggcggtcttttaagtctgatgtgaaagccttcggcttaaccggagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacagg

SEQ ID NO: 244 - DNA, 429 bp, Artificial Sequence
  Operational Taxonomic Unit (OTU) consensus sequence 272
tggggaatcttccgcaatggacgaaagtctgacggagcaacgccgcgtgagtgatgaaggccttcgggttgtaaaactctgttgtcagggacgaacgtgctgatttacaatacacttcagcagtgacggtacctgacgaggaagccacggctaactacgtgccagcagccgcggtaatacgtaggtggcaagcgttgtccggaattattgggcgtaaagagcatgtaggcgggcttttaagtccgacgtgaaaatgcggggcttaaccccgtatggcgttggatactggaagtcttgagtgcaggagaggaaaggggaattcccagtgtagcggtgaaatgcgtagatattgggaggaacaccagtggcgaaggcgcctttctggactgtgtctgacgctgagatgcgaaagccagggtagcaaacggg

SEQ ID NO: 245 - DNA, 405 bp, Artificial Sequence
  Operational Taxonomic Unit (OTU) consensus sequence 276
tggggaatattgcacaatgggggaaaccctgatgcagcgacgccgcgtgaaggaagaagtatctcggtatgtaaacttctatcagcagggaagaattaggacggtacctgactaagaagccccggctaactacgtgccagcagccgcggtaatacgtagggggcaagcgttatccggatttactgggtgtaaagggagcgtagacggatggacaagtctgatgtgaaaggctggggctcaaccccgggactgcattggaaactgcccgtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 246 - DNA, 404 bp, Artificial Sequence
  Operational Taxonomic Unit (OTU) consensus sequence 277
tggggaatattgcacaatgggggaaaccctgatgcagcaacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgcaggcggtctggcaagtctgatgtgaaatcccggggctcaaccttggaactgcattggaaactgtcagactagagtgccggagaggtaagtggaattcctagtgtagcggtgaaatgcgtagatattaggaggaacaccagtggcgaaggcggcttactggacggtaactgacgctgaggctcgaaagcgtggggagcaaacagg

SEQ ID NO: 247 - DNA, 405 bp, Artificial Sequence
  Operational Taxonomic Unit (OTU) consensus sequence 267
tagggaattttgcgcaatgggcgaaagcctgacgcagcaacgccgcgtgattgataaagcccttcggggtgtaaagatctgtcagtggggacgaaacttgacggtacccacagaggaagcaccggctaactccgtgccagcagccgcggtaatacggagggtgcaagcgttgtccggaatcattgggcgtaaagagttcgtaggtggtttgttaagtttggtgttaaatgcagaggctcaacttctgttcggcatcggatactggcagactagaatgcggtagaggtaaagggaattcctggtgtagcggtgaaatgcgtagatatcaggaggaacatcggtggcgtaagcgctttactgggccgtaattgacactgaggaacgaaagccagggtagcaaatggg

SEQ ID NO: 248 - DNA, 429 bp, Artificial Sequence
  Operational Taxonomic Unit (OTU) consensus sequence 279
tggggaattttggacaatgggggcaaccctgatccagccatgccgcgtgcgggaagaaggccttcgggttgtaaaccgcttttgtcagggacgaaaagctccggagtaatatgccggagtgctgacggtacctgaagaataagcaccggctaactacgtgccagcagccgcggtaatacgtagggtgcgagcgttaatcggaattactgggcgtaaagcgtgcgcaggcggttgggtaagacagatgtgaaatccccgggcttaacctgggaactgcatttgtgactgtccgactggagtacgtcagaggggggtggaattccacgtgtagcagtgaaatgcgtagatatgtggaagaacaccgatggcgaaggcagccccctgggacgcaactgacgctcatgcacgaaagcgtggggagcaaacagg

SEQ ID NO: 249 - DNA, 424 bp, Artificial Sequence
  Operational Taxonomic Unit (OTU) consensus sequence 295
tgaggaatattggtcaatggacgaaagtctgaaccagccaagtagcgtgcaggaagacggccctctgggttgtaaactgcttttagttgggaataaaacgcggtacgtgtaccgccttgtatgtaccatcagaaaaaggaccggctaattccgtgccagcagccgcggtaatacggaaggttcgggcgttatccggatttattgggtttaaagggagcgcaggcggacttttaagtcagctgtgaaatctggcggctcaaccgtcagactgcagttgatactggaagtcttgagtgcacacagggatgctggaattcatggtgtagcggtgaaatgctcagatatcatgaagaactccaatcgcgaaggcaggcatccggggtgcaactgacgctgaggctcgaaagtgcgggtatcaaacagg

SEQ ID NO: 250 - DNA, 425 bp, Artificial Sequence
  Operational Taxonomic Unit (OTU) consensus sequence 300
tgaggaatattggtcaatgggcggaagcctgaaccagccatgccgcgtgcgggaaggaggccctatgggctgtgaaccgcttttgcctgggggcaataagggcgtcgcgcacgtccgatgagagtaccaggcgaataagcatcggctaactccgtgccagcagccgcggtaatacgggggatgcgagcgttatccggattcattgggtttaaagggtgcgtaggctgtgcgtcaagtcgggggtgaaattccggtgctcaacaccggggctgcccttgatactgtcgcgctggagtgcggatgccgccggaggaatgagtggtgtagcggtgaaatgcttagatatcactcagaacaccgattgcgaaggcatctggcgaatccgtaactgacgctgaggcacgaaagcgtggggatagaacagg

SEQ ID NO: 251 - DNA, 407 bp, Artificial Sequence
  Operational Taxonomic Unit (OTU) consensus sequence 307
tggggaatattgcacaatgggggaaaccctgatgcagcaacgccgcgtgaaggaagaaggttttcggatcgtaaacttctatcaatagggacgaacaaatgacggtacctaaataagaagccccggctaactacgtgccagcagccgcggtaatacgtagggggcaagcgttatccggaattactgggtgtaaagggagcgtaggcggcatggtaagtaagatgtgaaagcccgaggcttaacctcgaggattgcattttaaactatcaagctagagtacaggagaggaaagcggaattcctagtgtagcggtgaaatgcgtagatattaggaagaacaccagtggcgaaggcggctttctggactgaaactgacgctgaggctcgaaagcgtggggagcgaacagg

SEQ ID NO: 252 - DNA, 424 bp, Artificial Sequence
  Operational Taxonomic Unit (OTU) consensus sequence 302
tgaggaatattggtcaatgggcgagagcctgaaccagccaagtagcgtgagggatgaccgccctacgggtcgtaaacctcttttataagggaataaagataagtacgcgtacttagttgcatgtaccttatgaataagcatcggctaactccgtgccagcagccgcggtaatacggaggatgcgagcgttatccggatttattgggtttaaagggagcgcagacgggactttaagtcagctgtgaaattttccggctcaaccgggaaactgcagttgatactggcgtccttgagtacggtcgaggcaggcggaattcgtggtgtagcggtgaaatgcttagatatcacgaagaaccccgattgcgaaggcagcctgccagaccgcaactgacgttcatgctcgaaagtgcgggtatcaaacagg

SEQ ID NO: 253 - DNA, 429 bp, Artificial Sequence
  Operational Taxonomic Unit (OTU) consensus sequence 314
tggggaatcttccgcaatggacgaaagtctgacggagcaacgccgcgtgagtgatgacggccttcgggttgtaaagctctgttaatcgggacgaatggtctttgtgtgaataatgcaaagatttgacggtaccggaatagaaagccacggctaactacgtgccagcagccgcggtaatacgtaggtggcaagcgttgtccggaattattgggcgtaaagcgcgcgcaggcggtttcataagtctgtcttaaaagtgcggggcttaaccccgtgaggggatggaaactatggaactggagtatcggagaggaaagcggaattcctagtgtagcggtgaaatgcgtagatattaggaagaacaccagtggcgaaggcggctttctggacgacaactgacgctgaggcgcgaaagccaggggagcgaacggg

SEQ ID NO: 254 - DNA, 424 bp, Artificial Sequence
  Operational Taxonomic Unit (OTU) consensus sequence 322
tgaggaatattggtcaatgggcgagagcctgaaccagccaagtagcgtgaaggatgaaggctctatgggtcgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 255 - DNA, 404 bp, Artificial Sequence
  Operational Taxonomic Unit (OTU) consensus sequence 325
tggggaatattgcacaatgggggaaaccctgatgcagcgacgccgcgtgagtgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggaacagcaagtctgatgtgaaaacccggggctcaaccccgggactgcattggaaactgttgatctagagtgtcggagaggtaagtggaattcctagtgtagcggtgaaatgcgtagatattaggaggaacaccagtggcgaaggcggcttactggacgatgactgacgttgaggctcgaaagcgtggggagcaaacagg

SEQ ID NO: 256 - DNA, 424 bp, Artificial Sequence
  Operational Taxonomic Unit (OTU) consensus sequence 326
tgaggaatattggtcaatggacgcaagtctgaaccagccaagtagcgtgcaggacgacggccctccgggttgtaaactgcttttagttgggaataaagtgcagctcgtgagctgttttgtatgtaccatcagaaaaaggaccggctaattccgtgccagcagccgcggtaatacggaaggtccgggcgttatccggatttattgggtttaaagggagcgcaggcggactcttaagtcagttgtgaaatacggcggctcaaccgtcggactgcagttgatactgggagtcttgagtgcacacagggatgctggaattcatggtgtagcggtgaaatgctcagatatcatgaagaactccgatcgcgaaggcaggtatccggggtgcaactgacgctgaggctcgaaagtgcgggtatcaaacagg

SEQ ID NO: 257 - DNA, 424 bp, Artificial Sequence
  Operational Taxonomic Unit (OTU) consensus sequence 332
tgaggaatattggtcaatggacgcgagtctgaaccagccaagtagcgtgaaggatgactgccctatgggttgtaaacttcttttatatgggaataaagttgtccacgtgtggatttttgtatgtaccatatgaataaggatcggctaactccgtgccagcagccgcggtaatacggaggatccgagcgttatccggagttattgggtttaaagggagcgtaggcggattgttaagtcagttgtgaaagtttgcggctcaaccgtaaaattgcagttgatactggcagtcttgagtgcagtagaggtgggcggaattcgtggtgtagcggtgaaatgcttagatatcacgaagaactccgattgcgaaggcagctcactggagtgtaactgacgctgatgctcgaaagtgtgggtatcaaacagg

SEQ ID NO: 258 - DNA, 404 bp, Artificial Sequence
  Operational Taxonomic Unit (OTU) consensus sequence 336
tagggaatattgcacaatgggggaaaccctgatgcagcgacgccgcgtgaaggaagaagtatctcggtatgtaaacttctatcagcagggaagacaatgacggtacctgactaagaagccccggctaactacgtgccagcagccgcggtaatacgtagggggcaagcgttatccggatttactgggtgtaaagggagcgtagacggacgggcaagtctgaagtgaaaggcaggggctcaactcctggactgctttggaaactgtccatctagagtgccggagaggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 259 - DNA, 424 bp, Artificial Sequence
  Operational Taxonomic Unit (OTU) consensus sequence 339
tgaggaatattggtcaatggacgagagtctgaaccagccaagtagcgtgcaggaggacggccctatgggttgtaaactgctttagtatgggaataaagtcatccacgtgtggatgtttgcatgtaccataagaataaggaccggctaattccgtgccagcagccgcggtaatacggaaggtccgggcgttatccggatttattgggtttaaagggagcgtaggcggatttttaagtcagttgtgaaagttcacggcccaaccgtgaaattgcagttgaaactgaaagtcttgagtgcacgcagggatgctggaattcgtggtgtagcggtgaaatgcttagatatcacgaagaactccgatcgcgaaggcatgtgtccggagtgcaactgacgctgaggctcgaaagtgtgggtatcaaacagg

SEQ ID NO: 260 - DNA, 424 bp, Artificial Sequence
  Operational Taxonomic Unit (OTU) consensus sequence 343
tgaggaatattggtcaatgggcgagagcctgaaccagccaagtagcgtgaaggaagactgccctatgggttgtaaacttcttttatacgggaataaagtcatccacgtgtggatgtttgtatgtaccgtatgaataaggatcggctaactccgtgccagcagccgcggtaatacggaggatccgagcgttatccggatttattgggtttaaagggagcgtaggcgggcttttaagtcagttgtgaaagtttgcggctcaaccgtaaaattgcagttgatactggaagccttgagtacagtataggcaggcggaattcgtggtgtagcggtgaaatgcttagatatcacgaagaactccgattgcgaaggcagcttgctggactgtaactgacgctgatgctcgaaagtgtgggtatcaaacagg

SEQ ID NO: 261 - DNA, 424 bp, Artificial Sequence
  Operational Taxonomic Unit (OTU) consensus sequence 358
tgaggaatattggtcaatgggcggaagcctgaaccagccaagtagcgtgcgggacgacggccctatgggttgtaaaccgctttttcacggggataaagggcgtcacgtgtggcgctttgcaggtaccgtgcgaataaggaccggctaattccgtgccagcagccgcggtaatacggaaggtccgggcgttatccggaatcattgggtttaaagggagcgtaggccgcatgtcaagcgtgctgtgaaatcccggggctcaaccccggaagcgcagcgcgaactggcgtgcttgagttgcatcgaggcaggcggaattcgtggtgtagcggtgaaatgcttagatatcacgaagaaccccgattgcgaaggcagcctgccagttgcacactgacgctgatgctcgaaggcgcgggtatcgaacagg

SEQ ID NO: 262 - DNA, 423 bp, Artificial Sequence
  Operational Taxonomic Unit (OTU) consensus sequence 369
tgaggaatattggtcaatgggcgggagcctgaaccagccaagtagcgtgaaggacgacggccctaggggttgtaaacttcttttataagggaataaagtgcgttacgtgtaatgttttgtatgtaccttatgaataagcatcggctaattccgtgccagcagccgcggtaatacggaagatgcgagcgttatccggatttattgggtttaaagggagcgtaggcgggcttttaagtcagcggtcaaatgtcgtggctcaaccatgtcaagccgttgaaactgcaagccttgagtctgcacagggcacatggaattcgtggtgtagcggtgaaatgcttagatatcacgaagaactccgatcgcgaaggcattgtgccggggcataactgacgctgaggctcgaaagtgcgggtatcaaacagg

SEQ ID NO: 263 - DNA, 424 bp, Artificial Sequence
  Operational Taxonomic Unit (OTU) consensus sequence 373
tgaggaatattggtcaatggacgagagtctgaaccagccaagtagcgtgcaggaagacggccctatgggttgtaaactgcttttataagggaataaagtgggagtcgtgactccttttgcatgtaccttatgaataaggatcggctaattccgtgccagcagccgcggtaatacggaaggtccgggcgttatccggatttattgggtttaaagggagcgtaggccggagattaagcgtgttgtgaaatgtagatgctcaacatctgaactgcagcgcgaactggtttccttgagtacgcacaaagtgggcggaattcgtggtgtagcggtgaaatgcttagatatcacgaagaactccgattgcgaaggcagctcactggagcgcaactgacgctgaagctcgaaagtgcgggtatcgaacagg

SEQ ID NO: 264 - DNA, 424 bp, Artificial Sequence
  Operational Taxonomic Unit (OTU) consensus sequence 380
tgaggaatattggtcaatgggcgaaagcctgaaccagccaagtcgcgtggaggaagacggccctacgggttgtaaacttcttttacctgggaataacgggcgctacgtgtagcgctgtgcatgtaccaggcgaataagcatcggctaattccgtgccagcagccgcggtaatacggaagatgcgagcgttatccggatttattgggtttaaagggtgcgtaggcggaaggataagtcagcggtgaaatgcttcagctcaactggagaattgccgatgaaactgtttttctagagtataaaagaggtatgcggaatgcgtggtgtagcggtgaaatgcatagatatcacgcagaaccccgattgcgaaggcagcatactgggctataactgacgctgaagcacgaaagcgtgggtatcgaacagg

SEQ ID NO: 265 - DNA, 423 bp, Artificial Sequence
  Operational Taxonomic Unit (OTU) consensus sequence 388
tgaggaatattggtcaatgggcgagagcctgaaccagccaagtagcgtggaggatgaatgccctacgggttgtaaactccttttggcggaggataaagattgccacgtgtggcaagctgcaggtatccgccgaataagggccggctaattccgtgccagcagccgcggtaatacggaaggcccgagcgttatccggatttattgggtttaaagggagcgtaggcgggagatcaagtcagctgtgaaactgcgccgctcaacggcgccgagcagttgaaactggtttccttgagtccgcaagaggcgcgtggaattcgtggtgtagcggtgaaatgcatagatatcacgaagaactccgattgcgaaggcagcgcgctggggcgtcactgacgctgaagctcgaaggtgcgggtatcgaacagg

SEQ ID NO: 266 - DNA, 424 bp, Artificial Sequence
  Operational Taxonomic Unit (OTU) consensus sequence 402
tgaggaatattggtcaatgggcgagagcctgaaccagccaagtagcgtgcaggatgacggccctatgggttgtaaactgcttttatacggggataaagtggcgaacgtgtttgctattgcaggtaccgtatgaataaggaccggctaattccgtgccagcagccgcggtaatacggaaggtccaggcgttatccggatttattgggtttaaagggagcgtaggccgctgattaagcgtgttgtgaaatttggatgctcaacatctgaactgcagcgcgaactggttagcttgagtgtgcgcaacgcaggcggaatttgtggtgtagcggtgaaatgcttagatatcacgaagaactccgattgcgaaggcagcttgcgggagcacaactgacgctgaagctcgaaagcgcgggtatcgaacagg

SEQ ID NO: 267 - DNA, 424 bp, Artificial Sequence
  Operational Taxonomic Unit (OTU) consensus sequence 397
tgaggaatattggtcaatgggcgagagcctgaaccagccaagtagcgtgaaggatgaaggttctatggattgtaaacttcttttatacgggaataaaacctcccacgtgtgggagcttgtatgtaccgtatgaataagcatcggctaactccgtgccagcagccgcggtaatacggaggatgcgagcgttatccggatttattgggtttaaagggagcgcagacgggatgttaagtcagctgtgaaagtttgcggctcaaccgtaaaattgcagttgatactggcgtccttgagtgcggttgaggtgtgcggaattcgtggtgtagcggtgaaatgcttagatatcacgaagaaccccgattgcgaaggcagcacactaagccgtaactgacgttcatgctcgaaagtgtgggtatcaaacagg

SEQ ID NO: 268 - DNA, 429 bp, Artificial Sequence
  Operational Taxonomic Unit (OTU) consensus sequence 417
tggggaatattgcgcaatgggcgaaagcctgacgcagcgacgccgcgtgagggatgaaggtcttcggatcgtaaacctctgtcagaagggaagaacaagcactgcgctaatcaacagtgccctgacggtaccttcaaaggaagcaccggctaactccgtgccagcagccgcggtaatacggagggtgcaagcgttaatcggaattactgggcgtaaagcgcatgtaggctgtatggcaagttgggggtgaaatcccacggctcaaccgtggaactgccttcaaaactaccaaactagagtgcgagagaggatagcggaattccaggtgtaggagtgaaatccgtagatatctggaagaacatcagtggcgaaggcggctatctggctcgtaactgacgctgagatgcgaaagcgtgggtagcaaacagg

SEQ ID NO: 269 - DNA, 406 bp, Artificial Sequence
  Operational Taxonomic Unit (OTU) consensus sequence 458
tggggaatattgcacaatgggggaaaccctgatgcagcgacgccgcgtgagcgaagaagtatttcggtatgtaaagctctatcagcagggaagaagaaatgacggtacctgactaagaagcaccggctaaatacgtgccagcagccgcggtaatacgtatggtgcaagcgttatccggatttactgggtgtaaagggagcgcaggcggtacggcaagtctgatgtgaaagcccggggctcaaccccggtactgcattggaaactgtcggactagagtgtcggaggggtaagtggaattcctagtgtagcggtgaaatgcgtagatattaggaggaacaccagtggcgaaggcggcctactgggcaccaactgacgctgaggctcgaaagtgtgggtagcaaacagg

SEQ ID NO: 270 - DNA, 424 bp, Artificial Sequence
  Operational Taxonomic Unit (OTU) consensus sequence 497
tgaggaatattggtcaatgggcggaagcctgaaccagccaagtagcgtgcaggacgacggccctatgggttgtaaactgcttttgcagggggataaagtgagtcacgtgtgacttattgcaggtaccctgcgaataaggaccggctaattccgtgccagcagccgcggtaatacggaaggtccgggcgttatccggatttattgggtttaaagggagcgtaggccgtggattaagtgtgttgtgaaatgtaggcgctcaacgtctgacttgcagcgcatactggttcactagagtgcgcgcaacgcgggcggaatttgtcgtgtagcggtgaaatgcttagatatgacgaagaaccccgattgcgaaggcagctcgcgggagcgcaactgacgctgaagctcgaaagtgcgggtatcgaacagg

SEQ ID NO: 271 - DNA, 404 bp, Artificial Sequence
  Operational Taxonomic Unit (OTU) consensus sequence 500
tggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaagaacaccagtggcgaaggcggcttgctggacagtaactgacgttcaggctcgaaagcgtggggagcaaacagg

SEQ ID NO: 272 - DNA, 404 bp, Artificial Sequence
  Operational Taxonomic Unit (OTU) consensus sequence 517
tggggaatattgcacaatgggcgaaagcctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggctgtgcaagtctgaagtgaaaggcatgggctcaacctgtggactgctttggaaactgtgcggctagagtgtcggagaggtaagtggaattcctagtgtagcggtgaaatgcgtagatattaggaggaacaccagtggcgaaggcggcttactggacgatgactgacgttgaggctcgaaagcgtggggagcaaacagg

SEQ ID NO: 273 - DNA, 424 bp, Artificial Sequence
  Operational Taxonomic Unit (OTU) consensus sequence 522
tgaggaatattggtcaatgggcgcgagcctgaaccagccaagtagcgtgaaggatgactgccctatgggttgtaaacttcttttatattagaataaagtgcagtatgtatactgttttgtatgtataatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 274 - DNA, 404 bp, Artificial Sequence
  Operational Taxonomic Unit (OTU) consensus sequence 562
tggggaatattgcacaatgggggaaaccctgatgcagcaatgccgcgtgagtgatgacggccttcgggttgtaaagctctgtcttcagggacgataatgacggtacctgaggaggaagccacggctaactacgtgccagcagccgcggtaatacgtaggtggcaagcgttgtccggatttactgggcgtaaagggagcgtaggcggatttttaagtgggatgtgaaatacccgggcttaacctgggtgctgcattccaaactggaaatctagagtgcaggaggggaaagtggaattcctagtgtagcggtgaaatgcgtagagattaggaagaacaccggtggcgaaggcgactttctggactgtaactgacgctgaggctcgaaagcgtggggagcaaacagg

SEQ ID NO: 275 - DNA, 404 bp, Artificial Sequence
  Operational Taxonomic Unit (OTU) consensus sequence 561
tggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtagacggcgcagcaagtctgatgtgaaaggcaggggcttaacccctggactgcattggaaactgctgtgcttgagtgccggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 276 - DNA, 429 bp, Artificial Sequence
  Operational Taxonomic Unit (OTU) consensus sequence 590
tggggaatcttccgcaatgggcgaaagcctgacggagcaacgccgcgtgaacgatgaaggtcttaggatcgtaaagttctgttgttagggacgaaggataaggattataatacagtctttgtttgacggtacctaacgaggaagccacggctaactacgtgccagcagccgcggtaatacgtaggcggcaagcgttgtccggaattattgggcgtaaagggagcgcaggcgggaaactaagcggatcttaaaagtgcggggctcaaccccgtgatggggtccgaactggttttcttgagtgcaggagaggaaagcggaattcccagtgtagcggtgaaatgcgtagatattgggaagaacaccagtggcgaaggcggctttctggactgtaactgacgctgaagctcgaaagtgcgggtatcgaacagg

SEQ ID NO: 277 - DNA, 404 bp, Artificial Sequence
  Operational Taxonomic Unit (OTU) consensus sequence 592
tggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggcatcacaagtcagaagtgaaagcccggggctcaaccccgggactgcttttgaaactgtggagctggagtgcaggagaggcaagtggaattcctagtgtagcggtgaaatgcgtagatattaggaggaacaccagtggcgaaggcggcctactgggcaccaactgacgctgaggctcgaaagtgtgggtagcaaacagg

SEQ ID NO: 278 - DNA, 407 bp, Artificial Sequence
  Operational Taxonomic Unit (OTU) consensus sequence 599
tggggaatattgggcaatgggcgcaagcctgacccagcaacgccgcgtgaaggaagaaggctttcgggttgtaaacttcttttgtcagggacgaagcaagtgacggtacctgacgaataagccacggctaactacgtgccagcagccgcggtaatacgtaggtggcaagcgttatccggatttactgggtgtaaagggcgtgtaggcgggaaagcaagtcagatgtgaaaactgtgggctcaacccacagcctgcatttgaaactgtttttcttgagtactggagaggcagatggaattcctagtgtagcggtgaaatgcgtagatattaggaggaacaccagtggcgaaggcgatctgctggacagcaactgacgctgaggcgcgaaagcgtggggagcaaaaagg

SEQ ID NO: 279 - DNA, 424 bp, Artificial Sequence
  Operational Taxonomic Unit (OTU) consensus sequence 611
tgaggaatattggtcaatggacgcaagtctgaaccagccatgccgcgtgcaggatgacggctctatgagttgtaaactgcttttgtactagggtaaactcacctacgtgtaggtgactgaaagtatagtacgaataaggatcggctaactccgtgccagcagccgcggtaatacggaggattcaagcgttatccggagttattgggtttaaagggtgcgtaggcggtttgataagttagaggtgaaatgttagggctcaaccctgaaactgcctctaatactgttggactagagagtagttgcggtaggcggaatgtatggtgtagcggtgaaatgcttagagatcatacagaacaccgattgcgaaggcagcttaccaaactatatctgacgttgaggcacgaaagcgtggggagcaaacagg

SEQ ID NO: 280 - DNA, 404 bp, Artificial Sequence
  Operational Taxonomic Unit (OTU) consensus sequence 634
tggggaatattgcacaatgggggaaaccctgatgcagcaacgccgcgtgagtgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgcaggcggtttggcaagtctgatgtgaaaatccggggctcaactccggaactgcattggaaactgtcagactagagtgtcggagaggtaagtggaattcctagtgtagcggtgaaatgcgtagatattaggaggaacaccagtggcgaaggcggcttgctggacgataactgacgctgaggctcgaaagcgtggggagcaaacagg

SEQ ID NO: 281 - DNA, 424 bp, Artificial Sequence
  Operational Taxonomic Unit (OTU) consensus sequence 641
tgaggaatattggtcaatggacgagagtctgaaccagccaagtagcgtgcaggatgacggccctatgggttgtaaactgcttttataagggaataaagtgagagtcgtgactctttttgcatgtaccttatgaataaggaccggctaattccgtgccagcagccgcggtaatacggaaggtccgggcgttatccggatttattgggtttaaagggagcgtaggccggagattaagcgtgttgtgaaatgtagacgctcaacgtctgcactgcagcgcgaactggtttccttgagtacgcacaaagtgggcggaattcgtggtgtagcggtgaaatgcttagatatcacgaagaactccgattgcgaaggcagcttactggattgtaactgacgctgatgctcgaaagtgtgggtatcaaacagg

SEQ ID NO: 282 - DNA, 424 bp, Artificial Sequence
  Operational Taxonomic Unit (OTU) consensus sequence 646
tgaggaatattggtcaatggacggaagtctgaaccagccaagtagcgtgcaggaagacggccctatgggttgtaaactgcttttatacggggataaagtgagccacgtgtggcttattgcaggtaccgtatgaataaggaccggctaattccgtgccagcagccgcggtaatacggaaggtccgggcgttatccggatttattgggtttaaagggagcgtaggccgtttgttaagcgtgttgtgaaatgtcggggctcaacctgggcattgcagcgcgaactggcagacttgagtgcgcgggaagtaggcggaattcgtcgtgtagcggtgaaatgcttagatatgacgaagaactccgattgcgaaggcagcctgctgtagcgtaactgacgctgaagctcgaaagtgcgggtatcgaacagg

SEQ ID NO: 283 - DNA, 404 bp, Artificial Sequence
  Operational Taxonomic Unit (OTU) consensus sequence 664
tggggaatattgcacaatggaggaaactctgatgcagcgacgccgcgtgaaggaagaagtatctcggtatgtaaacttctatcagtagggaagaaaatgacggtacctgactaagaagccccggctaactacgtgccagcagccgcggtaatacgtagggggcaagcgttatccggatttactgggtgtaaagggagcgtagacggacgggcaagtctgatgtgaaagcccggggcttaaccccgggactgcattggaaactgtccatcttgagtgccgaagaggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 284 - DNA, 404 bp, Artificial Sequence
  Operational Taxonomic Unit (OTU) consensus sequence 684
tggggaatattgcacaatgggcgaaagcctgatgcagcgacgccgcgtgagcgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaggcaagtctgatgtgaaaacccggggctcaaccccgtgactgcattggaaactgttttgcttgagtgccggagaggtaagcggaattcctagtgtagcggtgaaatgcgtagatattaggaggaacaccagtggcgaaggcggcttactggacggcaactgacgttgaggctcgaaagcgtggggagcaaacagg

SEQ ID NO: 285 - DNA, 407 bp, Artificial Sequence
  Operational Taxonomic Unit (OTU) consensus sequence 744
tggggaatattgcacaatgggcgaaagcctgacccagcaacgccgcgtgaaggaagaaggccttcgggttgtaaacttcttttaccagggacgaaggacgtgacggtacctggagaaaaagcaacggctaactacgtgccagcagccgcggtaatacgtaggttgcaagcgttgtccggatttactgggtgtaaagggcgtgtaggcggagatgcaagttaggagtgaaatctatgggctcaacccataaactgcttctaaaactgtatcccttgagtatcggagaggcaagcggaattcctagtgtagcggtgaaatgcgtagatattaggaggaacaccagtggcgaaggcggcttgctggacgacaactgacgctgaggcgcgaaagcgtggggagcaaacagg

SEQ ID NO: 286 - DNA, 404 bp, Artificial Sequence
  Operational Taxonomic Unit (OTU) consensus sequence 757
tgggggatattgcacaatggaggaaactctgatgcagcgacgccgcgtgagtgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggatgggcaagtctgatgtgaaaacccggggctcaaccccgggactgcattggaaactgttcatctagagtgctggagaggtaagtggaattcctagtgtagcggtgaaatgcgtagatattaggaggaacaccagtggcgaaggcggcttactggacagtaactgacgttgaggctcgaaagcgtggggagcaaacagg

SEQ ID NO: 287 - DNA, 429 bp, Artificial Sequence
  Operational Taxonomic Unit (OTU) consensus sequence 770
tggggaatcttccgcaatgggcgaaagcctgacggagcaacgccgcgtgagtgatgacggccttcgggttgtaaagctctgtgatcggggacgaacggtcagcagacgaatactctgctgaagtgacggtacccgaatagcaagccacggctaactacgtgccagcagccgcggtaatacgtaggtggcaagcggtgtccggaattattgggcgtaaagcgcgcgcaggcggcttcttaagtccatcttaaaagtgcggggcttaaccccgtgatgggatggaaactgagaggctggagtatcggagaggaaagtggaattcctagtgtagcggtgaaatgcgtagagattaggaagaacaccggtggcgaaggcgactttctggacgacaactgacgctgaggcgcgaaagcgtggggagcaaacagg

SEQ ID NO: 288 - DNA, 404 bp, Artificial Sequence
  Operational Taxonomic Unit (OTU) consensus sequence 789
tggggaatattgcacaatgggggaaaccctgatgcagcaacgccgcgtgagtgaagaagtatttcggtatgtaaacttctatcagcagggaagatagtgacggtacctgactaagaagccccggctaactacgtgccagcagccgcggtaatacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 289 - DNA, 407 bp, Artificial Sequence
  Operational Taxonomic Unit (OTU) consensus sequence 839
tggggaatattgcacaatgggggaaaccctgatgcagcgacgccgcgtgagcgaagaagtatttcggtatgtaaagctctatcagcagggaagaaaaaaatgacggtacctgactaagaagccccggctaactacgtgccagcagccgcggtaatacgtagggggcaagcgttatccggatttactgggtgtaaagggagcgcaggcggtgcggcaagtctgatgtgaaagcccggggctcaaccccgggactgcattggaaactgtcgtacttgagtatcggagaggtaagtggaattcctagtgtagcggtgaaatgcgtagatattaggaggaacaccagtggcgaaggcggcttactggactgtaactgacgttgaggctcgaaagcgtggggagcaaacagg

SEQ ID NO: 290 - DNA, 425 bp, Artificial Sequence
  Operational Taxonomic Unit (OTU) consensus sequence 842
tgaggaatattggtcaatggacgagagtctgaaccagccaagtagcgtgaaggatgactgccctatgggttgtaaacttcttttatacgggaataaagtgttccacgtgtggaattttgtatgtaccgtatgaataaggatcggctaactccgtgccagcagccgcggtaatacggaggatccgagcgttatccggatttattgggtttaaagggagcgtaggtggaagattaagtcagcctgtgaaagtttgcggctcaaccgtaaaattgcagttgatactggttttcttgagtgcagtagaggtgggcggaattcgtggtgtagcggtgaaatgcttagatatcacgaagaactccgattgcgaaggcagctcactggactgtaactgacactgatgctcgaaagtgtgggtatcaaacagg

SEQ ID NO: 291 - DNA, 404 bp, Artificial Sequence
  Operational Taxonomic Unit (OTU) consensus sequence 882
tgggggatattgcacaatggaggaaactctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgcaggcggtgcggcaagtcagatgtgaaaacccggggctcaaccccgggactgcatttgaaactgtcggactagagtgccggagaggtaagtggaattcctagtgtagcggtgaaatgcgtagatattaggaggaacaccagtggcgaaggcggcttactggacggtaactgacgctgaggctcgaaagcgtggggagcaaacagg

SEQ ID NO: 292 - DNA, 404 bp, Artificial Sequence
  Operational Taxonomic Unit (OTU) consensus sequence 885
tggggaatattgcacaatgggggaaaccctgatgcagcaacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgagacaagtctgaagtgaaagcccggggctcaaccccgggactgctttggaaactgccttgctagagtgctggagaggtaagtggaattcctagtgtagcggtgaaatgcgtagatattaggaggaacaccagtggcgaaggcggcttactggacagtaactgacgttgaggctcgaaagtgcgggtatcgaacagg

SEQ ID NO: 293 - DNA, 404 bp, Artificial Sequence
  Operational Taxonomic Unit (OTU) consensus sequence 886
tggggaatattgcacaatggaggaaactctgatgcagcgacgccgcgtgagtgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgtaggtggctgtgcaagtcagaagtgaaagcccggggcttaaccccgggactgcttttgaaactgtgcggctggagtgcaggagaggtaagtggaattcctagtgtagcggtgaaatgcgtagatattaggaggaacaccggtggcgaaggcggcttactggactgtaactgaaactgaggctcgaaagcgtggggagcaaacagg

SEQ ID NO: 294 - DNA, 406 bp, Artificial Sequence
  Operational Taxonomic Unit (OTU) consensus sequence 924
tgaggaatattggtcaatgggcgcaagcctgacccagcaacgccgcgtgaaggaagaaggctttcgggttgtaaacttcttttaagagggaagagcagaagacggtacctcttgaataagccacggctaactacgtgccagcagccgcggtaatacgtaggtggcaagcgttgtccggatttactgggtgtaaagggcgtgcagccgggtgcgcaagtcagatgtgaaatctcagggctcaaccctgaaactgcatttgaaactgtgcatcttgagtgccggagaggtaatcggaattccttgtgtagcggtgaaatgcgtagatataaggaagaacaccagtggcgaaggcggattactggacggtaactgacggtgaggcgcgaaagcgtggggagcaaacagg

SEQ ID NO: 295 - DNA, 404 bp, Artificial Sequence
  Operational Taxonomic Unit (OTU) consensus sequence 936
tggggaatattgcacaatgggggaaaccctgatgcagcgacgccgcgtggaggaagaaggtcttcggattgtaaactcctgttgttggggaagataatgacggtacccaacaaggaagtgacggctaactacgtgccagcagccgcggtaaaacgtaggtcacaagcgttgtccggaattactgggtgtaaagggagcgcaggcgggaagacaagttggaagtgaaatctatgggctcaacccataaactgctttcaaaactgtttttcttgagtagtgcagaggtaggcggaattcccggtgtagcggtggaatgcgtagatatcgggaggaacaccagtggcgaaggcagcttactggattgtaactgacgctgatgctcgaaagtgtgggtatcaaacagg

SEQ ID NO: 296 - DNA, 404 bp, Artificial Sequence
  Operational Taxonomic Unit (OTU) consensus sequence 939
tggggaatattgcacaatgggcggaagcctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgacgcaagtctggagtgaaagcccggggcccaaccccgggactgctttggaaactgtgctgctggagtgcaggagaggtaagtggaattcctagtgtagcggtgaaatgcgtagatattaggaagaacaccagtggcgaaggcggcttgctggacagtaactgacgttcaggctcgaaagcgtggggagcaaacagg

SEQ ID NO: 297 - DNA, 429 bp, Artificial Sequence
  Operational Taxonomic Unit (OTU) consensus sequence 968
tggggaatattgcacaatgggcgcaagcctgatgcagccatgccgcgtgtatgaagaaggccttcgggttgtaaagtactttcagcgaggaggaaggtgttgtggttaataaccgcagcaattgacgttactcgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggctagagtcttgtagaggggggtagaattccaggtgtagcggtgaaatgcgtagagatctggaggaatacaggtggcgaaggcggcaccctggaaaaagactgacgctcaggtgcgaaagcgtggggagcaaacagg

SEQ ID NO: 298 - DNA, 404 bp, Artificial Sequence
  Operational Taxonomic Unit (OTU) consensus sequence 983
tggggaatattgcacaatgggggaaaccctgatgcagcgacgccgcgtgagcgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggaatggcaagtctgatgtgaaaggccggggctcaaccccgggactgcattggaaactgtcaatctagagtaccggaggggtaagtggaattcctagtgtagcggtgaaatgcgtagatattaggaggaacaccagtggcgaaggcggcttgctggactgtaactgacactgaggctcgaaagcgtggggagcaaacagg

SEQ ID NO: 299 - DNA, 404 bp, Artificial Sequence
  Operational Taxonomic Unit (OTU) consensus sequence 1011
tggggaatattgcacaatggaggaaactctgatgcagcgacgccgcgtgagtgaagaagtagttcgctatgtaaagctctatcagcagggaagatagtgacggtacctgactaagaagctccggctaaatacgtgccagcagccgcggtaatacgtatggagcaagcgttatccggatttactgggtgtaaagggagcgtagacggcagggcaagtctgatgtgaaaacccggggctcaaccccgggactgcattggaaactgtccggctggagtgcaggagaggtaagtggaattcctagtgtagcggtgaaatgcgtagatattaggaggaacaccagtggcgaaggcggcttactggactgtaactgacgttgaggctcgaaagcgtggggagcaaacagg

SEQ ID NO: 300 - DNA, 407 bp, Artificial Sequence
  Operational Taxonomic Unit (OTU) consensus sequence 1028
tggggaatattgggcaatgggggaaaccctgacccagcaacgccgcgtgaaggaagaaggctttcgggttgtaaacttcttttaccagggacgaagagagtgacggtacctggagaaaaagccacggctaactacgtgccagcagccgcggtaatacgtaggtggcaagcgttgtccggatttactgggtgtaaagggcgtgtaggcggagattcaagtcaggagtgaaatctatgggcttaacccataaactgcttttgaaactgaatcccttgagtatcggagaggcaggcggaattcctagtgtagcggtgaaatgcgtagatattaggaggaacacaagtggcgaaggcggcatgctggaagacaactgacgctgaggcgcgaaagcgtggggagcaaacagg

SEQ ID NO: 301 - DNA, 407 bp, Artificial Sequence
  Operational Taxonomic Unit (OTU) consensus sequence 1029
tggggaatattgggcaatgggggaaaccctgacccagcaacgccgcgtgaaggaagaaggctttcgggttgtaaacttcttttaccagggacgaagaaagtgacggtacctggagaaaaagccacggctaactacgtgccagcagccgcggtaatacgtaggtggcaagcgttgtccggatttactgggtgtaaagggcgtgtaggcggagatgcaagtcagatgtgaaatccccgggcttaacccgggaactgcatttgaaactgtatcccttgagtatcggagaggcaggcggaattcctagtgtagcggtgaaatgcgtagatattaggaggaacaccagtggcgaaggcggattgctggacgacaactgacggtgaggcgcgaaagcgtggggagcaaacagg

SEQ ID NO: 302 - DNA, 407 bp, Artificial Sequence
  Operational Taxonomic Unit (OTU) consensus sequence 1038
tgaggaatattggtcaatgggcgcaagcctgacccagcaacgccgcgtgaaggaagaaggctttcgggttgtaaacttcttttcttagggacgaagcaagtgacggtacctaaggaataagccacggctaactacgtgccagcagccgcggtaatacgtaggtggcaagcgttatccggatttactgggtgtaaagggcgtgtaggcgggattgcaagtcagatgtgaaaaccacgggctcaacctgtggcctgcatttgaaactgtagttcttgagtactggagaggcagacggaattcctagtgtagcggtgaaatgcgtagatattaggaggaacaccagtggcgaaggcggtctgctggacagcaactgacgctgaggcgcgaaagcgtggggagcaaacagg

SEQ ID NO: 303 - DNA, 404 bp, Artificial Sequence
  Operational Taxonomic Unit (OTU) consensus sequence 1084
tggggaatattgcacaatgggcgcaagcctgatgcagcgatgccgcgtggaggaagaaggttttcggattgtaaactcctgtcttaagggacgataatgacggtaccttaggaggaagctccggctaactacgtgccagcagccgcggtaatacgtagggagcgagcgttgtccggaattactgggtgtaaagggagcgtaggcgggattgcaagtcagatgtgaaaactatgggcttaacccatagactgcatttgaaactgtagttcttgagtgaagtagaggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 304 - DNA, 404 bp, Artificial Sequence
  Operational Taxonomic Unit (OTU) consensus sequence 1089
tggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaaacccagggctcaaccttgggattgcatttggaactgccaggctggagtgcaggagaggtaagcggaattcctagtgtagcggtgaaatgcgtagatattaggaggaacaccagtggcgaaggcggcttactggactgtaactgacgttgaggctcgaaagcgtggggagcaaacagg

SEQ ID NO: 305 - DNA, 429 bp, Artificial Sequence
  Operational Taxonomic Unit (OTU) consensus sequence 1092
tgaggaatattggtcaatgggcgagagcctgacggagcaacgccgcgtgaacgatgaaggtcttaggatcgtaaagttctgttgttagggacgaagggcaagggttataatacagcctttgtttgacggtacctaacgaggaagccacggctaactacgtgccagcagccgcggtaatacgtaggcggcaagcgttgtccggaattattgggcgtaaagggagcgcaggcgggaaactaagcggatcttaaaagtgcggggctcaaccccgtgatggggtccgaactggttttcttgagtgcaggagaggaaagcggaattcccagtgtagcggtgaaatgcgtagatattgggaagaacaccagtggcgaaggcggctttctggactgtaactgacgctgaggctcgaaagctagggtagcgaacggg

SEQ ID NO: 306 - DNA, 429 bp, Artificial Sequence
  Operational Taxonomic Unit (OTU) consensus sequence 1121
tggggaatattgcacaatgggggcaaccctgaccgagcaacgccgcgtgagtgaagaaggttttcggatcgtaaagctctgttgtaagagaagaacgagtgtgagagtggaaagttcacactgtgacggtaacttaccagaaagggacggctaactacgtgccagcagccgcggtaatacgtaggtcccgagcgttatccggatttattgggcgtaaagcgagcgcaggcggttagataagtctgaagttaaaggctgtggcttaaccatagtacgctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacagg

SEQ ID NO: 307 - DNA, 404 bp, Artificial Sequence
  Operational Taxonomic Unit (OTU) consensus sequence 1124
tggggaatattgcacaatgggggaaaccctgatgcagcgacgccgcgtgagtgaagaagtatttcggtatgtaaagctctatcagcagggaagagaatgacggtacctgactaagaagccccggctaactacgtgccagcagccgcggtaatacgtagggggcaagcgttatccggatttactgggtgtaaagggagcgtagacggcttagcaagtctgaagtgaaagcccggggctcaaccccgggactgctttggaaactgttaagctggagtgctggagaggtaagcggaattcctagtgtagcggtgaaatgcgtagatattaggaggaacaccagtggcgaaggcggcttactggacagtaactgacgttcatgctcgaaagtgtgggtatcaaacagg

SEQ ID NO: 308 - DNA, 404 bp, Artificial Sequence
  Operational Taxonomic Unit (OTU) consensus sequence 1125
tggggaatattgcacaatgggggaaaccctgatgcagcgacgccgcgtgagcgatgaagtatttcggtatgtaaagctctatcagcagggaagataatgacggtacctgactaagaagctccggctaaatacgtgccagcagccgcggtaatacgtatggagcaagcgttatccggatttactgggtgtaaagggagcgtaggcggtcctgcaagtctgatgtgaaaggccggggctcaaccccgggactgcattggaaactgtaggactagagtgtcggaggggtaagtggaattcctagtgtagcggtgaaatgcgtagatattaggaggaacaccagtggcgaaggcggctcactggactgtaactgacactgaggctcgaaagcgtggggagcaaacagg

SEQ ID NO: 309 - DNA, 424 bp, Artificial Sequence
  Operational Taxonomic Unit (OTU) consensus sequence 1134
tgaggaatattggtcaatgggcgcaggcctgaaccagccaagtagcgtgaaggatgactgccctatgggttgtaaacttcttttatacgggaataaagttagccacgtgtggttttttgcatgtaccgtatgaataaggatcggctaactccgtgccagcagccgcggtaatacggaggatccgagcgttatccggatttattgggtttaaagggagcgtaggcggggtattaagtcagttgtgaaagtttgcggctcaaccgtaaaattgcagttgatactggtatccttgagtgcagcagaggtgggcggaattcgtggtgtagcggtgaaatgcttagatatcacgaagaactccgattgcgaaggcagctcactggagtgtaactgacgctgatgctcgaaagtgtgggtatcaaacagg

SEQ ID NO: 310 - DNA, 404 bp, Artificial Sequence
  Operational Taxonomic Unit (OTU) consensus sequence 1162
tggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtgagacaagtctgaagtgaaatcccggggctcaaccccggaactgctttggaaactgcctgactagagtgcaggagaggtaagtggaattcctagtgtagcggtgaaatgcgtagatattaggaggaacaccagtggcgaaggcggcttgctggactgtaactgacactgaggctcgaaagcgtggggagcaaacagg

SEQ ID NO: 311 - DNA, 407 bp, Artificial Sequence
  Operational Taxonomic Unit (OTU) consensus sequence 1164
tggggaatattgcacaatgggggaaaccctgatgcagcgacgccgcgtgaaggaagaaggctttcgggttgtaaacttcttttaccagggacgaagaacgtgacggtacctggagaaaaagccacggctaactacgtgccagcagccgcggtaatacgtaggtggcaagcgttgtccggatttactgggtgtaaagggcgtgtaggcgggagagcaagtcagaagtgaaatctatgggcttaacccataaactgcttttgaaactgttcttcttgagtatcggagaggcaggcggaattcctagtgtagcggtgaaatgcgtagatattaggaggaacaccagtggcgaaggcggcctgctggacgacaactgacgctgaggcgcgaaagcgtggggagcaaacagg

SEQ ID NO: 312 - DNA, 404 bp, Artificial Sequence
  Operational Taxonomic Unit (OTU) consensus sequence 1166
tggggaatattgcacaatgggggaaaccctgatgcagcaacgccgcgtgagtgaagaagtatttcggtatgtaaacttctatcagcaaggaagaaaatgacggtacttgactaagaagccccggctaaatacgtgccagcagccgcggtaatacgtatggggcaagcgttatccggatttactgggtgtaaagggagcgtaggcggtaagacaagtcagaagtgaaaggctggggctcaaccctgggactgcttttgaaactgtctaactagagtgcaggagaggtaagtggaattcctagtgtagcggtgaaatgcgtagatattaggaggaacaccagtggcgaaggcggcttactggactgtaactgacgctgaggctcgaaagcgtggggagcaaacagg

SEQ ID NO: 313 - DNA, 404 bp, Artificial Sequence
  Operational Taxonomic Unit (OTU) consensus sequence 1170
tggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtaaagcaagtctgaagtgaaagcccgcggctcaactgcgggactgctttggaaactgtttaactggagtgtcggagaggtaagtggaattcctagtgtagcggtgaaatgcgtagatattaggaggaacaccagtggcgaaggcggcttgctggactgtaactgacactgaggctcgaaagcgtggggagcaaacagg

SEQ ID NO: 314 - DNA, 424 bp, Artificial Sequence
  Operational Taxonomic Unit (OTU) consensus sequence 1183
tgaggaatattggtcaatggacgagagtctgaaccagccaagtagcgtgcaggatgacggccctatgggttgtaaactgcttttataagggaataaagtgagctacgtgtagctttttgcatgtaccttatgaataaggaccggctaattccgtgccagcagccgcggtaatacggaaggtccgggcgttatccggatttattgggtttaaagggagcgtaggccgtcttataagcgtgttgtgaaatgtcggggctcaacctgggcattgcagcgcgaactgtgagacttgagtgcgcaggaagtaggcggaattcgtcgtgtagcggtgaaatgcttagatatgacgaagaactccgattgcgaaggcagcctgctgtagcgcaactgacgctgaagctcgaaagcgtgggtatcgaacagg

SEQ ID NO: 315 - DNA, 404 bp, Artificial Sequence
  Operational Taxonomic Unit (OTU) consensus sequence 1230
tggggaatattgcacaatgggcgaaagcctgatgcagcgacgccgcgtgagtgaagaagtatctcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaaggcggcttactggacgatgactgacgttgaggctcgaaagcgtggggagcaaacagg

SEQ ID NO: 316 - DNA, 424 bp, Artificial Sequence
  Operational Taxonomic Unit (OTU) consensus sequence 1238
tgaggaatattggtcaatgggcgagagcctgaaccagccaagtagcgtgcaggatgacggccctatgggttgtaaactgcttttatacggggataaagtgagccacgtgtggcttattgcaggtaccgtatgaataaggaccggctaattccgtgccagcagccgcggtaatacggaaggtccgggcgttatccggatttattgggtttaaagggagcgtaggccgtctgttaagcgtgttgtgaaatgtcggggctcaacctgggcattgcagcgcgaactggcagacttgagtgcgcaggaagtaggcggaattcgtcgtgtagcggtgaaatgcttagatatcacgaagaactccgattgcgaaggcagctcactggagcgcaactgacgctgaagctcgaaagtgcgggtatcgaacagg

SEQ ID NO: 317 - DNA, 405 bp, Artificial Sequence
  Operational Taxonomic Unit (OTU) consensus sequence 1244
tcgggaatattgcgcaatggaggaaactctgacgcagtgacgccgcgtataggatgaaggttttcggattgtaaactattgtcattagggaagataaaagacagtacctaaggaggaagctccggctaactacgtgccagcagccgcggtaatacgtagggagcaagcgttatccggatttattgggtgtaaagggtgcgtagacgggaaattaagttagttgtgaaagccctcggcttaactgaggaattgcaactaaaactggttttcttgagtgcaggagaggtaagtggaattcctagtgtagcggtgaaatgcgtagatattaggaggaacaccagtggcgaaggcgacttactggactgtaactgacgttgaggctcgaaagcgtggggagcaaacagg

SEQ ID NO: 318 - DNA, 429 bp, Artificial Sequence
  Operational Taxonomic Unit (OTU) consensus sequence 1261
tggggaattttggacaatgggggcaaccctgatccagccatgccgcgtgcgggatgaaggccttcgggttgtaaaccgcttttgtcagggacgaaaagggacgtgccaataccacgttctgctgacggtacctgaagaataagcaccggctaactacgtgccagcagccgcggtaatacgtagggtgcaagcgttaatcggaattactgggcgtaaagcgtgcgcaggcggtttcgtaagatagatgtgaaatccccgggctcaacctgggaattgcatttatgactgcgggactggagtttgtcagaggggggtggaattccaagtgtagcagtgaaatgcgtagatatttggaagaacaccgatggcgaaggcagccccctgggacatgactgacgctcatgcacgaaagcgtggggagcaaaaagg

SEQ ID NO: 319 - DNA, 404 bp, Artificial Sequence
  Operational Taxonomic Unit (OTU) consensus sequence 1290
tggggaatattgcacaatgggggaaaccctgatgcagcgacgccgcgtgagcgatgaagtatttcggtatgtaaagctctatcagcagggaagataatgacggtacctgactaagaagctccggctaaatacgtgccagcagccgcggtaatacgtatggagcaagcgttatccggatttactgggtgtaaagggagcgtaggcggtcctgcaagtctgatgtgaaaacccggggctcaaccccgggactgcattggaaactgtaggactagagtgtcggaggggtaagtggaattcctagtgtagcggtgaaatgcgtagatattaggaggaacaccagtggcgaaggcggcttactggacgaccactgacgctgaagctcgaaagtgcgggtatcgaacagg

SEQ ID NO: 320 - DNA, 404 bp, Artificial Sequence
  Operational Taxonomic Unit (OTU) consensus sequence 1292
tggggaatattgcacaatgggggaaaccctgatgcagcgacgccgcgtgaacgaagaagtatttcggtatgtaaagttctatcagcagggaagataatgacggtacctgactaagaagcaccggctaaatacgtgccagcagccgcggtaatacgtatggtgcaagcgttatccggatttactgggtgtaaagggtgcgtaggtggcaaggcaagtctgaagtgaaaatccggggctcaaccccggaactgctttggaaactgtttagctggagtacaggagaggtaagtggaattcctagtgtagcggtgaaatgcgtagatattaggaggaacaccagtggcgaaggcgacttactggactgctactgacactgaggcacgaaagcgtggggagcaaacagg

SEQ ID NO: 321 - DNA, 405 bp, Artificial Sequence
  Operational Taxonomic Unit (OTU) consensus sequence 1357
tggggaatattgcacaatgggggaaaccctgatgcagcgacgccgcgtgagcgatgaagtatttcggtatgtaaagctctatcagcagggaagaattaggacggtacctgactaagaagcaccggctaaatacgtgccagcagccgcggtaatacgtatggtgcaagcgttatccggatttactgggtgtaaagggagcgtagacggagaggcaagtctgatgtgaaaacccggggctcaaccccgggactgcattggaaactgtttttctagagtgtcggagaggtaagtggaattcctagtgtagcggtgaaatgcgtagatattaggaggaacaccagtggcgaaggcggcttgctggactgtaactgacactgaggctcgaaagcgtggggagcaaacagg

SEQ ID NO: 322 - DNA, 406 bp, Artificial Sequence
  Operational Taxonomic Unit (OTU) consensus sequence 1358
tggggaatattgcacaatgggggaaaccctgatgcagcgacgccgcgtgagcgaagaagtatttcggtatgtaaagctctatcagcagggaagaagaaatgacggtacctgactaagaagccccggctaactacgtgccagcagccgcggtaatacgtagggggcaagcgttatccggatttactgggtgtaaagggagcgcaggcggatggctaagtctgatgtgaaagcccggggctcaaccccgggactgcattggaaactggttatcttgagtgtcggagaggtaagtggaattcctagtgtagcggtgaaatgcgtagatattaggaggaacaccagtggcgaaggcggcttgctggactgtaactgacactgaggctcgaaagcgtggggagcaaacagg

SEQ ID NO: 323 - DNA, 429 bp, Artificial Sequence
  Operational Taxonomic Unit (OTU) consensus sequence 1365
tggggaatattgcacaatgggggaaaccctgaacgagcaatgccgcgtgagtgaggaaggtcttcggatcgtaaagctctgttgtaagagaaaaacggcactcatagggaatgatgagtgagtgatggtatcttaccagaaagtcacggctaactacgtgccagcagccgcggtaatacgtaggtggcgagcgttatccggaatgattgggcgtaaagggtgcgtaggtggcagatcaagtctggagtaaaaggtatgggctcaacccgtactggctctggaaactgatcagctagagaacagaagaggacggcggaactccatgtgtagcggtaaaatgcgtagatatatggaagaacaccggtggcgaaggcggccgtctggtctggattctgacactgaagcacgaaagcgtggggagcaaatagg

SEQ ID NO: 324 - DNA, 424 bp, Artificial Sequence
  Operational Taxonomic Unit (OTU) consensus sequence 1380
tgaggaatatttgtcaatgggcgagagcctgaaccagccaagtatcgtgcagtattacgtccctatgggttgtaaactgcttttataagggaataaagtgagcctcgtgaggctttttgcatgtaccttatgaataaggaccggctaattccgtgccatcatccgcggtaatacggaaggtccgggcgttatccggatttattgggtttaaagggagcgtaggccggagattaagcgtgttgtgaaatgtagatgctcaacatctgcactgcagcgcgaactggtttccttgagtacgcacaaagtgggcggaattcgtggtgtagcggtgaaatgcttagatatcacgaagaactcagattgcgaaggcagctcactggagcgcaactgacgctgaagctagaaagtgcgggtatcgaacagg

SEQ ID NO: 325 - DNA, 424 bp, Artificial Sequence
  Operational Taxonomic Unit (OTU) consensus sequence 1383
tgaggaatattggtcaatgggcgtaagcctgaaccagccaagtcgcgtgagggatgaaggttctatggatcgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacagg

SEQ ID NO: 326 - DNA, 424 bp, Artificial Sequence
  Operational Taxonomic Unit (OTU) consensus sequence 1390
tgaggaatattggtcaatgggcgatggcctgaaccagccaagtagcgtgaaggatgactgccctatgggttgtaaacttcttttatagggggataaagtgtggtacgtgtaccatattgcaggtaccctatgaataaggaccggctaattccgtgccagcagccgcggtaatacggaaggtccgggcgttatccggatttattgggtttaaagggagcgtaggccgtcttataagcgtgttgtgaaatgtcggggctcaacctgggcattgcagcgcgaactgtgagacttgagtgcgcaggaagtaggcggaattcgtcgtgtagcggtgaaatgcttagatatgacgaagaactccgattgcgaaggcagcctgctgtagcgcaactgacgctgaagctcgaaagcgtgggtatcgaacagg

SEQ ID NO: 327 - DNA, 424 bp, Artificial Sequence
  Operational Taxonomic Unit (OTU) consensus sequence 1393
tgaggaatattggtcaatgggcgagagcctgaaccagccaagtagcgtgcaggatgacggccctatgggttgtaaactgcttttataggggaataaagtgagagtcgtgactctttttgcatgtaccctatgaataaggaccggctaattccgtgccagcagccgcggtaatacggaaggtccgggcgttatccggatttattgggtttaaagggagcgtaggccggagattaagcgtgttgtgaaatgtagtggctcaacctctgcactgcagcgcgaactggtcttcttgagtacgcacaacgtgggcggaattcgtggtgtagcggtgaaatgcttagatatcacgaagaactccgattgcgaaggcagctcacgggagcgcaactgacgctgaagctcgaaagtgcgggtatcgaacagg

SEQ ID NO: 328 - DNA, 424 bp, Artificial Sequence
  Operational Taxonomic Unit (OTU) consensus sequence 1398
tgaggaatattggtcaatggacgggagtctgaaccagccaagtagcgtgcaggacgacggccctatgggttgtaaactgcttttatagggggataaagtgtgccacgtgtggcatattgcaggtaccctatgaataaggaccggctaattccgtgccagcagccgcggtaatacggaaggtccgggcgttatccggatttattgggtttaaagggagcgtaggccgtcttataagcgtgttgtgaaatgtcgtggctcaacctgggcattgcagcgcgaactgtgagacttgagtgcgcaggaagtaggcggaattcgtcgtgtagcggtgaaatgcttagatatgacgaagaactccgattgcgaaggcagctcactggagcgcaactgacgctgaagctcgaaagtgcgggtatcgaacagg

SEQ ID NO: 329 - DNA, 424 bp, Artificial Sequence
  Operational Taxonomic Unit (OTU) consensus sequence 1400
tgaggaatattggtcaatggacgagagtctgaaccagccaagtagcgtgcaggatgacggccctatgggttgtaaactgcttttataagggaataaagtgagctacgtgtagctttttgcatgtaccttatgaataaggaccggctaattccgtgccagcagccgcggtaatacggaaggtccgggcgttatccggatttattgggtttaaagggagcgtaggccggagattaagcgtgttgtgaaatgtagatgctcaacatctgcactgcagcgcgaactggtttccttgagtacgcacaaagtgggcggaattcgtggtgtagcggtgaaatgcttagatatcacgaagaactccgattgcgaaggcagctcactggagcgcaactgacgttcatgctcgaaagtgtgggtatcaaacagg

SEQ ID NO: 330 - DNA, 405 bp, Artificial Sequence
  Operational Taxonomic Unit (OTU) consensus sequence 1420
tgggggatattgcgcaatgggggcaaccctgacgcagcaacgccgcgtgaaggatgaaggttttcggattgtaaacttcttttattaaggacgaaatttgacggtacttaatgaataagctccggctaactacgtgccagcagccgcggtaatacgtagggagcaagcgttgtccggatttactgggtgtaaagggtgcgtaggcggctttacaagtcagatgtgaaatctatgggctcaacccataaactgcatttgaaactgtagagcttgagtgaagtagaggcaggcggaattccccgtgtagcggtgaaatgcgtagagatggggaggaacaccagtggcgaaggcggcctgctgggctttaactgacgctgaggctcgaaagtgtgggtatcaaacagg

SEQ ID NO: 331 - DNA, 407 bp, Artificial Sequence
  Operational Taxonomic Unit (OTU) consensus sequence 1461
tggggaatattgggcaatgggcgcaagcctgacccagcaacgccgcgtgaaggaagaaggctttcgggttgtaaacttcttttgtcagggacgaagcaagtgacggtacctgacgaataagccacggctaactacgtgccagcagccgcggtaatacgtaggtggcaagcgttatccggatttattgggtgtaaagggcgtgtaggcgggattgcaagtcagatgtgaaaactgggggctcaacctccagcctgcatttgaaactgtagttcttgagtgtcggagaggcaatcggaattccgtgtgtagcggtgaaatgcgtagatattaggaggaacaccagtggcgaaggcggcttactggacgataactgacgctgaggctcgaaagcgtggggagcaaacagg

SEQ ID NO: 332 - DNA, 404 bp, Artificial Sequence
  Operational Taxonomic Unit (OTU) consensus sequence 1488
tggggaatattgcacaatgggggaaaccctgatgcagcgacgccgcgtgagcgatgaagtatttcggtatgtaaagctctatcagcagggaagataatgacggtacctgactaagaagctccggctaaatacgtgccagcagccgcggtaatacgtatggagcaagcgttatccggatttactgggtgtaaagggagtgtaggtggcacagcaagtcagaagtgaaagcccggggctcaaccccgggactgcttttgaaactgttgtgctggagtgcaggagaggtaagtggaattcctagtgtagcggtgaaatgcgtagatattaggaggaacaccagcggcgaaggcggcttactggactgtaactgacactgaggctcgaaagcgtggggagcaaacagg

SEQ ID NO: 333 - DNA, 404 bp, Artificial Sequence
  Operational Taxonomic Unit (OTU) consensus sequence 1490
tggggaatattgcacaatgggggaaaccctgatgcagcgacgccgcgtgagcgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggctttgcaagtctgatgtgaaaggcgggggctcaacccctggactgcattggaaactgtgaggcttgagtgccggagaggtaagcggaattcctagtgtagcggtgaaatgcgtagatattaggaggaacaccagtggcgaaggcggctttctggactgaaactgacactgaggcacgaaagcgtggggagcaaacagg

SEQ ID NO: 334 - DNA, 404 bp, Artificial Sequence
  Operational Taxonomic Unit (OTU) consensus sequence 1491
tggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggtgttgcaagtctgatgtgaaaggcgggggctcaacccctggactgcattggaaactgtgatactcgagtgccggagaggtaagcggaattcctagtgtagcggtgaaatgcgtagatattaggaggaacaccagtggcgaaggcggcttgctggactgtaactgacactgaggctcgaaagcgtggggagcaaacagg

SEQ ID NO: 335 - DNA, 404 bp, Artificial Sequence
  Operational Taxonomic Unit (OTU) consensus sequence 1508
tggggaatattgcacaatggaggaaactctgatgcagcgacgccgcgtgagtgaagaagtaattcgttatgtaaagctctatcagcagggaagatagtgacggtacctgactaagaagcaccggctaaatacgtgccagcagccgcggtaatacgtatggtgcaagcgttatccggatttactgggtgtaaagggagcgtagacggagaggcaagtctgatgtgaaaacccggggctcaaccccgggactgcattggaaactgtttttctagagtgtcggagaggtaagtggaattcctagtgtagcggtgaaatgcgtagatattaggaggaacaccagtggcgaaggcggcttactggacgatgactgacgttgaggctcgaaagcgtggggagcaaacagg

SEQ ID NO: 336 - DNA, 407 bp, Artificial Sequence
  Operational Taxonomic Unit (OTU) consensus sequence 1511
tgaggaatattggtcaatgggggaaaccctgatgcagcaacgccgcgtgaaggaagacggttttcggattgtaaacttctatcaatagggacgaagaaagtgacggtacctaaataagaagccccggctaactacgtgccagcagccgcggtaatacgtagggggcaagcgttatccggaattactgggtgtaaagggtgagtaggcggcatgacaagtaagatgtgaaagcccgtggcttaaccacgggattgcattttaaactgttgagctagagtacaggagaggaaagcggaattcccagtgtagcggtgaaatgcgtagatattgggaagaacaccagtggcgaaggcggctttctggactgaaactgacgctgaggcacgaaagcgtggggagcaaacagg

SEQ ID NO: 337 - DNA, 424 bp, Artificial Sequence
  Operational Taxonomic Unit (OTU) consensus sequence 1520
tgaggaatattggtcaatgggcgagagcctgaaccagccaagtagcgtgcaggatgacggccctatgggttgtaaactgcttttataagggaataaagtgagcctcgtgaggctttttgcatgtaccttatgaataaggaccggctaattccgtgccagcagccgcggtaatacggaaggtccgggcgttatccggatgtattgggtttaaagggagcgtaggccggagattaagcgtgttgtgaaatgtagatgctcaacatctgcactgcagcgcgaactggtttccttgagtacgcacaaagtgggcggaattcgtggtgtagcggtgaaatgcttagatatcacgaagaactccgattgcgaaggcagcacactaatccgtaactgacgttcatgctcgaaagtgtgggtatcaaacagg

SEQ ID NO: 338 - DNA, 404 bp, Artificial Sequence
  Operational Taxonomic Unit (OTU) consensus sequence 1689
tggggaatattgcacaatgggcgaaagcctgatgcagcgacgccgcgtgagtgaagaagtatttcggtatgtaaagctctatcagcagggaagataatgacggtacctgactaagaagccccggctaactacgtgccagcagccgcggtaatacgtagggggcaagcgttatccggatttactgggtgtaaagggagcgtagacggcatggcaagtctgaagtgaaaacccagggctcaaccctgggactgctttggaaactgtcaagctagagtgcaggagaggtaagtggaattcctagtgtagcggtgaaatgcgtagatattaggaggaacaccagtggcgaaggcggcctactgggcaccaactgacgctgaggctcgaaagtgtgggtagcaaacagg

SEQ ID NO: 339 - DNA, 404 bp, Artificial Sequence
  Operational Taxonomic Unit (OTU) consensus sequence 1694
tggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagcgcaggcggtgcggcaagtctgatgtgaaagcccggggctcaaccccggtactgcattggaaactgtcgtactagagtgtcggaggggtaagtggaattcctagtgtagcggtgaaatgcgtagatattaggaggaacaccagtggcgaaggcggcttactggacagtaactgacgttgaggctcgaaagcgtggggagcaaacagg

SEQ ID NO: 340 - DNA, 424 bp, Artificial Sequence
  Operational Taxonomic Unit (OTU) consensus sequence 1729
tgaggaatattggtcaatggacgagagtctgaaccagccaagtagcgtgaaggatgaaggttctatggattgtaaacttcttttataagggaataaaacttcccacgtgtgggagcttgtatgtaccttatgaataagcatcggctaactccgtgccagcagccgcggtaatacggaggatgcgagcgttatccggatgtattgggtttaaagggagcgcagacgggggattaagtcagttgtgaaagtttggggctcaaccttaaaattgcagttgatactggttctcttgagtgcagttgaggtaggcggaattcgtggtgtagcggtgaaatgcttagatatcacgaagaaccccgattgcgaaggcagcttgctaaactgtaactgacgttcatgctcgaaagtgtgggtatcaaacagg

SEQ ID NO: 341 - DNA, 429 bp, Artificial Sequence
  Operational Taxonomic Unit (OTU) consensus sequence 1883
tggggaatcttccgcaatgggcgcaagcctgacggagcaacgccgcgtgagtgaagaagggtttcgactcgtaaagctctgttgtcggggacgaatgtggaggttgtgaataacagcttccaatgacggtacctgacgaggaagccacggctaactacgtgccagcagccgcggtaatacgtaggtggcgagcgttgtccggaattattgggcgtaaagggagcgcaggcgggaggtcaagtctatcttaaaagtgcggggctcaaccccgtgaggggatggaaactggtcttcttgagtgcaggagaggaaagcggaattcctagtgtagcggtgaaatgcgtagatattaggaggaacaccagtggcgaaggcggctttctggactgtaactgacgctgaggctcgaaagtgcgggtatcgaacagg

SEQ ID NO: 342 - DNA, 405 bp, Artificial Sequence
  Operational Taxonomic Unit (OTU) consensus sequence 2002
tggggaatattgcacaatggggggaaccctgatgcagcgacgccgcgtgagcgaagaagtatttcggtatgtaaagctctatcagcagggaagaagaacgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagtggaattcctagtgtagcggtgaaatgcgtagatattaggaggaacaccagtggcgaaggcggcttactggacgataactgacgctgaagctcgaaagtgcgggtatcgaacagg

SEQ ID NO: 343 - DNA, 405 bp, Artificial Sequence
  Operational Taxonomic Unit (OTU) consensus sequence 2020
tggggaatattgcacaatgggggaaaccctgatgcagcgacgccgcgtgagcgaagaagtatttcggtatgtaaagctctatcagcagggaagaagaatgacggtacctgactaagaagcaccggctaaatacgtgccagcagccgcggtaatacgtatggtgcaagcgttatccggatttactgggtgtaaagggagtgtaggtggccatgcaagtcagaagtgaaaatccggggctcaaccccggaactgcttttgaaactgtgaggctggagtgcaggaggggtgagtggaattcctagtgtagcggtgaaatgcgtagatattaggaggaacaccagtggcgaaggcggcttactggacggtaactgacgttgaggctcgaaagcgtggggagcaaacagg

SEQ ID NO: 344 - DNA, 429 bp, Artificial Sequence
  Operational Taxonomic Unit (OTU) consensus sequence 2157
tgaggaatattggtcaatggacgagagtctgacggagcaacgccgcgtgaacgatgacggccttcgggttgtaaagttctgttatacgggacgaatggcgtagcggtcaatacccgttacgagtgacggtaccgtaagagaaagccacggctaactacgtgccagcagccgcggtaatacgtaggtggcaagcgttgtccggaattattgggcgtaaagggcgcgcaggcggcgtcgtaagtcggtcttaaaagtgcggggcttaaccccgtgaggggaccgaaactgcgatgctagagtatcggagaggaaagcggaattcctagtgtagcggtgaaatgcgtagatattaggaggaacaccagtggcgaaagcggctttctggacgacaactgacgctgaggcgcgaaagccaggggagcaaacggg

SEQ ID NO: 345 - DNA, 404 bp, Artificial Sequence
  Operational Taxonomic Unit (OTU) consensus sequence 678
tggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagtctgaagtgaaatgcgggggctcaacccctgaactgctttggaaactgtcaggctggagtgcaggagaggtaagtggaattcctagtgtagcggtgaaatgcgtagatattaggaggaacaccagtggcgaaggcggcttactggacgataactgacgctgaggctcgaaagcgtggggagcaaacagg

SEQ ID NO: 346 - DNA, 17 bp, Artificial Sequence
  primer 341F
cctaygggrbgcascag

SEQ ID NO: 347 - DNA, 20 bp, Artificial Sequence
  primer 806R
  misc_feature (8)..(9): n is a, c, g, or t
ggactacnngggtatctaat

SEQ ID NO: 348 - DNA, 20 bp, Artificial Sequence
  primer 8F
agagtttgatcctggctcag

SEQ ID NO: 349 - DNA, 19 bp, Artificial Sequence
  primer U1492R
ggttaccttgttacgactt

SEQ ID NO: 350 - DNA, 23 bp, Artificial Sequence
  primer 928F
taaaactyaaakgaattgacggg

SEQ ID NO: 351 - DNA, 23 bp, Artificial Sequence
  primer 336R
actgctgcsycccgtaggagtct

SEQ ID NO: 352 - DNA, 15 bp, Artificial Sequence
  primer 1100F
yaacgagcgcaaccc

SEQ ID NO: 353 - DNA, 15 bp, Artificial Sequence
  primer 1100R
gggttgcgctcgttg

SEQ ID NO: 354 - DNA, 21 bp, Artificial Sequence
  primer 337F
gactcctacgggaggcwgcag

SEQ ID NO: 355 - DNA, 20 bp, Artificial Sequence
  primer 907R
ccgtcaattcctttragttt

SEQ ID NO: 356 - DNA, 18 bp, Artificial Sequence
  primer 785F
ggattagataccctggta

SEQ ID NO: 357 - DNA, 20 bp, Artificial Sequence
  primer 805R
gactaccagggtatctaatc

SEQ ID NO: 358 - DNA, 19 bp, Artificial Sequence
  primer 533F
gtgccagcmgccgcggtaa

SEQ ID NO: 359 - DNA, 16 bp, Artificial Sequence
  primer 518R
gtattaccgcggctgg

SEQ ID NO: 360 - DNA, 20 bp, Artificial Sequence
  primer 27F
agagtttgatcmtggctcag

SEQ ID NO: 361 - DNA, 20 bp, Artificial Sequence
  primer 1492R
cggttaccttgttacgactt

(19) United States
(12) Patent Application Publication
(10) Pub. No.: US 2020/0194119 A1
(43) Pub. Date: Jun. 18, 2020

(54) METHODS AND SYSTEMS FOR PREDICTING OR DIAGNOSING CANCER

(71) Applicant: Hangzhou New Horizon Health Technology Co., Ltd., Hangzhou (CN)

(72) Inventors: Ning LU, Hangzhou (CN); Yiyou CHEN, Hangzhou (CN)

(21) Appl. No.: 16/653,154

(22) Filed: Oct. 15, 2019

Related U.S. Application Data
(60) Provisional application No. 62/745,955, filed on Oct. 15, 2018.

(51) Int. Cl. G16H 50/20; G01N 33/574; G06N 20/20; G06N 5/00

(52) U.S. Cl. CPC G16H 50/20; G01N 33/57419; G16H 50/70; G06N 5/003; G06N 20/20

(57) ABSTRACT

The present disclosure provides methods, systems, compositions, and kits for evaluating cancer risk. The methods and systems comprise producing an Operational Taxonomic Unit (OTU) profile derived from a sample collected from a human subject in need thereof, and executing a trained machine learning classifier to predict the probability that the human subject has cancer based on the OTU profile. Also provided are methods for diagnosing and treating a human subject at risk of having cancer, among other things.

### CROSS-REFERENCE TO RELATED APPLICATIONS

The present application claims priority to, and the benefits of U.S. Provisional Patent Application No. 62/745,955, filed Oct. 15, 2018, which is herein incorporated by reference in its entirety.

### FIELD OF THE INVENTION

The present invention relates to compositions and methods for detecting Colorectal cancer (CRC) and its disease progression status in a subject, for the purpose of diagnosing and treating the condition.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is NEWH_002_01US_SeqList ST25.txt. The text file is about 251 KB, and was created on Nov. 27, 2019, and is being submitted electronically via EFS-Web.

### BACKGROUND OF THE INVENTION

Microbiota has been associated with different metabolic diseases (18, 24) and recently, linked to Colorectal and other types of cancer (3, 13, 14, 21, 27). The microbiota induced carcinogenesis may be attributed to mechanisms such as DNA damage, altered β-catenin signaling and engagement of pro-inflammatory pathways as the result of mucosal barrier breach (15).

Due to dynamic changes in host immune system, genotypes and changes in microbiota in different stages of neoplastic process, only a limited number of microbes were known to be carcinogenic to humans. For example, viruses like HPV and HBV and bacterium like Helicobacter pylori may directly cause the development of cancer according to International Agency for Cancer Research. Recently, the mechanism of pro-carcinogenic role of several bacteria has been revealed in mouse models. In familial adenomatous polyposis, a case of CRC with inherited mutation, pks+E. coli and Enterotoxigenic B. fragilis (ETBF) cocolonization enhances colon tumorgenesis compared to the monocolonization with either bacterium (10). The enhancement was manifested in cocolonization compared to monocolonization by several observations: a higher amount of total mucosal IL-17 producing cells, an increased fecal IgA response that was specific topks+E. coli in mice cocolonized with ETBF, an increased mucosal-adherent pks+E. coli, and mucus degradation by ETBF promotes enhanced pks+E. coli colonization but mucus degradation alone was insufficient to promote pks+E. coli colon carcinogenesis. These observations are consistent with sporadic CRC, where studying of ETBF in ApcMin mouse (6) showed that B. fragilis toxin act on colon epithelial cells and involves three major pro-inflammatory signaling pathways, NF-κB, Stat3, and IL-17R, that collectively triggers myeloid cell dependent distal colon tumorigenesis. The accumulation of myeloid derived immune suppressor cells (MDSC) may limit effector T cell accumulation, which in turn may result in ineffective immunotherapy (19). In another study of prevalent bacterial species in CRC (4), Fusobacterium has been shown to persists and co-occurs with other Gram-negative anaerobes in primary and matched metastatic tumors, including Bacteroides fragilis, Bacteroides thetaiotaomicron, Prevotella intermedia and Selenomonas sputigena.

Although these studies begin to reveal the tumorgenesis mechanisms of certain bacterial species, direct diagnostic of CRC by the presence of target microbes of interests remain challenging because these microbes also occur in normal individuals and some of them may not be present in all cancer patients (1). One such recent study (13) uses qPCR to directly assess the presence or absence of three cancer associated markers, clbA+bacteria haboring the pks pathogenicity island, afaC+diffusely adherent E. coli afa1 operon, and Fusobacterium nucleatum. Using a cohort of 238 individuals, the study showed using clbA+ or F. nucleatum alone has 81.5% specificity, 76.9% sensitivity and 76.9% specificity and 69.2% sensitivity, respectively. Whereas combining both gives 63.1% specificity and 84.6% sensitivity. However, a separate independent test dataset is necessary to validate the reported accuracy.

An alternative strategy that uses controlled study to inspect the differences in the microbiota composition between diseased and normal controls are more promising in the prediction of disease status. Baxter et al. (3) combined fecal immunochemical test (FIT) and microbiota to predict CRC and adenomas. However, the method described in Baxter used limited number of selected Operational Taxonomic Units (OTUs) as distinguishing features for prediction. The method did not validate on independent cohort, and did not handle confounding factors such as age and gender. Thus, further improvement is needed.

Therefore, there remains a need to improve ability to detect and classify CRC and its earlier stages for better treatment and management of the disease, with better sensitivity, specificity, and accuracy.

### DESCRIPTION OF THE TEXT FILE SUBMITTED ELECTRONICALLY

The contents of the text file submitted electronically are incorporated herein by reference in their entirety: A computer readable format copy of the Sequence Listing (filename: NEEWH_002_01US_SeqListST25.txt, date recorded: Oct. 14, 2019, file size˜251 kilobytes).

### SUMMARY OF THE INVENTION

The present disclosure provides methods for classifying a human subject as having colorectal cancer (CRC) or being normal (NM).

The present disclosure also provides methods for classifying a human subject as having colorectal cancer (CRC), colorectal adenomas (AD), or being normal (NM).

The present disclosure further provides methods for classifying a human subject as having colorectal cancer (CRC), polyps (PL), non-advanced adenomas (NA), advanced adenomas (AA), or being normal.

In some embodiments, the methods for classifying a human subject as having colorectal cancer (CRC) or being normal (NM) comprise (a) obtaining a fecal sample taken from the human subject. In some embodiments, the methods further comprises (b) producing an Operational Taxonomic Unit (OTU) profile of the sample in step (a). In some embodiments, the methods further comprises (c) providing the OTU profile to a trained machine learning classifier. In some embodiments, the methods further comprise (d) executing the trained machine learning classifier to predict the probability that the human subject has colorectal cancer or being normal.

In some embodiments, the methods for classifying a human subject as having colorectal cancer (CRC), colorectal adenomas (AD), or being normal (NM), comprise (a) obtaining a fecal sample taken from the human subject. In some embodiments, the methods further comprises (b) producing an Operational Taxonomic Unit (OTU) profile of the sample in step (a). In some embodiments, the methods further comprises (c) providing the OTU profile to a trained machine learning classifier. In some embodiments, the methods further comprises (d) executing the trained machine learning classifier to predict the probability that the human subject has colorectal cancer, colorectal adenomas, or being normal.

In some embodiments, the methods for classifying a human subject as having colorectal cancer (CRC), polyps (PL), non-advanced adenomas (NA), advanced adenomas (AA), or being normal comprise (a) obtaining a fecal sample taken from the human subject. In some embodiments, the methods further comprises (b) producing an Operational Taxonomic Unit (OTU) profile of the sample in step (a). In some embodiments, the methods further comprises (c) providing the OTU profile to a trained machine learning classifier. In some embodiments, the methods further comprises (d) executing the trained machine learning classifier to predict the probability that the human subject has colorectal cancer, polyps, non-advanced adenomas, advanced adenomas (AA), or being normal.

In some embodiments, the methods as described herein are computer-aided methods. In some embodiments, the methods comprise using a computer-readable storage device storing computer executable instructions that when executed by a computer control the computer to perform a method disclosed herein.

In some embodiments, methods described herein comprise a step of producing an Operational Taxonomic Unit (OTU) profile based on the fecal sample tested. In some embodiments, the OTU profile is produced by sequencing and quantifying hyper variable region(s) of microbial nucleic acid sequences present in the sample. In some embodiments, the methods comprise (1) amplifying one or more hyper variable regions of microbial nucleic acid sequences present in the sample. In some embodiments, the hyper variable region is a 16S rRNA region. In some embodiments, the 16S rRNA hyper variable region is the V3-V4 hyper variable region. In some embodiments, the methods further comprise (2) sequencing the amplified sequences. In some embodiments, the sequencing step comprises using a high-throughput method, such as a Next Generation Sequencing (NGS) method. In some embodiments, the methods further comprise (3) producing a list of unique microbial sequences present in the fecal sample based on the sequencing result of step (2) to form the OTU profile. In some embodiments, the list comprises abundance information of each unique microbial sequence.

In some embodiments, the OTUs profile produced in methods described herein comprises expression profile of one or more microbial nucleic acid sequences having at least 80%, 85%, 90%, 95%, 96%, 97%, 98%, 99% identity or more to a consensus sequence in SEQ ID NOs. 1-345.

In some embodiments, the machine learning classifier used in methods described herein is selected from the group consisting of decision tree classifier, K-nearest neighbor classifier (KNN), logistic regression classifier, nearest neighbor classifier, neural network classifier, Gaussian mixture model (GMM), Support Vector Machine (SVM) classifier, nearest centroid classifier, linear regression classifier and random forest classifier. In some embodiments, the machine learning classifier is random forest classifier.

In some embodiments, the machine learning classifier has been trained before it is used in methods described herein. In some embodiments, the training process comprises using a set of reference data. In some embodiments, the reference data is collected from human subject population with known labels (e.g., identified as having a certain cancerous condition or being normal). In some embodiments, the reference data is collected from human subject population comprising identified colorectal cancer human patients and normal human subjects. In some embodiments, the reference data is collected from a human subject population comprising identified colorectal cancer human patients, colorectal adenomas human patients, and normal human subjects. In some embodiments, the reference data is collected from a human subject population comprising identified colorectal cancer human patients, polyps human patients, non-advanced adenomas human patients, advanced adenomas human patients, and normal human subjects.

In some embodiments, the reference data for training the machine learning classifier is produced by a computer-aided process. In some embodiments, the process comprises (a) obtaining a collection of human subject fecal samples as training samples. In some embodiments, the training samples are collected from colorectal cancer human patients and normal human subjects. In some embodiments, the fecal samples are collected from colorectal cancer human patients, colorectal adenomas human patients, and normal human subjects. In some embodiments, the fecal samples are collected from colorectal cancer, polyps, non-advanced adenomas, advanced adenomas, and normal human subjects.

In some embodiments, for each fecal sample in the collection, a process as described below can be carried out to produce a reference data set for training the machine learning classifier. In some embodiments, the methods comprise (i) amplifying 16S rRNA hyper variable regions of bacterial nucleic acid sequences in the samples. In some embodiments, the methods further comprise (ii) sequencing the amplified sequences. In some embodiments, the methods further comprise (iii) producing a list of unique microbial sequences present in the sample. In some embodiments, the list comprises abundance information of each unique microbial sequence. In some embodiments, the process comprises grouping the lists of unique microbial sequences obtained to form a reference OTU matrix as the reference data set. In some embodiments, the reference matrix comprises abundance information of each unique microbial sequence for each fecal sample. In some embodiments, the abundance information is relevant abundance of each unique microbial sequence in each sample, such as probability of presence of each unique microbial sequence in each sample.

In some embodiments, the reference OTU matrix is normalized before it is used to train the machine learning classifier, such that the sum of sequence abundance for each sample is the same. In some embodiments, the sum of sequence abundance for each sample is set to a predetermined number, such as an integer. In some embodiments, the integer is about 1 to 1,000,000, such as 1,000 to 10,000, 10,000 to 100,000, 100,000 to 1,000,000, or more. In some embodiments, the integer is 50,000.

In some embodiments, the reference OTU matrix is simplified by reducing the number of OTUs through feature selection. In some embodiments, the feature selection is to remove low abundant OTUs across training samples. In some embodiments, low abundant OTUs are those having a relevant abundancy less than 0.05%, 0.04%, 0.03%, 0.02%, 0.01%, or even less.

In some embodiments, the machine learning classifier is a random forest classifier. In some embodiments, hyperparameters of the random forest are tuned using cross validation method. In some embodiments, the hyperparameters to be tuned comprise the number of trees, number of maximum features used for each split of tree, and minimum samples per leaf.

In some embodiments, the methods for classifying a human subject as having colorectal cancer (CRC) or being normal (NM) has an accuracy of at least 85%, 86%, 87%, 88%, 89%, 90%, 91%, 92%, 93%, 94%, 95%, 96%, 97%, 98%, 99%, or more.

In some embodiments, the methods for classifying a human subject as having colorectal cancer (CRC), colorectal adenomas (AD), or being normal (NM) has an accuracy of at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more.

In some embodiments, the methods for classifying a human subject as having colorectal cancer (CRC), polyps (PL), non-advanced adenomas (NA), advanced adenomas (AA), or being normal has an accuracy of at least 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more.

In some embodiments, the machine learning classifier automatically determines the list of the most relevant OTUs in the OTU profile associated with a certain condition of interest. In some embodiments, the OTU profile comprises one or more OTUs selected from the group consisting of:

| Otu | Annotation L |
|---|---|
| Otu101 | d: Bacteria, p: Bacteroidetes, c: Bacteroidia, o: Bacteroidales, f: Prevotellaceae, g: Prevotella, |
| | s: Prevotella_{—}intermedia |
| Otu169 | d: Bacteria, p: Bacteroidetes, c: Bacteroidia, o: Bacteroidales, f: Porphyromonadaceae, g: Porphyromonas |
| Otu172 | d: Bacteria, p: Firmicutes, c: Clostridia, o: Clostridiales, f: Peptostreptococcaceae, g: Peptostreptococcus, |
| | s: Peptostreptococcus_{—}stomatis |
| Otu121 | d: Bacteria, p: Bacteroidetes, c: Bacteroidia, o: Bacteroidales, f: Bacteroidaceae, g: Bacteroides, |
| | s: Bacteroides_{—}nordii |
| Otu185 | d: Bacteria, p: Firmicutes, c: Clostridia, o: Clostridiales, f: Clostridiales_Incertae_Sedis_XI, g: Parvimonas, |
| | s: Parvimonas_{—}micra |
| Otu168 | d: Bacteria, p: Firmicutes, c: Negativicutes, o: Selenomonadales, f: Veillonellaceae, g: Dialister, |
| | s: Dialister_{—}pneumosintes |
| Otu147 | d: Bacteria, p: Fusobacteria, c: Fusobacteriia, o: Fusobacteriales, f: Fusobacteriaceae, g: Fusobacterium |
| Otu47 | d: Bacteria, p: Firmicutes, c: Clostridia, o: Clostridiales, f: Peptostreptococcaceae, g: Romboutsia, |
| | s: Romboutsia_{—}sedimentorum |
| Otu142 | d: Bacteria, p: Bacteroidetes, c: Bacteroidia, o: Bacteroidales, f: Porphyromonadaceae, g: Porphyromonas, |
| | s: Porphyromonas_{—}endodontalis |
| Otu10 | d: Bacteria, p: Firmicutes, c: Clostridia, o: Clostridiales, f: Lachnospiraceae |

In some embodiments, the OTU profile comprises one or more OTUs selected from SEQ ID NO. 1-345. In some embodiments, the OTU profile comprises one or more OTUs having about 70%, 75%, 80%, 85%, 90%, 91%, 92%, 93%, 94%, 95%, 96%, 97%, 98%, 99% or more identity to a sequence of SEQ ID NO. 1-345.

In some embodiments, the collection of human subject fecal samples contains samples collected from at least about 20, 25, 30, 35, 40, 45, 50, 55, 60, 65, 70, 75, 80, 85, 90, 95, 100, 150, 200, 250, 300, 350, 400, 450, 500 human subjects, or more.

In some embodiments, the sequencing step of methods described herein comprises sequencing at least 100, 200, 300, 400, 500, 600, 700, 800, 900, 1000, 2000, 3000, 4000, 5,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, or more amplified fragments for each fecal sample.

The present disclosure also provides methods for identifying an increased chance of colorectal adenomas or colorectal cancer in a human subject. In some embodiments, the methods are computer-aided. In some embodiments, the methods comprise executing a trained machine learning classifier as described herein to predict the probability that the human subject has increased chance of colorectal adenomas colorectal cancer.

The present disclosure also provides methods for the detection of abnormalities in a human subject's fecal sample. In some embodiments, the methods comprises executing the trained machine learning classifier to predict the presence or absence of abnormalities in the patient's fecal sample. In some embodiments, the abnormalities include colorectal cancer (CRC), polyps (PL), non-advanced adenomas (NA), advanced adenomas (AA),

The present disclosure further provides methods for generating a personalized treatment plan for to a human subject having colorectal adenomas or colorectal cancer. In some embodiments, the methods comprise (1) ordering a diagnostic test of the human subject's fecal sample. In some embodiments, the test comprises (a) obtaining a fecal sample taken from the human subject. In some embodiments, the test further comprises (b) producing an Operational Taxonomic Unit (OTU) profile of the sample in step (a). In some embodiments, the test further comprises (c) providing the OTU profile to a trained machine learning classifier. In some embodiments, the test further comprises (d) executing the trained machine learning classifier to predict the probability that the human subject has colorectal adenomas or colorectal cancer. In some embodiments, the methods comprise (2) generating the personalized treatment plan to the human patient based on the test results.

The present disclosure further provides methods for diagnosing and treating a human subject at risk of colorectal adenomas or colorectal cancer. In some embodiments, the methods comprise (1) ordering a diagnostic test of the human subject's fecal sample. In some embodiments, the test comprises (a) obtaining a fecal sample taken from the human subject. In some embodiments, the test further comprises (b) producing an Operational Taxonomic Unit (OTU) profile of the sample in step (a). In some embodiments, the test further comprises (c) providing the OTU profile to a trained machine learning classifier. In some embodiments, the test further comprises (d) executing the trained machine learning classifier to predict the probability that the human subject has colorectal adenomas or colorectal cancer. In some embodiments, the methods further comprise (2) treating the human subject based on the diagnostic test results of step (1).

In some embodiments, the methods comprise methods of monitoring progression of colorectal adenomas or colorectal cancer in a human subject. In some embodiments, the methods comprise (a) obtaining a fecal sample taken from the human subject. In some embodiments, the methods further comprise (b) producing an Operational Taxonomic Unit (OTU) profile of the sample in step (a). In some embodiments, the methods further comprise (c) providing the OTU profile to a trained machine learning classifier. In some embodiments, the methods further comprise (d) executing the trained machine learning classifier to predict the stage of colorectal adenomas or colorectal cancer in the human subject. Optionally, the methods further comprise (e) repeating steps (a) to (d) periodically.

In some embodiments, the present disclosure also provides methods for distinguishing colorectal cancer (CRC) patients and normal human subjects. In some embodiments, the present disclosure also provides methods for distinguishing colorectal cancer (CRC) patients, colorectal adenomas patients, and normal human subjects. In some embodiments, the present disclosure also provides methods for distinguishing colorectal cancer, colorectal polyps (PL), non-advanced colorectal adenomas (NA), and advanced colorectal adenomas (AA). In some embodiments, the methods as mentioned herein comprise executing the trained machine learning classifier as described herein.

### BRIEF DESCRIPTION OF THE FIGURES

FIG. 1 depicts the number and percentage of sequence fragments as input, after merging and quality filtering steps.

FIG. 2A and FIG. 2B depict age (FIG. 2A) and gender (FIG. 2B) distribution among five groups of all three batches.

FIG. 3 depicts CR and NM classification using age and gender. Out-of-bag (OOB) error is indicated by the middle line whereas the misclassification errors for individual groups are represented by other lines.

FIG. 4 depicts accuracy of multi-group prediction with spike-ins. The classifier is built from the first batch (batch 2 samples) plus an increasing number (specified by x-axis) of spike-in samples from the second batch (batch 3 samples). Predictions were made for the remaining samples in the second batch.

FIG. 5 depicts theoretical composition of ZymoBIOMICS™ Microbial Community DNA Standard with the known mixture which is used as positive control.

FIG. 6A depicts Pearson and Spearman correlations among three samples on genus level.

FIG. 6B depicts Pearson and Spearman correlations among three samples on species level.

FIG. 7A depicts number of observed genus and species and the overlaps with the truth (last column) on genus level. FIG. 7B depicts number of observed genus and species and the overlaps with the truth (last column) on species level.

FIG. 8 depicts contaminations in the sequencing data relative abundance of contamination on genus and species levels.

FIG. 9 depicts misclassification errors for individual groups when different number of trees are used for training the classifier which is used to predict CR and NM.

FIG. 10 depicts Mean Decrease Accuracy and Mean Decrease in Gini Coefficient associated with OTUs selected by the trained the classifier which is used to predict CR and NM. Mean Decrease in Gini Coefficient is a measure of how each variable contributes to the homogeneity of the nodes and leaves in the resulting random forest. Variables that result in nodes with higher purity have a higher Decrease in Gini Coefficient.

FIG. 11 depicts misclassification errors for individual groups when different number of trees are used for training the classifier which is used to predict CR (cancer) and JK (normal) in NuoHui 999 combined with batch 2 and batch 3 stool microbiome samples.

FIG. 12 depicts Mean Decrease Accuracy and Mean Decrease in Gini Coefficient associated with OTUs selected by the trained classifier which is used to predict CR (cancer) and JK (normal) in NuoHui 999 combined with batch 2 and batch 3 stool microbiome samples.

FIG. 13 depicts misclassification errors for individual groups when different number of trees are used for training the classifier which is used to predict CR (cancer), JZ (progression), FJ (non-progression), XR (polypus), and JK (normal) in NuoHui 999 combined with batch 2 and batch3 stool microbiome samples.

FIG. 14 depicts Mean Decrease Accuracy and Mean Decrease in Gini Coefficient associated with OTUs selected by the trained classifier which is used to predict CR (cancer), JZ (progression), FJ (non-progression), XR (polypus), and JK (normal) in NuoHui 999 combined with batch 2 and batch3 stool microbiome samples.

FIG. 15 depicts misclassification errors for individual groups when different number of trees are used for training the classifier which is used to predict adenoma (including JZ (progression) and FJ (non-progression)) vs. the remaining groups (CR (cancer), XR (polypus), and JK (normal)) in NuoHui 999 combined with batch 2 and batch3 stool microbiome samples.

FIG. 16 depicts Mean Decrease Accuracy and Mean Decrease in Gini Coefficient associated with OTUs selected by the trained classifier which is used to predict adenoma (including JZ (progression) and FJ (non-progression)) vs. the remaining in NuoHui 999 combined with batch 2 and batch3 stool microbiome samples.

FIG. 17 depicts misclassification errors for individual groups when different number of trees are used for training the classifier which is used to predict adenoma (including JZ (progression) and FJ (non-progression)) vs. non-diseased groups (XR (polypus) and JK (normal)) in NuoHui 999 combined with batch 2 and batch3 stool microbiome samples.

FIG. 18 depicts Mean Decrease Accuracy and Mean Decrease in Gini Coefficient associated with OTUs selected by the trained classifier which is used to predict adenoma (including JZ (progression) and FJ (non-progression)) vs. non-diseased groups (XR (polypus) and JK (normal)) in NuoHui 999 combined with batch 2 and batch3 stool microbiome samples.

FIG. 19 depicts Multi-Dimensional Scaling Plot (MDSplot) Of Proximity Matrix From RandomForest in multi-group prediction using independent training and test samples. JZ (progression), CR (cancer), JK (normal).

FIG. 20 depicts changes of sensitivity when different numbers of samples of each the five groups (CR, JZ, FJ, XR, JK) in the second batch were spiked-in with the samples in the first batch (the reference batch).

FIG. 21 depicts changes of specificity when different numbers of samples of each the five groups (CR, JZ, FJ, XR, JK) in the second batch were spiked-in with the samples in the first batch (the reference batch).

FIG. 22 depicts changes of accuracy when different numbers of samples of each the five groups (CR, JZ, FJ, XR, JK) in the second batch were spiked-in with the samples in the first batch (the reference batch).

### DETAILED DESCRIPTION OF THE INVENTION

The present disclosure, in some embodiments, relates to cancer diagnosis and treatment. More particularly, the present disclosure relates to, but not exclusively, methods and systems of classifying digestive system related condition in a human subject, such as detecting the present of a cancerous condition, determining stage of cancer, or evaluating a risk of cancer. In some embodiments, the cancer is colorectal cancer, bowel cancer, colon cancer, rectum cancer, lower gastrointestinal tract cancer, ceum cancer, large intestine cancer, etc.

Methods and systems of the present disclosure may be applied to any human subjects in need thereof. In some embodiments, the human subjects are suspected to have cancer or at risk of having cancer. In some embodiments, the human subjects are exposed to risk factors include but not limited to, a personal or family history of colorectal cancer or polyps, a diet high in red meats and processed meats, inflammatory bowel disease (Crohn's disease or ulcerative colitis), inherited conditions such as familial adenomatous polyposis and hereditary non-polyposis colon cancer, obesity, smoking, physical inactivity, heavy alcohol use, Type 2 diabetes, being African-American, older age, male gender, high intake of fat, or having particular genetic disorders. In some embodiments, the human subjects have one or more symptoms related to colorectal cancer, including but not limited to, a persistent change in bowel habits (such as constipation or diarrhea), blood on or in the stool, worsening constipation, abdominal discomfort, unexplained weight loss, decrease in stool caliber (thickness), loss of appetite, and nausea or vomiting and anemia. In some embodiments, the human subjects are up to a regular health examination.

In some embodiments, methods and systems of the present disclosure may be applied to any human subjects in need thereof for cancer classification solely based on Operational Taxonomic Unit (OTU) profile of the sample obtained from a human subject, without knowing other information, so that the disntinguishing features in a classifer only consists of OTUs. In some embodiments, the OTU was not manually screened other than certain quality control, such as those aminig to avoid rare OTUs and to reduce potential contamination and improve model bias. In some embodiments, the methods and systems can be applied together with other test, including but not limited to, genetic test of the human subject, macroscopy. microscopy, immunochemistry, in situ detection, and micrographs, such as colonoscopy, fecal occult blood testing, and flexible sigmoidoscop.

According to some embodiments of the present disclosure, there are provided methods and systems of evaluating cancer risk, such as colorectal cancer, by analyzing a sample of a target individual. For colorectal cancer, in some embodiments, the sample is a fecal sample. Non-limiting exemplary methods and devices for fecal sample collection and handling are described in U.S. Pat. Nos. 8,008,036, 8,053,203, 7,449,340, 4,333,734, 6,727,073, 9,410,962, 7,816,077, and 5,344,762, each of which is incorporated by reference in its entirety for all purposes.

Methods and systems of the present disclosure in some embodiments comprise one or more machine learning classifiers. Such classifiers can be generated according to the procedure described herein.

Optionally, the one or more classifiers are adapted to one or more characteristics of the human subject being tested. Optionally, the classifiers are selected to match one or more characteristics of the human subject being tested. In such embodiments, different classifiers may be used according to factors including but not limited to gender, age, race, genetic background, living style, geographic locates, etc.

According to some embodiments of the present disclosure, there are provided methods and systems of generating one or more classifiers that can be used to perform the tasks as described herein, such as classifying colorectal condition of a human subject in need. In some embodiments, the methods and systems for generating the classifiers are based on analysis of a plurality of sampled individuals. The dataset is used to generate, train and output one or more classifiers. The classifiers may be provided as modules for execution on client terminals or used as an online service for evaluating cancer risk of target individuals based on the sample collected from the human subject in need thereof.

The sampled individuals for generating and training a classifier can be selected based on the purpose of the classifier, and/or tasks to be performed using the classifier after it is generated.

In some embodiments, the task to be performed is to classify a human subject as having colorectal cancer, or being normal (i.e., non-cancer). In some embodiments, the sampled individuals as a reference human subject population for generating and training a classifier comprise human subjects already identified as having colorectal cancer, and normal human subjects (e.g., having no colorectal cancer). The population size of the sampled individuals can be determined and optimized based on the purpose of the tasks, and/or accuracy as needed. In some embodiments, the population has at least 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10,000, or more. In some embodiments, the ratio of human subjects already identified as having colorectal cancer to normal human subjects is about 1.0, such as about 1.1, 1.2, 1.3, or about 0.9, 0.8, 0.7, but variations are allowed as long as a desired accuracy can be achieved. In some embodiments, the ratio of human subjects already identified as having colorectal cancer to normal human subjects is about 10:1, 9:1, 8:1, 7:1, 6:1, 5:1, 4:1, 3:1, 2:1, 1:2, 1:3, 1:4, 1:5, 1:6, 1:7, 1:8, 1:9, or 1:10. Different ratio can be used as long as a desired prediction accuracy is achieved.

In some embodiments, the task to be performed is to classify a human subject as having colorectal cancer (CRC), colorectal adenomas (AD), or being normal (NM). In some embodiments, the sampled individuals as a reference human subject population for generating and training a classifier comprise human subjects already identified as having colorectal cancer, human subjects already identified as having colorectal adenomas, and normal human subjects (e.g., having no colorectal cancer or colorectal adenomas). The population size of the sampled individuals can be determined and optimized based on the purpose of the tasks, and/or accuracy as needed. In some embodiments, the population has at least 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10,000, or more.

In some embodiments, the ratio among human subjects already identified as having colorectal cancer, human subjects already identified as having CRC, AD, and normal human subjects is about 1:1:1, but variations are allowed as long as a desired accuracy can be achieved.

In some embodiments, the task to be performed is to classify a human subject as having colorectal cancer (CRC), polyps (PL), non-advanced adenomas (NA), advanced adenomas (AA), or being normal. In some embodiments, the sampled individuals as a reference human subject population for generating and training a classifier comprise human subjects already identified as having colorectal cancer, human subjects already identified as having polyps, human subjects already identified as having non-advanced adenomas, human subjects already identified as having advanced adenomas, and normal human subjects (e.g., having no CRC, PL, NA, or AA). The population size of the sampled individuals can be determined and optimized based on the purpose of the tasks, and/or accuracy as needed. In some embodiments, the population has at least 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10,000, or more. In some embodiments, the ratio among human subjects already identified as having colorectal cancer, human subjects already identified as having CRC, PL, NA, AA, and normal human subjects is about 1:1:1:1:1, but variations are allowed as long as a desired accuracy can be achieved.

In some embodiments, for the methods described herein, samples collected from the reference human subject population are processed together (spiked-in) with one or more samples collected from target individuals (e.g., human subjects in need thereof whose health conditions are to be determined). In some embodiments, said processing step comprises amplifying and sequencing microbial sequences in the samples. In some embodiments, said processing step comprises simplifying, normalizing, and/filtering the sequencing results. In some embodiments, said processing step comprises producing OTU profiles for each sample. In some embodiments, the spiked-in samples collected from target individuals (e.g., human subjects in need thereof whose health conditions are to be determined) comprise about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90% or more of the total samples being processed together. In some embodiments, the number of spiked-in samples collected from target individuals (e.g., human subjects in need thereof whose health conditions are to be determined) in total samples being process together is about 10, 15, 20, 25, 30, 35, 40, 45, 50, 55, 60, 65, 70, 75, 80, 85, 90, 95, 100, or more).

### OTUs

Methods of systems of the present disclosure use Operational Taxonomic Unit (OTU) profile. In some embodiments, OTUs in the OTU profile for classifying cancer conditions according to the procedure described herein comprise OTUs determined by the machine learning classifier. In this case, the machine learning classifier is viewed as a black-box, and the selection of OTUs is not manipulated by any outside factors.

These OTUs selected by the machine learning classifier relate to cancer conditions and can be used in cancer detection or classification. In some embodiments, OTUs of the present disclosure include those nucleic acid sequences in the Sequence Listing, such as nucleic acids having sequences in SEQ ID NOs. 1 to 345. It is understood that variants of these sequences, such as those having at least 70%, 75%, 80%, 85%, 90%, 91%, 92%, 93%, 94%, 95%, 96%, 97%, 98%, 99%, or higher identity compares to a sequence in the Sequence Listing, or being capable of hybridizing to a sequence in the Sequence Listing under stringent hybridization conditions. The variant may be a complement of the referenced nucleotide sequence. The variant may also be a nucleotide sequence that is substantially identical to the referenced nucleotide sequence or the complement thereof. The variant may also be a nucleotide sequence which hybridizes under stringent conditions to the referenced nucleotide sequence, complements thereof, or nucleotide sequences substantially identical thereto.

In some embodiments, methods of systems of the present disclosure comprise a reference OTU profile that can be used to generate and train a machine learning classifier of the present disclosure.

To produce a reference OTU profile, a collection of human subject samples is obtained as training samples. In some embodiments, the training samples are fecal samples. As used herein, the term fecal samples include treated or un-treated stool of sampled individuals, as long as the nucleic acid compositions of microbiota are preserved. In some embodiments, the training samples are diverse enough to capture group variance.

For each fecal sample, ribosomal RNA (rRNA) gene sequences are used for determining microbiota in the sample. In some embodiments, the small-subunit (SSU) and large-subunit (LSU) rRNA genes and the internal transcribed spacer (ITS) region that separates the two rRNA genes can be used. In some embodiments, the rRNA genes can be 23S rRNA or 16S RNA. In some embodiments, 16S RNA sequences are used.

In some embodiments, their entire or one or more parts of 16S rRNA in the sample are amplified. To amplify the 16S RNA sequences, any suitable primer pair can be used, such as 27F and 1492R described in Weisburg et al. (Journal of Bacteriology. 173 (2): 697-703), or 27F/8F-534R covering V1 to V3 used for 454 sequencing. More examples are provided in the table below. It is understood that primers having high identity to the primers listed below, such as those having at least 80%, 85%, 90%, 95%, or more can also be used.

| Primer | | SEQ ID |
|---|---|---|
| name | Sequence (5′-3′) | NO. |
| 341F | CCTAYGGGRBGCASCAG | 346 |
| 806R | GGACTACNNGGGTATCTAAT | 347 |
| 8F | AGA GTT TGA TCC TGG CTC AG | 348 |
| U1492R | GGT TAC CTT GTT ACG ACT T | 349 |
| 928F | TAA AAC TYA AAK GAA TTG ACG GG | 350 |
| 336R | ACT GCT GCS YCC CGT AGG AGT CT | 351 |
| 1100F | YAA CGA GCG CAA CCC | 352 |
| 1100R | GGG TTG CGC TCG TTG | 353 |
| 337F | GAC TCC TAC GGG AGG CWG CAG | 354 |
| 907R | CCG TCA ATT CCT TTR AGT TT | 355 |
| 785F | GGA TTA GAT ACC CTG GTA | 356 |
| 805R | GAC TAC CAG GGT ATC TAA TC | 357 |
| 533F | GTG CCA GCM GCC GCG GTA A | 358 |
| 518R | GTA TTA CCG CGG CTG G | 359 |
| 27F | AGA GTT TGA TCM TGG CTC AG | 360 |
| 1492R | CGG TTA CCT TGT TAC GAC TT | 361 |

In some embodiments, one or more hyper variable regions of 16S rRNA nucleic acid sequences are amplified and sequenced. The bacterial 16S gene contains nine hypervariable regions (V1-V9) ranging from about 30-100 base pairs long that are involved in the secondary structure of the small ribosomal subunit. In theory, one or more hypervariable regions thereof can be used for the purpose of methods described in the present disclosure. In some embodiments, Primers targeting fragment of V3, V4, or V3-V4 regions of 16S rRNA are used. For example, the primer pair comprises 341F (CCTAYGGGRBGCASCAG, SEQ ID NO. 346) and 806R (GGACTACNNGGGTATCTAAT, SEQ ID NO. 347). In some embodiments, primers targeting other regions can be used, such as the V6 region of 16S rRNA. It is understood that for certain bacterial taxonomic studies, species may share up to 99% sequence similarity across the 16S gene. In such cases, sequences other than 16S rRNA can be introduced.

A suitable sequencing method can be used. DNA sequencing techniques include classic dideoxy sequencing reactions (Sanger method) using labeled terminators or primers and gel separation in slab or capillary, single molecule sequencing, sequencing by synthesis using reversibly terminated labeled nucleotides, pyrosequencing, 454 sequencing, Illumina sequencing, SMRT sequencing, nanopore sequencing, Chemical-Sensitive Field Effect Transistor Array Sequencing, Sequencing with an Electron Microscope, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of the separated molecules has more recently been demonstrated by sequential or single extension reactions using polymerases or ligases as well as by single or sequential differential hybridizations with libraries of probes.

In some embodiments, the sequencing technique can generate least 1000 reads per run, at least 10,000 reads per run, at least 100,000 reads per run, at least 500,000 reads per run, or at least 1,000,000 reads per run. In some embodiments, the sequencing technique can generate about 30 bp, about 40 bp, about 50 bp, about 60 bp, about 70 bp, about 80 bp, about 90 bp, about 100 bp, about 110, about 120 bp per read, about 150 bp, about 200 bp, about 250 bp, about 300 bp, about 350 bp, about 400 bp, about 450 bp, about 500 bp, about 550 bp, or about 600 bp per read. In some embodiments, the sequencing technique used in the methods of the provided invention can generate at least 30, 40, 50, 60, 70, 80, 90, 100, 110, 120, 150, 200, 250, 300, 350, 400, 450, 500, 550, or 600 bp per read. In some embodiments, the sequencing technique used in the methods of the provided invention can generate at least 100, 200, 300, 400, 500, 600 bp, 700, 800, 900, 1000, 2000, 3000, 4000, 5000, 6000, 7000, 8000, 9000, 10000 bp per read, or more.

Once the sequencing results are obtained, it can be compared to one or more 16S rRNA databases to obtain annotations at different taxonomic rank. Such databases include, but are not limited to, SILVA (23), Ribosomal Database Project (RDP) (7), EzTaxon-e (Chun et al., International Journal of Systematic and Evolutionary Microbiology. 57 (Pt 10): 2259-61, 2007), and GreenGenes (DeSantis et al., Applied and Environmental Microbiology. 72 (7): 5069-72. 2006), and NCBI.

In some embodiments, while the amplified nucleic acids are sequenced, the abundance of each sequence (e.g., absolute abundance or relative abundance) can be determined as well, according to methods known in the art.

For each fecal sample, after sequence and abundance information of each amplified nucleic acids are available, a list of unique microbial sequences present in the sample is created, which comprises abundance information of each unique microbial sequence. Accordingly, for each sample of an individual, a list comprising identities information of unique microbial sequences (e.g., taxonomy information of the microbes from which the sequences are derived from) and abundance information of each unique microbial sequence is produced. Then the lists derived from a plurality of samples can be combined to form a reference OTU matrix as a reference data set. The reference matrix comprises abundance information of each unique microbial sequence for each fecal sample. A typical reference matrix may look like the one below:

$$A = {[ \begin{matrix}{a}_{11} & {a}_{12} & {a}_{13} & {a}_{14} & … & {a}_{1 n} \\ {a}_{21} & {a}_{22} & {a}_{23} & {a}_{24} & … & {a}_{2 n} \\ {a}_{31} & {a}_{32} & {a}_{33} & {a}_{34} & … & {a}_{3 n} \\ . & . & . & . & … & . \\ . & . & . & {a}_{ij} & … & . \\ . & . & . & . & … & . \\ {a}_{m 1} & . & . & . & … & {a}_{mn}\end{matrix} ]}_{m \times n} or A = {[ {a}_{ij} ]}_{m \times n} ,$$

Wherein each row of the matrix represents abundance of given unique microbial sequences (OTUs) in each fecal sample. For example, aij in the matrix represents the abundance of OTUi in sample j.

In some embodiments, sequencing results are passed through a filter to remove less desired sequencing results. In some embodiments, the filter is based on sequencing quality. In some embodiments, fragments passed the filter are further merged to form unique sequences list and their abundances are obtained. In some embodiments, the unique sequences are clustered using a predetermined similarity threshold, such as about 90%, 91%, 92%, 93%, 94%, 95%, 96%, 97%, 98%, 99%, or more. For each OTU, a consensus sequence is selected. In some embodiments, the consensus sequence is selected from SEQ ID NOs. 1-345, or having high similarity thereof.

For convenience of computation, the matrix can be normalized, so that the sum of sequence abundance for each sample j would be the same. The sum can be chosen as needed. In some embodiments, the chosen sum can be close to total number of sequenced nucleic acid population. For example, when about 50,000 sequences are obtained from the sequencing step, the sum of the normalized matrix can be set to 50,000. Alternatively, different sum can be chosen.

Once the reference OTU matrix is available, it can be used to generate and train a classifier which ultimately can be used to predict if a given sample associates with cancer.

### Classifiers

The present disclosure also provides machine learning classifiers that can be used to classify if a given sample is associated with a cancerous condition. Such machine learning classifiers include, but are not limited to, decision tree classifier, K-nearest neighbor classifier (KNN), logistic regression classifier, nearest neighbor classifier, neural network classifier, Gaussian mixture model (GMM), Support Vector Machine (SVM) classifier, nearest centroid classifier, linear regression classifier and random forest classifier.

Before a machine learning classifier is used to perform a task as described herein, the classifier can be trained.

In some embodiments, each sample is represented by a vector of relative OTU abundances, serving as the “features” used in a classifier.

In some embodiments, the classifier is a random forest classifier. Random forest classifier is an ensemble tool which takes a subset of observations and a subset of variables to build a decision tree. It builds multiple such decision trees and amalgamate them together to get a more accurate and stable prediction. This is direct consequence of the fact that by maximum voting from a panel of independent judges, one can get the final prediction better than the best judge.

For implementation, a software package containing a random forest algorithm can be used. Such software package include, but are not limited to, The Original RF by Breiman and Culter written in Fortran; ALGLIB in C#, C++, Pascal, VBA; party implementation based on the conditional inference trees in R; RandomForest for classification and regression in R; Python implementation with examples in scikit-learn; Orange data mining suite includes random forest learner and can visualize the trained forest; Matlab implementation; SQP software uses random forest algorithm to predict the quality of survey questions, depending on formal and linguistic characteristics of the question; Weka RandomForest in Java library and GUI; and ranger (C++ implementation of random forest for classification, regression, probability and survival).

Hyperparameters in random forest are either to increase the predictive power of the model or to make it easier to train the model. Optionally, before a machine learning classifier is used to perform a task as described herein, one or more hyperparameters of the classifier can be tuned. The hyperparameter tuning methods relate to how one can sample possible model architecture candidates from the space of possible hyperparameter values. This is often referred to as “searching” the hyperparameter space for the optimum values.

In some embodiments, depending on the software package to be used, the hyperparameters to be tuned include, but are not limited to, the number of trees, number of maximum features used for each split of tree, minimum samples per leaf, degree of polynomial features, maximum depth allowed, number of neurons in the neural network, number of layers in the neural network, learning rate, etc.

In some embodiments, when a random forest classifier is used, such as the random forest package in R, certain values can be set.

In some embodiments, mtry is set to be square root of the total parameters.

In some embodiments, the number of trees is set to be about 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, or more. In some embodiments, each tree is allowed to grow to full size. In some embodiments, each tree is not allowed to grow to full size.

In some embodiments, features used in the random tree classifier are reduced. In some embodiments, only features satisfying certain criteria are retained. In some embodiments, the criteria include that each feature occurs in at least among p % (e.g., p=1, 2, 3, 4, 5, 6, 7, 8, 9, 10, or more) of samples with relative abundance at least f % (e.g., f=0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, or more). In some embodiments, in order to avoid removing a real discriminative signal, random permutation is first applied to shuffle the samples. In some embodiments, the number of features after reduction becomes comparable to the number of training samples, which reduce run time significantly.

Classifiers according to present disclosure may be used in many ways. In some embodiments, methods for aiding in the prediction of cancer in a subject is based upon one or more of the classifiers, alone or in combination with another feature profile, such as a symptom profile. In certain embodiments, the classifier is a machine learning classifier. The machine learning classifier can be selected from the group consisting of a random forest (RF), classification and regression tree (C&RT), boosted tree, neural network (NN), support vector machine (SVM), general chi-squared automatic interaction detector model, interactive tree, multiadaptive regression spline, machine learning classifier, and combinations thereof. Preferably, the learning statistical classifier system is a tree-based statistical algorithm (e.g., RF, C&RT, etc.) and/or a NN (e.g., artificial NN, etc.).

In addition to using the classifiers for prediction of cancerous conditions in human subjects, other methods are also provided. For example, methods for identifying an increased chance of cancer in a human subject are provided. In some embodiments, human patients identified as having an early stage cancerous condition are provided, and samples are collected from said human patients periodically, such as every year, every half year, every month, every week, etc., and the information related to cancer development stage is also provided to each sample. The samples are processed according to the procedure described herein to produce a reference data set, which is used to train a classifier to distinguish from human subjects that had worsened cancer conditions and human subjects that had no worsened cancer conditions. In some embodiments, the methods comprise executing the trained machine learning classifier to predict the probability that the human subject has increased chance of colorectal adenomas or colorectal cancer.

Methods for the detection of abnormalities in a human subject's sample are also provided. As used herein, the term abnormalities refer to any condition that a healthy human subject does not have. In some embodiments, the abnormalities related to the digestive system. In some embodiments, the abnormalities related to the colorectal part. In some embodiments, a machine learning classifier is used, wherein the machine learning classifier has been trained using samples of human subjects identified as being normal, and human subjects identified as having at least one abnormality. In some embodiments, the methods comprise executing the trained machine learning classifier to predict the presence or absence of abnormalities in the patient's fecal sample.

Method for generating a personalized treatment plan for to a human subject having cancer or at risk of developing cancer. The methods may be initiated by a medical practitioner such as a doctor by ordering a diagnostic test of the human subject's sample. The sample is processed according to the procedure described herein to produce a personalized medical profile. Accordingly, a trained machine learning classifier is employed to classify the personalized medical profile to a particular cancerous or non-cancerous condition. Based on the determined condition, a personalized treatment plan to the human patient is recommended, such as if any suitable treatment should be prescribed. For the same practice, methods for diagnosing and treating a human subject at risk of cancer are also provided, in which the human subject receives the prescribed treatment based on the classification results. The personalized treatment plan facilitates the timely, efficient, and accurate application of cancer therapy, or other treatment modalities. In one embodiment, the training data set may be divided into at least two groups, including those patients who did not experience cancer recurrence, and those patients who experienced cancer recurrence. In one embodiment, the classifier is trained to distinguish from patients who did not experience cancer recurrence, and those patients who experienced cancer recurrence. Accordingly, such a classifier can be used to process a sample collected from the human patient experienced cancer and predict if there is cancer recurrence risk in said human patient. In one embodiment, a threshold score may be computed such that a percentage of recurrence patients have quantitative risk scores less than the threshold score. The threshold score may be user adjustable. Thus, a quantitative risk score less than the threshold score indicates a low-risk of cancer recurrence, and example methods and apparatus may generate a personalized treatment plan for the patient after surgery that indicates that no adjuvant chemotherapy should be part of the treatment plan. Quantitative risk scores above the threshold score indicate a higher risk of cancer recurrence, suggesting that adjuvant chemotherapy should be part of a personalized treatment plan for the patient. Thus, in one embodiment, upon detecting a quantitative risk score less than a threshold score, a personalized treatment plan that indicates no adjuvant chemotherapy should be administered to the patient is generated. Upon detecting a quantitative risk score equal to or greater than the threshold score, a personalized treatment plan that indicates that adjuvant chemotherapy should be administered to the patient is generated.

Methods for monitoring progression of cancer in a human subject are also provided. In some embodiments, a sample is taken from the human subject periodically, such as such as every year, every half year, every month, every week, etc., and subjected to the process as described herein to produce a set of OTU profiles of the human subject. The profiles are analyzed by the trained machine learning classifier to monitor the development of a cancerous condition in the human subject to determine if health condition in the patient has changed.

Methods for predicting recurrence of a cancerous condition in a human subject are also provided. In some embodiments, a sample is taken from the human subject once had a cancerous condition periodically, such as such as every year, every half year, every month, every week, etc., and subjected to the process as described herein to produce a set of OTU profiles of the human subject. The profiles are analyzed by the trained machine learning classifier to determine if recurrence of the cancer happens. In some embodiments, the machine learning classifier computes the probability that a subject will experience cancer recurrence based, at least in part, on the OTU profiles.

In some embodiments, a diagnostic test of the present disclosure can be ordered and performed by a same party. In some embodiments, the test can be ordered and performed by two or more different parties. In some embodiments, the test can be ordered and/or performed by the subject himself/herself, by a doctor, by a nurse, by a test lab, by a healthcare provider, or any other parties capable of doing the test. The test results can be then analyzed by the same party or by a second party, such as the subject himself/herself, a doctor, a nurse, a test lab, a healthcare provider, a physician, a clinical trial personnel, a hospital, a lab, a research institute, or any other parties capable of analyzing the results using methods as described herein.

### Prediction

In some embodiments, once a classifier is trained, it can be used directly to predict if a given sample collected from a human subject in need thereof associates with cancerous condition or risk of cancerous condition. In this case, the reference samples of known labels (e.g., samples derived from the reference human subject population identified as having a cancerous condition or being normal) are processed to produce a training data set independently without a new sample collected from a human subject in need thereof.

In some embodiments, a new sample collected from a human subject in need thereof is processed together with the reference samples of known labels (e.g., samples derived from the reference human subject population identified as having a cancerous condition or being normal), using the procedure as described herein. The results associated with the reference human subject population are used to train a classifier, which is then used for making prediction. Such a process give the new sample the same set of OTU labels as the samples used for building the classifier, and increase prediction accuracy due to batch effects.

In some embodiments, in order for the new sample being tested to have consistent OTU labeling, the new sample is compared against the consensus sequences corresponding to the reference OTU matrix. In that case, when an existing OTU label is absent in the new sample, it is set to be empty.

In some embodiments, a spike-in strategy is used, wherein samples with known labels (e.g., the samples collected from the reference human subject population each of which is identified as having cancer or being normal) for training the classifier are processed (e.g., amplified and sequenced) together with one or more new samples of human subjects in need thereof (e.g., human subjects whose health conditions are to be predicted). The results of the reference human subject population are used to train the classifier. Such a spike-in strategy may control for batch effects and lead to higher prediction accuracy. In some embodiments, at least 1, 2, 3, 4, 5, 6, 7, 8, 9, 10, 15, 20, 25, 20, 35, 40, 45, 50, 55, 60, 65, 70, 75, 80, 85, 90, 95, 100 or more new samples of human subjects in need thereof are processed together (spiked-in) with the reference human subject population.

The classifiers of the present disclosure provide an unprecedented high specificity and accuracy for predicting colorectal cancerous conditions in human subjects, particularly when abundances of OTUs are the only distinguishing features used in the classifiers, without the need to include other information of the human subjects being tested. In some embodiments, the methods for classifying a human subject as having colorectal cancer (CRC) or being normal (NM) has an accuracy of at least 85%, 86%, 87%, 88%, 89%, 90%, 91%, 92%, 93%, 94%, 95%, 96%, 97%, 98%, 99%, or more. In some embodiments, the methods for classifying a human subject as having colorectal cancer (CRC), colorectal adenomas (AD), or being normal (NM) has an accuracy of at least 65%, 70%, 75, 80%, 85%, 86%, 87%, 88%, 89%, 90%, 91%, 92%, 93%, 94%, 95%, 96%, 97%, 98%, 99%, or more. In some embodiments, the methods for classifying a human subject as having colorectal cancer (CRC), polyps (PL), non-advanced adenomas (NA), advanced adenomas (AA), or being normal has an accuracy of at least 50%, 55%, 65%, 70%, 75%, 80%, 85%, 86%, 87%, 88%, 89%, 90%, 91%, 92%, 93%, 94%, 95%, 96%, 97%, 98%, 99%, or more.

### Systems

Systems utilizing the classifiers of the present disclosures are also provided. In some embodiments, the systems include one or more medical record databases. In some embodiments, the systems are connected to a medical record database interface. In some embodiments, the databases include a plurality of individual records of individual human subjects, based on analysis of individual samples collected from the human subjects. The databases can be selected based on purpose of the systems and tasks to be performed by the systems. In some embodiments, the database comprises a plurality of OTU vectors, wherein each OTU vector describes abundances of OTUs in an individual sample collected from an individual human subject with identified health condition (e.g., having a certain stage of cancer or being normal). In some embodiments, cancerous condition of the individual human subject is known (labeled). In some embodiments, the database comprises a reference OTU matrix that can be, or has been used to train the classifier. In some embodiments, the reference OTU matrix is generated by a method described herein.

In some embodiment, the methods and systems described herein involve controlling a computer aided diagnosis (CADx) system to classify a human subject's colorectal condition. For example, implementation of the method and/or system of the present disclosure for classifying can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

Hardware for performing a method of the present disclosure could be implemented as a chip or a circuit. As software, selected tasks according to embodiments of the present disclosure could be implemented as one or more software instructions being executed by a computer using a suitable operating system. In some embodiments, one or more steps in a method as described herein are performed by a data processor, such as a computing platform for executing one or more instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

In some embodiments, implementation of the methods and systems of the present disclosure comprises using one or more classifiers, such as one or more machine learning classifiers. A machine learning classifier can be generated according to the process as described herein. In some embodiments, the classifiers include, but are not limited to, the classifier algorithm is selected from the group consisting of decision tree classifier, K-nearest neighbor classifier (KNN), logistic regression classifier, nearest neighbor classifier, neural network classifier, Gaussian mixture model (GMM), Support Vector Machine (SVM) classifier, nearest centroid classifier, linear regression classifier and random forest classifier.

In some embodiments, training the classifier may include retrieving electronic data from a computer memory, receiving a computer file over a computer network, or other computer or electronic based action. In one embodiment, the classifier is a random forest classifier. In other embodiments, other types, combinations, or configurations of automated deep learning classifiers may be employed.

In some embodiments, the classifier(s) are outputted, optionally as a module that allows classifying a human subject in need thereof, by an interface unit. In some embodiments, one or more classifiers are generated and trained according to different demographic characteristics the human subject, such as age, gender, race, genetic mutations, etc.

In some embodiments, the classifier(s) can be hosted in a web server that receives OTU data of a human subject in need thereof, such that a module using the classifier(s) may predict cancerous condition of the human subject. The human subject data may be received through a communication network, such as the internet, from a client terminal, such as a laptop, a desktop, a Smartphone, a tablet and/or the like, which provides raw sequencing data or OTU data. The data may be inputted manually by a user, using an interface (e.g., a graphical user interface), selected by a user, optionally using the interface, and/or provided automatically, for example by a computer aided diagnosis (CAD) module and/or system.

In some embodiments, a system of the present disclosure may include a processor, a memory, an input/output (I/O) interface, a set of circuits, and an interface that connects the processor, the memory, the I/O interface, and the set of circuits. In some embodiments, the system includes a display circuit. In some embodiments, the system includes a training circuit. In some embodiments, the system includes a normalization circuit. In some embodiments, the system comprises dual microprocessor and other multi-processor architectures. In some embodiments, the memory may include volatile memory and/or non-volatile memory. A disk may be operably connected to computer via, for example, an input/output interface (e.g., card, device) and an input/output port. Disk may include, but is not limited to, devices like a magnetic disk drive, a tape drive, a Zip drive, a solid state device (SSD), a flash memory card, a shingled magnetic recording (SMR) drive, or a memory stick. Furthermore, disk may include optical drives like a CD-ROM or a digital video ROM drive (DVD ROM). Memory can store processes or data, for example. Disk or memory can store an operating system that controls and allocates resources of computer. Computer may interact with input/output devices via I/O interfaces and input/output ports. Input/output ports can include but are not limited to, serial ports, parallel ports, or USB ports. Computer may operate in a network environment and thus may be connected to network devices via I/O interfaces or I/O ports. Through the network devices, computer may interact with a network. Through the network, computer may be logically connected to remote computers. The networks with which computer may interact include, but are not limited to, a local area network (LAN), a wide area network (WAN), a WiFi network, or other networks.

### Treatments

Methods of the present disclosure in some embodiments comprise treating the human patients in need after the human patients are classified to having colorectal cancer or adenoma. In some embodiments, the treating include, but are not limited to, surgery, chemotherapy, radiation therapy, immunotherapy, palliative care, exercise.

As used herein the phrase “treatment regimen” refers to a treatment plan that specifies the type of treatment, dosage, schedule and/or duration of a treatment provided to a subject in need thereof (e.g., a subject diagnosed with a pathology). The selected treatment regimen can be an aggressive one which is expected to result in the best clinical outcome (e.g., complete cure of the pathology) or a more moderate one which may relieve symptoms of the pathology yet results in incomplete cure of the pathology. It will be appreciated that in certain cases the treatment regimen may be associated with some discomfort to the subject or adverse side effects (e.g., damage to healthy cells or tissue). The type of treatment can include a surgical intervention (e.g., removal of lesion, diseased cells, tissue, or organ), a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

In some embodiments, the treatments include, but is not limited to, fluorouracil, capecitabine, oxaliplatin, irinotecan, UFT, FOLFOX, FOLFOXIRI, and FOLFIRI, antiangiogenic drugs such as bevacizumab, and epidermal growth factor receptor inhibitors (e.g., cetuximab and panitumumab).

### Kits

Kits are also provided in the present disclosure for predicting cancer in a human subject in need thereof. In some embodiments, the kits may comprise a nucleic acid described herein together with any or all of the following: assay reagents, buffers, probes and/or primers, and sterile saline or another pharmaceutically acceptable emulsion and suspension base. In addition, the kits may include instructional materials containing directions (e.g., protocols) for the practice of the methods described herein. The kits may further comprise a software package for data analysis of nucleic acid profiles. For example, the kits may include a classifier of the present disclosure, which can be trained or have been trained. In some embodiments, the kits may include a reference OTU matrix of the present disclosure, and/or samples and reagents that can be used to produce the reference OTU matrix according to methods as described herein.

In some embodiments, the kit may be a kit for the amplification, detection, identification or quantification of nucleic acid sequences in a sample. The kit may comprise a poly (T) primer, a forward primer, a reverse primer, and a probe.

Any of the compositions described herein may be comprised in a kit. In a non-limiting example, reagents for isolating, labeling, and/or evaluating a DNA and/or RNA populations are included in a kit. It may also include one or more buffers, such as reaction buffer, labeling buffer, washing buffer, or a hybridization buffer, compounds for preparing the DNA sample, components hybridization and components for isolating DNA.

In some embodiments, a kit of the present disclosure includes a software package for data analysis of the nucleic acid profiles, such as an OTU profile obtained from the sample. The software package may include a machine learning classifier. The machine learning classifier may have been trained already by a reference data set, or the software package include one or more suitable reference data sets for training the machine learning classifier, depending on the purpose of the kit.

### Definition

Random forests or random decision forests are an ensemble learning method for classification, regression and other tasks, that operate by constructing a multitude of decision trees at training time and outputting the class that is the mode of the classes (classification) or mean prediction (regression) of the individual trees. Random decision forests correct for decision trees' habit of overfitting to their training set. Random forests are a way of averaging multiple deep decision trees, trained on different parts of the same training set, with the goal of reducing the variance. Non-limiting examples of method for using random forest classifier are described in U.S. Pat. Nos. 9,747,527, 8,802,599, 10,049,770, 9,068,232, 9,474,490, 10,055,839, 9,482,672, 9,852,501, 9,642,586, 9,096,906, 9,498,138, 9,235,278, 9,922,269, 8,463,721, 9,971,959, 9,898,811, 9,342,794, 9,918,686, 9,280,724, 8,811,666, 9,741,116, 10,063,582, 9,697,472, 9,978,142, 9,910,986, 9,690,938, 9,779,492, 9,208,323, 9,460,367, 9,430,829, 9,747,687, 9,014,422, 9,025,863, 9,946,936, 9,171,403, 9,615,878, 9,639,902, 10,025,819, 9,661,025, 9,978,425, 9,076,056, 9,609,904, 9,418,310, 9,911,219, and 10,037,603, each of which is herein incorporated by reference in its entirety for all purposes.

Classification is the process of predicting the class of given data points, e.g., identifying to which of a set of categories (sub-populations) a new observation belongs, on the basis of a training set of data containing observations (or instances) whose category membership is known. Classes are sometimes called as targets/labels or categories. Classification predictive modeling is the task of approximating a mapping function (f) from input variables (X) to discrete output variables (y). Classifier is an algorithm that implements classification, especially in a concrete implementation. The term “classifier” sometimes also refers to the mathematical function, implemented by a classification algorithm, that maps input data to a category. A classifier utilizes some training data to understand how given input variables relate to the class. In some embodiments, a classifier algorithm that can be used is selected from the group consisting of a decision tree classifier, K-nearest neighbor classifier (KNN), logistic regression classifier, nearest neighbor classifier, neural network classifier, Gaussian mixture model (GMM), Support Vector Machine (SVM) classifier, nearest centroid classifier, linear regression classifier and random forest classifier.

Operational Taxonomic Units (OTUs) refers to clusters of organisms, grouped by DNA sequence similarity of a specific taxonomic marker gene. In other words, OTUs are pragmatic proxies for microbial “species” at different taxonomic levels, in the absence of traditional systems of biological classification as are available for macroscopic organisms. OTUs have been the most commonly used units of microbial diversity, especially when analyzing small subunit 16S or 18S rRNA marker gene sequence datasets. Sequences can be clustered according to their similarity to one another, and operational taxonomic units are defined based on the similarity threshold (e.g., about 90%, 95%, 96%, 97%, 98%, 99% similarity or more) set by the researcher. Typically, OTUs are based on similar 16S rRNA sequences. OTUs can be calculated differently when using different algorithms or thresholds.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

“Computer-readable storage device”, as used herein, refers to a non-transitory computer-readable medium that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, a data storage device, and other media from which a computer, a processor or other electronic device can read.

“Nucleic acid” or “oligonucleotide” or “polynucleotide”, as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine Nucleic acids may be obtained by chemical synthesis methods or by recombinant methods.

“Variant” as used herein referring to a nucleic acid means (i) a portion of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto.

“Stringent hybridization conditions” as used herein mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Substantially complementary” as used herein means that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

“Substantially identical” as used herein means that a first and a second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

As used herein the term “diagnosing” refers to classifying pathology, or a symptom, determining a severity of the pathology (e.g., grade or stage), monitoring pathology progression, forecasting an outcome of pathology and/or prospects of recovery.

As used herein the phrase “subject in need thereof” refers to an animal or human subject who is known to have cancer, at risk of having cancer (e.g., a genetically predisposed subject, a subject with medical and/or family history of cancer, a subject who has been exposed to carcinogens, occupational hazard, environmental hazard) and/or a subject who exhibits suspicious clinical signs of cancer (e.g., blood in the stool or melena, unexplained pain, sweating, unexplained fever, unexplained loss of weight up to anorexia, changes in bowel habits (constipation and/or diarrhea), tenesmus (sense of incomplete defecation, for rectal cancer specifically), anemia and/or general weakness). Additionally or alternatively, the subject in need thereof can be a healthy human subject undergoing a routine well-being check up.

As used herein the term “about” refers to +10%.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

“Computer-readable storage device”, as used herein, refers to a non-transitory computer-readable medium that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, a data storage device, and other media from which a computer, a processor or other electronic device can read.

“Circuit”, as used herein, includes but is not limited to hardware, firmware, software in execution on a machine, or combinations of each to perform a function(s) or an action(s), or to cause a function or action from another circuit, method, or system. Circuit may include a software controlled microprocessor, a discrete logic (e.g., ASIC), an analog circuit, a digital circuit, a programmed logic device, a memory device containing instructions, and other physical devices. Circuit may include one or more gates, combinations of gates, or other circuit components. Where multiple logical circuits are described, it may be possible to incorporate the multiple logics into one physical logic or circuit. Similarly, where a single logical circuit is described, it may be possible to distribute that single logic between multiple logics or circuits.

### Examples

Human microbiota has been linked to a variety of metabolic diseases and recently, the mechanisms that lead to carcinoma have been identified for certain microbes. Colorectal cancer (CRC), when identified early, can be treated effectively. CRC prevalence is high in China, especially in the southwestern regions, likely due to dietary preferences and the reluctance for health checkups. Amplicon sequencing of variable regions of 16S rRNA have shown high potential in diagnosing CRC. We have collected microbiota information from a large Chinese cohort comprised of both normal individuals and patients in different stages of progression to CRC. Using sequence information from V3-V4 regions of 16S rRNA, we developed a model to differentiate patients with CRC from normal individuals with high accuracy, and further validated the model using independent test set. In adenomas cohort, we have demonstrated very promising classification results in the absence of independent cohort and further revealed such a strategy may be impacted by data overfitting. This is a common problem due to small sample size in the study. All samples are used as the training set and test set may come from the same batch of results, and as such, it is critical to mitigate the effect of overfitting (1). We further proposed a strategy to partially overcome the challenges of test cohort that may have different properties from the training set due to batch effects or contaminations for different experimental runs. Using non-invasive microbiota diagnosis of CRC holds promises as a prescreening strategy that could guide individuals with predicted high risk for developing CRC further checkups and may help lower the overall death rate as the result of earlier detection.

In the present disclosure, we are investigating the potential for using fecal microbiota as a non-invasive method to stratify disease status of Colorectal adenomas and CRC which complements other types of non-invasive methods such as FIT (20). Comparable to most of the existing strategies (1, 8, 26), we also use 16S rRNA sequencing (V3-V4 region) for surveying the microbiota content with the understanding of the limitation that species level resolution may not be achieved. To avoid the differences in the annotations of different reference databases (2), we use relative abundances of operational taxonomic units (OTUs) as the features for classification. Different from multi-bacterial prediction models, we do not preselect most predictive OTUs as our features for downstream classification but use all OTUs passing the quality control criteria. We have used random forest classifier as our model as it is known to capture the non-linear relationships in the data.

Independent test cohort has been used to report sensitivity, specificity and overall accuracy of our prediction. For cancer and non-cancer cohort, we have demonstrated the comparable performances of classification in the training and independent test set. Like many of the existing strategies when the independent test set was not used, we were also able to obtain highly accurate results differentiating adenomas and healthy cohorts as well. We further show that such good accuracy may have resulted from the overfitting of the data and an independent validation is a must to validate the model. We demonstrated that differentiating adenoma patients from normal individuals using microbiota data is more challenging to achieve, possibly due to a much weaker discriminant signals between these groups, insufficient number of training samples, and other experimental variations such as batch effects and contaminations. However, such limitations may be partially overcome in a diagnostic setting by resequencing certain number of known samples with samples with unknown labels.

In summary, we have developed a model that can be used to predict class labels of cancer versus non-cancer samples with high accuracy and demonstrated a practical strategy to model for batch effects and predict patients with adenomas. We have also corroborated that many of the top discriminative OTUs used by the random forest model were annotated to species or genus that were previously found in the association studies in CRC.

### Materials and Methods

### Fecal Sample Collection and Storage

Fecal samples were collected using the fecal pretreatment equipment (New Horizon Health Technology Co., Ltd. Beijing, China) at two sites in China: The Second Affiliated Hospital, Zhejiang University School of Medicine, Zhejiang and Jiashan Tumour Prevention & Cure Station, Jiaxing. The inclusion criteria for patients in the current study include (1) age between 40-75, (2) availability of colonoscopy biopsies and pathological examination results, and (3) no clinical treatment has been applied, such as surgery, chemotherapy.

Fecal samples were obtained from individuals with empty stomach prior to colonoscopy screening. For individuals post-colonoscopy screening but without colonic polyps removal, samples were collected at least one week post-screening and right before the removal procedure. Care was taken to avoid urine contamination. For each individual, 5 g stool sample was obtained and preserved in a tube with preservative buffer, which keeps bacteria alive but not growing. Fecal samples were allowed to be stored at the room temperature for a maximum of seven days before being processed. For long term storage, fecal samples were stored at −80° C. All patient have signed the study consent form.

### Sample Grouping

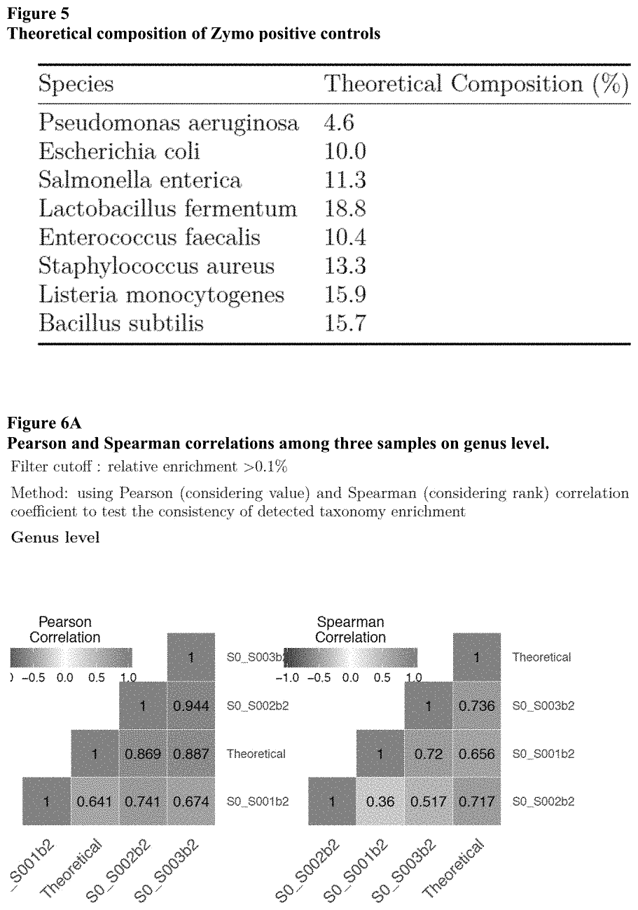

Although the disease progresses in a continuous fashion, we divide them into five discreet groups from normal to severe form in the following order: normal (NM), polyps (PL), non-advanced adenomas (NA), advanced adenomas (AA), and colorectal cancer (CR), according to the following histopathological criteria: CR is defined as all stages of colorectal cancer (specific stages have not been defined); AA is defined as adenoma with high grade dysplasia or adenoma ≥1 cm in size or has significant villous growth pattern ≥25%, serrated lesion with ≥1.0 cm in size; NA is defined as >3 adenomas, <10 mm in size, non-advanced; PL is defined as 1 or 2 adenoma(s), ≤5 mm in size, non-advanced; normal is defined as having no neoplastic findings. The samples had been collected in three batches, where the number of groups per batch are given in table 1. In batch 1, only CR and NM samples were obtained and in both the second and the third batch, we collected all five groups in a balanced number. In addition, we have obtained ZymoBIOMICS™ Microbial Community DNA Standard with the known mixture as the positive control in the third batch (FIG. 5).

TABLE 1
The number of samples collected in three batches for each group.
Samples are sequenced in three batched, where batch 1 has only
cancer (CR) and normal (NM) samples, batch 2 and batch 3 consist
of in addition three more groups: Polyps (PL), non-advanced
adenomas (NA), and advanced adenomas (AA). In addition, we
included three positive control samples in batch 3.

| | | | | | | #POSITIVE |
|---|---|---|---|---|---|---|
| BATCH | #CR | #AA | #NA | #PL | #NM | CONTROL |
| 1 | 57 | — | — | — | 129 | — |
| 2 | 102 | 96 | 106 | 96 | 100 | — |
| 3 | 100 | 100 | 100 | 100 | 99 | 3 |

### Library Preparation and Sequencing

Total genomic DNA of fecal samples were extracted and purified using the nucleic acid extraction and purification kits (New Horizon Health Technology Co., Ltd., Beijing, China). DNA concentration and purity were measured on 1% agarose gel (1%, w/v) and diluted to 1 ng/μl using sterile water.

The V3-V4 hyper variable regions of the 16S rRNA gene were amplified using primer pair 341F (CCTAYGGGRBGCASCAG, SEQ ID NO. 346) and 806R (GGACTACNNGGGTATCTAAT, SEQ ID NO. 347). PCR reactions were carried out in 30 μl reactions with 15 μl of Phusion® High-Fidelity PCR Master Mix (New England Biolabs); 0.2 μM of forward and reverse primers, and about 10 ng template DNA. Thermal cycling condition consisted of initial denaturation at 98° C. for 1 min, followed by 30 cycles of denaturation at 98° C. for 10 s, annealing at 50° C. for 30 s, and elongation at 72° C. for 30 s, and finally 72° C. for 5 min.

PCR products were separated by electrophoresis in agarose gels (2%, w/v) and samples with bright main strip between 400-500 bp were chosen to be pooled in equidensity ratios, then purified with GeneJET Gel Extraction Kit (Thermo Scientific). Sequencing libraries were prepared using a TruSeq® DNA PCR-Free Sample Preparation Kit (Illumina) following the manufacturer's recommendations. Library quality was assessed on the Qubit® 2.0 Fluorometer (Thermo Scientific) and Agilent Bioanalyzer 2100 system. The libraries were sequenced on Illumina HiSeq2500 using 250PE protocol by Novogene Bioinformatics Technology Co., Ltd. (Beijing, China) in three batches. The number and types of samples for each batch are given in Table 1. The target mean number of fragments per sample is 50K.

### Pipeline

The analysis pipeline consists of a combination of public available programs and in house programs to reduce run-time and memory usage. We have conducted the processing and analysis of all samples on a desktop computer (3 GHz Intel Core i5 CPU, 16 GB 2400 MHz DDR4 RAM).

Briefly, each input sample consists of a paired FASTQ gz files. FLASH v2.2.00 (https://ccb.jhu.edu/software/FLASH/) was used to merge each read pair to a fragment allowing a minimum overlap of 10 bp. Each resulting fragment represents the sequence of V3-V4 region. Fragments are filtered based on quality using usearch program v10.0.240 (12). Pass filter fragments are further merged to form unique sequences and their abundances were obtained. Clustering of unique sequences using 97% similarity threshold resulted in the final clusters of Operational Taxonomic Units (OTUs), meanwhile, chimeric sequences were filtered out using UParse (12). For each OTU, a consensus sequence was selected. Given the constructed OTU consensus sequences, input samples were then reprocessed by comparing the raw sequences to the consensus sequences to generate OTU table/matrix, which represent the relative OTU abundances per sample. In the OTU table, each row denotes a unique OTU label and each column corresponds to a sample. The OTU table is normalized for differences in sequencing depth (by default 50,000). The resulting OTU table were further processed by SINTAX (11) program to obtain annotations at different taxonomic rank using one of the SILVA (23) or RDP (7) (by default) as the reference database. For between group comparisons, we use linear discriminant analysis effect size (LEfSe) (25) tool to identify discriminative biomarkers on different taxonomic level.

### Classification

Random forest classifier has been successfully applied to genomic applications (e.g. (3, 5)) due to its ability to capture non-linear relationships in the data and handle much larger number of features compared to the number of samples, the typical situations in genomics applications. Briefly, the method starts out by constructing decisions trees where each tree is built from a subset of samples from the training set. When considering splitting an internal node, only a subset of features among the total features are considered. The classification result for each given sample is taken as the majority vote of decisions made by all trees in the forest. Random forest significantly improves upon the performance of a decision tree by maintaining a low bias while reducing variance.

In the current context, we represent each sample by a vector of relative OTU abundances, serving as features. As the number of features may be an order of magnitude larger compared to the number of samples and the relationships between the features and the disease states may be non-linear, random forest serves as a reasonable model for classification. To measure model accuracy, we use ˜80% data as training set and report prediction accuracy on the remaining test set instead of resorting to cross validation as the random forest model is an ensemble learning method.

For implementation, “randomForest” package (v4.6-12) in R was used with the following values: mtry is set to be square root of the total parameters, the number of trees was set to 1000, and we allow each tree to grow to the full size. As can be seen in the results, the out-of-bag error typically stabilizes before 1000 trees were reached. Even though in some cases, we have over 5,000 features, which seems to be large, the model was able to choose relevant features on its own as many OTUs may correspond to the same species or genus and hence are not completed independent. We also observed that majority of features were present in only a small number of samples, likely due to batch effects or contaminations as indicated by the analysis of positive controls. Hence, we retained only features satisfying the criteria that each feature occurs in at least among p % (default p=3) of samples with relative abundance at least f % (default f=0.05). However, when such features consistently present in a single group could be real discriminative signal. In order to avoid removing such features by mistake, random permutation was first applied to shuffle the samples, and we apply the above criteria and identify these features in a proportion (e.g. half) of input samples. After feature reduction, the number of features became comparable to the number of training samples and run time significantly reduced.

### Prediction: An Independent Validation

The general performance of the model requires independent test set that had no association with the samples that were used for model construction.

To predict the class labels for new samples, there are two viable solutions. The new samples can be reprocessed together with samples of known labels using the pipeline such that the new samples would have the same set of OTU labels as the samples used for building the classifier. Then the random forest model need to be rebuilt using the same set of known samples and predictions can then be made for the new samples. However, the major disadvantage of this approach is the run-time, dominated by OTU table construction step. One may notice that the random forest model may change slightly depending on samples included, however, the performance would not be affected as long as the training set is diverse enough to capture the group variance. Alternatively, we can directly apply the random forest model built using the training set for prediction. In order for the new samples to have consistent OTU labeling, we compare the new samples against the consensus sequences used for OTU table generation for the classifier and when an existing OTU label is absent in the new samples, it is set to be empty.

As is the general case for any machine learning method, the prediction accuracy depends on the variance and the bias of the built model. In the current application, the former depends on if OTU relative abundance can serve as a discriminative signal for different groups and the latter depends on the sample size and other technical variables such as assay reproducibility, which is a known issue in the field of microbiome studies where the results of the same set of samples may differ when processed by different facilities, different computational pipelines and other technical challenges such as batch effects and contaminations. In some cases, the bias is hard to overcome in practice and both of the aforementioned strategies for prediction is difficult to generalize to independent samples when technical variations (termed as batch effects for simplicity) are strong, particularly for multiple-group classification. These batch effects may be hardly correctable by computational methods (16). In those cases, a spike-in strategy can be used to introduce samples with known labels which are resequenced with the new samples and identified the model performance as a function of the number of samples required for the model to capture the batch effects.

### Results

### Sequencing and Meta Data

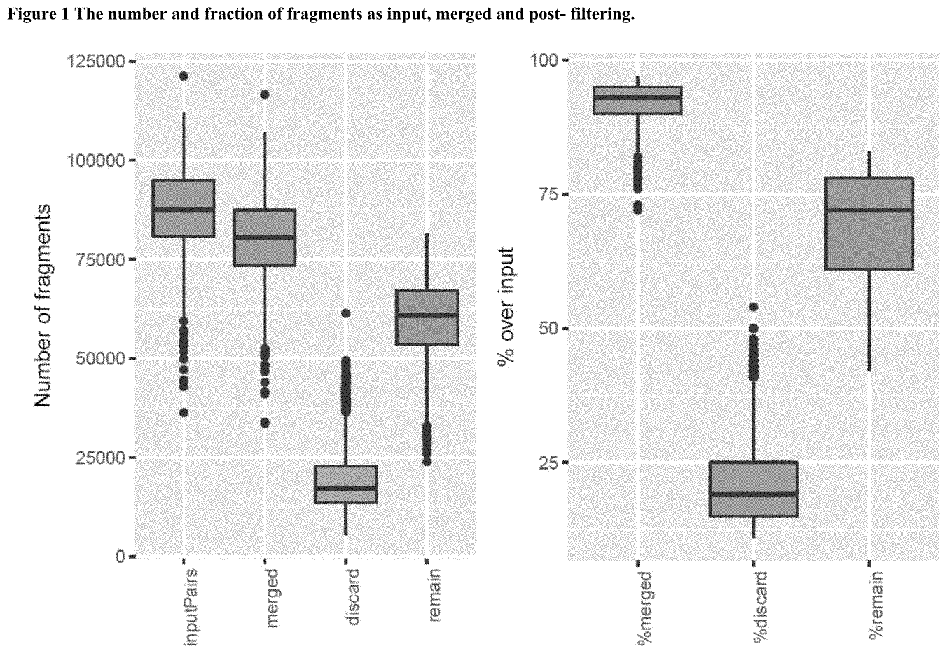

Although the target sequencing depth is 50K, we have obtained in average 80K fragments per sample (FIG. 1). The number and percentage of fragments after merging and quality filtering are shown in FIG. 1. We have obtained an average of over 60K effective fragments for downstream analysis.

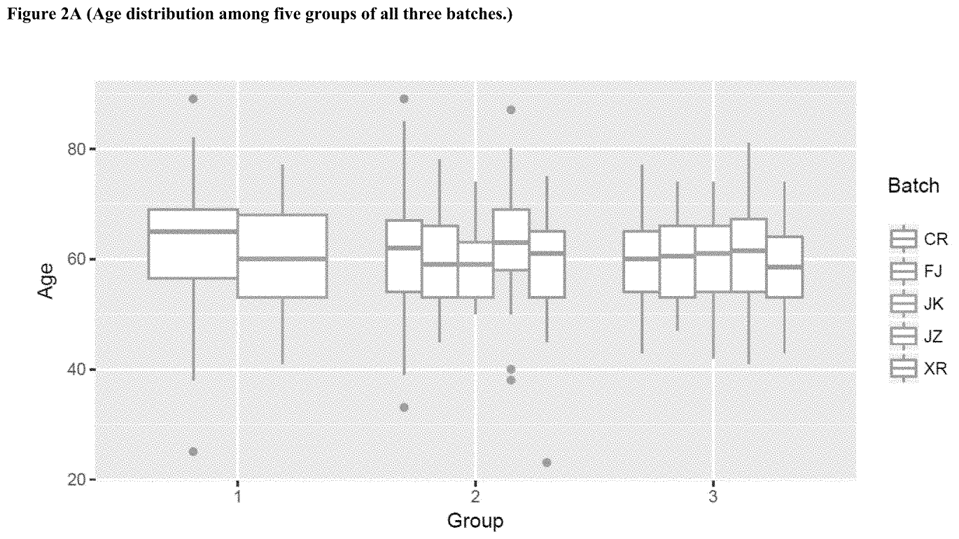

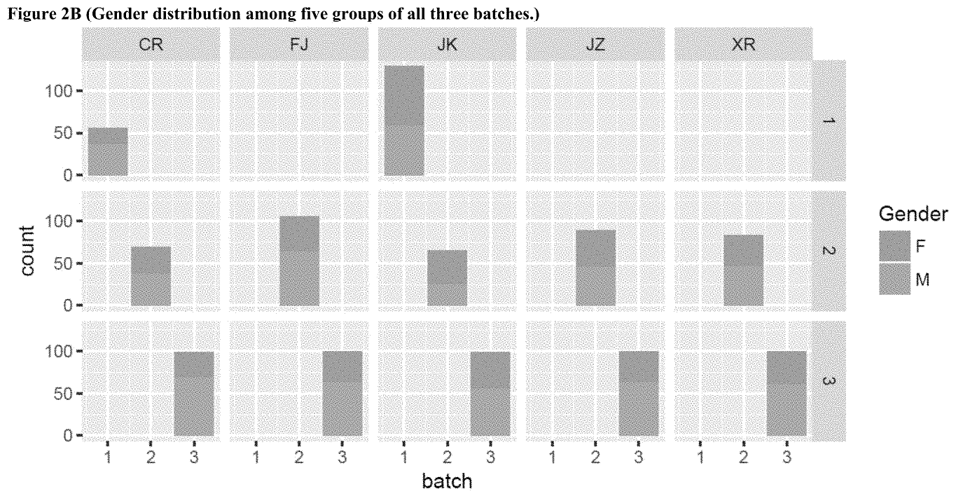

As age and gender are factors that may affect microbiota composition and distort classification results, we summarized these two factors for all three batches in FIG. 2. The mean age for different groups centered around 60 and overall, we have sampled more males than females. For batch 3, we explicitly controlled the matching of age and gender, therefore, these two factors are better balanced compared to batch 1 and 2. Given the observed distribution, we do not expect them to confound the classification results.

### Batch Effects Revealed by Positive Control Samples

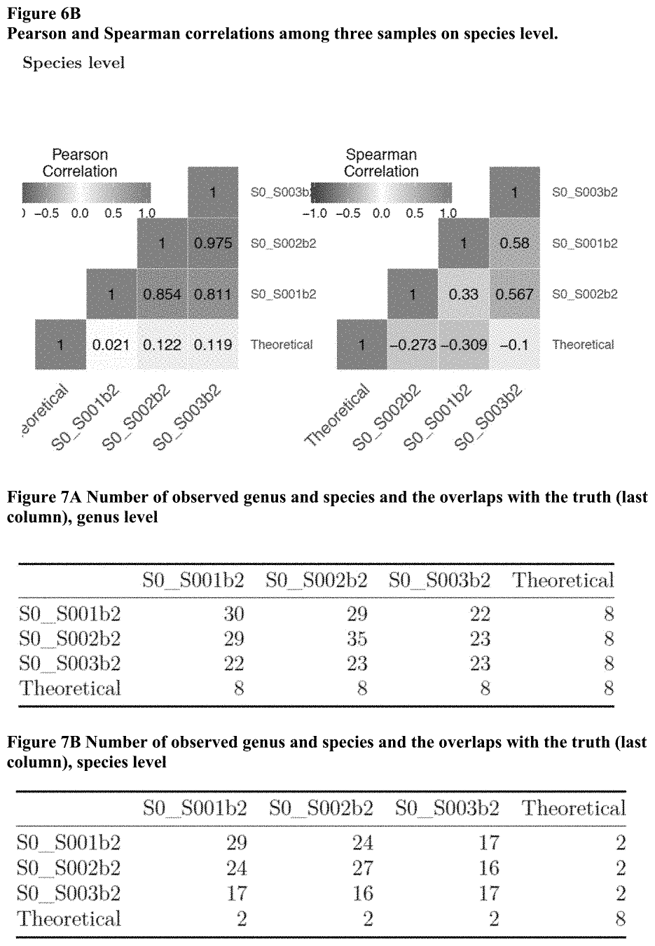

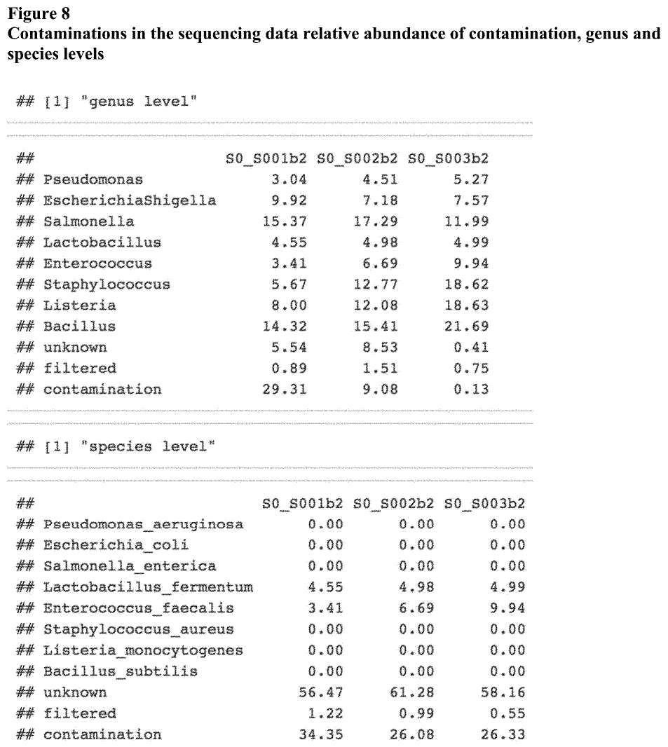

We measured the batch effects by comparing the sequencing results of positive controls samples. Mainly, we measured the Pearson correlation of relative abundances of annotated genus/species, the number of genus/species overlapping with the truth, and the contamination rate. The detailed results are summarized below. In summary, all metrics at the genus level were better compared to when measured at the species level. At the genus level, we observed Pearson correlations ranging from 0.64 to 0.95 (FIG. 6A and FIG. 6B). The number of observed genus range from 22-35 as compared to the theoretical value of 8 (FIG. 7A and FIG. 7B). Three levels of contamination rates were observed: 0.1%, 9.1% and a very high level of 29.3% in one of the samples due to a major contaminant of Bacteroides (FIG. 8). The deviation of these metrics from the true values appeared to be mostly due to the contamination in the sample although the limitation of the annotation method and the database used may also be contributing factors. Note that, the contamination measures do not prove run-wide contamination event but does reflect the prevalence and severity of such event in practice.

### Classification: Cancer (CR) and Normal (NM)

As we have a relatively large collection of normal and cancer samples, we can measure the classification accuracy given different number of training samples. This provides a guidance on when we may have sufficient number of samples to capture the discriminative signals in differentiating two groups. We pooled all CR (259) and NM (328) samples from three batches of sequencing and obtained the results for using 80%, 60%, 40% and 20% randomly selected proportion as training data and the remaining as the test data. Within both the training and the test data, the ratios of normal and cancer samples are consistent with the overall distribution. The sensitivity, specificity and accuracy are reported in table 2, where the sensitivity is the proportion of cancer patients correctly identified, the specificity is the proportion of normal patients correctly identified, and the accuracy is the proportion of correctly predicted samples.

TABLE 2
Classification results on the test set for CR and NM groups
with different number of samples used as the training set.

| Training | | Test | | | | |
|---|---|---|---|---|---|---|
| # CR | #NM | # CR | #NM | Sensitivity | Specificity | Accuracy |
| 207 | 271 | 52 | 57 | 0.981 | 1.000 | 0.991 |
| 160 | 201 | 99 | 127 | 0.990 | 0.992 | 0.991 |
| 99 | 127 | 160 | 201 | 0.981 | 1.000 | 0.992 |
| 52 | 57 | 207 | 271 | 0.986 | 0.993 | 0.990 |

We observed a comparable performance in all metrics in the test set even when the number of training samples for CR and NM reduced to around 50 s. This observation indicates that good discriminative signals have been captured by OTUs between cancer and normal groups. The details can be found below.

### Classification of Three Batches of CR/JK Microbiome Samples

### Background

We classify CR (cancer) and JK (normal) samples pooled from three batches of sequencing data. First, we establish a classifier for CR and JK using 80% of each category then test on the remaining 20%. The feature selection is applied.

Random Forest Classification Using Normalized OTU table

1. Converting input tsv file into proper format and assign class labels.
## [1] “path: 2018-03-23_cr_jk_c_b1_b2/otutab_norm.txt”
##
##
## | sample_size | num_OTUs |
## |:-----------:|:--------:|
## | 587 | 5260 |
##
## Table: Total number of samples and OTUs
2. Feature Selection
We select OTUs satisfying that it occurs in at least 3% of samples with relative abundance >
0.05%. Given that the normalized counts per sample is 50,000, the latter is > 25 counts.
##
##
## | sample_size | num_OTUs |
## |:-----------:|:--------:|
## | 587 | 374 |
##
## Table: After Feature Selection, total number of samples and OTUs
3. Prepare training and test data
##
##
## | sample_labels | num_samples |
## |:-------------:|:-----------:|
## | training_data | 478 |
## | test_data | 109 |
##
## Table: The number of CR-JK training and test samples
4. Information of the model and training results
##
## Call:
## randomForest(formula = Type ~., data = trainData, importance = TRUE, ntree = 1000)

| | |
|---|---|
| ## | Type of random forest: classification |

| | |
|---|---|
| ## | Number of trees: 1000 |

## No. of variables tried at each split: 19
##

| | |
|---|---|
| ## | OOB estimate of error rate: 0.84% |

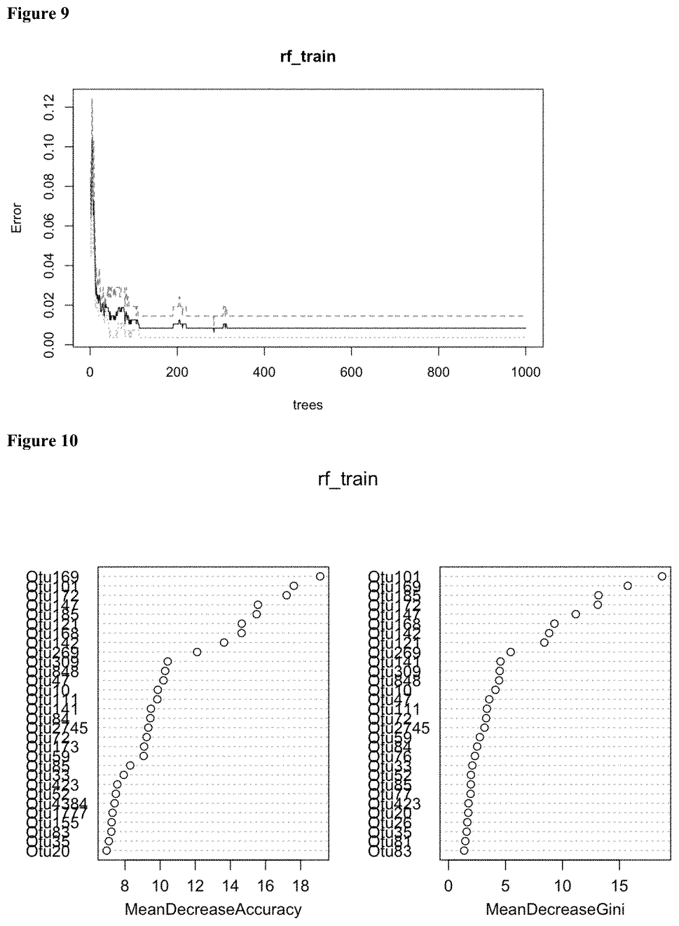

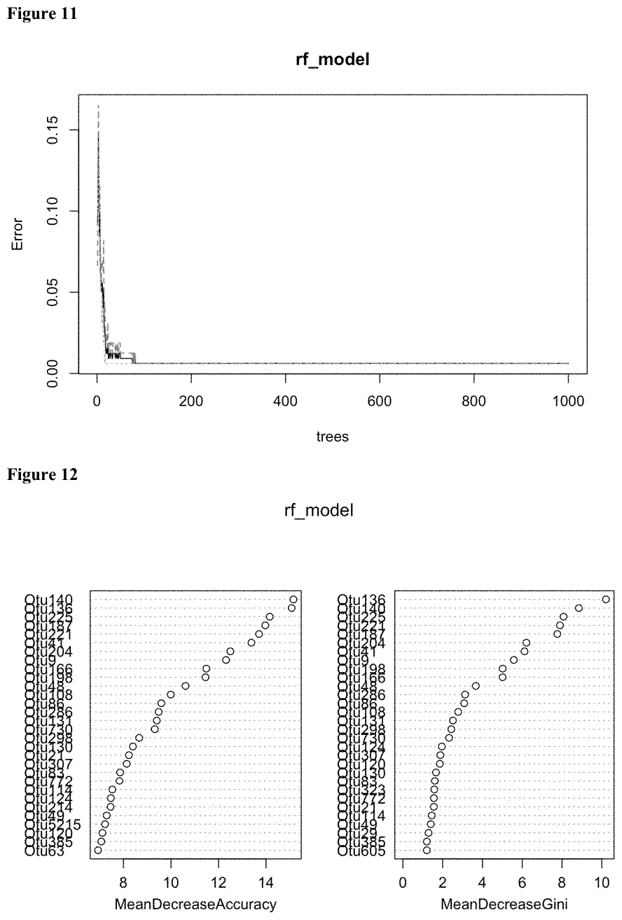

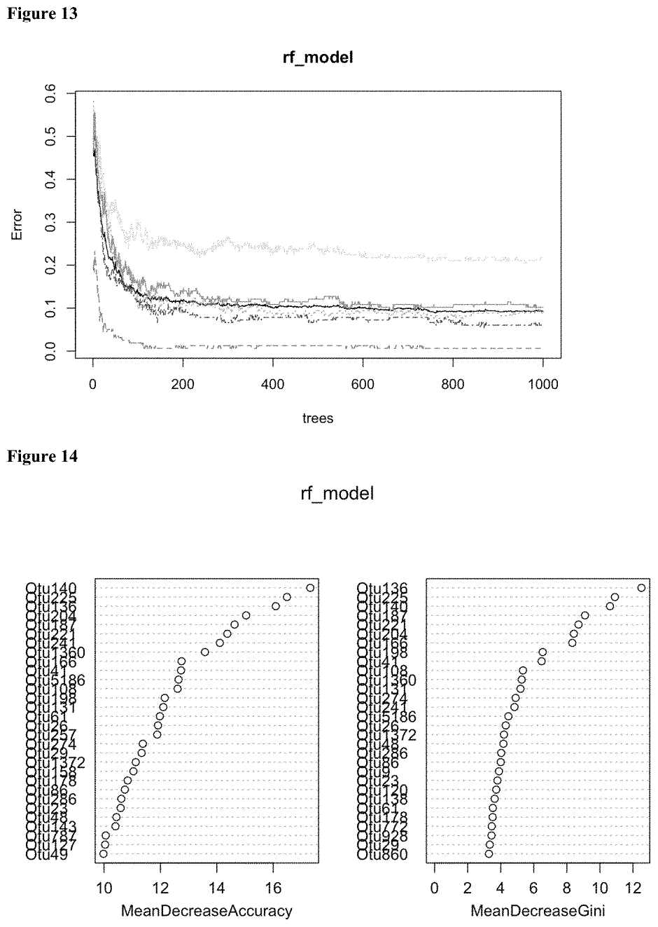

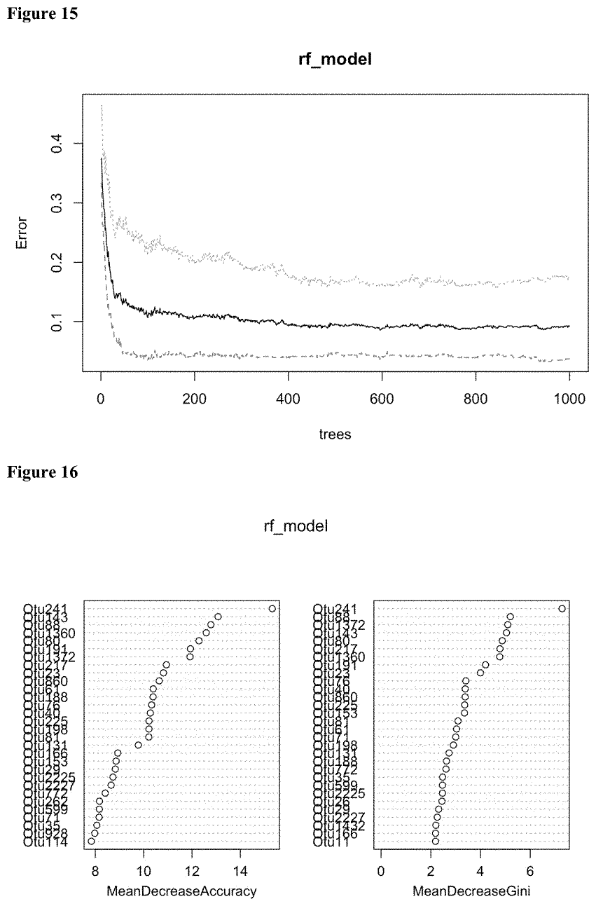

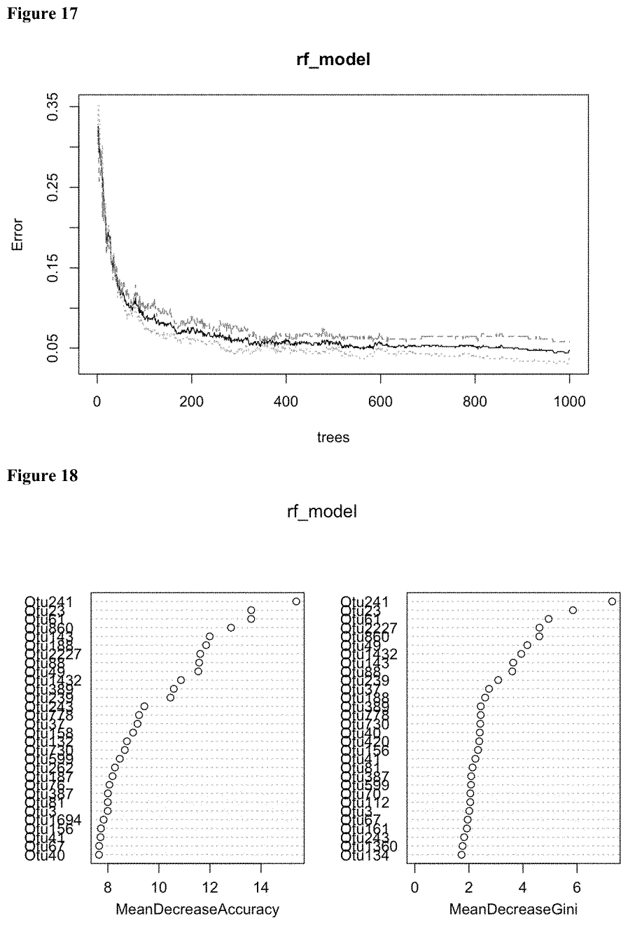

## Confusion matrix:
## CR JK class.error
## CR 204 3 0.014492754
## JK 1 270 0.003690037
##
##
## | CR | JK | MeanDecreaseAccuracy | MeanDecreaseGini | OtuName |
## |:-----:|:-----:|:--------------------:|:----------------:|:-------:|
## | 14.8 | 18.07 | 19.11 | 15.72 | Otu169 |
## | 14.65 | 16.76 | 17.61 | 18.74 | Otu101 |
## | 12.95 | 15.68 | 17.2 | 13.09 | Otu172 |
## | 12.39 | 14.22 | 15.57 | 11.17 | Otu147 |
## | 11.5 | 14.29 | 15.49 | 13.16 | Otu185 |
## | 12.26 | 12.66 | 4.65 | 8.406 | Otu121 |
## | 10.92 | 12.86 | 4.64 | 9.293 | Otu168 |
## | 10.32 | 13.37 | 13.64 | 8.828 | Otu142 |
## | 7.594 | 11.44 | 12.11 | 5.452 | Otu269 |
## | 9.924 | 6.921 | 10.43 | 4.488 | Otu309 |
##
## Table: Top 10 most important variables by mean decrease accuracy
(Also see FIGS. 9 and 10)
5. Predictions on the remaining 20% test CR JK data
##
##
## | &nbsp; | CR | JK |
## |:------:|:--:|:--:|
## | **CR** | 51 | 0 |
## | **JK** | 1 | 57 |
##
## Table: Predicting on test CR, JK samples
##
##
## | metrics | value |
## |:-----------:|:-----:|
## | accuracy | 0.991 |
## | sensitivity | 0.981 |
## | specificity | 1.000 |
##
## Table: Accuracy
6. Measure the Effect of Training Sample Size on Classification Results:
For the purpose of measure the accuracy with respect to the number of samples used, we use 80%,
60%, 40% and 20% of the original input sample and then measure the performance.
## Downsampling training set to fraction: 0.6
##
## | sample_size | num_OTUs |
## |:-----------:|:--------:|
## | 587 | 374 |
##
## Table: Total number of samples and OTUs
##
##
##
## | &nbsp; | nTrain | nTest |
## |:------------:|:------:|:-----:|
## | **cr.FALSE** | 160 | 99 |
## | **jk.TRUE** | 201 | 127 |
##
## Table: The number of training and test number of samples
##
##
##
## | sample_labels | num_samples |
## |:-------------:|:-----------:|
## | training_data | 361 |
## | test_data | 226 |
##
## Table: The number of CR-JK training and test samples
##
##
##
## | CR | JK | MeanDecreaseAccuracy | MeanDecreaseGini | OtuName |
## |:-----:|:-----:|:--------------------:|:----------------:|:-------:|
## | 14.13 | 17.26 | 18.09 | 13.94 | Otu101 |
## | 13.77 | 17 | 17.67 | 13.53 | Otu169 |
## | 10.6 | 14.86 | 15.64 | 11.29 | Otu172 |
## | 11.89 | 13.4 | 15.04 | 7.694 | Otu147 |
## | 10.78 | 12.05 | 13.76 | 7.281 | Otu185 |
## | 11.3 | 11.4 | 13.02 | 6.595 | Otu121 |
## | 8.432 | 12.64 | 12.72 | 6.704 | Otu142 |
## | 9.79 | 10.73 | 11.9 | 7.317 | Otu168 |
## | 7.176 | 10.57 | 11.18 | 4.067 | Otu269 |
## | 8.04 | 9.096 | 10.34 | 3.59 | Otu848 |
##
## Table: Top 10 most important variables by mean decrease accuracy
##
##
##
## | &nbsp; | CR | JK |
## |:------:|:--:|:---:|
## | **CR** | 98 | 1 |
## | **JK** | 1 | 126 |
##
## Table: Predicting on test CR, JK samples
##
##
##
## | metrics | value |
## |:-----------:|:-----:|
## | accuracy | 0.991 |
## | sensitivity | 0.990 |
## | specificity | 0.992 |
##
## Table: Accuracy
##
## Downsampling training set to fraction: 0.4
##
## | sample_size | num_OTUs |
## |:-----------:|:--------:|
## | 587 | 374 |
##
## Table: Total number of samples and OTUs
##
##
##
## | &nbsp; | nTrain | nTest |
## |:------------:|:------:|:-----:|
## | **cr.FALSE** | 99 | 160 |
## | **jk.TRUE** | 127 | 201 |
##
## Table: The number of training and test number of samples
##
##
##
## | sample_labels | num_samples |
## |:-------------:|:-----------:|
## | training_data | 226 |
## | test_data | 361 |
##
## Table: The number of CR-JK training and test samples
##
##
##
## | CR | JK | MeanDecreaseAccuracy | MeanDecreaseGini | OtuName |
## |:-----:|:-----:|:--------------------:|:----------------:|-------:|
## | 11.99 | 13.75 | 14.44 | 7.69 | Otu101 |
## | 10.79 | 13.05 | 13.54 | 5.687 | Otu172 |
## | 10.54 | 12.95 | 13.31 | 5.934 | Otu169 |
## | 9.98 | 11.41 | 12.9 | 4.598 | Otu168 |
## | 8.909 | 11.33 | 12.08 | 4.178 | Otu185 |
## | 9.39 | 10.99 | 11.94 | 3.899 | Otu121 |
## | 8.232 | 11.49 | 11.56 | 4.031 | Otu142 |
## | 10.73 | 10.27 | 11.51 | 4.626 | Otu147 |
## | 8.56 | 6.709 | 9.224 | 2.004 | Otu309 |
## | 6.566 | 7.512 | 8.611 | 1.992 | Otu10 |
##
## Table: Top 10 most important variables by mean decrease accuracy
##
##
##
## | &nbsp; | CR | JK |
## |:------:|:---:|:---:|
## | **CR** | 157 | 0 |
## | **JK** | 3 | 201 |
##
## Table: Predicting on test CR, JK samples
##
##
##
## | metrics | value |
## |:-----------:|:-----:|
## | accuracy | 0.992 |
## | sensitivity | 0.981 |
## | specificity | 1.000 |
##
## Table: Accuracy
##
## Downsampling training set to fraction: 0.2
##
## | sample_size | num_OTUs |
## |:-----------:|:--------:|
## | 587 | 374 |
##
## Table: Total number of samples and OTUs
##
##
##
## | &nbsp; | nTrain | nTest |
## |:------------:|:------:|:-----:|
## | **cr.FALSE** | 52 | 207 |
## | **jk.TRUE** | 57 | 271 |
##
## Table: The number of training and test number of samples
##
##
##
## | sample_labels | num_samples |
## |:-------------:|:-----------:|
## | training_data | 109 |
## | test_data | 478 |
##
## Table: The number of CR-JK training and test samples
##
##
##
## | CR | JK | MeanDecreaseAccuracy | MeanDecreaseGini | OtuName |
## |:-----:|:-----:|:--------------------:|:----------------:|:-------:|
## | 9.483 | 11.55 | 11.79 | 3.107 | Otu169 |
## | 8.626 | 10.52 | 10.62 | 2.916 | Otu101 |
## | 7.899 | 9.749 | 10.04 | 2.255 | Otu172 |
## | 7.981 | 9.202 | 9.839 | 2.057 | Otu168 |
## | 7.313 | 9.554 | 9.755 | 2.25 | Otu185 |
## | 8.626 | 8.475 | 9.192 | 2.261 | Otu147 |
## | 6.588 | 8.642 | 8.809 | 1.642 | Otu121 |
## | 6.953 | 7.696 | 8.642 | 1.614 | Otu47 |
## | 4.057 | 7.326 | 7.357 | 0.8975 | Otu142 |
## | 5.312 | 6.891 | 7.279 | 1.118 | Otu10 |
##
## Table: Top 10 most important variables by mean decrease accuracy
##
##
##
## | &nbsp; | CR | JK |
## |:------:|:---:|:---:|
## | **CR** | 204 | 2 |
## | **JK** | 3 | 269 |
##
## Table: Predicting on test CR, JK samples
##
##
##
## | metrics | value |
## |:-----------:|:-----:|
## | accuracy | 0.990 |
## | sensitivity | 0.986 |
## | specificity | 0.993 |
##
## Table: Accuracy

### Prediction: CR and NM

Batch 2 and batch 3 samples are independently sequenced in separate time points, serving as independent test set. We built the classifier using one of the full batch 2 or batch 3 samples and used the classifier to predict the class labels on the other batch. This removed the potential batch effects and other technical noises such as contaminations that may potentially confound the model performance. As shown in Table 3, the performance of the classifier built from either batch 2 or batch 3 are comparable. As expected, the sensitivity, specificity and accuracy all reduced 2-3% when compared to using the pooled data (Table 2). The slight better performance when samples were pooled together was likely because of the batch effects were captured by the model. However, the real biological signal was stronger compared to the batch effects such that good result was achieved for the prediction task. The details of prediction can be found below.

TABLE 3
Classification results for CR and NM with training
and test data from independent sequencing batches.

| Training | Test | | | |
|---|---|---|---|---|
| # CR | # CR | Sensitivity | Specificity | Accuracy |
| batch2 | bach3 | 0.9600 | 0.9596 | 0.9600 |
| batch3 | bach2 | 0.9608 | 0.9600 | 0.9604 |

Prediction Using CR/JK, Five Group, Three Group, CR/NC and AD/NM
Classifier

| | |
|---|---|
| 1. | Prediction on Flemer2017 samples |
| ## | Confusion Matrix and Statistics |
| ## | |

| | |
|---|---|
| ## | Reference |

| | | | |
|---|---|---|---|
| ## | Prediction | CR | JK |
| ## | CR | 6 | 0 |
| ## | JK | 37 | 37 |

| | |
|---|---|
| ## | |
| ## | Accuracy : 0.5375 |

| | |
|---|---|
| ## | 95% CI : (0.4224, 0.6497) |

| | |
|---|---|
| ## | No Information Rate : 0.5375 |
| ## | P-Value [Acc > NIR] : 0.5457 |
| ## | |

| | |
|---|---|
| ## | Kappa : 0.1304 |

| | |
|---|---|
| ## | Mcnemar's Test P-Value : 3.252e−09 |
| ## | |

| | |
|---|---|
| ## | Sensitivity : 0.1395 |
| ## | Specificity : 1.0000 |

| | |
|---|---|
| ## | Pos Pred Value : 1.0000 |
| ## | Neg Pred Value : 0.5000 |

| | |
|---|---|
| ## | Prevalence : 0.5375 |

| | |
|---|---|
| ## | Detection Rate : 0.0750 |

| | |
|---|---|
| ## | Detection Prevalence : 0.0750 |

| | |
|---|---|
| ## | Balanced Accuracy : 0.5698 |
| ## | |
| ## | ‘Positive’ Class : CR |
| ## | |
| 2. | CR/JK prediction using classifier built from b1 on b2 samples. |
| ## | Confusion Matrix and Statistics |
| ## | |

| | |
|---|---|
| ## | Reference |

| | | | |
|---|---|---|---|
| ## | Prediction | CR | JK |
| ## | CR | 96 | 4 |
| ## | JK | 4 | 95 |

| | |
|---|---|
| ## | |
| ## | Accuracy : 0.9598 |

| | |
|---|---|
| ## | 95% CI : (0.9223, 0.9825) |

| | |
|---|---|
| ## | No Information Rate : 0.5025 |
| ## | P-Value [Acc > NIR] : <2e−16 |
| ## | |

| | |
|---|---|
| ## | Kappa: 0.9196 |

| | |
|---|---|
| ## | Mcnemar's Test P-Value : 1 |
| ## | |

| | |
|---|---|
| ## | Sensitivity : 0.9600 |
| ## | Specificity : 0.9596 |

| | |
|---|---|
| ## | Pos Pred Value : 0.9600 |
| ## | Neg Pred Value : 0.9596 |

| | |
|---|---|
| ## | Prevalence : 0.5025 |

| | |
|---|---|
| ## | Detection Rate : 0.4824 |

| | |
|---|---|
| ## | Detection Prevalence : 0.5025 |

| | |
|---|---|
| ## | Balanced Accuracy : 0.9598 |
| ## | |
| ## | ‘Positive’ Class : CR |

| | |
|---|---|
| ## | |
| 3. | CR/JK prediction using classifier built from b2 on b1 samples. |
| ## | Confusion Matrix and Statistics |
| ## | |

| | |
|---|---|
| ## | Reference |

| | | | |
|---|---|---|---|
| ## | Prediction | CR | JK |
| ## | CR | 98 | 4 |
| ## | JK | 4 | 96 |

| | |
|---|---|
| ## | |
| ## | Accuracy : 0.9604 |

| | |
|---|---|
| ## | 95% CI : (0.9235, 0.9827) |

| | |
|---|---|
| ## | No Information Rate : 0.505 |
| ## | P-Value [Acc > NIR] : <2e−16 |
| ## | |

| | |
|---|---|
| ## | Kappa: 0.9208 |

| | |
|---|---|
| ## | Mcnemar's Test P-Value : 1 |
| ## | |

| | |
|---|---|
| ## | Sensitivity : 0.9608 |
| ## | Specificity : 0.9600 |

| | |
|---|---|
| ## | Pos Pred Value : 0.9608 |
| ## | Neg Pred Value : 0.9600 |

| | |
|---|---|
| ## | Prevalence : 0.5050 |

| | |
|---|---|
| ## | Detection Rate : 0.4851 |

| | |
|---|---|
| ## | Detection Prevalence : 0.5050 |

| | |
|---|---|
| ## | Balanced Accuracy : 0.9604 |
| ## | |
| ## | ‘Positive’ Class : CR |
| ## | |

| | |
|---|---|
| 4. | Prediction using three group classifier built from b1 samples on b2 |
| | samples. |
| ## | Confusion Matrix and Statistics |
| ## | |

| | |
|---|---|
| ## | Reference |

| | | | | |
|---|---|---|---|---|
| ## | Prediction | CR | S1_XR_JK | S2_JZ_FJ |
| ## | CR | 90 | 3 | 7 |
| ## | S1_XR_JK | 1 | 31 | 14 |
| ## | S2_JZ_FJ | 9 | 165 | 179 |

| | |
|---|---|
| ## | |
| ## | Overall Statistics |
| ## | |

| | |
|---|---|
| ## | Accuracy : 0.6012 |

| | |
|---|---|
| ## | 95% CI: (0.5567, 0.6445) |

| | |
|---|---|
| ## | No Information Rate : 0.4008 |
| ## | P-Value [Acc > NIR] : <2.2e−16 |
| ## | |

| | |
|---|---|
| ## | Kappa: 0.3764 |

| ## | Mcnemar's Test P-Value : <2.2e−16 | | | |
|---|---|---|---|---|
| ## | | | | |
| ## | Statistics by Class: | | | |
| ## | | | | |
| | | | Class: | Class: |
| ## | | Class: CR | S1_XR_JK | S2_JZ_FJ |
| ## | Sensitivity | 0.9000 | 0.15578 | 0.8950 |
| ## | Specificity | 0.9749 | 0.95000 | 0.4181 |
| ## | Pos Pred Value | 0.9000 | 0.67391 | 0.5071 |
| ## | Neg Pred Value | 0.9749 | 0.62914 | 0.8562 |
| ## | Prevalence | 0.2004 | 0.39880 | 0.4008 |
| ## | Detection Rate | 0.1804 | 0.06212 | 0.3587 |
| ## | Detection Prevalence | 0.2004 | 0.09218 | 0.7074 |
| ## | Balanced Accuracy | 0.9375 | 0.55289 | 0.6565 |

| | |
|---|---|
| 5. | Prediction using three group classifier built from half of pooled b1 |
| | and b2 samples on the other half. |
| ## | Confusion Matrix and Statistics |
| ## | |

| | |
|---|---|
| ## | Reference |

| | | | | |
|---|---|---|---|---|
| ## | Prediction | CR | S1_XR_JK | S2_JZ_FJ |
| ## | CR | 73 | 2 | 3 |
| ## | S1_XR_JK | 3 | 130 | 63 |
| ## | S2_JZ_FJ | 26 | 64 | 133 |

| | |
|---|---|
| ## | |
| ## | Overall Statistics |
| ## | |

| | |
|---|---|
| ## | Accuracy : 0.6761 |

| | |
|---|---|
| ## | 95% CI : (0.633, 0.7171) |

| | |
|---|---|
| ## | No Information Rate : 0.4004 |
| ## | P-Value [Acc > NIR] : <2.2e−16 |
| ## | |

| | |
|---|---|
| ## | Kappa: 0.4879 |

| ## | Mcnemar's Test P-Value : 0.0003553 | | | |
|---|---|---|---|---|
| ## | | | | |
| ## | Statistics by Class: | | | |
| ## | | | | |
| | | | Class: | Class: |
| ## | | Class: CR | S1_XR_JK | S2_JZ_FJ |
| ## | Sensitivity | 0.7157 | 0.6633 | 0.6683 |
| ## | Specificity | 0.9873 | 0.7807 | 0.6980 |
| ## | Pos Pred Value | 0.9359 | 0.6633 | 0.5964 |
| ## | Neg Pred Value | 0.9308 | 0.7807 | 0.7591 |
| ## | Prevalence | 0.2052 | 0.3944 | 0.4004 |
| ## | Detection Rate | 0.1469 | 0.2616 | 0.2676 |
| ## | Detection Prevalence | 0.1569 | 0.3944 | 0.4487 |
| ## | Balanced Accuracy | 0.8515 | 0.7220 | 0.6832 |

| | |
|---|---|
| 6. | CR/NC prediction using classifier built from b1 on b2 samples. |
| ## | Confusion Matrix and Statistics |
| ## | |

| | |
|---|---|
| ## | Reference |

| | | | |
|---|---|---|---|
| ## | Prediction | CR | NC |
| ## | CR | 91 | 7 |
| ## | NC | 9 | 193 |

##

| | |
|---|---|
| ## | Accuracy : 0.9467 |

| | |
|---|---|
| ## | 95% CI : (0.9148, 0.9692) |

| | |
|---|---|
| ## | No Information Rate : 0.6667 |
| ## | P-Value [Acc > NIR] : <2e−16 |
| ## | |

| | |
|---|---|
| ## | Kappa : 0.8794 |

| | |
|---|---|
| ## | Mcnemar's Test P-Value : 0.8026 |
| ## | |

| | |
|---|---|
| ## | Sensitivity : 0.9100 |
| ## | Specificity : 0.9650 |

| | |
|---|---|
| ## | Pos Pred Value : 0.9286 |
| ## | Neg Pred Value : 0.9554 |

| | |
|---|---|
| ## | Prevalence : 0.3333 |

| | |
|---|---|
| ## | Detection Rate : 0.3033 |

| | |
|---|---|
| ## | Detection Prevalence : 0.3267 |

| | |
|---|---|
| ## | Balanced Accuracy : 0.9375 |
| ## | |
| ## | ‘Positive’ Class : CR |
| ## | |

| | |
|---|---|
| 7. | AD/NM prediction using classifier built from b1 on b2 samples. |
| ## | Confusion Matrix and Statistics |
| ## | |

| | |
|---|---|
| ## | Reference |

| | | | |
|---|---|---|---|
| ## | Prediction | AD | NM |
| ## | AD | 183 | 165 |
| ## | NM | 17 | 34 |

##

| | |
|---|---|
| ## | Accuracy : 0.5439 |

| | |
|---|---|
| ## | 95% CI : (0.4936, 0.5935) |

| | |
|---|---|
| ## | No Information Rate : 0.5013 |
| ## | P-Value [Acc > NIR] : 0.04919 |
| ## | |

| | |
|---|---|
| ## | Kappa: 0.086 |

| | |
|---|---|
| ## | Mcnemar's Test P-Value : <2e−16 |
| ## | |

| | |
|---|---|
| ## | Sensitivity : 0.9150 |
| ## | Specificity : 0.1709 |

| | |
|---|---|
| ## | Pos Pred Value : 0.5259 |
| ## | Neg Pred Value : 0.6667 |

| | |
|---|---|
| ## | Prevalence : 0.5013 |

| | |
|---|---|
| ## | Detection Rate : 0.4586 |

| | |
|---|---|
| ## | Detection Prevalence : 0.8722 |

| | |
|---|---|
| ## | Balanced Accuracy : 0.5429 |
| ## | |
| ## | ‘Positive’ Class: AD |
| ## | |

### Confounding Factors

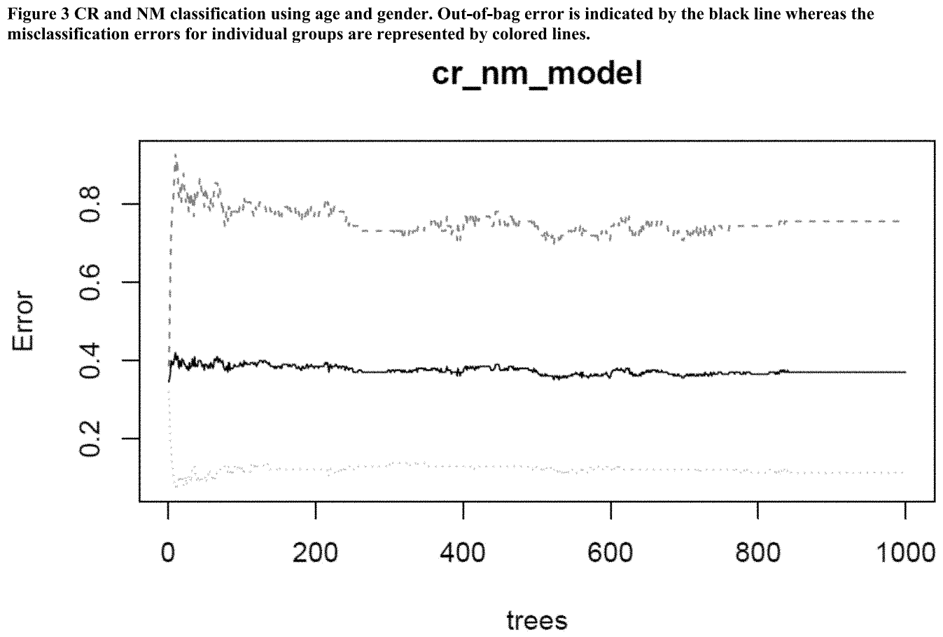

Confounding factors could potentially bias or even invalidate the classification results. In microbiome studies, age and gender are two major confounding factors (1). Though we specifically controlled and balanced these two factors in batch 3 (FIG. 2), the overall distribution was still distorted in the combined dataset. Therefore, we carried out cancer and normal classification using all data using these two factors alone and the result in FIG. 3 showed a large out-of-bag error rate of 37%, which reassures that the good performances of our model was not confounded by age or gender.

### Annotations of the Most Discriminative OTUs Between CR and NM

We analyzed the taxonomic annotations of OTUs ranked by the decreasing order of MeanDecreaseAccuracy value in the random forest classifier model. This metric indicates the importance of the feature in determination of model accuracy. Therefore, it serves as a reasonable measure to judge the relative significance of OTUs. Only OTUs with an arbitrarily chosen cutoff value of 1% were considered. As a result, the number of OTUs in three different models, i.e. trained using 80% pooled, batch 2, and batch 3 samples, were 295, 270, and 276, respectively. 172 OTUs were shared among the three. These OTUs were then annotated against RDP database and the results can be found in the Sequence Listing.

For illustration purpose, we only included top ten OTUs with the highest average MeanDecreaseAccuracy in Table 4. In the table, the first column denotes the OTU ID, the second column denotes the RDP annotation, and the third column denotes the literature concordance as described below.

TABLE 4
The annotations of the top ten most discriminative OTUs shared across three models trained using 80% of
pooled, batch 2, and batch 3 samples. OTUs are ordered by the decreasing average of MeanDecreaseAccuracy.
o, f, g, s stand for order, family, genus, and species. If specified, the last column specifies the lowest
taxonomic rank of the corresponding Otu listed in the review article by Amitay et al. (1) Table 3.

| Otu | Annotation | Literature |
|---|---|---|
| Otu101 | d: Bacteria, p: Bacteroidetes, c: Bacteroidia, o: Bacteroidales, f: Prevotellaceae, g: Prevotella, | — |
| | s: Prevotella intermedia | |
| Otu169 | d: Bacteria, p: Bacteroidetes, c: Bacteroidia, o: Bacteroidales, f: Porphyromonadaceae, g: Porphyromonas | g |
| Otu172 | d: Bacteria, p: Firmicutes, c: Clostridia, o: Clostridiales, f: Peptostreptococcaceae, g: Peptostreptococcus, | s |
| | s: Peptostreptococcus stomatis | |
| Otu121 | d: Bacteria, p: Bacteroidetes, c: Bacteroidia, o: Bacteroidales, f: Bacteroidaceae, g: Bacteroides, | g |
| | s: Bacteroides nordii | |
| Otu185 | d: Bacteria, p: Firmicutes, c: Clostridia, o: Clostridiales, f: Clostridiales Incertae Sedis XI, g: Parvimonas, | s |
| | s: Parvimonas micra | |
| Otu168 | d: Bacteria, p: Firmicutes, c: Negativicutes, o: Selenomonadales, f: Veillonellaceae, g: Dialister, | f |
| | s: Dialister pneumosintes | |
| Otu147 | d: Bacteria, p: Fusobacteria, c: Fusobacteriia, o: Fusobacteriales, f: Fusobacteriaceae, g: Fusobacterium | g |
| Otu47 | d: Bacteria, p: Firmicutes, c: Clostridia, o: Clostridiales, f: Peptostreptococcaceae, g: Romboutsia, | f |
| | s: Romboutsia sedimentorum | |
| Otu142 | d: Bacteria, p: Bacteroidetes, c: Bacteroidia, o: Bacteroidales, f: Porphyromonadaceae, g: Porphyromonas, | g |
| | s: Porphyromonas endodontalis | |
| Otu10 | d: Bacteria, p: Firmicutes, c: Clostridia, o: Clostridiales, f: Lachnospiraceae | o |

Additional OTUs are provided in Table 4.1 below.

TABLE 4.1
OtuName & Annotation & AverageMeanDecAcc & AverageMeanDecGini

Otu101 & d: Bacteria, p: “Bacteroidetes”, c: “Bacteroidia”, o: “Bacteroidales”, f: “Prevotellaceae”, g: Prevotella,
s: Prevotella_{—}intermedia & 13.7943412899552 & 9.83248647017192
Otu169 & d: Bacteria, p: “Bacteroidetes”, c: “Bacteroidia”, o: “Bacteroidales”, f: “Porphyromonadaceae”, g: Porphyromonas &
13.7600435495905 & 8.12128975132281
Otu172 & d: Bacteria, p: Firmicutes, c: Clostridia, o: Clostridiales, f: Peptostreptococcaceae, g: Peptostreptococcus,
s: Peptostreptococcus_{—}stomatis & 13.6778234428472 & 7.36773046283307
Otu121 & d: Bacteria, p: “Bacteroidetes”, c: “Bacteroidia”, o: “Bacteroidales”, f: Bacteroidaceae, g: Bacteroides,
s: Bacteroides_{—}nordii & 12.602462030566 & 5.40850402965016
Otu185 & d: Bacteria, p: Firmicutes, c: Clostridia, o: Clostridiales, f: Clostridiales_Incertae_Sedis_XI, g: Parvimonas,
s: Parvimonas_{—}micra & 11.761749579234 & 6.96865363352588
Otu168 & d: Bacteria, p: Firmicutes, c: Negativicutes, o: Selenomonadales, f: Veillonellaceae, g: Dialister,
s: Dialister_{—}pneumosintes & 11.2576402472093 & 4.90345046638003
Otu147 & d: Bacteria, p: “Fusobacteria”, c: Fusobacteriia, o: “Fusobacteriales”, f: “Fusobacteriaceae”, g: Fusobacterium &
10.9798502944643 & 5.53237578286622
Otu47 & d: Bacteria, p: Firmicutes, c: Clostridia, o: Clostridiales, f: Peptostreptococcaceae, g: Romboutsia,
s: Romboutsia_{—}sedimentorum & 10.1753917813117 & 3.81119243257835
Otu142 & d: Bacteria, p: “Bacteroidetes”, c: “Bacteroidia”, o: “Bacteroidales”, f: “Porphyromonadaceae”, g: Porphyromonas,
s: Porphyromonas_{—}endodontalis & 10.1416113538782 & 4.65257117837514
Otu10 & d: Bacteria, p: Firmicutes, c: Clostridia, o: Clostridiales, f: Lachnospiraceae & 9.69010898213964 & 3.46458888547762
Otu269 & d: Bacteria, p: Firmicutes, c: Bacilli, o: Bacillales, f: Bacillales_Incertae_Sedis_XI, g: Gemella &
8.47014884120977 & 2.43732800289972
Otu72 & d: Bacteria, p: Firmicutes, c: Clostridia, o: Clostridiales, f: Clostridiaceae_1, g: Clostridium_{—}sensu_{—}stricto &
7.89194137307301 & 2.50748599176825
Otu848 & d: Bacteria, p: Firmicutes, c: Clostridia, o: Clostridiales, f: Lachnospiraceae, g: Ruminococcus2,
s: Ruminococcus_{—}torques & 7.80390019103822 & 2.46576850165491
Otu141 & d: Bacteria, p: Firmicutes, c: Clostridia, o: Clostridiales, f: Lachnospiraceae, g: Lachnospiracea_{—}incertae_{—}sedis,
s: Eubacterium_{—}hallii & 7.73321972215815 & 2.51220647076684
Otu309 & d: Bacteria, p: Firmicutes, c: Clostridia, o: Clostridiales, f: Ruminococcaceae, g: Butyricicoccus,
s: Butyricicoccus_{—}pullicaecorum & 7.6800820554995 & 2.24980167781013
Otu85 & d: Bacteria, p: “Bacteroidetes”, c: “Bacteroidia”, o: “Bacteroidales”, f: “Porphyromonadaceae”, g: Odoribacter,
s: Odoribacter_{—}splanchnicus & 7.35446389470393 & 1.3979364158731
Otu111 & d: Bacteria, p: “Bacteroidetes”, c: “Bacteroidia”, o: “Bacteroidales”, f: “Porphyromonadaceae”, g: Parabacteroides,
s: Parabacteroides_{—}goldsteinii & 7.30192582164287 & 1.67450745344268
Otu84 & d: Bacteria, p: Firmicutes, c: Clostridia, o: Clostridiales, f: Lachnospiraceae, g: Clostridium_XIVb &
7.27172325900029 & 1.80487391969814
Otu59 & d: Bacteria, p: Firmicutes, c: Clostridia, o: Clostridiales, f: Lachnospiraceae & 6.44853680333582 & 1.32138594220709
Otu52 & d: Bacteria, p: Firmicutes, c: Clostridia, o: Clostridiales, f: Lachnospiraceae & 6.4160996927843 & 1.16261064298115
Otu423 & d: Bacteria, p: “Bacteroidetes”, c: “Bacteroidia”, o: “Bacteroidales”, f: “Porphyromonadaceae”, g: Parabacteroides &
6.25151810459073 & 1.33645322210194
Otu173 & d: Bacteria, p: “Fusobacteria”, c: Fusobacteriia, o: “Fusobacteriales”, f: “Fusobacteriaceae”, g: Fusobacterium,
s: Fusobacterium_{—}equinum & 6.24608499354993 & 0.891834073083887
Otu26 & d: Bacteria, p: Firmicutes, c: Clostridia, o: Clostridiales, f: Lachnospiraceae, g: Blautia,
s: Blautia_{—}wexlerae & 6.12695291174358 & 1.10524243371151
Otu271 & d: Bacteria, p: “Bacteroidetes”, c: “Bacteroidia”, o: “Bacteroidales”, f: “Porphyromonadaceae”, g: Porphyromonas,
s: Porphyromonas_{—}somerae & 5.96932923671922 & 0.809478873317209
Otu20 & d: Bacteria, p: “Bacteroidetes”, c: “Bacteroidia”, o: “Bacteroidales”, f: Bacteroidaceae, g: Bacteroides,
s: Bacteroides_{—}fragilis & 5.9646209916872 & 1.31438877628573
Otu33 & d: Bacteria, p: “Verrucomicrobia”, c: Vemicomicrobiae, o: Vemicomicrobiales, f: Verrucomicrobiaceae, g: Akkermansia,
s: Akkermansia_{—}muciniphila & 5.8989902784533 & 1.1344669200008
Otu81 & d: Bacteria, p: Firmicutes, c: Clostridia, o: Clostridiales, f: Ruminococcaceae & 5.82374608835491 & 1.54889847520407
Otu2745 & d: Bacteria, p: “Bacteroidetes”, c: “Bacteroidia”, o: “Bacteroidales”, f: “Prevotellaceae”, g: Prevotella,
s: Prevotella_{—}stercorea & 5.66871908025159 & 1.28437240850829
Otu4384 & d: Bacteria, p: Firmicutes, c: Negativicutes, o: Selenomonadales, f: Acidaminococcaceae, g: Phascolarctobacterium,
s: Phascolarctobacterium_{—}faecium & 5.52043749491481 & 0.420271701946243
Otu148 & d: Bacteria, p: Firmicutes, c: Clostridia, o: Clostridiales, f: Peptostreptococcaceae, g: Intestinibacter,
s: Intestinibacter_{—}bartlettii & 5.41945049407486 & 0.842883283253836
Otu1777 & d: Bacteria, p: “Bacteroidetes”, c: “Bacteroidia”, o: “Bacteroidales”, f: “Prevotellaceae”, g: Prevotella,
s: Prevotella_{—}copri & 5.33503317698889 & 0.648348328905093
Otu4342 & d: Bacteria, p: Firmicutes, c: Clostridia, o: Clostridiales, f: Peptostreptococcaceae, g: Terrisporobacter,
s: Terrisporobacter_{—}glycolicus & 5.33274424863514 & 0.710046587499439
Otu76 & d: Bacteria, p: Firmicutes, c: Negativicutes, o: Selenomonadales, f: Acidaminococcaceae, g: Phascolarctobacterium,
s: Phascolarctobacterium_{—}succinatutens & 5.32415139654529 & 1.07287902798243
Otu155 & d: Bacteria, p: “Synergistetes”, c: Synergistia, o: Synergistales, f: Synergistaceae, g: Pyramidobacter,
s: Pyramidobacter_{—}piscolens & 5.30041145292807 & 0.532092720378172
Otu106 & d: Bacteria, p: “Bacteroidetes”, c: “Bacteroidia”, o: “Bacteroidales”, f: Bacteroidaceae, g: Bacteroides,
s: Bacteroides_{—}salyersiae & 5.27691156894213 & 0.704064927855818
Otu82 & d: Bacteria, p: “Proteobacteria”, c: Betaproteobacteria, o: Burkholderiales, f: Sutterellaceae, g: Sutterella &
5.2437877972519 & 0.916433764419022
Otu35 & d: Bacteria, p: “Bacteroidetes”, c: “Bacteroidia”, o: “Bacteroidales”, f: “Rikenellaceae”, g: Alistipes,
s: Alistipes_{—}onderdonkii & 5.18360405074251 & 0.76182460502378
Otu3312 & d: Bacteria, p: Firmicutes, c: Clostridia, o: Clostridiales, f: Clostridiaceae_1, g: Clostridium_{—}sensu_{—}stricto &
5.12448018510061 & 1.2995460402096
Otu253 & d: Bacteria, p: Firmicutes, c: Clostridia, o: Clostridiales, f: Ruminococcaceae, g: Ruminococcus,
s: Runiinococcus_{—}flavefaciens & 5.01593910842362 & 0.950489489552967
Otu351 & d: Bacteria, p: “Bacteroidetes”, c: “Bacteroidia”, o: “Bacteroidales”, f: “Porphyromonadaceae”, g: Butyricimonas,
s: Butyricimonas_{—}faecihominis & 4.94622364446024 & 0.772092262070063
Otu98 & d: Bacteria, p: “Bacteroidetes”, c: “Bacteroidia”, o: “Bacteroidales”, f: “Rikenellaceae”, g: Alistipes,
s: Alistipes_{—}shahii & 4.9265290619132 & 0.484605626680004
Otu77 & d: Bacteria, p: “Bacteroidetes”, c: “Bacteroidia”, o: “Bacteroidales”, f: “Prevotellaceae”, g: Prevotella &
4.86175121992317 & 1.20142046245559
Otu317 & d: Bacteria, p: “Bacteroidetes”, c: “Bacteroidia”, o: “Bacteroidales”, f: “Porphyromonadaceae”, g: Butyricimonas,
s: Butyricimonas_{—}paravirosa & 4.78124294124035 & 1.08675849249154
Otu153 & d: Bacteria, p: Firmicutes, c: Clostridia, o: Clostridiales, f: Ruminococcaceae & 4.77621244980273 & 0.505182479173224
Otu83 & d: Bacteria, p: Firmicutes, c: Clostridia, o: Clostridiales, f: Lachnospiraceae, g: Coprococcus,
s: Coprococcus_{—}eutactus & 4.62649902286053 & 0.579988780285664
Otu60 & d: Bacteria, p: “Proteobacteria”, c: Deltaproteobacteria, o: Desulfovibrionales, f: Desulfovibrionaceae, g: Bilophila,
s: Bilophila_{—}wadsworthia & 4.58228432357164 & 0.482910634332228
Otu287 & d: Bacteria, p: Firmicutes, c: Clostridia, o: Clostridiales, f: Ruminococcaceae, g: Oscillibacter &
4.3480408468567 & 0.627989174153698
Otu78 & d: Bacteria, p: Firmicutes, c: Clostridia, o: Clostridiales & 4.25273477261076 & 0.345090535435327
Otu2074 & d: Bacteria, p: Firmicutes, c: Clostridia, o: Clostridiales, f: Lachnospiraceae & 4.19168565814693 & 0.833783613563489
Otu118 & d: Bacteria, p: Firmicutes, c: Clostridia, o: Clostridiales, f: Lachnospiraceae, g: Blautia &
4.10119372513613 & 0.393811168404519
Otu23 & d: Bacteria, p: Firmicutes, c: Clostridia, o: Clostridiales, f: Lachnospiraceae & 4.1001842535131 & 0.422732522859675
Otu18 & d: Bacteria, p: “Bacteroidetes”, c: “Bacteroidia”, o: “Bacteroidales”, f: “Rikenellaceae”, g: Alistipes &
4.05704708781915 & 0.467682866630194
Otu264 & d: Bacteria, p: “Actinobacteria”, c: Actinobacteria, o: Actinomycetales, f: Nocardiaceae, g: Nocardia,
s: Nocardia_{—}coeliaca & 4.04731217339991 & 0.828711662376662
Otu218 & d: Bacteria, p: “Bacteroidetes”, c: “Bacteroidia”, o: “Bacteroidales”, f: “Prevotellaceae”, g: Prevotella,
s: Prevotella_{—}stercorea & 4.02023860335542 & 0.604243441207422
Otu97 & d: Bacteria, p: Firmicutes, c: Clostridia, o: Clostridiales, f: Lachnospiraceae, g: Clostridium_XIVa &
3.90813842505155 & 0.387375128776727
Otu191 & d: Bacteria, p: Firmicutes, c: Clostridia, o: Clostridiales, f: Ruminococcaceae, g: Anaerotruncus,
s: Anaerotruncus_{—}colihominis & 3.89915867132865 & 0.570306115817279
Otu175 & d: Bacteria, p: Firmicutes, c: Clostridia, o: Clostridiales & 3.89077367715736 & 0.38844488215353
Otu265 & d: Bacteria, p: Firmicutes, c: Clostridia, o: Clostridiales, f: Ruminococcaceae, g: Ruminococcus &
3.88089562006944 & 0.344105771852526
Otu727 & d: Bacteria, p: Firmicutes, c: Clostridia, o: Clostridiales, f: Ruminococcaceae & 3.8758534592987 & 0.484685400173847
Otu266 & d: Bacteria, p: “Bacteroidetes”, c: “Bacteroidia”, o: “Bacteroidales” & 3.86783248378869 & 0.19799633775168
Otu723 & d: Bacteria, p: Firmicutes, c: Clostridia, o: Clostridiales, f: Lachnospiraceae & 3.85242756965532 & 0.282801172808673
Otu7 & d: Bacteria, p: “Bacteroidetes”, c: “Bacteroidia”, o: “Bacteroidales”, f: Bacteroidaceae, g: Bacteroides,
s: Bacteroides_{—}unifomiis & 3.8065043922493 & 0.329438846721559
Otu21 & d: Bacteria, p: Firmicutes, c: Clostridia, o: Clostridiales, f: Lachnospiraceae, g: Lachnospiracea_{—}incertae_{—}sedis,
s: Eubacterium_{—}eligens & 3.80126351761255 & 0.444516015697381
Otu22 & d: Bacteria, p: Firmicutes, c: Negativicutes, o: Selenomonadales, f: Veillonellaceae, g: Megamonas,
s: Megamonas_{—}funiformis & 3.71766759392569 & 0.195933894693333
Otu224 & d: Bacteria, p: Firmicutes, c: Bacilli, o: Lactobacillales, f: Streptococcaceae, g: Streptococcus &
3.71020513681508 & 0.25581950882642
Otu2109 & d: Bacteria, p: Firmicutes, c: Clostridia, o: Clostridiales & 3.70216652149231 & 0.365839982738123
Otu2060 & d: Bacteria, p: Firmicutes, c: Clostridia, o: Clostridiales, f: Lachnospiraceae & 3.69633802060259 & 0.395815871333106
Otu90 & d: Bacteria, p: Firmicutes, c: Clostridia, o: Clostridiales, f: Lachnospiraceae & 3.65702177036977 & 0.299636570294157
Otu348 & d: Bacteria, p: “Bacteroidetes”, c: “Bacteroidia”, o: “Bacteroidales”, f: “Porpliyromonadaceae”, g: Butyricimonas &
3.65525080958422 & 0.222183262159006
Otu3254 & d: Bacteria, p: “Bacteroidetes”, c: “Bacteroidia”, o: “Bacteroidales”, f: “Rikenellaceae”, g: Alistipes,
s: Alistipes_{—}finegoldii & 3.64447212313583 & 0.338448240628326
Otu316 & d: Bacteria, p: “Bacteroidetes”, c: “Bacteroidia”, o: “Bacteroidales”, f: Bacteroidaceae, g: Bacteroides,
s: Bacteroides_{—}xylanisolvens & 3.64238523653699 & 0.53266003775059
Otu1264 & d: Bacteria, p: Firmicutes, c: Clostridia, o: Clostridiales, f: Lachnospiraceae & 3.58565897976223 & 0.460049748834728
Otu164 & d: Bacteria, p: Firmicutes, c: Clostridia, o: Clostridiales, f: Ruminococcaceae & 3.51368756410499 & 0.514723500523881
Otu15 & d: Bacteria, p: “Bacteroidetes”, c: “Bacteroidia”, o: “Bacteroidales”, f: Bacteroidaceae, g: Bacteroides,
s: Bacteroides_{—}thetaiotaomicron & 3.44288627468682 & 0.52939450434855
Otu1168 & d: Bacteria, p: Firmicutes, c: Clostridia, o: Clostridiales, f: Lachnospiraceae & 3.38497643190079 & 0.215602689462476
Otu105 & d: Bacteria, p: “Actinobacteria”, c: Actinobacteria, o: Bifidobacteriales, f: Bifidobacteriaceae, g: Bifidobacterium &
3.37211346365296 & 0.327187921839971
Otu248 & d: Bacteria, p: Firmicutes, c: Clostridia, o: Clostridiales, f: Ruminococcaceae & 3.32214409123697 & 0.425238478381044
Otu410 & d: Bacteria, p: Firmicutes, c: Clostridia, o: Clostridiales, f: Ruminococcaceae & 3.30288192561728 & 0.125663216048697
Otu177 & d: Bacteria, p: “Bacteroidetes”, c: “Bacteroidia”, o: “Bacteroidales”, f: Bacteroidaceae, g: Bacteroides &
3.27044511626177 & 0.223118179430504
Otu274 & d: Bacteria & 3.16780822565938 & 0.0803245187481717
Otu704 & d: Bacteria, p: Firmicutes, c: Clostridia, o: Clostridiales, f: Lachnospiraceae & 3.15847365410314 & 0.1451100410588
Otu36 & d: Bacteria, p: “Bacteroidetes”, c: “Bacteroidia”, o: “Bacteroidales”, f: Bacteroidaceae, g: Bacteroides,
s: Bacteroides_{—}caccae & 3.15801571908562 & 0.185221033755153
Otu160 & d: Bacteria, p: Firmicutes, c: Negativicutes, o: Selenomonadales, f: Veillonellaceae, g: Veillonella,
s: Veillonella_{—}magna & 3.12333106757157 & 0.084711377604504
Otu336 & d: Bacteria, p: “Bacteroidetes”, c: “Bacteroidia”, o: “Bacteroidales”, f: “Prevotellaceae”, g: Prevotella &
3.09684587237006 & 0.112261991219131
Otu235 & d: Bacteria, p: “Bacteroidetes”, c: “Bacteroidia”, o: “Bacteroidales” & 3.09438367534219 & 0.232199026269785
Otu2231 & d: Bacteria, p: Firmicutes, c: Clostridia, o: Clostridiales, f: Ruminococcaceae, g: Anaerotruncus,
s: Anaerotruncus_{—}colihominis & 3.04296587460515 & 0.158223508241415
Otu107 & d: Bacteria, p: Firmicutes, c: Clostridia, o: Clostridiales, f: Lachnospiraceae, g: Anaerostipes,
s: Eubacterium_{—}hadrum & 2.98593610168943 & 0.232812008400764
Otu96 & d: Bacteria, p: Firmicutes, c: Clostridia, o: Clostridiales, f: Lachnospiraceae, g: Lachnospiracea_{—}incertae_{—}sedis &
2.98225575498437 & 0.105427685386433
Otu79 & d: Bacteria, p: Firmicutes & 2.98120624114534 & 0.106896245872236
Otu93 & d: Bacteria, p: “Bacteroidetes”, c: “Bacteroidia”, o: “Bacteroidales”, f: “Porphyromonadaceae” &
2.9479410810479 & 0.2765692890981
Otu89 & d: Bacteria, p: Firmicutes, c: Clostridia, o: Clostridiales, f: Eubacteriaceae, g: Eubacterium,
s: Eubacterium_{—}coprostanoligenes & 2.93433072901629 & 0.254358672819042
Otu16 & d: Bacteria, p: Firmicutes, c: Clostridia, o: Clostridiales, f: Ruminococcaceae & 2.92181685324236 & 0.148790353205781
Otu3 & d: Bacteria, p: “Bacteroidetes”, c: “Bacteroidia”, o: “Bacteroidales”, f: “Prevotellaceae”, g: Prevotella,
s: Prevotella_{—}copri & 2.90120890308239 & 0.278575486425403
Otu174 & d: Bacteria, p: Firmicutes, c: Clostridia, o: Clostridiales, f: Ruminococcaceae, g: Ruminococcus,
s: Ruminococcus_{—}champanellensis & 2.86991039022236 & 0.161845949318228
Otu34 & d: Bacteria, p: Firmicutes, c: Clostridia, o: Clostridiales, f: Ruminococcaceae & 2.86277209414093 & 0.136104587463048
Otu450 & d: Bacteria, p: “Bacteroidetes”, c: “Bacteroidia”, o: “Bacteroidales”, f: “Porphyromonadaceae”, g: Butyricimonas &
2.84990574675875 & 0.104419029056058
Otu4397 & d: Bacteria, p: “Bacteroidetes”, c: “Bacteroidia”, o: “Bacteroidales”, f: Bacteroidaceae, g: Bacteroides,
s: Bacteroides_{—}plebeius & 2.83725087022718 & 0.182106886898651
Otu122 & d: Bacteria, p: Firmicutes, c: Clostridia, o: Clostridiales, f: Clostridiaceae_1, g: Clostridium_{—}sensu_{—}stricto &
2.82856887827566 & 0.108670043639969
Otu967 & d: Bacteria, p: “Bacteroidetes”, c: “Bacteroidia”, o: “Bacteroidales”, f: “Prevotellaceae”, g: Prevotella &
2.80817869556781 & 0.173643923405744
Otu1944 & d: Bacteria, p: Firmicutes, c: Clostridia, o: Clostridiales, f: Clostridiaceae_1, g: Clostridium_{—}sensu_{—}stricto,
s: Clostridium_{—}paraputrificum & 2.71023404713693 & 0.100466624560385
Otu1941 & d: Bacteria, p: Firmicutes, c: Clostridia, o: Clostridiales, f: Lachnospiraceae & 2.69838743711004 & 0.142278127176266
Otu39 & d: Bacteria, p: “Bacteroidetes”, c: “Bacteroidia”, o: “Bacteroidales”, f: “Prevotellaceae”, g: Prevotella,
s: Prevotella_{—}stercorea & 2.63842518186387 & 0.141027507352634
Otu135 & d: Bacteria, p: “Fusobacteria”, c: Fusobacteriia, o: “Fusobacteriales”, f: “Fusobacteriaceae”, g: Cetobacterium,
s: Cetobacterium_{—}somerae & 2.61968268548529 & 0.0831505189137432
Otu2059 & d: Bacteria, p: Firmicutes, c: Bacilli, o: Lactobacillales, f: Streptococcaceae, g: Streptococcus &
2.61413664120766 & 0.175922168709985
Otu2666 & d: Bacteria, p: Firmicutes, c: Clostridia, o: Clostridiales & 2.58883232060338 & 0.112654703184687
Otu6 & d: Bacteria, p: Firmicutes, c: Clostridia, o: Clostridiales, f: Lachnospiraceae & 2.58310675012197 & 0.177798986648724
Otu1226 & d: Bacteria, p: Firmicutes, c: Clostridia, o: Clostridiales, f: Lachnospiraceae, g: Clostridium_XIVa,
s: Clostridium_{—}aldenense & 2.55929498462539 & 0.221048689629986
Otu1013 & d: Bacteria, p: Firmicutes, c: Clostridia, o: Clostridiales, f: Lachnospiraceae & 2.55055552177418 & 0.143658469390376
Otu12 & d: Bacteria, p: “Bacteroidetes”, c: “Bacteroidia”, o: “Bacteroidales”, f: Bacteroidaceae, g: Bacteroides,
s: Bacteroides_{—}stercoris & 2.51708008793652 & 0.103915012493887
Otu3144 & d: Bacteria, p: Firmicutes, c: Clostridia, o: Clostridiales, f: Lachnospiraceae & 2.51673692049532 & 0.165227082965755
Otu237 & d: Bacteria, p: “Bacteroidetes”, c: “Bacteroidia”, o: “Bacteroidales”, f: “Prevotellaceae”, g: Prevotella &
2.51117802646258 & 0.226025083820349
Otu279 & d: Bacteria, p: “Bacteroidetes”, c: “Bacteroidia”, o: “Bacteroidales”, f: “Porphyromonadaceae”, g: Parabacteroides,
s: Parabacteroides_{—}gordonii & 2.48048095113267 & 0.100806236371619
Otu64 & d: Bacteria, p: “Bacteroidetes”, c: “Bacteroidia”, o: “Bacteroidales”, f: “Prevotellaceae”, g: Paraprevotella,
s: Paraprevotella_{—}clara & 2.46395765375973 & 0.0690878515368844
Otu25 & d: Bacteria, p: Firmicutes, c: Clostridia, o: Clostridiales, f: Lactmospiraceae & 2.45023659597359 & 0.214516967460789
Otu19 & d: Bacteria, p: “Bacteroidetes”, c: “Bacteroidia”, o: “Bacteroidales”, f: “Porphyromonadaceae”, g: Parabacteroides,
s: Parabacteroides_{—}merdae & 2.44204192953914 & 0.152688966441248
Otu2406 & d: Bacteria, p: Firmicutes, c: Clostridia, o: Clostridiales, f: Lachnospiraceae, g: Coprococcus,
s: Coprococcus_{—}eutactus & 2.388647764166 & 0.179625343318508
Otu2441 & d: Bacteria, p: “Bacteroidetes”, c: “Bacteroidia”, o: “Bacteroidales”, f: “Prevotellaceae”, g: Prevotella,
s: Prevotella_{—}stercorea & 2.36221022347778 & 0.0860287788041391
Otu4383 & d: Bacteria, p: “Bacteroidetes”, c: “Bacteroidia”, o: “Bacteroidales”, f: “Prevotellaceae” &
2.30917215168753 & 0.169677409577486
Otu785 & d: Bacteria, p: Firmicutes, c: Clostridia, o: Clostridiales & 2.2979764524382 & 0.120920186197908
Otu184 & d: Bacteria, p: “Proteobacteria”, c: Alphaproteobacteria & 2.2953335860093 & 0.125357854092819
Otu529 & d: Bacteria, p: Firmicutes, c: Clostridia, o: Clostridiales & 2.28626290793623 & 0.0591800476336016
Otu211 & d: Bacteria, p: “Bacteroidetes”, c: “Bacteroidia”, o: “Bacteroidales”, f: “Prevotellaceae”, g: Prevotella &
2.27530944518009 & 0.0825446930662444
Otu1285 & d: Bacteria, p: “Bacteroidetes”, c: “Bacteroidia”, o: “Bacteroidales”, f: “Rikenellaceae”, g: Alistipes &
2.27216170398856 & 0.10048598114358
Otu154 & d: Bacteria, p: “Proteobacteria”, c: Betaproteobacteria, o: Burkholderiales, f: Sutterellaceae, g: Sutterella,
s: Sutterella_{—}wadsworthensis & 2.26681317274378 & 0.095794761955645
Otu73 & d: Bacteria, p: “Bacteroidetes”, c: “Bacteroidia”, o: “Bacteroidales”, f: Bacteroidaceae, g: Bacteroides,
s: Bacteroides_{—}eggerthii & 2.23490099723446 & 0.100177500333695
Otu110 & d: Bacteria, p: Firmicutes, c: Erysipelotrichia, o: Erysipelotrichales, f: Erysipelotrichaceae, g: Holdemanella,
s: Holdemanella_{—}bifomiis & 2.21687067076921 & 0.0810713870408617
Otu323 & d: Bacteria, p: “Bacteroidetes”, c: “Bacteroidia”, o: “Bacteroidales”, f: “Prevotellaceae”, g: Prevotella &
2.21189156399316 & 0.0498167164045447
Otu30 & d: Bacteria, p: Firmicutes, c: Clostridia, o: Clostridiales, f: Lachnospiraceae & 2.20972306269567 & 0.124888017222478
Otu197 & d: Bacteria, p: Firmicutes, c: Clostridia, o: Clostridiales, f: Ruminococcaceae, g: Ruminococcus &
2.19787510012812 & 0.0688095464180803
Otu325 & d: Bacteria, p: Firmicutes & 2.19765719927231 & 0.0724881781650027
Otu92 & d: Bacteria, p: Firmicutes, c: Clostridia, o: Clostridiales & 2.19754290190436 & 0.0977614715791891
Otu137 & d: Bacteria, p: “Bacteroidetes”, c: “Bacteroidia”, o: “Bacteroidales”, f: Bacteroidaceae, g: Bacteroides,
s: Bacteroides_{—}fluxus & 2.19259587590723 & 0.0957227663704627
Otu398 & d: Bacteria, p: Firmicutes, c: Clostridia, o: Clostridiales, f: Lachnospiraceae, g: Clostridium_XIVb,
s: Clostridium_{—}lactatifemientans & 2.16619612097008 & 0.13243012390506
Otu24 & d: Bacteria, p: Firmicutes, c: Clostridia, o: Clostridiales, f: Lachnospiraceae, g: Fusicatenibacter,
s: Fusicatenibacter_{—}saccharivorans & 2.13601207826098 & 0.109004618099555
Otu1310 & d: Bacteria, p: Firmicutes, c: Clostridia, o: Clostridiales, f: Lachnospiraceae, g: Clostridium_XIVa,
s: Clostridium_{—}lavalense & 2.10031266330233 & 0.0681859590894292
Otu61 & d: Bacteria, p: Firmicutes, c: Clostridia, o: Clostridiales, f: Ruminococcaceae & 2.06621226238679 & 0.0812814627693076
Otu341 & d: Bacteria, p: “Bacteroidetes”, c: “Bacteroidia”, o: “Bacteroidales”, f: Bacteroidaceae, g: Bacteroides &
2.05394025479534 & 0.0660563999551188
Otu181 & d: Bacteria, p: Firmicutes, c: Clostridia, o: Clostridiales, f: Ruminococcaceae & 2.04844656233313 & 0.0571401007980638
Otu143 & d: Bacteria, p: “Bacteroidetes”, c: “Bacteroidia”, o: “Bacteroidales”, f: “Porphyromonadaceae”, g: Butyricimonas,
s: Butyricimonas_{—}virosa & 2.03243584288693 & 0.0970020028567559
Otu67 & d: Bacteria, p: “Proteobacteria”, c: Betaproteobacteria, o: Burkholderiales, f: Sutterellaceae, g: Parasutterella,
s: Parasutterella_{—}excrementihominis & 2.03180324746581 & 0.0936881467159242
Otu252 & d: Bacteria, p: “Bacteroidetes”, c: “Bacteroidia”, o: “Bacteroidales”, f: “Porphyromonadaceae”, g: Butyricimonas &
2.02940489409138 & 0.070616655927486
Otu492 & d: Bacteria, p: “Bacteroidetes”, c: “Bacteroidia”, o: “Bacteroidales”, f: Bacteroidaceae,
g: Bacteroides & 2.02849125631133 & 0.0961577655297611
Otu102 & d: Bacteria, p: Firmicutes, c: Clostridia, o: Clostridiales, f: Ruminococcaceae & 2.02671995711953 & 0.0547494767351553
Otu844 & d: Bacteria, p: Firmicutes, c: Clostridia, o: Clostridiales, f: Lachnospiraceae & 2.01976446057376 & 0.103854802087175
Otu167 & d: Bacteria, p: Firmicutes, c: Clostridia, o: Clostridiales, f: Ruminococcaceae, g: Ruminococcus,
s: Runiinococcus_{—}callidus & 2.00637176738852 & 0.0686186701834018
Otu268 & d: Bacteria, p: “Bacteroidetes”, c: “Bacteroidia”, o: “Bacteroidales”, f: “Porphyromonadaceae”, g: Coprobacter,
s: Coprobacter_{—}fastidiosus & 1.99552235062283 & 0.12422248748126
Otu53 & d: Bacteria, p: Firmicutes, c: Clostridia, o: Clostridiales, f: Ruminococcaceae, g: Flavonifractor,
s: Flavonifractor_{—}plautii & 1.98477602820225 & 0.154388346573957
Otu134 & d: Bacteria, p: Firmicutes, c: Clostridia, o: Clostridiales, f: Ruminococcaceae, g: Runiinococcus,
s: Runiinococcus_{—}broniii & 1.943819299683 & 0.078283004968428
Otu162 & d: Bacteria, p: Firmicutes, c: Clostridia, o: Clostridiales, f: Ruminococcaceae & 1.90030595960624 & 0.0563884110984546
Otu100 & d: Bacteria, p: Firmicutes, c: Clostridia, o: Clostridiales & 1.82797703408088 & 0.0738899503135034
Otu4152 & d: Bacteria, p: “Actinobacteria”, c: Actinobacteria, o: Bifidobacteriales, f: Bifidobacteriaceae, g: Bifidobacterium,
s: Bifidobacterium_{—}bifidum & 1.82566704030467 & 0.099354472367359
Otu777 & d: Bacteria, p: “Bacteroidetes”, c: “Bacteroidia”, o: “Bacteroidales”, f: “Porphyromonadaceae”, g: Parabacteroides &
1.7657225582824 & 0.0325864924110219
Otu54 & d: Bacteria, p: Firmicutes, c: Clostridia, o: Clostridiales, f: Ruminococcaceae, g: Oscillibacter &
1.7519877374647 & 0.0847745772082939
Otu1438 & d: Bacteria, p: Firmicutes, c: Clostridia, o: Clostridiales, f: Lachnospiraceae, g: Lachnospiracea_{—}incertae_{—}sedis &
1.73280842049184 & 0.0526217992535465
Otu51 & d: Bacteria, p: “Proteobacteria”, c: Betaproteobacteria, o: Burkliolderiales & 1.72804826925365 & 0.12269085994415
Otu111 & d: Bacteria, p: Firmicutes, c: Clostridia, o: Clostridiales, f: Lachnospiraceae, g: Coprococcus,
s: Coprococcus_{—}comes & 1.71550934616673 & 0.144405921174456
Otu405 & d: Bacteria, p: “Bacteroidetes”, c: “Bacteroidia”, o: “Bacteroidales”, f: Bacteroidaceae, g: Bacteroides,
s: Bacteroides_{—}bamesiae & 1.70880833677066 & 0.0246207576224092
Otu213 & d: Bacteria, p: Firmicutes, c: Negativicutes, o: Selenomonadales, f: Veillonellaceae, g: Dialister,
s: Dialister_{—}succinatiphilus & 1.70144938188134 & 0.0816118396027724
Otu2399 & d: Bacteria, p: Firmicutes, c: Clostridia, o: Clostridiales & 1.69365497194395 & 0.041528439217283
Otu40 & d: Bacteria, p: Firmicutes, c: Clostridia, o: Clostridiales, f: Lachnospiraceae, g: Ruminococcus2,
s: Ruminococcus_{—}faecis & 1.68166001885592 & 0.106539911906408
Otu115 & d: Bacteria, p: Firmicutes, c: Negativicutes, o: Selenomonadales, f: Veillonellaceae, g: Megasphaera &
1.64501381637878 & 0.0824926787147221
Otu1576 & d: Bacteria, p: Firmicutes, c: Negativicutes, o: Selenomonadales, f: Veillonellaceae, g: Megamonas,
s: Megamonas_{—}funiformis & 1.61456104357672 & 0.066220021010319
Otu1214 & d: Bacteria, p: “Bacteroidetes”, c: “Bacteroidia”, o: “Bacteroidales”, f: “Porphyromonadaceae”, g: Parabacteroides,
s: Parabacteroides_{—}gordonii & 1.60397148374387 & 0.053135067964
Otu128 & d: Bacteria, p: “Proteobacteria”, c: Alphaproteobacteria & 1.60113768726192 & 0.047269458772049
Otu32 & d: Bacteria, p: “Bacteroidetes”, c: “Bacteroidia”, o: “Bacteroidales”, f: Bacteroidaceae, g: Bacteroides,
s: Bacteroides_{—}coprophilus & 1.5704063903467 & 0.0688575737639849
Otu1386 & d: Bacteria, p: Firmicutes, c: Clostridia, o: Clostridiales, f: Lachnospiraceae & 1.53353997109029 & 0.0442083115662555
Otu2 & d: Bacteria, p: Firmicutes, c: Clostridia, o: Clostridiales, f: Ruminococcaceae, g: Faecalibacterium,
s: Faecalibacterium_{—}prausnitzii & 1.51051364783698 & 0.0746406775857877
Otu1841 & d: Bacteria, p: Firmicutes, c: Clostridia, o: Clostridiales, f: Lachnospiraceae, g: Clostridium_XIVa &
1.50471587369414 & 0.0457896807308778
Otu123 & d: Bacteria, p: “Bacteroidetes”, c: “Bacteroidia”, o: “Bacteroidales”, f: “Prevotellaceae”, g: Paraprevotella,
s: Paraprevotella_{—}xylaniphila & 1.45542839323159 & 0.03049862573998
Otu346 & d: Bacteria, p: Firmicutes, c: Clostridia, o: Clostridiales & 1.38676304035384 & 0.014614966160068
Otu156 & d: Bacteria, p: Firmicutes, c: Clostridia, o: Clostridiales, f: Lachnospiraceae & 1.36952366127748 & 0.0474515503949865
Otu144 & d: Bacteria, p: Firmicutes, c: Clostridia, o: Clostridiales, f: Lachnospiraceae, g: Clostridium_XIVa &
1.33968420287925 & 0.0568146633936392

Consistent with the existing studies, g:Fusobacterium is found to be one of the top discriminative features. B. fragilis, although not shown in the table, has the 25th largest MeanDecreaseAccuracy value. To demonstrate the relevance of the remaining ones shown in the table, we compared these annotations against the bacteria list summarized by Amitay et al. (1). In their study, a comprehensive survey was carried out to summarize as many relevant literatures as possible that studied differences in microbiota composition between CRC and normal controls. They recorded a list of bacteria and their annotations that occurred in at least two of such literature studies and were found to be discriminative.

The comparison showed concordant results, recorded in the third column of Table 4. The taxonomic rank, when specified, denotes the lowest consistent annotation between the two. All but Otu101 were found. Notably, Otu101, annotated as g:Prevotella, was identified as one of the most discriminative feature in the current study but was absence in the summary list of Amitay et al. study. With further investigation, we identified multiple recent studies demonstrating the association of g:Prevotella with CRC. In an attempt to associate microbiota with different molecular subtypes of CRC (22), Prevotella has been shown to strongly associated with CMS2, one of the dominant subtype shown to have the prevalence of 37% among CRC patients. Prevotella intermedia has also been shown to be co-occur with Fusobacterium in matched and metastatic tumors (4). And a more recent study (9) across four different cohort identified Prevotella intermedia as one of the seven CRC-enriched biomarkers. Next, we investigate whether the summary list in Amitay et al. study were identified in the current cohort. At the genus level, all but Roseburia, Leptotrichia, Atopobium have been found in Table 4.1.

### Classification: Multi-Group

Given that we collected a balanced number of samples in both batch 2 and batch 3, we use only these two batches for multi-group classification.

We first generated the classification of three intermediate Groups (AA, NA, PL) using the classifier built from Cancer (CR) and normal (NM). The classifier was built using 80% of CR and NM samples, and classifications were made on the remaining.

TABLE 5
Classification Results for CR, NM, AA,
NA, PL with model trained on CR, NM

| Prediction | CR | AA | NA | PL | NM |
|---|---|---|---|---|---|
| CR | 41 | 45 | 1 | 3 | 0 |
| NM | 2 | 151 | 205 | 193 | 35 |

As shown in table 5, the classifications on cancer and normal samples were comparable as previously seen. For the other three groups, about a quarter of advanced adenoma (AA) samples were been labeled as cancer, whereas almost all samples from non-advanced adenoma (NA) and polyps (PL) were labeled as non-cancer. This results indicate the microbiome composition of AA group may have higher resemblance to the cancer and the less advanced disease groups have more resemblance to the normal. This could also indicates a shift in microbiome composition when reaching a severe disease status.

Next, we generated classification results for all five groups and finally, according to disease status, we combined samples from AA and NA to be ademona group (AD) and combined PL and NM to be the non-diseased group (NP), and applied classification on these three groups. The results are summarized in Table 6.

TABLE 6
Multigroup classification results. Groups are separated. The combined
three groups are considered as cancer (CR), adenoma, denoted
by AD (AA, NA), and non-adenoma, denoted by NP (NM, PL).

| Groups | Class | Sensitivity | Specificity | Accuracy |
|---|---|---|---|---|
| CRAANA | CR | 0.954 | 0.962 | 0.890 |
| PLNM | AA | 0.714 | 0.974 | |
| | NA | 0.889 | 0.951 | |
| | PL | 0.949 | 0.994 | |
| | NM | 1.000 | 0.982 | |
| CR AD | CR | 0.954 | 0.968 | 0.935 |
| NP | (AA, NA) | 0.894 | 0.983 | |
| | (PL, NM) | 0.972 | 0.953 | |

We achieved 89% overall accuracy for the five group classification and 93.5% accuracy for the three group classification. A detailed inspection revealed that for five groups, the sensitivities of AA and NA are much lower compared to the others, largely due to many misclassified cases from AA to CR and NA, and NA to AA. This observation supported the idea that overlapping signals are shared across different disease status, and the disease progression may occur in a continuous fashion as indicated by the observation that the misclassification mostly occur between adjacent status. Therefore, as expected, it is more challenging to accurately identify at which disease progression status a patient was when a larger number of grouping were used according to histopathological criteria. The detailed classification results can be found below.

### Classification of NuoHui 999 Combined Batch2 and Batch3 Stool Microbiome Samples

### 1. Background

Two independent batches of stool microbiome samples have been collected. For each batch, five categories have been defined: CR (cancer), JZ (progression), FJ (non-progression), XR (polypus), JK (normal), where each category has ˜100 samples. First, we build classifier using 80% CR/JK samples, then make predictions on the remaining 20% CR/JK samples. Then using the same model, we make predictions on JZ/FJ/XR samples. Next, we build five group classifiers using 80% of the data then apply validation on the remaining 20%. Finally, we merge the five group into three groups: cancer (CR), adenomas (JZ/FJ), normal (XR/JK), and use the same 80% and 20% for training and validation.

| ## | [1] “input: 2018-03-01_nhb1-b2-999 /otutab_norm.txt” | |
|---|---|---|
| ## | | |
| ## | | |
| ## | \| sample_size\| | num_OTUs \| |
| ## | \|:-----------:\| | \|:--------:\| |
| ## | 999 | 6269 |

| | |
|---|---|
| ## | |
| ## | Table: Total number of samples and OTUs |

### Feature Selection

We select OTUs satisfying that it occurs in at least 3% of samples with relative abundance >0.05%. Given that the normalized counts per sample is 50,000, the latter is >25 counts.

| ## | | |
|---|---|---|
| ## | | |
| ## | \| sample_size \| | num_OTUs \| |
| ## | \|:-----------:\| | \|:--------:\| |
| ## | \| 999 \| | 341 \| |

| | |
|---|---|
| ## | |
| ## | Table: After Feature Selection, total number of samples and OTUs |

### 2. Random Forest Classification Using Cancer (CR) and Normal (JK)

Random forest model is built using 80% of the CR/JK data, then classification are made for (1) 20% of the remaining CR/JK data and (2) all non-CR/JK data.

### 3. Multi-Class Classification

We first test the classification on five stages of progression then further collapse the data into three stages according to disease progression: Normal (JK), intermediate stage (FJ, XR) and advanced stage (JZ, CR).

### Prediction: Multi-Group

Similar to the prediction on CR and NM, we built the multi-group classifier using batch 2 alone and generated prediction results on batch 3 samples, which were independently obtained. The performance of the classifier dropped significantly to an overall accuracy of 0.601 from 0.935 in the classification (table 6). The sensitivities for CR, AD, and NP dropped to 0.9, 0.156 and 0.9, respectively and specificities dropped to 0.975, 0.950 and 0.418.

The significant drop in performance of the multi-group classifier when applied to independent samples is in striking contrast to the CR and NM classifier, which had a low bias. Indeed, differentiating adenomas from the cancer and normal is in general a harder problem (17). On top of that, we had a small number of samples to build the classifier from and relatively large batch effects as shown earlier. When samples were pooled together for multi-group classification, the high accuracy was most likely attributed to the fact that the classifier was able to capture the batch effects, which was a more dominant discriminative feature compared to features representing biological signals.

To address the problem of batch effects, we applied a recently developed methods (16) that specifically targeting batch effects for case-control microbiome studies. Unfortunately, the method showed little effect in the current study.

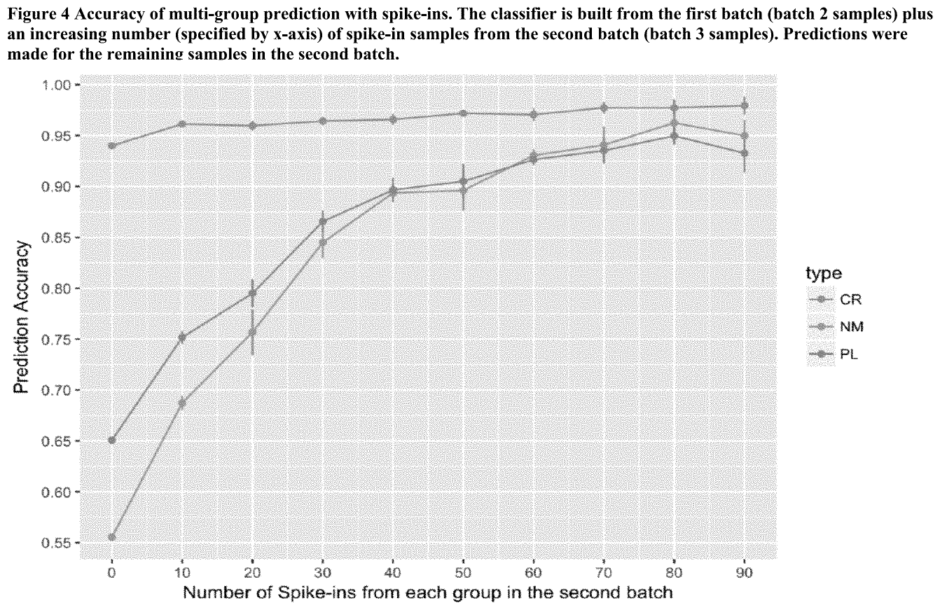

Next, inspired by the multi-group classification study, we explored the viability for a spike in strategy where we use certain number of samples with known labels to be processed together with new samples to be predicted. This way, we can directly include the batch effects in our model. FIG. 4 showed the effects of including an increasing number of samples from each groups on the overall accuracy. The accuracy for CR group was consistently high, and NM and PL predictions consistently became better and the performance flattened out around 60 spike in samples per group. This results showed a potential method of addressing the issues of batch effects at the cost of resequencing a certain number of known samples together with every batch of new samples. The detailed analysis of spike-in experiments is given below.

### Multi-Group Prediction Using Independent Training and Test Samples

1. Random Forest Classification Using otutab_norm.txt, Building Model Using the First Batch then Predict on the Second:

| ## | | |
|---|---|---|
| ## | \| \| | |
| ## | \|:-------------------:\| | |
| ## | \| batch1_otu_norm.txt \| | |
| ## | | |
| ## | Table: Normalized OTU Table Path | |
| ## | | |
| ## | | |
| ## | \|sample_size | \| num_OTUs \| |
| ## | \|:-----------:\| | \|:--------:\| |
| ## | \|500 \| | 341\| |

| | |
|---|---|
| ## | |
| ## | Table: After Feature Selection, total number of samples and OTUs |
| ## | |
| ## | Call: |
| ## | randomForest(formula = Type ~ ., data = train_data, importance = TRUE, ntree = |
| | 1000, proximity = TRUE) |

| | |
|---|---|
| ## | Type of random forest: classification |

| | |
|---|---|
| ## | Number of trees: 1000 |

| | |
|---|---|
| ## | No. of variables tried at each split: 18 |
| ## | |

| | |
|---|---|
| ## | OOB estimate of error rate: 3% |

| ## | Confusion matrix: | | | | |
|---|---|---|---|---|---|
| ## | | CR | JK | JZ | class.error |
| ## | CR | 97 | 0 | 3 | 0.03 |
| ## | JK | 0 | 190 | 10 | 0.05 |
| ## | JZ | 0 | 2 | 198 | 0.01 |

| | | | | | | |
|---|---|---|---|---|---|---|
| ## | | Sensitivity | Specificity | Pos Pred Value | Neg Pred Value | Precision |
| ## | Class: CR | 0.9100000 | 0.9699248 | 0.8834951 | 0.9772727 | 0.8834951 |
| ## | Class: JK | 0.1809045 | 0.9300000 | 0.6315789 | 0.6312217 | 0.6315789 |
| ## | Class: JZ | 0.8600000 | 0.4414716 | 0.5073746 | 0.8250000 | 0.5073746 |

| | | | | | |
|---|---|---|---|---|---|
| ## | | Recall | F1 | Prevalence | Detection Rate |
| ## | Class: CR | 0.9100000 | 0.8965517 | 0.2004008 | 0.18236473 |
| ## | Class: JK | 0.1809045 | 0.2812500 | 0.3987976 | 0.07214429 |
| ## | Class: JZ | 0.8600000 | 0.6382189 | 0.4008016 | 0.34468938 |

| | | | |
|---|---|---|---|
| ## | | Detection Prevalence | Balanced Accuracy |
| ## | Class: CR | 0.2064128 | 0.9399624 |
| ## | Class: JK | 0.1142285 | 0.5554523 |
| ## | Class: JZ | 0.6793587 | 0.6507358 |

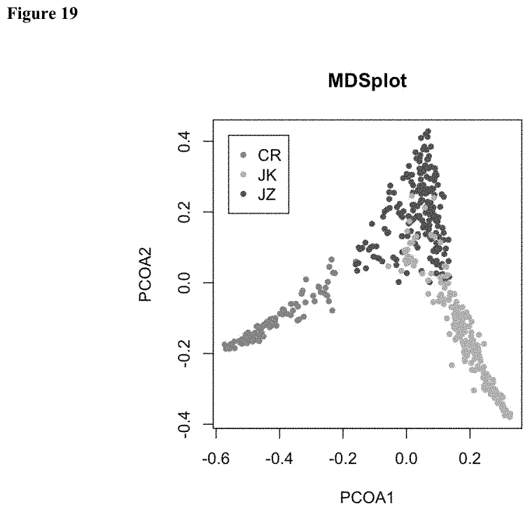

(Also see Figure 19)

### 2. Spike-in Prediction

The models are built using the first batch with a spike-in of an increment often additional samples of each of five groups (CR, JZ, FJ, XR, JK) from the second batch, then predictions are made to the remaining samples in the second batch. This measures the effect of capturing the batch effects by the model.

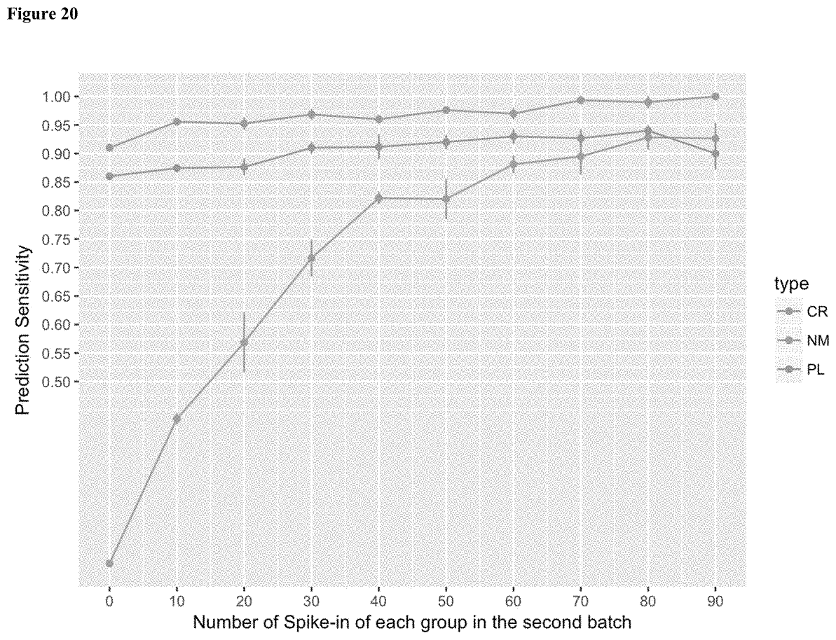

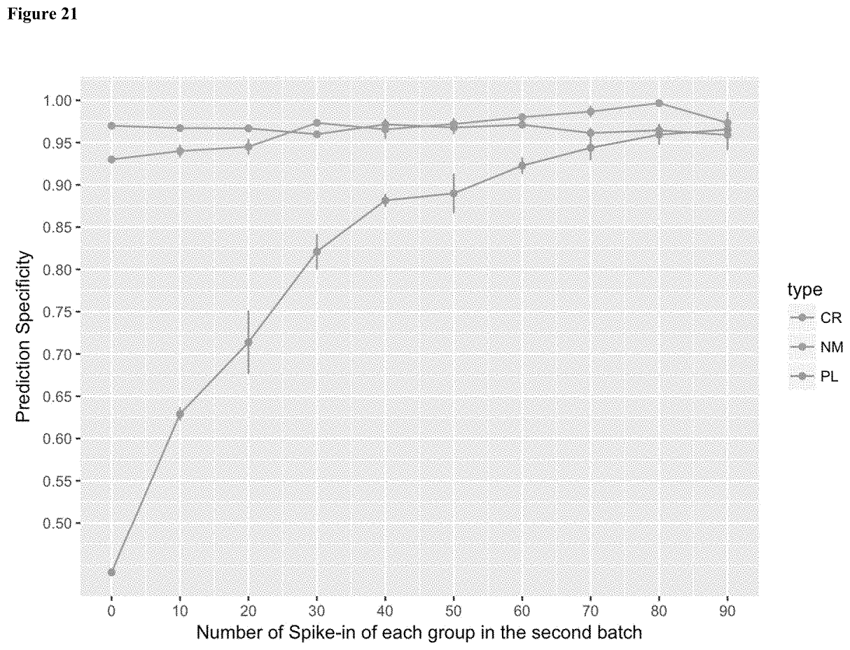

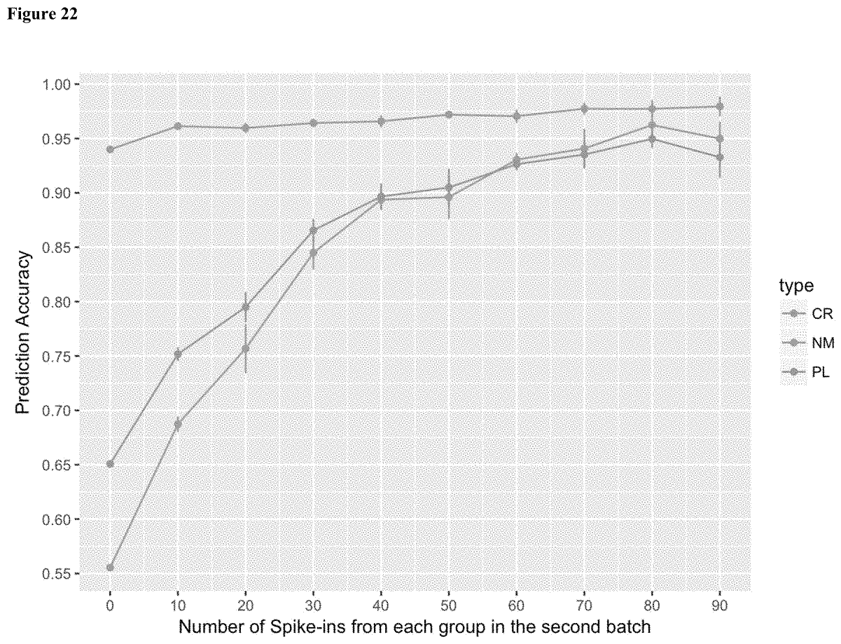

Change of sensitivity, change of specificity, and change of overall accuracy are shown in FIGS. 20 to 22, respectively.

### DISCUSSION

In this work, we have developed a binary classifier for CRC versus healthy solely based on OTU composition and demonstrated that this classifier works well on independent data, achieving an accuracy of 96%. Meanwhile, we showed this result was not confounded by age and gender which may confounders in the study. These results were distinct from most of the previous studies in three aspects: the features consist of OTU only and was not manually screened other than certain quality control aiming to avoid rare OTUs and reduce the potential of contamination (hence improving model bias), the classifier was tested on complete independent data, and we controlled for the obvious confounders. We further analyzed the taxonomic annotations of the most discriminative OTUs, which are mostly consistent with the literature discoveries.

We further showed that when data were pooled together from different batches, the multi-group classifier achieved a high accuracy. But we further showed that this is confounded by batch effects, which in the current scenario dwarf the real biological signal. This result indicates that it is more difficult compared to binary classification between cancer and normal, and for another, on top of that we may need more samples to properly train the classifier, there's significant batch effects as reflected by the analysis of positive control samples.

Assay reproducibilities and batch effects were frequent issues in microbiome studies and sometimes, the batch effects were not easily correctable. We proposed a spike-in strategy to address the batch effects by processing a set of known samples together with each new batch of samples to be predicted, though this strategy certainly drives up the processing cost. We acknowledge that this strategy needs further validation.

In summary, assay reproducibility and eliminating batch effects are critical factors in diagnosis using microbiome content, and any classification method requires independent validation to avoid overfitted results. With the improvement of assay stability, our proposed strategy serves as a promising method for detecting CRC and its earlier stages.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### REFERENCES

1. E. L. Amitay, A. Krilaviciute, and H. Brenner. Systematic review: Gut microbiota in fecal samples and detection of colorectal neoplasms. Gut microbes, pages 1-25, March 2018.
2. M. Balvociute and D. H. Huson. Silva, rdp, greengenes, ncbi and ott—how do these taxonomies compare?BMC genomics, 18:114, March 2017.
3. N. T. Baxter, M. T. Ruffin, M. A. M. Rogers, and P. D. Schloss. Microbiota-based model improves the sensitivity of fecal immunochemical test for detecting colonic lesions. Genome medicine, 8:37, April 2016.
4. S. Bullman, C. S. Pedamallu, E. Sicinska, T. E. Clancy, X. Zhang, D. Cai, D. Neuberg, K. Huang, F. Guevara, T. Nelson, O. Chipashvili, T. Hagan, M. Walker, A. Ramachandran, B. Diosdado, G. Serna, N. Mulet, S. Landolfi, S. Ramon Y Cajal, R. Fasani, A. J. Aguirre, K. Ng, E. lez, S. Ogino, J. Tabernero, C. S. Fuchs, W. C. Hahn, P. Nuciforo, and M. Meyerson. Analysis of fusobacterium persistence and antibiotic response in colorectal cancer. Science (New York, N.Y.), 358:1443-1448, December 2017.
5. D. Capper, D. T. W. Jones, M. Sill, V. Hovestadt, D. Schrimpf, and et al. DNA methylation-based classification of central nervous system tumours. Nature, 555:469-474, March 2018.
6. L. Chung, E. T. Orberg, A. L. Geis, J. L. Chan, K. Fu, C. E. DeStefano Shields, C. M. Dejea, P. Fathi, J. Chen, B. B. Finard, A. J. Tam, F. McAllister, H. Fan, X. Wu, S. Ganguly, A. Lebid, P. Metz, S. W. Van Meerbeke, D. L. Huso, E. C. Wick, D. M. Pardoll, F. Wan, S. Wu, C. L. Sears, and F. Housseau. Bacteroides fragilis toxin coordinates a pro-carcinogenic inflammatory cascade via targeting of colonic epithelial cells. Cell host & microbe, 23:421, March 2018.
7. J. R. Cole, Q. Wang, J. A. Fish, B. Chai, D. M. McGarrell, Y. Sun, C. T. Brown, A. Porras-Alfaro, C. R. Kuske, and J. M. Tiedje. Ribosomal database project: data and tools for high throughput rrna analysis. Nucleic acids research, 42:D633-D642, January 2014.
8. H. M. P. Consortium. Structure, function and diversity of the healthy human microbiome. Nature, 486:207-214, June 2012.
9. Z. Dai, O. O. Coker, G. Nakatsu, W. K. K. Wu, L. Zhao, Z. Chen, F. K. L. Chan, K. Kristiansen, J. J. Y. Sung, S. H. Wong, and J. Yu. Multi-cohort analysis of colorectal cancer metagenome identified altered bacteria across populations and universal bacterial markers. Microbiome, 6:70, April 2018.
10. C. M. Dejea, P. Fathi, J. M. Craig, A. Boleij, R. Taddese, A. L. Geis, X. Wu, C. E. DeStefano Shields, E. M. Hechenbleikner, D. L. Huso, R. A. Anders, F. M. Giardiello, E. C. Wick, H. Wang, S. Wu, D. M. Pardoll, F. Housseau, and C. L. Sears. Patients with familial adenomatous polyposis harbor colonic biofilms containing tumorigenic bacteria. Science (New York, N.Y.), 359:592-597, February 2018.
11. R. Edgar. Sintax: a simple non-bayesian taxonomy classifier for 16 s and its sequences. Technical report, 2016.
12. R. C. Edgar. Uparse: highly accurate otu sequences from microbial amplicon reads. Nature methods, 10:996-998, October 2013.
13. V. Eklof, A. Lofgren-Burstrom, C. Zingmark, S. Edin, P. Larsson, P. Karling, O. Alexeyev, J. Rutegard, M. L. Wikberg, and R. Palmqvist. Cancer-associated fecal microbial markers in colorectal cancer detection. International journal of cancer, 141:2528-2536, December 2017.
14. R. M. Ferreira, J. Pereira-Marques, I. Pinto-Ribeiro, J. L. Costa, F. Carneiro, J. C. Machado, and C. Figueiredo. Gastric microbial community profiling reveals a dysbiotic cancer-associated microbiota. Gut, 67:226-236, February 2018.
15. W. S. Garrett. Cancer and the microbiota. Science (New York, N.Y.), 348:80-86, April 2015.
16. S. M. Gibbons, C. Duvallet, and E. J. Alm. Correcting for batch effects in case-control microbiome studies. PLoS computational biology, 14:e1006102, April 2018.
17. V. L. Hale, J. Chen, S. Johnson, S. C. Harrington, T. C. Yab, T. C. Smyrk, H. Nelson, L. A. Boardman, B. R. Druliner, T. R. Levin, D. K. Rex,
18. D. J. Ahnen, P. Lance, D. A. Ahlquist, and N. Chia. Shifts in the fecal microbiota associated with adenomatous polyps. Cancer epidemiology, biomarkers & prevention: a publication of the American Association for Cancer Research, cosponsored by the American Society of Preventive
19. J. A. Joyce and D. T. Fearon. T cell exclusion, immune privilege, and the tumor microenvironment. Science (New York, N.Y.), 348:74-80, April 2015.
20. J. S. Lin, M. A. Piper, L. A. Perdue, C. M. Rutter, E. M. Webber, E. O'Connor, N. Smith, and E. P. Whitlock. Screening for colorectal cancer: Updated evidence report and systematic review for the us preventive services task force. JAMA 4, 315:2576-2594, June 2016.
21. G. Nakatsu, X. Li, H. Zhou, J. Sheng, S. H. Wong, W. K. K. Wu, S. C. Ng, H. Tsoi, Y. Dong, N. Zhang, Y. He, Q. Kang, L. Cao, K. Wang, J. Zhang, Q. Liang, J. Yu, and J. J. Y. Sung. Gut mucosal microbiome across stages of colorectal carcinogenesis. Nature communications, 6:8727, October 2015.
22. R. V. Purcell, M. Visnovska, P. J. Biggs, S. Schmeier, and F. A. Frizelle. Distinct gut microbiome patterns associate with consensus molecular subtypes of colorectal cancer. Scientific reports, 7:11590, September 2017.
23. C. Quast, E. Pruesse, P. Yilmaz, J. Gerken, T. Schweer, P. Yarza, J. Peplies, and F. O. Glckner. The silva ribosomal ma gene database project: improved data processing and web-based tools. Nucleic acids research, 41:D590-D596, January 2013.
24. Y. Sanz, M. Olivares, A'. Moya-Pe'rez, and C. Agostoni. Understanding the role of gut microbiome in metabolic disease risk. Pediatric research, 77(1-2):236, 2014.
25. N. Segata, J. Izard, L. Waldron, D. Gevers, L. Miropolsky, W. S. Garrett, and C. Huttenhower. Metagenomic biomarker discovery and explanation. Genome biology, 12:R60, June 2011.
26. L. R. Thompson, J. G. Sanders, D. McDonald, A. Amir, J. Ladau, and et al. A communal catalogue reveals earth's multiscale microbial diversity. Nature, 551:457-463, November 2017.
27. C. Urbaniak, G. B. Gloor, M. Brackstone, L. Scott, M. Tangney, and G. Reid. The microbiota of breast tissue and its association with breast cancer. Applied and environmental microbiology, 82:5039-5048, August 2016.

1. A computer-aided method for classifying a human subject in need thereof as having colorectal cancer (CRC) or being normal (NM), comprising the steps of:
(a) obtaining a fecal sample taken from the human subject;
(b) producing an Operational Taxonomic Unit (OTU) profile of the sample in step (a),
(c) providing the OTU profile to a trained machine learning classifier;
(d) executing the trained machine learning classifier to predict the probability that the human subject has colorectal cancer or is normal.

2. A computer-aided method for classifying a human subject in need thereof as having colorectal cancer (CRC), colorectal adenomas (AD), or being normal (NM), comprising the steps of:
(a) obtaining a fecal sample taken from the human subject;
(b) producing an Operational Taxonomic Unit (OTU) profile of the sample in step (a),
(c) providing the OTU profile to a trained machine learning classifier;
(d) executing the trained machine learning classifier to predict the probability that the human subject has colorectal cancer, has colorectal adenomas, or is normal.

3. A computer-aided method for classifying a human subject in need thereof as having colorectal cancer (CRC), polyps (PL), non-advanced adenomas (NA), advanced adenomas (AA), or being normal, comprising the steps of:
(a) obtaining a fecal sample taken from the human subject;
(b) producing an Operational Taxonomic Unit (OTU) profile of the sample in step (a),
(c) providing the OTU profile to a trained machine learning classifier;
(d) executing the trained machine learning classifier to predict the probability that the human subject has colorectal cancer, has polyps, has non-advanced adenomas, has advanced adenomas, or is normal.

4. The method of claim 3, wherein the OTU profile is produced by (1) amplifying a 16S rRNA hyper variable region of microbial nucleic acid sequences present in the sample, (2) sequencing the amplified sequences; (3) producing a list of unique microbial sequences present in the fecal sample based on the sequencing result of step (2) to form the OTU profile, wherein the list comprises abundance information of each unique microbial sequence.

5. The method of claim 4, wherein the 16S rRNA hyper variable region is the V3-V4 hyper variable region.

6. The method of claim 3, wherein the OTUs profile of step b) comprises expression profile of one or more microbial nucleic acid sequences having at least 95% identity to a consensus sequence in SEQ ID NOs. 1-345.

7. The method of claim 3, wherein the machine learning classifier is selected from the group consisting of decision tree classifier, K-nearest neighbor classifier (KNN), logistic regression classifier, nearest neighbor classifier, neural network classifier, Gaussian mixture model (GMM), Support Vector Machine (SVM) classifier, nearest centroid classifier, linear regression classifier and random forest classifier.

8-9. (canceled)

10. The method of claim 3, wherein the machine learning classifier has been trained using a set of reference data of a reference human subject population comprising colorectal cancer, polyps, non-advanced adenomas, advanced adenomas, and normal human subjects.

11-12. (canceled)

13. The method of claim 10, wherein the reference data is produced by a process comprising the following steps:
(1) obtaining a collection of human subject fecal samples as training samples, wherein the fecal samples are collected from colorectal cancer, polyps, non-advanced adenomas, advanced adenomas, and normal human subjects,
(2) for each fecal sample in the collection,
(i) amplifying 16S rRNA hyper variable region of bacterial nucleic acid sequences,
(ii) sequencing the amplified sequences; and
(iii) producing a list of unique microbial sequences present in the sample, wherein the list comprises abundance information of each unique microbial sequence;
(3) grouping the lists of unique microbial sequences obtained in step (2) to form a reference OTU matrix as the reference data, wherein the reference matrix comprises abundance information of each unique microbial sequence for each fecal sample.

14. The method of claim 13, wherein the reference OTU matrix is normalized such that the sum of sequence abundance for each sample is the same.

15. The method of claim 13, wherein the reference OTU matrix is simplified by reducing the number of OTUs through feature selection.

16. The method of claim 15, wherein the feature selection is to remove low abundant OTUs across training samples.

17. The method of claim 3, wherein the machine learning classifier is a random forest classifier.

18. The method of claim 17, wherein hyperparameters of the random forest are tuned using cross validation method.

19. The method of claim 18, wherein the hyperparameters to be tuned comprise the number of trees, number of maximum features used for each split of tree, and minimum samples per leaf.

20-21. (canceled)

22. The method of claim 3, wherein the classifying method has an accuracy of at least 60%.

23. (canceled)

24. The method of claim 13, wherein the collection of human subject fecal samples contains samples collected from at least about 50 human subjects.

25. The method of claim 4, wherein the sequencing step comprises sequencing at least 5,000 amplified fragments for each fecal sample.

26-30. (canceled)

31. The method of claim 10, wherein nucleic acid sequences in the samples collected from the reference human subject population are processed together with the sample collected from the human subject in need thereof for amplification and sequencing, to produce a set of reference data for training the classifier.
